(19) United States
(12) Patent Application Publication
(10) Pub. No.: US 2026/0357283 A1
(43) Pub. Date: Nov. 12, 2026

(54) NOVEL RESISTANCE GENES ASSOCIATED WITH DISEASE RESISTANCE IN SOYBEANS

(71) Applicant: Syngenta Crop Protection AG, Basel (CH)

(72) Inventors: Qingli Liu, Durham, NC (US); Euihwan Chung, Seoul (KR); Andrew David Farmer, Sante Fe, NM (US); John Daniel Hipskind, Durham, NC (US); Yinping Lucy Qin, Durham, NC (US); Xiaoping Tan, Durham, NC (US)

(73) Assignee: Syngenta Crop Protection AG, Basel (CH)

(21) Appl. No.: 19/129,714

(22) PCT Filed: Nov. 10, 2023
(86) PCT No.: PCT/US2023/079324

§ 371 Date: May 14, 2025

Related U.S. Application Data
(60) Provisional application No. 63/383,609, filed on Nov. 14, 2022.
(60) Provisional application No. 63/426,524, filed on Nov. 18, 2022.
(60) Provisional application No. 63/509,586, filed on Jun. 22, 2023.

(51) Int. Cl. C12N 15/82; C07K 14/415

(52) U.S. Cl. CPC C12N 15/8282; C07K 14/415; C12N 15/8239

(57) ABSTRACT

Methods and compositions for identifying, selecting and/or producing a pathogen resistant plant, plant cell or seed (e.g., a legume or soybean plant, plant cell or seed) are provided. Polynucleotides and polypeptides are provided, which when expressed in a plant increase disease and/or pathogen resistance (e.g. to Asian Soy Rust and/or Powdery mildew) of the plant. Plants, plant cells and seed are provided comprising such polypeptides and polynucleotides, as are methods for their use.

### RELATED APPLICATIONS

This application claims priority to U.S. Provisional Patent Application No. 63/383,609, filed Nov. 14, 2022, U.S. Provisional Patent Application No. 63/426,524, filed 18 Nov. 2022, and U.S. Provisional Patent Application No. 63/509,586, filed 22 Jun. 2023, the contents of each of which are incorporated by reference herein in their entirety.

### FIELD OF THE INVENTION

The present invention relates to compositions and methods for identifying, selecting and producing enhanced disease and/or pathogen resistant plants using novel resistance genes.

### STATEMENT REGARDING ELECTRONIC SUBMISSION OF A SEQUENCE LISTING

A Sequence Listing in XML format, approximately 140 kb in size, submitted under 37 C.F.R. § 1.831-1.835, entitled 82721_PCT.xml, generated on Oct. 30, 2023, and filed via EFS-Web is provided in lieu of a paper copy. This Sequence Listing is hereby incorporated by reference in its entirety into the specification for its disclosures.

### BACKGROUND

Plant pathogens are known to cause considerable damage to important crops, resulting in significant agricultural losses with widespread consequences for both the food supply and other industries that rely on plant materials. As such, applicant desires to reduce the incidence and/or impact of agricultural pathogens on crop production.

Several pathogens have been associated with damage to soybeans, which individually and collectively have the potential to cause significant yield losses in the United States and throughout the world. Exemplary pathogens include, but are not limited to fungi (e.g., genus Phytophthora and Asian Soybean rust Phakopsora _{pahyrhizi}), nematodes (e.g., genus Meloidogyne, particularly, Meloidogyne javanica), and bacteria (e.g., Pseudomonas syringae). Given the significant threat to global food supplies that these pathogens present as well as the time and expense associated with treating soybean crops to prevent yield loss, new methods for producing pathogen resistant soybean cultivars are needed. What is needed is novel resistance genes (herein, “R-Genes”) that can be introduced into plants to control pathogens.

### SUMMARY OF THE INVENTION

Compositions and methods are provided to increase disease resistance and/or pathogen resistance of a plant, particularly legume plants, and more particularly soybean plants. The disclosure provides TIRA and TIRB polypeptides, variants and active fragments of TIRA and TIRB polypeptides, and fusion proteins of TIRA and TIRB polypeptides capable of enhancing disease resistance. The disclosure also provides TIRA and/or TIRB polypeptides modified to reduce their NADase activity that are capable of enhancing disease resistance. The disclosure further provides nucleic acids encoding the disease resistance enhancing polypeptides, as well as plants expressing the same.

Methods are also provided for enhancing disease resistance by providing plants with nucleic acids encoding the disclosed polypeptides. The disclosure further relates to methods of enhancing disease resistance and improving agronomic performance of plants by rebalancing the relative levels of TIRA and TIRB polypeptides in plants through engineered genomic modifications. Methods are also provided for genetically modifying TIRA and/or TIRB polypeptides so that their expression is balanced in plants, thereby providing plants with enhanced disease resistance and improved agronomic performance.

The foregoing and other objects and aspects of the present invention are explained in detail in the drawings and specification set forth below.

### BRIEF DESCRIPTION OF THE DRAWINGS

FIG. 1 illustrates that transgenic soybean events comprising the pair of dumb-bell TIR proteins confer enhanced resistance to ASR. Leaves from TO events comprising the pair of TIR polypeptides show strong ASR resistance.

FIG. 2 is a bar graph showing the low transformation efficiency of constructs comprising both Tir genes (TirA and TirB) as compared to constructs comprising a single Tir gene.

FIG. 3 depicts the negative growth phenotypes of plants grown from events comprising both Tir genes (TirA and TirB). Plants grown from events comprising both Tir genes showed delayed germination, stunted growth, and/or lethality at the young seedling stage.

FIG. 4 depicts the negative growth phenotypes of plants grown from events comprising both Tir genes (specifically, events generated from construct 24217). T1 plants at mature stage (Panel A: age 10 days after planting (DAP); Panel B: ~70 DAP) showed short stature and severe sterility in comparison to control plants (comprising a null event).

FIG. 5 is a map displaying the balanced and similar transcript levels of TIRA and TIRB polypeptides in wild Glycine spp. (donor lines indicated by arrows).

FIG. 6 graphically depicts the imbalanced transcript levels of TIRA and TIRB polypeptides in the transgenic events. As estimated using qRT-PCR, the transgenic events display a >20× higher level of TIRB transcription as compared to TIRA while the transcript levels are comparable in the corresponding controls.

FIG. 7 depicts leaves from events generated from constructs comprising modified promoter combinations, modified gene coding sequences, and modified orientations. All the events showed improved rust resistance relative to the control. Events generated from construct 24953 (Panel B) expressing the TirA and TirB in an orientation with the TirA inserted upstream of TirB showed strong rust resistance when compared to construct 24217 (panel A) as control. Events generated from construct 24915 wherein a number of introns in the coding sequence of the TirA and TirB genes was modified (Panel C) also showed strong rust resistance relative to the control. Events generated from construct 25337 wherein expression of the TirB gene was driven by a rust reactive promoter while expression of the TirA gene was driven by a constitutive Medicago promoter (Panel D) also showed strong rust resistance relative to the control.

FIG. 8 depicts leaves from events generated from constructs comprising the TirA and TirB genes fused together and transcribed via a common protomer and terminator. All the events showed strong rust resistance. Events generated from construct 25046 (Panel A), 25047 (Panel B) and 25135 (Panel C) expressing the fusion proteins of TIRA-TIRB all showed strong rust resistance. Events generated from these constructs had expression of both TirA and TirB genes driven by a common Medicago promoter and terminator.

FIG. 9 is a graph depicting the relative expression of Tir polypeptides in T1 plants generated from construct 25046. The events showed about twice the amount of TIRB polypeptide expression relative to TIRA polypeptide expression.

FIG. 10 depicts T1 plants generated from construct 25337 expressing a TIRATIRB fusion protein. The plants grow normally and do not display adverse growth phenotypes at any stage of growth. Panels A, B and C show normal growth of T1 events from seedling to R7+ stage (panel A at 7DAP, panel B at 16 DAP). Panels D and E show that the growth of T1 events from seedling to seed set stage is not distinguishable from null events with a normal seed number being produced per plant (panel D: ~70 days old plant). Panel F shows that the transcript levels of the TIRA polypeptide and TIRB polypeptide are comparable in the T1 homozygous events

FIG. 11 depicts leaves from T1 homozygous events generated from construct 25337 expressing a TIRATIRB fusion protein. The plants showed higher levels of resistance to a wide range of rust isolates (BRS, SUL, and RTP1) as compared to the control.

FIG. 12 illustrates an amino acid alignment of the TIRA1, TIRA2, TIRB1 and TIRB2 domains. The alignment suggests that the TIRB2 NADase catalytic residue (Glu, E) is required for soy resistance. Three of the 4 TIR domains (TIRA1, TIRA2, TIRB1 and TIRB2 highlighted in gray) contain Glutamic acid (E), highlighted in the black rectangle, when aligned with known TIR domains (AtRBA1, AtRPS4_TIR, AtRRS1_TIR and L6_TIR). TIRA2 contains valine (V) instead of E residue, as indicated by the red arrowhead within the black rectangle.

FIG. 13 depicts leaves from events generated from constructs comprising one or more mutations in the NADase sites of the TIR domains. The events show varying degrees of rust resistance. Panel A: Events generated from construct 25313 comprising a single mutant E87A in the TIRB2 domain showed strong rust resistance compared to the susceptible control. Panel B: Events generated from construct 25312 (Panel B) comprising a single gain-of-function mutation, V251E, in the TIRA1 domain showed strong rust resistance compared to the susceptible control. Panel C: Events generated from construct 25315 comprising a triple mutation (E87A in the TIRB1 domain, E85A in the TIRA1 domain, and E257A in the TIRB2 domain) showed comparable levels of susceptibility relative to the susceptible control. Panel D: Events generated from construct 25988 comprising a double mutation (E87A in the TIRB1 domain and E85A in the TIRA1 domain) showed strong rust resistance compared to the susceptible control.

FIG. 14 depicts leaves from events generated from constructs comprising one or more mutations in the NADase sites of the TIRA1 and TIRB1 domains. The mutations do not appear to negatively affect rust resistance in the events, when compared to a susceptible control(S). Events generated from construct 25988 comprising a double mutation (E87A in the TIRB1 domain and E85A in the TIRA1 domain) exhibit a higher level of resistance that events generated from construct 25046 (corresponding control) against soy rust 21BR08 (panel A), soy rust 21BRM (panel C), and soy rust RTP1 (panel E). Corresponding bar graphs of measured tubulin mRNA levels also show events from construct 25988 exhibit lower fungal biomass. The bar graphs show the relative expression (y-axis) of soybean rust β-tubulin gene of the indicated events at 14 days post inoculation with rust population 21BR08 (panel B), 21BRM (panel D), and RTP1 (panel F). Levels of resistance were measured molecularly with fungal β-tubulin via qRT-PCR on the events. The quantitative measurement is consistent with the phenotypic observations of the levels of resistance of the events (FIG. 13).

FIG. 15 shows leaves from events generated from constructs expressing wild-type TIR proteins. The events display resistance to powdery mildew (when expressing constructs 24953, 25046, or 25047) while constructs expressing mutant TIR proteins are susceptible to powdery mildew (when expressing constructs 25311, 25313, or 25988).

FIG. 16 shows a table of ratings of multiple T1 homozygous events generated from constructs 24192, 24205, and 24217. Soybean transgenic events at TO and T1 generations, created from constructs 24192, 24205, and 24217, were characterized for their resistance against soybean rust. Leaves from primary events comprising the individual Tir genes and the molecular stack were placed in a petri dish on a moist paper towel and then inoculated with a spore suspension of soybean rust isolates. Leaves from null events served as negative control. The results use standard soy rust rating scales with Reddish-brown (RB) types indicative of being resistant while Tan ratings are indicative of being susceptible. Numbers after the RB ratings are based on a combination of density of lesions or size of the lesions with a 1-4 scale from high to intermediate resistance, and indication of no sporulation (NSP) or very little sporulation (SPL). Numbers after Tan ratings are based on a combination of density of pustules and level of sporulation with 1-5 scale from low to high sporulation. TO and T1 soybean transgenic events expressing either the TIRA polypeptide (24205) or TIRB polypeptide (24192) alone did not confer resistance to soybean rust. However, soybean resistance was observed when both TIRA and TIRB polypeptides were co-expressed (24217).

FIG. 17 shows a table of soy resistance ratings of multiple T1 homozygous events generated from construct wherein TirB expression is driven by a rust inducible promoter (construct 25337).

FIG. 18 shows a table of soy resistance ratings of multiple T1 homozygous events generated from molecular stacks comprising different promoter combinations and orientations of the TirA and TirB genes (constructs 24915, 24953 and 25337).

FIG. 19 shows a table of soy resistance ratings of multiple T1 homozygous events generated from expression constructs comprising different fusions of the TirA and TirB gene products (constructs 25046, 25047, and 25135).

FIG. 20 shows phenotypes, including agronomic performance severity, rust resistance and transformation efficiency, of multiple T1 homozygous events generated from constructs comprising alternate promoters (construct 25337), alternate Tir gene orientations (constructs 25943 and 24915), and fusion proteins (constructs 25046 and 25047). Comparisons are made to the control comprising construct 24217.

FIG. 21 shows phenotypes, including agronomic performance severity, rust resistance and transformation efficiency, of multiple T1 homozygous events generated from constructs comprising alternate promoters (construct 25337), alternate Tir gene orientations (constructs 25943 and 24915), and fusion proteins (constructs 25046 and 25047). Comparisons are made to the control comprising construct 24217.

FIG. 22 shows a table of soy resistance ratings of multiple T1 homozygous events generated from constructs comprising different mutations in the TIR domains (constructs 25046, 25311, 25313, 25314, 25315, and 25988). The ratings show that the active NADase residue in the TIRB2 domain is required for soy rust resistance.

FIG. 23 illustrates expression from a novel bidirectional promoter (SEQ ID NO: 49). Comparison of GUS expression, and corresponding staining patterns, for expression driven by a sequence of the bidirectional promoter in the sense strand relative and the antisense strand shows that both sequences have equivalent GUS staining compared to a strong constitutive promoter (herein soybean ubiquitin; SEQ ID NO: 50). The data confirms the bidirectional activity of the promoter and suggests that the bidirectional promoter can be used to drive expression of the TIRA and TIRB polypeptides to confer disease resistance.

FIG. 24 compares the resistance profile of wild-type TIRA and TIRB proteins to mutants comprising mutations in the NADase sites of TIRA1 domain (mutation E85A), TIRA2 (mutation V253E), TIRB1 (mutation E87A), and/or TIRB2 (mutation E257A) domains. Resistance is assessed to three different pathogens: Asian soy rust (ASR), powdery mildew (PM), and Pseudomonas. (Nt=not tested).

FIG. 25 depicts leaves from events generated from constructs comprising a mutation in the NADase sites of each of the TIRA1 and TIRB1 domains, and compares them to a susceptible control (S) and an event comprising the wild type TIR proteins. An increased level of resistance is observed in the double mutant. Events generated from construct 25988 comprising a double mutation (E87A in the TIRB1 domain and E85A in the TIRA1 domain) exhibit a higher level of resistance that events generated from construct 25046 (corresponding control) against soy rust 21BR08 (panel F), soy rust 21BRM (panel D), and soy rust RTP1 (panel B). Corresponding bar graphs of measured tubulin mRNA levels also show events from construct 25988 exhibit lower fungal biomass. The bar graphs show the relative expression (y-axis) of soybean rust β-tubulin gene of the indicated events at 14 days post inoculation with rust population 21BR08 (panel E), 21BRM (panel C), and RTP1 (panel A). Levels of resistance were measured molecularly with fungal β-tubulin via qRT-PCR on the events. The quantitative measurement is consistent with the phenotypic observations of the levels of resistance of the events.

FIGS. 26 and 27A-B compare the ASR resistance profile of wild-type TIRA and TIRB proteins to mutants comprising mutations in the cNMP synthetase sites of TIRA1 domain (mutation C82A), TIRA2 (mutation C248A), TIRB1 (mutation C84A), or TIRB2 (mutation C254A) domains. Resistance ratings: R—resistant; S—susceptible; IR—intermediate resistance; IS—intermediate susceptible. Construct 28587 comprising a mutation in the TIRA1 had the highest ASR resistance and lowest disease rating. Four samples of each event were used for four replicates, with two samples infected with rust isolate 21BR08 (BR_A/B) and two leaves infected with isolate RTP22 (RTP_A/B). Disease symptoms on the sampled leaves were rated on a scale of 1 to 9.5 with the lower rating equating to lower disease levels. The susceptible control, 06 KG, was included from each replicate and had both the highest disease rating (FIG. 27, Panel A) and highest average tubulin level (FIG. 27, Panel B). Among the constructs that were evaluated, construct 28587 comprising a mutation in the TIRA1 had the highest ASR resistance and lowest disease rating in all replicates. The other constructs showed varying levels of intermediate resistance and increased tubulin levels, but clearly less disease and less tubulin than the susceptible control.

FIG. 28 illustrates an ASR effector protein and R-protein interaction assay in a heterologous assay system. Presence of interaction is confirmed via detection of a Hypertensive Response in Plant cells. Young tobacco leaves are inoculated with Agrobacterium cultures comprising a control construct, R-gene construct, Effector protein construct, or combinations thereof, using a blunt syringe. Cell death is observed 3-5 h after infiltration. Presence of circles with slight discoloration at site of inoculation indicate no significant cell death. Presence of circles with significant discoloration at site of inoculation correspond to strong cell death response. Agrobacterium strains containing R-genes TIRA and/or TIRB or a given soy rust effector were grown in Luria-Bertani agar plates, supplemented with 50 μg/mL spectinomycin and 50 μg/mL kanamycin, overnight at 22 °C in a dark chamber. The cultures were scooped with a toothpick and diluted in induction medium (5 mM MgSO₄, 2 mg/ml MES, and 10 μM acetosyringone, adjusted to pH 5.6 with HCl). Bacterial concentrations were measured and adjusted with induction medium to OD₆₀₀=0.1 (for construct 25046) or 0.8 (for effector and GUS constructs). Resulting cultures were pre-induced for 2 to 3 h at room temperature. For co-infiltrations, cultures carrying individual constructs were induced separately and mixed in a 1:1 ratio just before infiltration. Photographs show interaction of soy rust effectors SPE-87, 130, 196 (top row) and SPE-248 and 335 (bottom row) expressed in N. tabacum via Agrobacterium mediated transient assay with TIRA and TIRB. Reference diagram shows the site of inoculation with constructs comprising TIRA-TIRB only (R-gene+GUS, top of leaf), effector only (Effector+GUS, mid-leaf) or co-infiltration of both the TIRA-TIRB proteins and the effector (R-gene+Effector, bottom of leaf). Each of SPE-87, 130, 196, 335 and 248 specifically triggers a hypersensitive localized cell death response at the site of inoculation when co-infiltrated with construct 25046. Assays were repeated 3 times. 6/6 means infiltrated spots all showed HR. Co-infiltration of only effector protein or only R-protein with the control construct expressing GUS does not trigger a hypersensitive localized cell death response at the site of inoculation.

FIG. 29 illustrates a Pseudomonas effector protein and R-protein interaction assay in a heterologous assay system. Presence of interaction is confirmed via detection of a Hypertensive Response in Plant cells. Agrobacterium strains containing R-genes TIRA and/or TIRB or a given soy rust effector were grown in Luria-Bertani agar plates, supplemented with ₅₀ μg/mL spectinomycin and 50 μg/mL kanamycin, overnight at 22 °C in a dark chamber. The cultures were scooped with a toothpick and diluted in induction medium (5 mM MgSO₄, 2 mg/ml MES, and 10 μM acetosyringone, adjusted to pH 5.6 with HCl). Bacterial concentrations were measured and adjusted with induction medium. For co-infiltrations, cultures carrying individual constructs were induced separately and mixed in a 1:1 ratio just before infiltration. Photographs show interaction of HopT1 family of effectors from Pseudomonas syringae, HopT1-1 and HopT1-2, expressed in N. tabacum via Agrobacterium mediated transient assay with TIRA and TIRB. Reference diagram shows the site of inoculation with constructs comprising TIRA-TIRB only (R-gene+GUS, top of leaf), effector only (Effector+GUS, mid-leaf) or co-infiltration of both the TIRA-TIRB proteins and the effector (R-gene+Effector, bottom of leaf). Each of HopT1-1 and HopT1-2 specifically triggers a hypersensitive localized cell death response at the site of inoculation when co-infiltrated with construct 25046. Co-infiltration of only effector protein or only R-protein with the control construct expressing GUS does not trigger a hypersensitive localized cell death response at the site of inoculation.

FIG. 30 illustrates HopT1 effectors trigger TIRA and TIRB dependent HR in transgenic soybean leaves. Photographs show HR response indicative of interaction of HopT1 family of effectors conjugated into Pseudomonas syringae pv. glycines, when infiltrated into GM soybean leaves expressing TIRA and TIRB. When Pseudomonas syringae pv. glycines, which does not naturally contain HopT1 effectors, is infiltrated (without conjugated effectors), however, no HR response is seen.

FIG. 31 illustrates bacterial growth for Pseudomonas syringae pv. Glycines infiltrated on leaves from GM events expressing TIRA and TIRB (as previously shown in FIG. 30). Little to no bacterial growth is seen when Pseudomonas syringae pv. Glycines conjugated with and carrying HopT1 effectors is infiltrated on leaves from GM events expressing TIRA and TIRB (NegCK). In contrast, nearly 100 fold higher growth of Pseudomonas syringae pv. Glycines is observed on wild-type soybeans without TIRA and TIRB, or Pseudomonas syringae pv. Glycines without HopT1 effectors. FIG. 32 shows disease progression for the constructs (at 0-dpi versus 4-dpi).

FIG. 33 illustrates that TIRA and TIRB confer resistance to Pseudomonas syringae pv. tabaci via interaction with a HopT1 effector. HR response is not observed in wild-type soybean leaves that do not express the TIRA and TIRB proteins. A strong HR response is seen in soybean leaves expressing TIRA and TIRB when Pseudomonas syringae pv. Tabaci is coinfiltrated with HopT1-2. Reference image shows sites of infiltration.

FIG. 34 illustrates a powdery mildew effector protein and R-protein interaction assay in a heterologous assay system. Photographs show interaction of two powdery mildew effectors from Erysiphe pisi, EPCSEP-66 and 99, expressed in N. tabacum via Agrobacterium mediated transient assay with TIRA and TIRB. Reference diagram shows the site of inoculation with constructs comprising TIRA-TIRB only (R-gene+GUS, top of leaf), effector only (Effector+GUS, mid-leaf) or co-infiltration of both the TIRA-TIRB proteins and the effector (R-gene+Effector, bottom of leaf). Each of EPCSEP-66 and 99 specifically triggers a hypersensitive localized cell death response at the site of inoculation when co-infiltrated with construct 25046. Co-infiltration of only effector protein or only R-protein with the control construct expressing GUS does not trigger a hypersensitive localized cell death response at the site of inoculation.

FIG. 35 illustrates a oomycete effector protein and R-protein interaction assay in a heterologous assay system. Photographs show interaction of two oomycete effectors from Plasmopara halstedii, DMCEP-46 and 84, expressed in N. tabacum via Agrobacterium mediated transient assay with TIRA and TIRB. Reference diagram shows the site of inoculation with constructs comprising TIRA-TIRB only (R-gene+GUS, top of leaf), effector only (Effector+GUS, mid-leaf) or co-infiltration of both the TIRA-TIRB proteins and the effector (R-gene+Effector, bottom of leaf). Each of DMCEP-46 and 84 specifically triggers a hypersensitive localized cell death response at the site of inoculation when co-infiltrated with construct 25046. Co-infiltration of only effector protein or only R-protein with the control construct expressing GUS does not trigger a hypersensitive localized cell death response at the site of inoculation.

FIG. 36 illustrates a soybean cyst nematode effector protein and R-protein interaction assay in a heterologous assay system. Photographs show interaction of two oomycete effectors from soybean cyst nematode, SSNE-5, 18, and 28, expressed in N. tabacum via Agrobacterium mediated transient assay with TIRA and TIRB. Reference diagram shows the site of inoculation with constructs comprising TIRA-TIRB only (R-gene+GUS, top of leaf), effector only (Effector+GUS, mid-leaf) or co-infiltration of both the TIRA-TIRB proteins and the effector (R-gene+Effector, bottom of leaf). Each of SSNE-5, 18, and 28 specifically triggers a hypersensitive localized cell death response at the site of inoculation when co-infiltrated with construct 25046. Co-infiltration of only effector protein or only R-protein with the control construct expressing GUS does not trigger a hypersensitive localized cell death response at the site of inoculation.

FIG. 37 shows a comparison of effector recognition between TIRA and TIRB and other distant functional homologs. Effector recognition is compared across a variety of effector proteins including soy rust effectors, cyst nematode effectors, and Pseudomonas syringae effectors. Results show an identical spectra of recognition between TIRA and TIRB and its functional homolog from Cajanus cajun.

### BRIEF DESCRIPTION OF THE SEQUENCE LISTING

SEQ ID NO: 1 is the amino acid sequence for a TIRA polypeptide derived from the TirA gene of Glycine canescens.

SEQ ID NO: 2 is the amino acid sequence for a TIRB polypeptide derived from the TirA gene of Glycine canescens.

SEQ ID NO: 3 is the genomic sequence for the TirA gene. SEQ ID NO: 4 is the cDNA sequence for the TirA gene. SEQ IsD NO: 5 is an intronless version of the genomic sequence of the TirA gene. Each of SEQ ID NOS: 3-5 encodes the protein of SEQ ID NO: 1.

SEQ ID NO: 6 is the genomic sequence for the TirB gene. SEQ ID NO: 7 is the cDNA sequence for the TirB gene. SEQ ID NO: 8 is an intron-modified version of the genomic sequence of the TirB gene. Each of SEQ ID NOS: 6-8 encodes the protein of SEQ ID NO: 2.

SEQ ID NOS: 9-12 is the amino acid sequence for TIRATIRB fusion proteins comprising a TIRA and a TIRB polypeptide.

SEQ ID NO: 13-16 is the coding sequence for the TIRATIRB fusion proteins of SEQ ID NOS: 9-12.

SEQ ID NO: 17 is the coding sequence for a TIRATIRB fusion protein comprising an E85A loss of function mutation at a position corresponding to position 85 of the TIRA polypeptide (SEQ ID NO: 1).

SEQ ID NO: 18 is the coding sequence for a TIRATIRB fusion protein comprising an V251E gain of function mutation at a position corresponding to position 251 of the TIRA polypeptide (SEQ ID NO: 1).

SEQ ID NO: 19 is the coding sequence for a TIRATIRB fusion protein comprising an E87A loss of function mutation at a position corresponding to position 87 of the TIRB polypeptide (SEQ ID NO: 2).

SEQ ID NO: 20 is the coding sequence for a TIRATIRB fusion protein comprising an E85A loss of function mutation at a position corresponding to position 85 of the TIRA polypeptide (SEQ ID NO: 1), and E87A and E257A loss of function mutations at a position corresponding to positions 87 and 257 of the TIRB polypeptide (SEQ ID NO: 2).

SEQ ID NO: 21 is the coding sequence for a TIRATIRB fusion protein comprising an E85A loss of function mutation at a position corresponding to position 85 of the TIRA polypeptide (SEQ ID NO: 1), and an E87A loss of function mutation at a position corresponding to position 87 of the TIRB polypeptide (SEQ ID NO: 2).

SEQ ID NO: 22 is a plant active constitutive promoter, prMt12344, derived from Medicago truncatula.

SEQ ID NO: 23 is a plant active constitutive promoter, prMt51866, derived from Medicago truncatula.

SEQ ID NO: 24 is a plant active constitutive promoter, prMt15303, derived from Medicago truncatula.

SEQ ID NO: 25 is a plant active rust inducible promoter, prLuFIS1, derived from Flaxseed.

SEQ ID NO: 26 is a plant active native promoter, prGcaRG3a, of the TirA gene from G. canescens.

SEQ ID NO: 27 is a plant active native promoter, prGcaRG3b, of the TirB gene from G. canescens.

SEQ ID NO: 28 is a native terminator, tGcaRG3a, of the TirA gene from G. canescens.

SEQ ID NO: 29 is a native terminator, tGcaRG3b, of the TirB gene from G. canescens.

SEQ ID NO: 30 is a plant active terminator, tMt12344, derived from Medicago truncatula.

SEQ ID NO: 31 is a plant active terminator, tMt51866, derived from Medicago truncatula.

SEQ ID NO: 32 is the first intron, iAtBAF60, of an Arabidopsis thaliana gene (AtBAF60) that is similar to the human Brahma Associated Protein, BAF60.

SEQ ID NO: 33 is the nucleotide sequence for a synthetic linker peptide, xLinker. SEQ ID NO: 36 is the linker peptide encoded by SEQ ID NO. 33.

SEQ ID NO: 34 is the nucleotide sequence for a self-cleavage peptide linker, xT2ALinker-04. SEQ ID NO: 37 is the linker peptide encoded by SEQ ID NO. 34.

SEQ ID NO: 35 is the nucleotide sequence for a self-cleavage peptide linker, xT2ALinker-03. SEQ ID NO: 38 is the linker peptide encoded by SEQ ID NO. 35.

SEQ ID NOS: 39-40 are the genomic sequences of a first set of allelic variants, RG6a and RG6b, of the TirA and TirB genes, respectively. SEQ ID NOS: 41-42 are the genomic sequences of a second set of allelic variants, RG7a and RG7b, of the TirA and TirB genes, respectively. SEQ ID NOS: 43-44 are the genomic sequences of a third set of allelic variants, RG8a and RG8b, of the TirA and TirB genes, respectively. All allelic variants are derived from G. clandestina.

SEQ ID NO: 45 is the nucleotide sequence of a TirA gene ortholog derived Cajanus cajun.

SEQ ID NO: 45 encodes the TIRA polypeptide ortholog of SEQ ID NO: 46.

SEQ ID NO: 47 is the nucleotide sequence of a TirB gene ortholog derived Cajanus cajun.

SEQ ID NO: 48 encodes the TIRB polypeptide ortholog of SEQ ID NO: 47.

SEQ ID NO: 49 is the nucleotide sequence of a bidirectional promoter derived from a genomic locus comprising the R-protein genes Rg32 and Rg34.

SEQ ID NO: 50 is the nucleotide sequence of a plant active constitutive promoter, prUBQ3, derived from the Ubiquitin3 gene of Arabidopsis thaliana.

SEQ ID NO: 51 is the polynucleotide acid sequence for effector protein SPE-335 from Phakopsora pachyrhizi.

SEQ ID NO: 52 is the polynucleotide acid sequence for effector protein SPE-087 from Phakopsora pachyrhizi.

SEQ ID NO: 53 is the polynucleotide acid sequence for effector protein SPE-130 from Phakopsora pachyrhizi.

SEQ ID NO: 54 is the polynucleotide acid sequence for effector protein SPE-196 from Phakopsora pachyrhizi.

SEQ ID NO: 55 is the polynucleotide acid sequence for effector protein SPE-248 from Phakopsora pachyrhizi.

SEQ ID NO: 56 is the polynucleotide acid sequence for effector protein SSNE-05 from a plant parasitic nematode.

SEQ ID NO: 57 is the polynucleotide acid sequence for effector protein SSNE-18 from a plant parasitic nematode.

SEQ ID NO: 58 is the polynucleotide acid sequence for effector protein SSNE-28 from a plant parasitic nematode.

SEQ ID NO: 59 is the polynucleotide acid sequence for effector protein HopT1-1 from Pseudomonas syringae pathovars tomato strain DC3000. SEQ ID NO: 61 is the amino acid sequence of the effector.

SEQ ID NO: 60 is the polynucleotide acid sequence for effector protein HopT1-2 from Pseudomonas syringae pathovars tomato strain DC3000. SEQ ID NO: 62 is the amino acid sequence of the effector.

SEQ ID NO: 63 is the polynucleotide acid sequence for effector protein EPCSEP-66 from Erysiphe pisi. SEQ ID NO: 64 is the amino acid sequence of the effector.

SEQ ID NO: 65 is the polynucleotide acid sequence for effector protein EPCSEP-99 from Erysiphe pisi. SEQ ID NO: 66 is the amino acid sequence of the effector.

SEQ ID NO: 67 is the polynucleotide acid sequence for oomycete effector protein DMCEP-46. SEQ ID NO: 68 is the amino acid sequence of the effector.

SEQ ID NO: 69 is the polynucleotide acid sequence for oomycete effector protein DMCEP-84. SEQ ID NO: 70 is the amino acid sequence of the effector.

SEQ ID NO: 71 is the polynucleotide sequence for an expression cassette (29466) driving the endogenous expression of TIRA and TIRB.

SEQ ID NO: 72 is the polynucleotide sequence for an expression cassette (VC30523) driving the endogenous expression of TIRA and TIRB.

### DETAILED DESCRIPTION OF THE INVENTION

### 1. Definitions

Unless defined otherwise, all technical and scientific terms used herein have the same meaning as commonly understood to one of ordinary skill in the art to which the presently disclosed subject matter belongs.

Although the following terms are believed to be well understood by one of ordinary skill in the art, the following definitions are set forth to facilitate understanding of the presently disclosed subject matter.

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

Nucleotide sequences provided herein are presented in the 5′ to 3′ direction, from left to right and are presented using the standard code for representing nucleotide bases as set forth in 37 CFR §§ 1.821-1.825 and and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage, application rate, or time period and the like, is meant to encompass variations of ±20%, ±10%, +5%, ±1%, ±0.5%, or even±0.1% of the specified amount. As used herein, phrases such as “between about X and Y” mean “between about X and about Y” and phrases such as “from about X to Y” mean “from about X to about Y.”

As used herein, phrases such as “between about X and Y”, “between about X and about Y”, “from X to Y” and “from about X to about Y” (and similar phrases) should be interpreted to include X and Y, unless the context indicates otherwise.

As used herein, “agronomic performance” or “improved agronomic performance” refers to the presence of phenotypes (and underlying genetic elements) in a given plant that contribute to yield over the course of a growing season. Agronomic performance includes emergence vigor, germination efficiency, vegetative vigor, stress tolerance, stature, height, stalk or stem width, disease resistance, branching, flowering, seed set, seed size, seed density, standability, threshability and the like. A plant exhibiting improved agronomic performance has genetic elements that result in the plant having one or more or all of increased germination efficiency, taller stature,

As used herein, a “coding sequence” or “CDS” is a nucleic acid sequence that is transcribed into RNA such as mRNA, IRNA, tRNA, snRNA, sense RNA or antisense RNA. In embodiments, the RNA is then translated to produce a protein. In example embodiments, the CDS is derived from a cDNA sequence and includes the sequence of spliced exons of a transcript in DNA notation and does not include any intron or 5′ or 3′-untranslated regions (UTRs). In other example embodiments, the CDS is derived from a genomic DNA sequence and includes the sequence of spliced exons of a transcript in DNA notation as well as one or more introns, and 5′ and/or 3′-untranslated regions (UTRs).

As used herein, a “codon optimized” nucleotide sequence means a nucleotide sequence of a recombinant, transgenic, or synthetic polynucleotide wherein the codons are chosen to reflect the particular codon bias that a host cell or organism may have. This is typically done in such a way as to preserve the amino acid sequence of the polypeptide encoded by the codon optimized nucleotide sequence. In certain embodiments, a nucleotide sequence is codon optimized for the cell (e.g., an animal, plant, fungal or bacterial cell) in which the construct is to be expressed. For example, a construct to be expressed in a plant cell can have all or parts of its sequence codon optimized for expression in a plant. See, for example, U.S. Pat. No. 6,121,014. In embodiments, the polynucleotides provided herein are codon-optimized for expression in a plant cell (e.g., a dicot cell, a monocot cell, a soybean cell) or bacterial cell.

The term “comprise”, “comprises” or “comprising,” when used in this specification, indicates the presence of the stated features, integers, steps, operations, elements, or components, but does not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim “and those that do not materially alter the basic and novel characteristic(s)” of the claimed invention. Thus, the term “consisting essentially of” when used in a claim of this invention is not intended to be interpreted to be equivalent to “comprising.”

“Expression cassette” as used herein means a nucleic acid molecule capable of directing expression of at least one polynucleotide of interest, such as a polynucleotide encoding a TIRA polypeptide and/or a TIRB polypeptide and/or a TIRATIRB fusion protein, or active variants or fragments thereof in an appropriate host cell, and comprises a promoter operably linked to the polynucleotide of interest which is operably linked to a termination signal. An “expression cassette” can comprise additional polynucleotides to facilitate proper translation of the polynucleotide of interest. The expression cassette may comprise other polynucleotides not related to the expression of a polynucleotide of interest, but which are present due to convenient restriction sites for removal of the cassette from an expression vector. In embodiments, at least one of the components in the expression cassette may be heterologous (i.e., foreign or modified from its native form in composition and/or genomic locus) with respect to at least one of the other components (e.g., a heterologous promoter, a terminator, an intron, and/or any regulatory element operatively associated with a polynucleotide of interest). In other embodiments, the expression cassette may be naturally occurring and comprises the native regulatory elements, native introns and native genomic DNA to allow for the expression of the TIRA and/or TIRB polypeptide and/or TIRATIRB fusion protein, or active variants or fragments thereof. The expression cassette can be heterologous with respect to the host, i.e., the expression cassette (or even the polynucleotide of interest) does not occur naturally in the host cell and has been introduced into the host cell by a transformation process or a breeding process.

In embodiments of the present invention, expression cassettes are provided that can direct the expression of only a TIRA polypeptide (or an active variant or fragment thereof), only a TIRB polypeptide (or an active variant or fragment thereof), or each of the TIRA and TIRB polypeptides (or an active variant or fragment of either polypeptide). In an example embodiment, a first expression cassette is provided for expressing the TIRA polypeptide (or an active variant or fragment thereof), the expression of the TIRA polypeptide driven by a first heterologous promoter (e.g., a plant active promoter) while a second expression cassette is provided for expressing the TIRB polypeptide (or an active variant or fragment thereof), the expression of the TIRB polypeptide driven by a second, different heterologous promoter (e.g., another plant active promoter). In another example embodiment, a single expression cassette is provided for expressing each of the TIRA polypeptide (or an active variant or fragment thereof) and the TIRB polypeptide (or an active variant or fragment thereof), the expression of the TIR polypeptides driven by a common promoter (e.g., a plant active promoter).

In embodiments, a single expression cassette is provided for expression of the TIRA and TIRB polypeptides linked together as a fusion protein wherein expression of the TIRA polypeptide (or an active variant or fragment thereof) and the TIRB polypeptide (or an active variant or fragment thereof) is driven by a common promoter (e.g., a plant active promoter). Based on the presence of any linking sequences between the nucleotide sequences, the polypeptides can be expressed as a single fusion protein or as distinct proteins. In example embodiments, expression cassettes are provided that can direct the expression of a fusion protein comprising the TIRA polypeptide linked to the TIRB polypeptide via a linker, wherein expression of both polypeptides in the fusion protein is driven by a common, single heterologous promoter and terminator. In embodiments where the linker is self-cleavable, following expression of the fusion protein, the protein may be cleaved into the constituent TIRA and TIRB polypeptides. Based on the order of the nucleotide sequence encoding the TIRA polypeptide and the TIRB polypeptide in the expression cassette, the fusion protein may comprise the TIRA polypeptide at the N-terminus of the fusion protein (such as where the nucleotide sequence encoding the TIRA polypeptide is positioned upstream of the nucleotide sequence encoding the TIRB polypeptide), or the fusion protein may comprise the TIRB polypeptide at the N-terminus of the fusion protein (such as where the nucleotide sequence encoding the TIRB polypeptide is positioned upstream of the nucleotide sequence encoding the TIRA polypeptide).

In embodiments of the present invention, a cell is provided that comprises a TIRA polypeptide (or an active variant or fragment thereof), a TIRB polypeptide (or an active variant or fragment thereof), or each of the TIRA and TIRB polypeptides (or an active variant or fragment of either polypeptide). In particular embodiments, the cell is a plant cell, wherein said polynucleotide is stably integrated into the genome of the cell. In specific embodiments, the plant cell has an increased level of expression of the polypeptide and the plant cell has increased disease resistance relative to a control plant cell.

The term “introduced” or “introducing” defines a process of altering the content of a cell or a plant through the use of traditional breeding or recombinant transformation techniques. Any means can be used to introduce polynucleotides into a cell or a plant cell, including methods that result in stable transformation, transient transformation, or a gene edit. These include the use of calcium phosphate transfection, polybrene, protoplast fusion, PEG, electroporation, ultrasonic methods (e.g., sonoporation), liposomes, microinjection, naked DNA, plasmid vectors, viral vectors, both episomal and integrative, introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs) and any of the other well-known methods for introducing cloned genomic DNA, cDNA, synthetic DNA or other foreign genetic material into a host cell.

As used herein, the term “wild glycine” refers to a perennial Glycine plant, for example any one of G. canescens, G. argyrea, G. clandestine, G. latrobeana, G. albicans, G. aphyonota, G. arenaria, G. curvata, G. cyrtoloba, G. dolichocarpa, G. falcate, G. gracei, G. hirticaulis, G. lactovirens, G. latifolia, G. microphylla, G. montis-douglas, G. peratosa, G. pescadrensis, G. pindanica, G. pullenii, G. rubiginosa, G. stenophita, G. syndetika, or G. tomentella.

As used herein, the term “allele” refers to one of two or more different nucleotides or nucleotide sequences that occur at a specific locus.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” or “enhanced disease resistance” refers to a marker whose presence or absence can be used to predict whether and/or to what extent a plant will display a pathogen resistant or disease resistant phenotype.

A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art (e.g., using PCR).

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “desired allele”, “favorable allele” and “allele of interest” are used interchangeably to refer to an allele associated with a desired trait (e.g. ASR resistance).

As used herein, “disease resistance gene” or “resistance gene” or “R-gene” refers to a nucleic acid having a nucleotide sequence (e.g., DNA sequence) encoding a polypeptide, R-protein, or Resistance protein, that when expressed in a plant cell, is capable of enhancing or improving or increasing a defense or immune response in the plant cell, thereby conferring the plant with increased resistance to one or more plant pathogens. In specific embodiments, the TirA and TirB genes of the present invention are disease resistance genes, or R-genes, encoding polypeptides that confer enhanced pathogen resistance when co-expressed in a plant cell. The encoded TIRA and TIRB polypeptides, or their active variants or fragments, can be expressed in plants to enhance pathogen resistance to a plant pathogen, such as a fungal pathogen, a bacterial pathogen, a nematode, or a sucking pest. As non-limiting examples, the R-genes of the present invention, and their encoded polypeptides, can be used to enhance resistance to the fungal pathogen Phytopthora, Asian Soybean Rust. In other examples, the R-genes of the present invention and their encoded polypeptides can be used to enhance resistance to other fungal pathogens, such as those responsible for Powdery mildew, as well as nematodes, such as soybean cyst nematode (SCN) and root knot nematode. Further still, the R-genes of the present invention and their encoded polypeptides can be used to enhance resistance to bacterial pathogens such as Pseudomonas syringae.

In still other examples, the R-genes of the present invention and their encoded polypeptides can be used to enhance resistance to sucking and piercing pests, including but not limited to, aphids, stinkbugs, whiteflies, etc. Sucking and piercing pests are known in the art. For example, see Vleeshouwers and Oliver, 2014, Effectors as tools in disease resistance breeding against biotrophic, hemibiotrophic, and necrotrophic plant pathogens, MPMI Vol. 27, No. 3, (2014), pp. 196-206; Jayaraman et al. Effector-assisted breeding for bacterial wilt resistance in horticultural crops. Hortic. Environ. Biotechnol. 57, 415-423 (2016); Carolan et al., Predicted Effector Molecules in the Salivary Secretome of the Pea Aphid (Acyrthosiphon pisum): A Dual Transcriptomic/Proteomic Approach, J. of Proteome Res, 10,4 (2011) pp. 1505-1518; Shan et al., A salivary secretory protein from Riptortus pedestris facilitates pest infestation and soybean staygreen syndrome, Molecular Plant Pathology, (2023); Luo et al., Molecular Advances in Breeding for Durable Resistance against Pests and Diseases in Wheat: Opportunities and Challenges, Agronomy, 13, 3, pp628 (2023); Fu et al., Two salivary proteins Sm10 and SmC002 from grain aphid Sitobion miscanthi modulate wheat defense and enhance aphid performance, Frontiers in Plant Science, 14, (2023); Xu et al., A salivary effector enables whitefly to feed on host plants by eliciting salicylic acid-signaling pathway, PNAS, 116 (2) 490-495 (2018); and Naalden et al., Spotlight on the Roles of Whitefly Effectors in Insect-Plant Interactions, Front. Plant Sci., 2 July (2021); and Sec. Plant Pathogen Interaction, Volume 12 (2021), the contents of each of which are incorporated by reference herein in their entirety.

R-gene embodiments may comprise one or more motifs that correlate with one or more domains of the corresponding R-protein. Embodiments of an R-gene, such as the TirA and TirB genes of the present disclosure, encode a polypeptide comprising a TIR motif comprising a Toll/Interleukin-1 Receptor domain. In other embodiments, an R-gene may encode polypeptides having a TNL motif comprising a Toll/Interleukin-I receptor (TIR) domain, a nucleotide-binding site (NBS), and a leucine rich-repeat (LRR) domain. In still other embodiments, an R-gene may encode polypeptides having a CNL motif comprising a coiled coil (CC) domain, a nucleotide-binding site (NBS), and a leucine rich-repeat (LRR) domain. In further embodiments, the R-genes may encode polypeptides having one or more additional domains and motifs, such as a kinase domain, and a WRKY domain. In other embodiments, R-genes may additionally or alternatively encode polypeptides having domains of unknown function, wherein the function of the domains is unknown at the time. In still further embodiments, R-genes may additionally or alternatively encode polypeptides having putative effector binding domains and/or one or more putative transmembrane helices.

In embodiments, the nucleic acid sequence of the R-gene is derived from a wild plant exhibiting increased resistance to the pathogen and includes, at least, a coding sequence encoding the resistance conferring polypeptide. The nucleic acid sequence of the R-gene may further comprise nucleic acid sequences corresponding to one or more native regulatory elements (such as native introns, native promoters, native UTRs), one or more heterologous regulatory elements (such as a heterologous promoter and introns), and combinations thereof. Insertion of the R-gene into a plant that has decreased resistance to the pathogen (e.g., no resistance or partially or fully susceptible), at a chromosomal location (e.g., stably integrated into the plant genome) or extra-chromosomal location (e.g., on a vector or plasmid) results in conferring of the wild plant-derived pathogen resistance to the recipient plant. For example, in representative embodiments, an R-gene of the present invention is derived from Glycine tomentella, Glycine canescens, or Glycine clandestina and can be inserted into Glycine max plants to confer or enhance resistance of G. max plants to Asian Soy Rust and/or powdery mildew.

As used herein, “variants” of a polynucleotide sequence encoding the disease resistance R-gene and/or “variants” of a polypeptide sequence encoding a corresponding R-protein conferring disease resistance include, as non-limiting examples, annotation and splice variants. As used herein, annotation variants refer to sequences that differ from one another due to differences in annotation of regulatory sequences, including but not limited to transcription start site, position of starting ATG codon, position of splice sites, position of introns and/or exons, etc. In embodiments, a first annotation variant of a polypeptide may be longer than a second annotation variant due to the selection of an upstream start codon (e.g., an upstream Methionine annotated as the start codon in the first annotation variant while a downstream Methionine is annotated as the start codon in the second annotation variant). Annotation variants have the same activity as the corresponding R-protein, including the ability to confer increased disease resistance.

As used herein, “splice variants” refer to sequences that differ from a reference sequence due to alterations in the DNA sequence that occur during splicing at a splice site. As used herein, “splicing” refers to the process by which a pre-mRNA transcript is transformed into a mRNA molecule that can be translated into protein. Typically, this occurs by the removal of introns and the splicing back together of exons of the transcript. However, genetic alterations of the sequence can occur at the boundary of an exon and an intron, that is, at a splice site. The inclusion of one or more introns, or removal of one or more exons, during splicing can result in the creation of different mRNA molecules from the same gene, which in turn results in correspondingly different protein sequences being expressed from a single gene. These differing sequences, at the DNA, RNA and/or protein level, are referred to herein as “alternate splice variants”. Splice variant polypeptides encoded by alternative splicing of a gene sequence can include variants having a larger or smaller number of amino acids in the sequence and/or variants having alternate amino acids at particular positions within the sequence. In embodiments, alternate splice variants of a gene may be predicted based on the differing annotation of regions of a gene sequence as an exon or an intron (e.g., via analysis of a sequence using a sequence predicting software). Splice variants have the same activity as the corresponding R-protein, including the ability to confer increased disease resistance.

As used herein, the terms “disease tolerance”, “disease resistance”, “disease tolerant” or “disease resistant” refers to a plant's ability to endure and/or thrive despite being infected with a respective disease. Thus “disease tolerance” or “disease resistance” means a statistically significant increase in disease tolerance and/or a statistically significant decrease or the absence in one or more disease symptoms of a plant caused by a plant pathogen when compared to an appropriate control plant. In some embodiments, an increase in disease tolerance or resistance can be (1) measured by a plant's ability to endure and/or thrive despite being infected with a respective disease; (2) measured by infected disease resistant legume or soybean plants yielding as well as (or nearly as well) as uninfected legume or soybean plants; or (3) measured by a delay or the prevention of proliferation of a pathogen (e.g., fungi), including a delay or the prevention in disease related symptoms. In still other embodiments, a plant or germplasm can be labeled as “disease resistant” if it displays “enhanced or increased pathogen resistance” when compared to a control plant.

As used herein, the terms “enhanced pathogen resistance”, “enhanced disease resistance”, “increased resistance to a pathogen,” or “confers pathogen resistance” refers to an improvement, enhancement, or increase in a plant's ability to endure and/or thrive despite being infected with a pathogen or disease (e.g., Asian soybean rust) as compared to one or more control plants. Enhanced disease resistance includes a reduction in the symptoms indicative of infection for a disease such as Asian soybean rust (“ASR”), soybean cyst nematode, Phytophthora, root knot nematode, bacterial diseases, or sucking pests, etc. An enhanced plant pathogen resistance may comprise any statistically significant increase in resistance to the plant pathogen, including, for example, an increase of at least 1%, 5%, 10%, 20%, 30%, 40%, 50%, 60%, 70%, 80%, 90%, 100% or higher. Conferring or enhancing or increasing resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include a statistically significant reduction in the number, size, and/or density of lesions, change in the color of lesions (such as from a tan coloration to a reddish-brown coloration), reduction in number and density of pustule formation, reduction in sporulation, reduction in defoliation, a reduction in yield loss, or any combination thereof. In further embodiments, enhanced pathogen resistance can include a statistically significant reduction in the number, size, and/or density of cysts. Further, enhanced pathogen resistance can include the prevention or delay of proliferation of a pathogen (e.g., fungus) in the plant.

A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean.

As used herein, “cisgenic” or “cisgenesis” involves the insertion, optionally into a genome (e.g., a plant genome), of one or more genes of the same or a related species, or from a crossable donor. As used herein a “cisgenic construct” is a recombinant nucleic acid sequence present in a cell, and optionally integrated into the cell's genome, wherein the recombinant nucleic acid sequence comprises a regulatory element operably linked to a nucleic acid sequence for a gene of interest, wherein the regulatory element and gene of interest are both native to the plant, or from a related species, or from a crossable donor, and are operably linked in the native cell at a genomic location different from the genomic location where they are integrated as the cisgenic construct. The introduction of specific alleles/genes present in the gene pool, without any DNA sequence change, via cisgenesis accelerates the breeding of species with long reproduction cycles with no linkage drag.

An “elite” plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

The terms “agronomically elite” as used herein, means a genotype that has a culmination of many distinguishable traits such as emergence, vigor, vegetative vigor, disease resistance, seed set, standability, yield and threshability which allows a producer to harvest a product of commercial significance.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in, or endogenous to, the organism.

The terms “nucleic acid,” “nucleic acid molecule,” “nucleotide sequence,” “oligonucleotide”, “polynucleic acids” and “polynucleotide” are used interchangeably herein, unless the context indicates otherwise, and refer to a heteropolymer of nucleotides. These terms include, without limitation, DNA and RNA molecules, including cDNA, genomic DNA, synthetic (e.g., chemically synthesized) DNA and RNA, plasmid DNA, mRNA, anti-sense RNA, and RNA/DNA hybrids, any of which can be linear or branched, single stranded or double stranded, or a combination thereof. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence, means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter, intron, terminator, enhancer) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to or “operatively associated” with the nucleotide sequence.

As used herein, the term “endogenous” refers to materials originating from within an organism or cell. “Exogenous” refers to materials originating from outside of an organism or cell. This typically applies to nucleic acid molecules used in producing transformed or transgenic host cells and plants. For example, a nucleic acid molecule encoding a TIRA polypeptide or active variant or fragment thereof, or encoding a TIRB polypeptide or active variant or fragment thereof, or encoding both TIR polypeptides or a fusion protein comprising both TIR polypeptides, is an exogenous nucleic acid used to confer or enhance pathogen resistance in a plant cell transformed with the nucleic acid molecule.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “genome” as it applies to plant cells encompasses not only chromosomal DNA found within the nucleus, but organelle DNA found within subcellular components of the cell.

The term “gene” means a nucleic acid that comprises chromosomal DNA, genomic DNA, plasmid DNA, cDNA, an artificial DNA polynucleotide, or other DNA encoding a polypeptide of interest. In particular embodiments, the nucleic acid sequence of the gene encodes a protein that, when expressed, is responsible, at least in part, for a particular characteristic or trait. In embodiments, the gene may be native, modified (e.g., by directed recombination or site-specific mutation), or synthetic. In example embodiments, the gene is transcribed into an RNA molecule (e.g., an mRNA) in a cell wherein the RNA may encode a peptide, polypeptide, or protein of interest, and in some examples may also encode genetic elements flanking the coding sequence that are involved in the regulation of expression of the mRNA or polypeptide of the present invention. A gene may thus comprise several operably linked sequences, such as a promoter sequence, a 5′ leader sequence comprising, for example, sequences involved in translation initiation, a (protein) coding region (comprising cDNA or genomic DNA), a 3′ non-translated sequence comprising, for example, transcription termination sequence sites, introns (e.g., one or more native, foreign, or modified introns). In example embodiments, the nucleic acid sequence of the isolated gene may include introns, exons, 5′ or 3′-untranslated regions (UTRs), and native regulatory elements (such as native promoters). In other example embodiments, the gene comprises a coding sequence for a polypeptide of interest without including any regulatory elements. As such the nucleic acids encoding the TIRA polypeptide, TIRB polypeptide, or active variants or fragment thereof, can lack all native or foreign/heterologous introns, can have one, two, three or more or all of the native introns replaced with foreign or modified introns, or have one or more of the native regulatory elements (promoters, 5′ UTRs, 3′ UTR and/or terminators) replaced with foreign or modified regulatory elements (promoters, 5′ UTRs, 3′ UTRs or and/or terminators), or any combination thereof.

As used herein, “heterologous” in reference to a polypeptide or polynucleotide sequence is a sequence that originates from a foreign species; or, if from the same species, is substantially modified from its native form in composition and/or genomic locus by deliberate human intervention. As such, heterologous sequences are in a configuration not found in nature.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, a TIRA polypeptide, a TIRB polypeptide, both TIR polypeptides, a TIRATIRB fusion protein, or active variants or fragments thereof, or markers associated with enhanced ASR tolerance or resistance, may be introgressed from a donor into a recurrent parent that is not disease resistant. The resulting offspring could then be repeatedly backcrossed and selected until the progeny possess the ASR tolerance allele(s) in the recurrent parent background.

As used herein, an “isolated” nucleic acid molecule or gene is substantially separated away from other nucleic acid or gene sequences with which the nucleic acid is normally associated, such as, from the chromosomal or extrachromosomal DNA of a cell in which the nucleic acid or gene naturally occurs. A nucleic acid molecule is an isolated nucleic acid molecule when it comprises a transgene or part of a transgene present in the genome of another organism. The term also embraces nucleic acids that are biochemically purified to substantially remove contaminating nucleic acids and other cellular components.

A polypeptide is “isolated” if it has been separated from the cellular components (nucleic acids, lipids, carbohydrates, and other polypeptides) that naturally accompany it or that is chemically synthesized or recombinant. A polypeptide molecule is an isolated polypeptide molecule when it is expressed from a transgene in another organism. A monomeric polypeptide is isolated when at least 60% by weight of a sample is composed of the polypeptide, preferably 90% or more, more preferably 95% or more, and most preferably more than 99%. Protein purity or homogeneity is indicated, for example, by polyacrylamide gel electrophoresis of a protein sample, followed by visualization of a single polypeptide band upon staining the polyacrylamide gel; high pressure liquid chromatography; or other conventional methods. Proteins can be purified by any of the means known in the art, for example as described in Guide to Protein Purification, ed. Deutscher, Meth. Enzymol. 185, Academic Press, San Diego, 1990; and Scopes, Protein Purification: Principles and Practice, Springer Verlag, New York, 1982.

A “locus” is a position on a chromosome where a gene or marker or allele is located. In some embodiments, a locus may encompass one or more nucleotides.

A “non-naturally occurring variety of legume or soybean” is any variety of legume or soybean that does not naturally exist in nature. A “non-naturally occurring variety of legume or soybean” may be produced by any method known in the art, including, but not limited to, transforming a legume or soybean plant or germplasm, transfecting a legume or soybean plant or germplasm and crossing a naturally occurring variety of legume or soybean with a non-naturally occurring variety of soybean. In some embodiments, a “non-naturally occurring variety of legume or soybean” may comprise one or more heterologous nucleotide sequences. In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines such as polynucleotides from wild glycine species).

As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, or an electromechanical assay. In some cases, a phenotype or trait is directly controlled by a single gene or genetic locus, i.e., a “single gene trait.” In other cases, a phenotype or trait is the result of several genes.

As used herein, the term “plant” may refer to a whole plant, any part thereof, or a cell or tissue culture derived from a plant. Thus, the term “plant” can refer to any of: whole plants, plant components or organs (e.g., roots, stems, leaves, buds, flowers, pods, etc.), plant tissues, seeds and/or plant cells. A plant cell is a cell of a plant, taken from a plant, or derived through culture from a cell taken from a plant. Thus, the term “soybean plant” may refer to a whole soybean plant, one or more parts of a soybean plant (e.g., roots, root tips, stems, leaves, buds, flowers, pods, seeds, cotyledons, etc.), soybean plant cells, soybean plant protoplasts and/or soybean plant calli.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. In embodiments, the plant cell is non-propagating and/or cannot regenerate a whole plant.

A “plant cell culture” means a culture of plant units such as, for example, protoplasts, cell culture cells, cells in plant tissues, pollen, pollen tubes, ovules, embryo sacs, zygotes and embryos at various stages of development.

“Plant material” or “plant part” refers to leaves, stems, roots, flowers or flower parts, fruits, pollen, egg cells, zygotes, seeds, cuttings, cell or tissue cultures, or any other part or product of a plant. In embodiments, plant part may refer to the whole plant.

A “plant organ” is a distinct and visibly structured and differentiated part of a plant such as a root, stem, leaf, flower bud, or embryo.

As used herein, the term “plant part” includes but is not limited to single cells and tissues from embryos, pollen, ovules, egg cells, seeds, leaves, flowers, flower parts, branches, fruit, stems, stalks, roots, root tips, anthers, cuttings and seeds, zygotes, anthers, shoots, scions, rootstocks, and/or plant cells including plant cells that are intact in plants and/or parts of plants, plant protoplasts, plant tissues, plant cell tissue cultures, plant calli, plant clumps, and the like. In some embodiments, the plant part or plant cell can be regenerated into a plant, while in other embodiments, the plant part or plant cell cannot be regenerated into a plant.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural or functional units. The use of this term in conjunction with, or in the absence of, any specific type of plant tissue as listed above or otherwise embraced by this definition is not intended to be exclusive of any other type of plant tissue.

“Plant pathogen” is used herein to mean a pathogen that can infect and cause disease in a plant. In embodiments, the plant pathogen is a fungal plant pathogen. In specific embodiments, the fungal pathogen is from the genus Phakopsora, including the species Phakopsora pachyrhizi and Phakopsora meibomiae. These species are known to cause ASR in plants. In other specific embodiments, the plant pathogen is an ascomycete fungal pathogen responsible for causing Powdery Mildew. In still other embodiments, the plant pathogen is a nematode, such as soybean cyst nematode (SCN) or a root knot nematode (RKN). In another embodiment, the plant pathogen is a bacterial plant pathogen such as Pseudomonas syringae. In still another embodiment, the plant pathogen is a sucking or piercing pest such as an aphd, stinkbug, or whitefly.

“Polyadenylation signal” or “polyA signal” refers to a nucleic acid sequence located 3′ to a coding region that causes the addition of adenylate nucleotides to the 3′ end of the mRNA transcribed from the coding region.

“Polymerase chain reaction (PCR)” refers to a DNA amplification method that uses an enzymatic technique to create multiple copies of one sequence of nucleic acid (amplicon). Copies of a DNA molecule are prepared by shuttling a DNA polymerase between two amplimers. The basis of this amplification method is multiple cycles of temperature changes to denature, then re-anneal amplimers (DNA primer molecules), followed by extension to synthesize new DNA strands in the region located between the flanking amplimers. Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence include, for example, cloning and restriction of appropriate sequences and direct chemical synthesis. Chemical synthesis methods can include, for example, the phospho di-or tri-ester method, the diethylphosphoramidate method and the solid support method disclosed in U.S. Pat. No. 4,458,066. Primers can be labeled, if desired, by incorporating detectable moieties by for instance spectroscopic, fluorescence, photochemical, biochemical, immunochemical, or chemical moieties. Primers that are diagnostic for ASR resistance (i.e., able to identify or select based on presence of ASR resistant alleles) can be created to any favorable SNP. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

As used herein, the terms “progeny” and “progeny plant” refer to a plant generated from a vegetative or sexual reproduction from one or more parent plants. A progeny plant may be obtained by cloning or selfing a single parent plant (i.e., the same plant acts as the donor of both male and female gametes), or by crossing two parental plants. The descendant(s) can be, for example, of the F1, the F2, or any subsequent generation.

The term “promoter” or “promoter region” refers to a polynucleic acid molecule that functions as a regulatory element, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant DNA construct, as demonstrated by its ability to produce mRNA. In some embodiments, the vector constructs or expression constructs or nucleic acid sequences disclosed herein comprise a promoter that is heterologous to the nucleic acid sequence encoding the TIRA or TIRB polypeptides or TIRATIRB fusion proteins, or active variants or fragments thereof. In other examples, the vector constructs, expression constructs or nucleic acid sequences comprise a promoter that is native or endogenous to the nucleic acid sequence encoding the TIRA or TIRB polypeptides, or active variants or fragments thereof.

In still other examples, the vector constructs, expression constructs or nucleic acid sequences comprise a promoter that is native or endogenous to the nucleic acid sequence encoding a different R-protein than the TIR polypeptides, or active variants or fragments thereof. In specific embodiments, the vector constructs, expression constructs or nucleic acid sequences comprise a native bidirectional promoter derived from a genomic locus comprising Rg32 and Rg34 genes (disclosed in U.S. provisional application 63/426,524 and 63/509,586 as SEQ ID NO: 7 and incorporated by reference herein in its entirety). The native bidirectional promoter is capable of driving expression of genes in both directions, that is, via the promoter sequence in both the sense orientation and the promoter sequence in the antisense orientation or via the reverse complement sequence. In particular embodiments, the bidirectional promoter is operably coupled to each of a nucleic acid sequence encoding the TIRA polypeptide and a nucleic acid sequence encoding the TIRB polypeptide. In one specific example embodiment, the nucleic acid sequence encoding the TIRA polypeptide is operably coupled downstream of the bidirectional promoter while the nucleic acid sequence encoding the TIRB polypeptide is operably coupled upstream of the bidirectional promoter, when viewing the sequence in the sense orientation. In another specific example embodiment, the nucleic acid sequence encoding the TIRA polypeptide is operably coupled upstream of the bidirectional promoter while the nucleic acid sequence encoding the TIRB polypeptide is operably coupled downstream of the bidirectional promoter, when viewing the sequence in the sense orientation.

As used herein, the term “recombinant” refers to a non-naturally occurring DNA, protein, cell, seed, or organism that is the result of genetic engineering and as such would not normally be found in nature. A “recombinant DNA molecule” is a DNA molecule comprising a DNA sequence that is not naturally found in nature and as such is the result of human intervention, such as a DNA molecule comprised of at least two DNA molecules heterologous to each other. An example of a recombinant DNA molecule is a DNA molecule provided herein encoding the TIRA and/or TIRB polypeptides, or the TIRATIRB fusion protein, or active variants or fragments thereof, operably linked to a heterologous regulatory element, such as a heterologous promoter, heterologous terminator or comprising one or more heterologous intron or the deletion of one or more native introns. An example of a recombinant DNA molecule is a DNA molecule provided herein encoding the TIRA and/or TIRB polypeptides, or a TIRATIRB fusion protein, or active variants or fragments thereof, operably linked to a heterologous regulatory element, such as a heterologous promoter, heterologous terminator or comprising one or more heterologous introns or the deletion of one or more native introns. A “recombinant protein” is a protein comprising an amino acid sequence that does not naturally occur and as such is the result of human intervention, such as an engineered protein or a chimeric protein. A recombinant cell, seed, or organism is a cell, seed, or organism comprising transgenic DNA, for example a transgenic cell, seed, plant, or plant part comprising a recombinant DNA molecule and therefore produced as a result of plant transformation.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using a sequence comparison algorithm or by visual inspection. In certain embodiments, substantially identical sequences have at least about 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity at the nucleotide or amino acid level. In certain embodiments, substantial identity exists over a region of the sequences that is at least about 50 amino acid residues, 100 amino acid residues, 150 amino acid residues, 200 amino acid residues, 250 amino acid residues, 300 amino acid residues, 350 amino acid residues, 400 amino acid residues, 450 amino acid residues, 500 amino acid residues, 525 amino acid residues, 526, amino acid residues 527 amino acid residues, 528 amino acid residues, 529 amino acid residues, 530 amino acid residues, 531 amino acid residues, 532 amino acid residues, 533 amino acid residues, 534 amino acid residues, 535 amino acid residues, 536 amino acid residues or more with respect to the protein sequence or the nucleotide sequence encoding the same.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47 (W1): W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions.

The terms “complementary” or “complementarity” (and similar terms), as used herein, refer to the natural binding of polynucleotides under permissive salt and temperature conditions by base-pairing. For example, the sequence “A-G-T” binds to the complementary sequence “T-C-A.” Complementarity between two single-stranded molecules may be partial, in which only some of the nucleotides bind, or it may be complete when total complementarity exists between the single stranded molecules. The degree of complementarity between nucleic acid strands has significant effects on the efficiency and strength of hybridization between the molecules. As used herein, the term “substantially complementary” (and similar terms) means that two nucleic acid sequences are at least about 50%, 60%, 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein).

As used herein, “specifically” or “selectively” hybridizing (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Specifically or selectively hybridizing sequences typically are at least about 40% complementary and are optionally substantially complementary or even completely complementary (i.e., 100% identical).

For DNA-DNA hybrids, the Tm can be approximated from the equation of Meinkoth and Wahl, Anal. Biochem., 138:267-84 (1984): Tm=81.5 °C.+16.6 (log M)+0.41 (% GC)-0.61 (% formamide)-500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1 °C for each 1% of mismatching; thus, Tm, hybridization and/or wash conditions can be adjusted to hybridize to sequences of the desired degree of identity. For example, if sequences with >90% identity are sought, the Tm can be decreased 10 °C. Generally, stringent conditions are selected to be about 5 °C lower than the thermal melting point (Tm) for the specific sequence and its complement at a defined ionic strength and pH. However, highly stringent conditions can utilize a hybridization and/or wash at the thermal melting point (Tm) or 1, 2, 3 or 4 °C lower than the thermal melting point (Tm); moderately stringent conditions can utilize a hybridization and/or wash at 6, 7, 8, 9 or 10 °C lower than the thermal melting point (Tm); low stringency conditions can utilize a hybridization and/or wash at 11, 12, 13, 14, 15 or 20 °C lower than the thermal melting point (Tm). If the desired degree of mismatching results in a Tm of less than 45 °C (aqueous solution) or 32 °C (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30 °C for short probes (e.g., 10 to 50 nucleotides) and at least about 60 °C for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37 °C and a wash in 1X to 2×SSC (20×SSC=3.0 M NaCl/0.3 M trisodium citrate) at 50 °C to 55 °C. Exemplary moderate stringency conditions include hybridization in 40% to 45% formamide, 1 M NaCl, 1% SDS at 37 °C and a wash in 0.5X to 1×SSC at 55 °C to 60 °C. Exemplary high stringency conditions include hybridization in 50% formamide, 1 M NaCl, 1% SDS at 37 °C and a wash in 0.1×SSC at 60 °C to 65 °C. A further non-limiting example of high stringency conditions include hybridization in 4×SSC, 5×Denhardt's, 0.1 mg/ml boiled salmon sperm DNA, and 25 mM Na phosphate at 65 °C and a wash in 0.1×SSC, 0.1% SDS at 65 °C. Another illustration of high stringency hybridization conditions includes hybridization in 7% SDS, 0.5 M NaPO₄, 1 mM EDTA at 50 °C with washing in 2×SSC, 0.1% SDS at 50 °C, alternatively with washing in 1×SSC, 0.1% SDS at 50 °C, alternatively with washing in 0.5×SSC, 0.1% SDS at 50 °C, or alternatively with washing in 0.1×SSC, 0.1% SDS at 50 °C, or even with washing in 0.1×SSC, 0.1% SDS at 65 °C. Those skilled in the art will appreciate that specificity is typically a function of post-hybridization washes, the relevant factors being the ionic strength and temperature of the final wash solution.

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions.

As used herein, the term “transgene” refers to a DNA molecule artificially incorporated into an organism's genome because of human intervention, such as a plant transformation method. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. Because of such genomic alteration, the transgenic plant is something distinctly different from the related wild-type plant and the transgenic trait is a trait not naturally found in the wild-type plant. Transgenic plants can comprise the recombinant DNA molecules and engineered proteins provided herein.

As used herein, the term “transgenic” and grammatical variations thereof refer to a plant, including any part derived from the plant, such as a cell, tissue or organ, in which a heterologous nucleic acid is integrated into the genome. In specific embodiments, the heterologous nucleic acid is a recombinant construct, vector or expression cassette comprising one or more nucleic acids.

The term “vector” refers to a composition for transferring, delivering or introducing a nucleic acid (or nucleic acids) into a cell. A vector comprises a nucleic acid molecule comprising the nucleotide sequence(s) to be transferred, delivered or introduced.

### 2. Polynucleotides and Polypeptides, and Compositions Thereof, that Confer Increased Disease Resistance

Compositions, polypeptides, fusion proteins, polynucleotides and active fragments and variants thereof that confer increased disease resistance are provided.

### I. TIRA Polypeptides and Polynucleotides Encoding TIRA Polypeptides

A TIRA polypeptide comprising SEQ ID NO: 1 or an active fragment or variant of SEQ ID NO: 1 is provided. Further provided are polynucleotides comprising a nucleotide sequence encoding the polypeptide of SEQ ID NO: 1 or an active variant or fragment thereof; and polynucleotides comprising any one of SEQ ID NOS: 3-5, or an active fragment or variant thereof.

The TIRA polypeptide (SEQ ID NO: 1) encodes a resistance protein (herein also referred to as an R-protein) that is 340 aa in length. The TIRA polypeptide (SEQ ID NO: 1) comprises several conserved domains including two Toll/Interleukin-1 Receptor (TIR) domains (pfam Accession No: 01582). The two conserved TIR domains of the TIRA polypeptide create a dumbbell structure with the first conserved TIR domain, herein referred to as the TIRA1 domain (amino acids 12 to 174 of SEQ ID NO: 1) separated from the second conserved TIR domain, herein referred to as the TIRA2 domain (amino acids 183-339 of SEQ ID NO: 1). The TIRA1 domain is separated from the TIRA2 domain by approximately 10 amino acids. The position of the conserved TIR domains (that is, TIRA1 and TIRA2 domains) within the TIRA polypeptide is listed at Table 1.

TIR domains of some R-proteins are known to function as NAD+ cleaving enzymes to trigger localized cell death, also known as the hypersensitive response (See PNAS (2017) 114 (10) E2053-2062; and Science August 2019:799-803). The TIRA1 domain of the TIRA polypeptide of SEQ ID NO: 1 has NADase activity. As used herein, NADase activity means the ability of a polypeptide, or active fragment or variant thereof, or domain thereof, to cleave the metabolic cofactor nicotinamide adenine dinucleotide (NAD+) into nicotinamide (Nam) and ADP-ribose (ADPR). In particular embodiments, the TIRA1 domain of the TIRA polypeptide (SEQ ID NO: 1), or an active variant or fragment thereof, comprises a conserved glutamic acid residue (e.g., amino acid 85 of SEQ ID NO: 1, or a position corresponding thereto) that is at least partially responsible for the catalytic cleavage which confers the TIRA1 domain with NADase activity. In particular embodiments, the TIRA2 domain of the TIRA polypeptide (SEQ ID NO: 1), or an active variant or fragment thereof, comprises a non-Glutamic acid residue, such as a valine residue (e.g., amino acid 251 of SEQ ID NO: 1, or a position corresponding thereto) as a result of which the TIRA2 domain does not have NADase activity. In embodiments, such as non-Glutamic acid residue can be mutated to a Glutamic acid residue to confer the TIRA2 domain with NADase activity. In other embodiments, variants of the TIRA polypeptide (SEQ ID NO: 1) comprise a glutamic acid residue (e.g., at amino acid 251 of SEQ ID NO: 1, or a position corresponding thereto) that is responsible for the catalytic cleavage which confers the TIRA2 domain of the variant with NADase activity.

Polynucleotides are provided comprising a coding sequence encoding the TIRA polypeptide (SEQ ID NO: 1) or an active variant or fragment thereof. In particular embodiments, the polynucleotide encoding the TIRA polypeptide (SEQ ID NO: 1) or an active variant or fragment thereof, has a coding sequence comprising, or derived from, a genomic sequence of the TirA gene, such as polynucleotides comprising SEQ ID NO: 3 or 5, or an active fragment or variant thereof. In other specific embodiments, the polynucleotide encoding the TIRA polypeptide (SEQ ID NO: 1) or an active variant or fragment thereof, has a coding sequence comprising or derived from a cDNA sequence of the TirA gene, such as polynucleotides comprising SEQ ID NO: 4, or an active fragment or variant thereof.

The TIRA polypeptide of SEQ ID NO: 1, or an active fragment or variant of SEQ ID NO: 1, confers disease resistance to a plant, plant part or seed, such as to a legume plant, legume plant part, or legume seed, when co-expressed with a TIRB polypeptide (SEQ ID NO: 2), or active fragment or variant of a TIRB polypeptide. In particular embodiments, expression of the TIRA polypeptide of SEQ ID NO: 1, or an active fragment or variant of SEQ ID NO: 1, confers ASR and/or powdery mildew resistance to a soybean plant, plant part or seed when co-expressed with the TIRB polypeptide of SEQ ID NO: 2, or active fragment or variant of SEQ ID NO: 2. In other particular embodiments, expression of the TIRA polypeptide of SEQ ID NO: 1, or an active fragment or variant of SEQ ID NO: 1, confers increased nematode resistance (e.g., root know nematode and/or soybean cysts nematode resistance), bacterial pathogen resistance (e.g., resistance to P. syringae), and/or sucking pest resistance to a soybean plant, plant part or seed when co-expressed with the TIRB polypeptide of SEQ ID NO: 2, or active fragment or variant of SEQ ID NO: 2.

Likewise, polynucleotides encoding the TIRA polypeptide of SEQ ID NO: 1, or an active fragment or variant of SEQ ID NO: 1, such as polynucleotides comprising any of SEQ ID NOS: 3-5, or an active fragment or variant thereof, confer disease resistance to a plant, plant part or seed, such as to a legume plant, legume plant part, or legume seed, when co-expressed with a polynucleotide encoding the TIRB polypeptide of SEQ ID NO: 2, or active fragment or variant of SEQ ID NO: 2, such as polynucleotides comprising any of SEQ ID NOS: 6-8. In particular embodiments, transcription and expression of the polynucleotide of any one of SEQ ID NOS: 3-5, or an active fragment or variant of any one of SEQ ID NOS: 3-5, confers ASR and/or powdery mildew resistance to a soybean plant, plant part or seed when co-expressed or co-transcribed with the polynucleotide of any one of SEQ ID NOS: 6-8, or an active fragment or variant of any one of SEQ ID NOS: 6-8. In other particular embodiments, transcription and expression of the polynucleotide of any one of SEQ ID NOS: 3-5, or an active fragment or variant of any one of SEQ ID NOS: 3-5, confers increased nematode resistance (e.g., root know nematode and/or soybean cysts nematode resistance), bacterial pathogen resistance (e.g., resistance to P. syringae), and/or sucking pest resistance to a soybean plant, plant part or seed when co-expressed or co-transcribed with the polynucleotide of any one of SEQ ID NOS: 6-8, or an active fragment or variant of any one of SEQ ID NOS: 6-8.

As described below, active fragments and variants of the TIRA polypeptide of SEQ ID NO: 1 include polypeptides comprising one or more mutations, truncations, insertions, and/or deletions to SEQ ID NO: 1 while maintaining the polypeptide's ability to confer disease resistance to a plant, plant part or seed, when co-expressed with a polynucleotide encoding the TIRB polypeptide of SEQ ID NO: 2, or active fragment or variant of SEQ ID NO: 2. In particular embodiments, the active variant or fragment may comprise one or more mutations to the NADase region of the TIRA polypeptide, such as one or more mutations in the TIRA1 domain and/or the TIRA2 domain, that result in a decrease in the polypeptide's NADase activity while still retaining the polypeptide's ability to confer disease resistance to a plant, plant part or seed, when co-expressed with a TIRB polypeptide.

### II. TIRB Polypeptides and Polynucleotides Encoding TIRB Polypeptides

A TIRB polypeptide comprising SEQ ID NO: 2 or an active fragment or variant of SEQ ID NO: 2 is provided. Further provided are polynucleotides comprising a nucleotide sequence encoding the polypeptide of SEQ ID NO: 2 or an active variant or fragment thereof; and polynucleotides comprising any one of SEQ ID NOS: 6-8, or an active fragment or variant thereof.

The TIRB polypeptide (SEQ ID NO: 2) encodes a resistance protein (herein also referred to as an R-protein) that is 367 aa in length. The TIRB polypeptide (SEQ ID NO: 2) comprises several conserved domains including two Toll/Interleukin-1 Receptor (TIR) domains (pfam Accession No: 01582). The two conserved TIR domains of the TIRB polypeptide create a dumbbell structure with the first conserved TIR domain, herein referred to as the TIRB1 domain (amino acids 8 to 174 of SEQ ID NO: 2) separated from the second conserved TIR domain, herein referred to as the TIRB2 domain (amino acids 183-344 of SEQ ID NO: 2). The TIRB1 domain is separated from the TIRB2 domain by approximately 10 amino acids. The position of the conserved TIR domains (that is, TIRB1 and TIRB2 domains) within the TIRB polypeptide is detailed at Table 1.

TIR domains of R-proteins are known to function as NAD+ cleaving enzymes to trigger localized cell death, also known as the hypersensitive response (See PNAS (2017) 114 (10) E2053-2062; and Science August 2019:799-803). Each of the TIRB1 and TIRB2 domains has NADase activity. As used herein, NADase activity means the ability of a polypeptide, or active fragment or variant thereof, or domain thereof, to cleave the metabolic cofactor nicotinamide adenine dinucleotide (NAD+) into nicotinamide (Nam) and ADP-ribose (ADPR). In particular embodiments, the TIRB1 domain of the TIRB polypeptide (SEQ ID NO: 2), or an active variant or fragment thereof, comprises a conserved glutamic acid residue (e.g., amino acid 87 of SEQ ID NO: 2, or a position corresponding thereto) that is at least partially responsible for the catalytic cleavage which confers the TIRB1 domain with NADase activity. Likewise, the TIRB2 domain of the TIRB polypeptide (SEQ ID NO: 2), or an active variant or fragment thereof, comprises a conserved Glutamic acid residue (e.g., amino acid 257 of SEQ ID NO: 2, or a position corresponding thereto) that is at least partially responsible for the catalytic cleavage which confers the TIRB2 domain with NADase activity.

Polynucleotides are provided comprising a coding sequence encoding the TIRB polypeptide (SEQ ID NO: 2) or an active variant or fragment thereof. In particular embodiments, the polynucleotide encoding the TIRB polypeptide (SEQ ID NO: 2) or an active variant or fragment thereof, has a coding sequence comprising or derived from a genomic sequence of the TirB gene, such as polynucleotides comprising any one of SEQ ID NOS: 6 and 8, or an active fragment or variant thereof. In other specific embodiments, the polynucleotide encoding the TIRB polypeptide (SEQ ID NO: 2) or an active variant or fragment thereof, has a coding sequence comprising or derived from a cDNA sequence of the TirB gene, such as polynucleotides comprising SEQ ID NO: 7, or an active fragment or variant thereof.

The TIRB polypeptide of SEQ ID NO: 2, or an active fragment or variant of SEQ ID NO: 2, confers disease resistance to a plant, plant part or seed, such as to a legume plant, legume plant part, or legume seed, when co-expressed with a TIRA polypeptide (SEQ ID NO: 1), or active fragment or variant of a TIRA polypeptide. In particular embodiments, expression of the TIRB polypeptide of SEQ ID NO: 2, or an active fragment or variant of SEQ ID NO: 2, confers ASR and/or powdery mildew resistance to a soybean plant, plant part or seed when co-expressed with the TIRA polypeptide of SEQ ID NO: 1, or active fragment or variant of SEQ ID NO: 1. In other particular embodiments, expression of the TIRB polypeptide of SEQ ID NO: 2, or an active fragment or variant of SEQ ID NO: 2, confers increased nematode resistance (e.g., root know nematode and/or soybean cysts nematode resistance), bacterial pathogen resistance (e.g., resistance to P. syringae), and/or sucking pest resistance to a soybean plant, plant part or seed when co-expressed with the TIRA polypeptide of SEQ ID NO: 1, or active fragment or variant of SEQ ID NO: 1.

Likewise, polynucleotides encoding the TIRB polypeptide of SEQ ID NO: 2, or an active fragment or variant of SEQ ID NO: 2, such as polynucleotides comprising any one of SEQ ID NOS: 6-8, or an active fragment or variant thereof, confer disease resistance to a plant, plant part or seed, such as to a legume plant, legume plant part, or legume seed, when co-expressed with a polynucleotide encoding the TIRA polypeptide of SEQ ID NO: 1, or active fragment or variant of SEQ ID NO: 1, such as polynucleotides comprising any one of SEQ ID NOS: 3-5. In particular embodiments, transcription and expression of the polynucleotide of any one of SEQ ID NOS: 6-8, or an active fragment or variant of any one of SEQ ID NOS: 6-8, confers ASR and/or powdery mildew resistance to a soybean plant, plant part or seed when co-expressed or co-transcribed with the polynucleotide of any one of SEQ ID NOS: 3-5, or an active fragment or variant of any one of SEQ ID NOS: 3-5. In other particular embodiments, transcription and expression of the polynucleotide of any one of SEQ ID NOS: 6-8, or an active fragment or variant of any one of SEQ ID NOS: 6-8, confers increased nematode resistance (e.g., root know nematode and/or soybean cysts nematode resistance), bacterial pathogen resistance (e.g., resistance to P. syringae), and/or sucking pest resistance to a soybean plant, plant part or seed when co-expressed or co-transcribed with the polynucleotide of any one of SEQ ID NOS: 3-5, or an active fragment or variant of any one of SEQ ID NOS: 3-5.

As described below, active fragments and variants of the TIRB polypeptide of SEQ ID NO: 2 include polypeptides comprising one or more mutations, truncations, insertions, and/or deletions to SEQ ID NO: 2 while maintaining the polypeptide's ability to confer disease resistance to a plant, plant part or seed, when co-expressed with a polynucleotide encoding the TIRA polypeptide of SEQ ID NO: 1, or active fragment or variant of SEQ ID NO: 1. In particular embodiments, the active variant or fragment may comprise one or more mutations to the NADase region of the TIRB polypeptide, such as one or more mutations in the TIRB1 domain and/or the TIRB2 domain, that result in a decrease in the polypeptide's NADase activity while still retaining the polypeptide's ability to confer disease resistance to a plant, plant part or seed, when co-expressed with a TIRA polypeptide.

Description of functional domains of the TIRA and TIRB polypeptides is summarized in Table 1, below.

One example of an algorithm that is suitable for identifying conserved domains, such as the listed PFAM domains of Table 1, is the Conserved Domain algorithm and associated database, which is described in Marchler-Bauer et al. (Nucleic Acids Res. (2015) 43 (D) 222-226; Nucleic Acids Res. (2017) 45 (D) 200-203) and Shennan Lu et al. (Nucleic Acids Res. (2020) 48 (D1) 265-268). Software for identifying conserved domains is publicly available through the National Center for Biotechnology Information (National Center for Biotechnology Information, U.S. National Library of Medicine, 8600 Rockville Pike, Bethesda, MD 20894 USA).

TABLE 1A
FUNCTIONAL ANNOTATION OF TIRA AND TIRB POLYPEPTIDES

| | | PFAM | | Alternate | | | |
|---|---|---|---|---|---|---|---|
| | | Domain | PFAM | Domain name | | | |
| | SEQ ID | Accession | Domain | as provided | | | |
| Polypeptide | NO: | No. | Name | herein | Start | End | E-value |
| TIRA | 1 | pfam01582 | TIR domain | TIRA1 domain | 12 | 174 | 2.95e−52 |
| TIRA | 1 | pfam01582 | TIR domain | TIRA2 domain | 183 | 339 | 4.12e−39 |
| TIRB | 2 | pfam01582 | TIR domain | TIRB1 domain | 8 | 174 | 5.08e−42 |
| TIRB | 2 | pfam01582 | TIR domain | TIRB2 domain | 183 | 344 | 2.27e−52 |

### III. Fragments and Variants of TIR Polypeptides and Polynucleotides

Active fragments and variants of a TIRA polypeptide (SEQ ID NO: 1) and/or active fragments or variants of a TIRB polypeptide (SEQ ID NO: 2) are also provided. Further provided are polynucleotides comprising a nucleotide sequence encoding an active fragment or variant of the polypeptide of any one of SEQ ID NOS: 1 or 2; and polynucleotides comprising any active variant or fragment of any one of SEQ ID NOS: 3-8.

In specific embodiments, the polynucleotide sequence (SEQ ID NOS: 3-5) and polypeptide sequence (SEQ ID NO: 1) of TIRA, and active variants and fragments thereof increase disease resistance in a plant when co-expressed in a plant, plant part or seed with the polynucleotide sequence (SEQ ID NOS: 6-8) and polypeptide sequence (SEQ ID NO: 2) of TIRB, and active variants and fragments thereof. In particular embodiments, when co-expressed in a plant, plant part or seed, the polynucleotide sequence (SEQ ID NOS: 3-5) and polypeptide sequence (SEQ ID NO: 1) of the TIRA polypeptide and active variants or fragments thereof, and the polynucleotide sequence (SEQ ID NO: 6-8) and polypeptide sequence (SEQ ID NO: 2) of the TIRB polypeptide and active variants or fragments thereof, increase the disease resistance of the plant when compared to an appropriate control plant. Various methods by which such an increase in disease resistance can be measured are provided in the Examples and are discussed elsewhere herein.

Fragments of the TIRA polypeptide that increase disease resistance when expressed in a plant, plant part, or seed include TIRA polypeptide fragments that are shorter than the full-length sequence and can comprise a truncation at either the N or C terminus or an internal deletion. An active fragment of a TIRA polypeptide when expressed in a plant can be a polypeptide that is, for example, 10, 25, 50, 100, 150, 200, 250 or more amino acids in length of SEQ ID NO: 1. Such biologically active portions can be prepared by recombinant techniques and evaluated for activity of being able to confer increased resistance when co-expressed with a TIRB polypeptide, or active variant or fragment thereof. As used herein, a fragment comprises at least 8 contiguous amino acids of SEQ ID NO: 1. Embodiments of an active fragment of the TIRA polypeptide include TIRA polypeptide fragments having increased NADase activity. Such fragments will have an increased NADase activity while retaining the ability of the TIRA polypeptide to (i) mount an immune response when expressed in a plant and (ii) increase disease resistance in a plant when co-expressed in a plant, plant part or seed with a TIRB polypeptide, or active fragment or variant thereof. Still other embodiments of an active fragment of the TIRA polypeptide include TIRA polypeptide fragments having decreased NADase activity. Such fragments will have a reduced NADase activity while retaining the ability of the TIRA polypeptide to (i) mount an immune response when expressed in a plant and (ii) increase disease resistance in a plant when co-expressed in a plant, plant part or seed with a TIRB polypeptide, or active fragment or variant thereof.

Fragments of the TIRB polypeptide that increase disease resistance when expressed in a plant, plant part, or seed include TIRB polypeptide fragments that are shorter than the full-length sequence and can comprise a truncation at either the N or C terminus or an internal deletion. An active fragment of a TIRB polypeptide when expressed in a plant can be a polypeptide that is, for example, 10, 25, 50, 100, 150, 200, 250 or more amino acids in length of SEQ ID NO: 2. Such biologically active portions can be prepared by recombinant techniques and evaluated for activity of being able to confer increased resistance when co-expressed with a TIRA polypeptide, or active variant or fragment thereof. As used herein, a fragment comprises at least 8 contiguous amino acids of SEQ ID NO: 2. Embodiments of an active fragment of the TIRB polypeptide include TIRB polypeptide fragments having NADase activity. Still other embodiments of an active fragment of the TIRB polypeptide include TIRB polypeptide fragments having decreased NADase activity. Such fragments will have a reduced NADase activity while retaining the ability of the TIRB polypeptide to (i) mount an immune response when expressed in a plant and (ii) increase disease resistance in a plant when co-expressed in a plant, plant part or seed with a TIRA polypeptide, or active fragment or variant thereof.

Variant TIRA polypeptides comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1. Such active variants will increase disease resistance in a plant when co-expressed in a plant, plant part or seed with a TIRB polypeptide, or active variant or fragment thereof.

In some embodiments, a variant polypeptide comprises a deletion and/or addition of one or more amino acids at one or more internal sites within the native polypeptide and/or a substitution (e.g., conservative substitution) of one or more amino acids at one or more sites in the native polypeptide. Particular embodiments of a variant of the TIRA polypeptide include TIRA polypeptide variants having one or more mutations in one or more of the NADase sites of the TIRA polypeptide. As used herein, “NADase site” refers to one or more positions within a polypeptide, such as within a conserved TIR domain of a TIR polypeptide (e.g., SEQ ID NO: 1 or 2), that is responsible for the NADase function of the polypeptide. In one example embodiment, a variant TIRA polypeptide comprises a mutation in an NADase site of the TIRA1 and/or TIRA2 domains, such as at a position corresponding to position 85 of SEQ ID NO: 1 and/or a position corresponding to position 251 of SEQ ID NO: 1. In particular embodiments, variants comprise a single Glutamic acid to Alanine mutation in the TIRA1 domain at a position corresponding to position 85 of SEQ ID NO: 1 (herein also referred to as E85A) or a single Valine to Glutamic acid mutation in the TIRA2 domain at a position corresponding to position 251 of SEQ ID NO: 1 (herein also referred to as V251E). In other particular embodiments, variants comprise a double mutation of Glutamic acid to Alanine mutation in the TIRA1 domain at a position corresponding to position 85 of SEQ ID NO: 1 and a Valine to Glutamic acid mutation in the TIRA2 domain at a position corresponding to position 251 of SEQ ID NO: 1 (herein also referred to as an E85A+V251E mutant). Such variants will have altered NADase activity (e.g., reduced NADase activity for loss of function mutations and increased NADase activity for gain of function mutations) while retaining the ability of the TIRA polypeptide to (i) mount an immune response when expressed in a plant and (ii) increase disease resistance in a plant when co-expressed in a plant, plant part or seed with a TIRB polypeptide, or active fragment or variant thereof.

Variant TIRB polypeptides comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 2. Such active variants will increase disease resistance in a plant when co-expressed in a plant, plant part or seed with a TIRA polypeptide, or active variant or fragment thereof.

In some embodiments, a variant polypeptide comprises a deletion and/or addition of one or more amino acids at one or more internal sites within the native polypeptide and/or a substitution (e.g., conservative substitution) of one or more amino acids at one or more sites in the native polypeptide. In example embodiments, a variant TIRA polypeptide comprises a deletion and/or addition of one or more amino acids at one or more internal sites within the native polypeptide of SEQ ID NO: 1 and/or a substitution (e.g., conservative substitution) of one or more amino acids at one or more sites in the native polypeptide of SEQ ID NO: 1. Similarly, a variant TIRB polypeptide comprises a deletion and/or addition of one or more amino acids at one or more internal sites within the native polypeptide of SEQ ID NO: 2 and/or a substitution (e.g., conservative substitution) of one or more amino acids at one or more sites in the native polypeptide of SEQ ID NO: 2.

Particular embodiments of a variant of the TIRB polypeptide include TIRB polypeptide variants having one or more mutations in one or more NADase sites of the TIRB polypeptide. In one example embodiment, a variant TIRB polypeptide comprises a loss of function mutation in an NADase site of the TIRB1 domain, such as at a position corresponding to position 87 of SEQ ID NO: 2. In a particular embodiment, a variant TIRB polypeptide comprises a Glutamic acid to Alanine mutation in the TIRB1 domain at a position corresponding to position 87 of SEQ ID NO: 2 (herein also referred to as E87A). In particular embodiments, a variant TIRB polypeptide comprises a loss of function mutation in an NADase site of the TIRB1 domain, while maintaining a functional NADase site of the TIRB2 domain, for example, a Glutamic acid to Alanine mutation in the TIRB1 domain at a position corresponding to position 87 of SEQ ID NO: 2 (herein also referred to as E87A) while maintaining the Glutamic acid residue in the TIRB2 domain at a position corresponding to position 257 of SEQ ID NO: 2. Such variants will have a reduced NADase activity while retaining the ability of the TIRB polypeptide to (i) mount an immune response when expressed in a plant and (ii) increase disease resistance in a plant when co-expressed in a plant, plant part or seed with a TIRA polypeptide, or active fragment or variant thereof.

In other instances, the TIRA polypeptide variant and/or TIRB polypeptide variant comprises a tag, such as a His tag. In still other instances, the polypeptide variant comprises a detectable marker, such as a detectable peptide marker.

In some embodiments, a variant TIRA polypeptide includes an annotation variant of the TIRA polypeptide comprising a different (e.g., larger or fewer) number of amino acids relative to the polypeptide of SEQ ID NO: 1. These annotation variants may occur due to annotation of the TirA gene sequence, genomic sequence, or annotation of the translated gene sequence or genomic sequence, with an alternate start codon. In example embodiments, annotation of the translated gene sequence with a relatively upstream start codon results in a variant having an additional number of amino acids while annotation of the translated gene sequence with a relatively downstream start codon results in a variant having a fewer number of amino acids. In embodiments, annotation variants of SEQ ID NO: 1 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1.

In some embodiments, a variant TIRB polypeptide includes an annotation variant of the TIRB polypeptide comprising a different (e.g., larger or fewer) number of amino acids relative to the polypeptide of SEQ ID NO: 2. These annotation variants may occur due to annotation of the TirB gene sequence, genomic sequence, or annotation of the translated gene sequence or genomic sequence, with an alternate start codon. In example embodiments, annotation of the translated gene sequence with a relatively upstream start codon results in a variant having an additional number of amino acids while annotation of the translated gene sequence with a relatively downstream start codon results in a variant having a fewer number of amino acids. In embodiments, annotation variants of SEQ ID NO: 2 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 2.

In still other embodiments, a variant TIRA polypeptide includes an alternative splice variant (or simple “splice variant”) of the TIRA polypeptide comprising a different (e.g., larger or fewer) number of amino acids relative to the polypeptide of SEQ ID NO: 1, and/or comprising one or more substitutions relative to the polypeptide of SEQ ID NO: 1. These splice variants may occur due to alternative splicing of exons and introns of the TirA gene or genomic sequence resulting in the creation of variant mRNA transcripts that are expressed to form variant protein sequences. In embodiments, splice variants of SEQ ID NO: 1 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1.

In still other embodiments, a variant TIRB polypeptide includes an alternative splice variant (or simple “splice variant”) of the TIRB polypeptide comprising a different (e.g., larger or fewer) number of amino acids relative to the polypeptide of SEQ ID NO: 2, and/or comprising one or more substitutions relative to the polypeptide of SEQ ID NO: 2. These splice variants may occur due to alternative splicing of exons and introns of the TirB gene or genomic sequence resulting in the creation of variant mRNA transcripts that are expressed to form variant protein sequences. In embodiments, splice variants of SEQ ID NO: 2 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 2.

Fragments and variants of a nucleotide sequence can encode protein fragments that retain the biological activity of natural proteins and have the ability to increase disease resistance. Alternatively, nucleotide sequence fragments or variants that can be used as hybridization probes or in recombinant DNA constructs designed for gene editing do not necessarily code protein fragments that maintain biological activity. Thus, the fragment of the nucleotide sequence may be in the range of at least about 15, 50, 100, 150, 200, 250, 300, 350, 400, 450, 500, 550, 600, 650, 700, 750, 800, 750, 900, 950, 1000 nucleotides or less than the full-length nucleotide sequence coding the protein disclosed herein (i.e, any one of SEQ ID NOS: 3-5 and 6-8).

A variant of the nucleotide sequence has as at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the nucleotide sequence of any one of SEQ ID NOS: 3-5 and 6-8. In specific embodiments, the variant nucleotide sequence encodes an active polypeptide of the invention. In other embodiments, the variant polynucleotide need not encode an active variant polypeptide and can be used as components of a gene editing construct or as probes or primers or other tools useful in generating the plants and seeds provided herein.

In some embodiments, fragments and variants of the polypeptides disclosed herein each comprise one or more conserved domains of the canonical polypeptide. In some embodiments, the active variant or fragment can comprise a polypeptide comprising at least 40%, 50%, 60%, at least 65%, at least 70%, at least 75%, at least 80%, at least 85%, at least 90%, at least 95%, at least 96%, at least 97%, at least 98%, at least 99% or 100% identity to one or more of the conserved domains in the canonical polypeptide sequence.

In one example, a variant or fragment of the TIRA polypeptide (SEQ ID NO: 1) may comprise one or more of the conserved TIRA1 domain (aa 12 to 174 of SEQ ID NO:1), and the TIRA2 domain (aa 183 to 339 of SEQ ID NO: 1). For example, active variants are provided whereby the amino acid sequence shares at least 80%, 85%, 90%, 91%, 92%, 93%, 94%, 95%, 96%, 97%, 98%, 99% sequence identity across the full length of SEQ ID NO: 1, and further comprises a region having at least 90%, 91%, 92%, 93%, 94%, 95%, 96%, 97%, 98%, 99%, or 100% identity to the consensus sequence of the given SEQ ID's PFAM domains as set forth in Table 1A. In other embodiments, active variants are provided whereby the amino acid sequence shares at least 80%, 85%, 90%, 91%, 92%, 93%, 94%, 95%, 96%, 97%, 98%, 99% sequence identity across the full length of SEQ ID NO: 1 and further comprises a region having at least 90%, 91%, 92%, 93%, 94%, 95%, 96%, 97%, 98%, 99%, or 100% identity to the corresponding region of SEQ ID NO: 1 that corresponds to the amino acid position of the consensus sequence of the SEQ ID's PFAM domains as set forth in Table 1A.

In one example, a variant or fragment of the TIRB polypeptide (SEQ ID NO: 2) may comprise one or more of the conserved TIRB1 domain (aa 8 to 174 of SEQ ID NO: 2), and the TIRB2 domain (aa 183 to 344 of SEQ ID NO: 2). For example, active variants are provided whereby the amino acid sequence shares at least 80%, 85%, 90%, 91%, 92%, 93%, 94%, 95%, 96%, 97%, 98%, 99% sequence identity across the full length of SEQ ID NO: 2, and further comprises a region having at least 90%, 91%, 92%, 93%, 94%, 95%, 96%, 97%, 98%, 99%, or 100% identity to the consensus sequence of the PFAM domain as set forth in Table 1A. In other embodiments, active variants are provided whereby the amino acid sequence shares at least 80%, 85%, 90%, 91%, 92%, 93%, 94%, 95%, 96%, 97%, 98%, 99% sequence identity across the full length of SEQ ID NO: 2 and further comprises a region having at least 90%, 91%, 92%, 93%, 94%, 95%, 96%, 97%, 98%, 99%, or 100% identity to the corresponding region of SEQ ID NO: 2 that corresponds to the amino acid position of the consensus sequence of the PFAM domains as set forth in Table 1A.

The term “corresponding to” in the context of nucleic acid sequences means that when the nucleic acid sequences of certain sequences are aligned with each other, the nucleic acids that “correspond to” certain enumerated positions in the present invention are those that align with these positions in a reference sequence, but that are not necessarily in those exact numerical positions relative to a particular nucleic acid sequence of the invention. Optimal alignment of sequences for comparison can be conducted by computerized implementations of known algorithms or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity and sequence similarity as well as for aligning sequences is the BLAST algorithm, which is described in Altschul et al, J. Mol. Biol. 215:403-410 (1990). Software for performing BLAST analyses is publicly available through the National Center for Biotechnology Information (National Center for Biotechnology Information, U.S. National Library of Medicine, 8600 Rockville Pike, Bethesda, MD 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990).

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The variants and fragments disclosed herein can be altered, for example, by including amino acid substitutions, deletions, truncations, and insertions. Methods for such manipulations are known in the art. For example, amino acid sequence variants and fragments of the TIR polypeptides can be prepared by mutations in the corresponding polynucleotide sequence. Methods for mutagenesis and polynucleotide alterations are known in the art.

Variant polynucleotides and polypeptides also encompass sequences and polypeptides derived from mutagenic or recombinogenic procedures, including and not limited to procedures such as DNA shuffling. Strategies for such DNA shuffling are known in the art.

Variants may be made by making random mutations in the TIR polypeptide sequences. In other embodiments, the variants may be specifically designed. In the case of designed mutants, it is possible to generate variants with similar biological activity to the original polypeptide when amino acid identity is maintained in regions of the polypeptide which account for biological activity or are involved in determining a three-dimensional configuration of the polypeptide which is responsible for the biological activity. It is also possible to retain the biological activity if conservative substitutions are made wherein amino acids of a given class are replaced with another amino acid of the same class. As such, it is known that amino acids can be placed in one of the following classes: aliphatic or cyclic (Glycine, Alanine, Valine, Leucine, Isoleucine, Proline), Aromatic (Phenylalanine, Tyrosine, Tryptophan), Acidic (Aspartic acid, Glutamic acid, Asparagine, Glutamine), Basic (Histidine, Lysine, Arginine), and Sulfur or hydroxyl containing groups (Serine, Cysteine, Methionine, Threonine). Conservative substitutions whereby an amino acid of one class is replaced with another amino acid of the same type are least likely to materially alter the biological activity of the variant.

Variants of polypeptide and polynucleotides also comprise sequences from other organisms, particularly other plants, isolated based on their sequence identity to the polypeptide and polynucleotide sequences disclosed herein. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode for a TIRA or TIRB polypeptide that confers or enhances disease resistance and that hybridize to the sequences disclosed herein, or to variants or fragments thereof, are encompassed by the present disclosure. Isolated polynucleotides that encode polypeptides having the conserved TIR domains in corresponding positions when aligned with the polypeptides disclosed herein, or to variants or fragments thereof, are also encompassed by the present disclosure. Example orthologs of TIRA and TIRB polypeptides are described herein at Example 5 and include the polypeptides encoded by the CcRpp2-R1 (SEQ ID NOS: 45-46) and CcRpp2-R3 (SEQ ID NO: 47-48) genes from Cajanus cajun. Additional orthologs identifiable based on sequence identity can be found at Tables 3-4 of WO2022/140257, the contents of which are incorporated by reference herein in their entirety.

Variants of polypeptides and polynucleotides also include annotation variants and splice variants of ortholog sequences from other organisms, particularly other plants, isolated based on their sequence identity to the polypeptide and polynucleotide sequences disclosed herein.

Variants of polypeptide and polynucleotides also comprise sequences from the same organism present at a different location of the genome, such as on a different chromosome, isolated based on their sequence identity to the polypeptide and polynucleotide sequences disclosed herein. Such sequences include sequences that are paralogs of the disclosed sequences. The term “paralogs” refers to gene copies created by a duplication event on the same genome. Genes found on the same genome are considered paralogs when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of paralogs can be conserved, though they may be expressed only under different selective pressures. Paralogs may also develop different functions due to missing selective pressure on the duplicated copy of a gene.

Variants of polypeptide and polynucleotides also comprise sequences that are allelic variants of the disclosed sequences. The term “allelic variants” refers to different variants of the same gene at a single gene locus that can cause the same or similar phenotypic expression (e.g., of increasing disease resistance). These include genes from the same locus of the same organism, as well as genes from the same locus of an organism belonging to the same genus. Such allelic variants are isolated based on their sequence identity to the polypeptide and polynucleotide sequences disclosed herein. Genes found at the same locus are considered allelic variants when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of allelic variants can be conserved. Example allelic variants of the TirA gene are described herein at Example 6 and include the RG6a gene (SEQ ID NO: 39) from G. clandestina as well as the RG7a gene (SEQ ID NO: 41) and RG8a gene (SEQ ID NO: 43) from G. canescens. Example allelic variants of the TirB gene are described herein at Example 6 and include the RG6b gene (SEQ ID NO: 40) from G. clandestina as well as the RG7b gene (SEQ ID NO: 42) and RG8b gene (SEQ ID NO: 44) from G. canescens.

Variants of the polypeptides and polynucleotides also comprise sequences from other organisms, particularly other plants, identified based on their sequence identity to the polypeptide and polynucleotide sequences disclosed herein, and having functional identity with the polypeptide and polynucleotide sequences disclosed herein. Such variants having “functional identity” are referred to herein as “functional identity variants”. As used herein, “functional identity” refers to the presence of a common functional activity, such as a common enzymatic activity or common mode of action. Polypeptides having functional identity may have low sequence identity, for example, their nucleotide sequences and/or their encoded polypeptide sequences may share about 40%, about 50%, about 60%, about 70%, about 80%, about 90%, about 95%, about 96%, about 97%, about 98%, about 99%, or greater sequence identity. However, they may share specificity or preference to the same substrate, share kinetic parameters, etc. In one example, variant polypeptides and polynucleotides of the TIRA and TIRB polypeptides, and polynucleotide sequences encoding such polypeptides, comprise proteins that interact with the same set of effector proteins to generate a hypersensitive response in a plant cell. Variants of polypeptide and polynucleotides also comprise annotation variants and/or splice variants of sequences having functional identity to a reference sequence and that can interact with the same set of effector proteins to generate a hypersensitive response in a plant cell.

As used herein, “effectors” or “effector proteins” or “plant pathogen effectors” refer to polypeptide molecules secreted by pathogens to counteract the endogenous defense system of a plant. Effector proteins interact (e.g., physically interact) with a plant-based resistance protein, or a polypeptide encoded by a plant-based resistance gene (e.g., an R-protein, such as a TIRA protein, a TIRB protein, or variants or active fragments thereof), to elicit a localized immune response comprising a hypersensitive programmed cell death response in infected plant cells/tissues. In one embodiment, the “hypersensitive response” includes increased electrolyte leakage from a site of infection. In another embodiment, the “hypersensitive response” includes an increase (e.g., partial increase or complete increase) in phenotypic characteristics associated with plant pathogen specific protease dependent cell death.

Typically, each R-protein interacts with a distinct set of effector proteins, thereby defining a distinct mode of action or site of action for conferring disease resistance. In one example embodiment, the TIRA polypeptide and TIRB polypeptide are R-proteins that interact with a distinct set of effector proteins to elicit a hypersensitive response in a plant cell. As used herein, the term “site of action” or “mode of action” refers to specific interactions of a effector polypeptide derived from a pathogen with a disease resistance protein (R-protein) derived from a plant.

In particular embodiments, variants of the polypeptides and polynucleotides comprise sequences from other organisms, particularly other plants, that when expressed in a plant and assayed, interact with most or all of the effector proteins recognized by the TIRA and/or TIRB polypeptides to generate a localized hypersensitive response. Assays for identifying the interaction of an expressed R-protein, or a variant or fragment thereof, with an effector protein, and identifying the presence of a localized hypersensitive response are disclosed herein Section 9 at Examples 8, and As elaborated below, variants of the polypeptides can also comprise fusion proteins generated by expressing a TIRA polypeptide linked to a TIRB polypeptide. In particular embodiments, the fusion protein comprises the TIRB polypeptide at the N-terminus of the fusion protein, as in the fusion protein of SEQ ID NOS: 9-11. In other embodiments, the fusion protein comprises the TIRA polypeptide at the N-terminus of the fusion protein, as in the fusion protein of SEQ ID NO: 12. In embodiments, the polypeptides may be linked to each other in the fusion protein by a cleavable linker that causes the TIRA polypeptide to be separated from the TIRB polypeptide following transcription and expression of the fusion protein. Example embodiments of fusion proteins comprising a self-cleavable linker include the fusion protein of SEQ ID NO: 10. Variant polynucleotides comprise nucleotides having a nucleotide sequence coding for such fusion proteins, such as polynucleotides having a nucleotide sequence encoding the fusion protein of any of SEQ ID NOS: 9-12, or an active variant or fragment thereof, or polynucleotides having the nucleotide sequence of any of SEQ ID NOS: 13-16, or active variants or fragments thereof.

### IV. Fusion Proteins Comprising TIRA and TIRB Polypeptides, and Polynucleotides Encoding Such Fusion Proteins

Fusion proteins comprising the TIRA polypeptide (SEQ ID NO: 1), or an active fragment or variant thereof, operably coupled to the TIRB polypeptide (SEQ ID NO: 2, or active fragment or variant thereof, are also provided herein. In particular embodiments, the fusion proteins comprise the polypeptide sequence of any of SEQ ID NOS: 9-12, or an active variant or fragment thereof. Further provided are polynucleotides encoding a fusion protein comprising SEQ ID NO: 1, or an active variant or fragment thereof, linked to SEQ ID NO: 2, or an active variant or fragment thereof; and polynucleotides comprising any one of SEQ ID NOS: 3-5, or an active variant or fragment thereof, operably linked to any one of SEQ ID NOS: 6-8, or an active variant or fragment thereof. In particular embodiments, polynucleotides are provided encoding a fusion protein comprising any of SEQ ID NOS: 9-12, or an active variant or fragment thereof, and polynucleotides comprising any one of SEQ ID NOS: 13-16 or variants and fragments thereof.

As used herein, “fusion proteins” “fusion polypeptides”, “TIRATIRB fusion proteins/polypeptides” or “TIRATIRB proteins/polypeptides” refer to polypeptide sequences that, at least when expressed, comprise the sequence of a TIRA polypeptide (or active variant or fragment thereof) in frame with the sequence of a TIRB polypeptide (or active variant or fragment thereof) in the same protein, such as where the sequence of the TIRA polypeptide (or active variant or fragment thereof) is linked to the sequence of the TIRB polypeptide (or active variant or fragment thereof) via a linker. Such fusion proteins are encoded by polynucleotides having a nucleotide sequence encoding the TIRA polypeptide (or active variant or fragment thereof) arranged transcriptionally and translationally in frame with a nucleotide sequence encoding a TIRB polypeptide (or active variant or fragment thereof).

In example embodiments, the fusion proteins provided herein comprise:
(i) the sequence of any TIRA polypeptide provided herein, including (a) the TIRA polypeptide of SEQ ID NO: 1, or (b) a variant or fragment of the TIRA polypeptide of SEQ ID NO: 1, such as the TIRA polypeptide of SEQ ID NO: 1 further comprising a loss of function mutation in an NADase site of the TIRA domain or a gain of function mutation in an NADase site of the TIRA2 domain, or (c) an ortholog of the TIRA polypeptide of SEQ ID NO: 1 (such as the ccRpp2-R1 polypeptide from Cajanus cajun, as provided in SEQ ID NO: 46, or any of the TIRA orthologs provided at Tables 3-4 of WO2022/140257, the contents of which are incorporated by reference herein in their entirety) or (d) an allelic variant of the TIRA polypeptide of SEQ ID NO: 1 (such as any one of the allelic variants encoded by the polynucleotide of SEQ ID NOS: 38, 40 and 42);
linked to:
(ii) the sequence of any TIRB polypeptide provided herein, including (a) the TIRB polypeptide of SEQ ID NO: 2, or (b) a variant or fragment of the TIRB polypeptide of SEQ ID NO: 2, such as the TIRB polypeptide of SEQ ID NO: 2 further comprising a loss of function mutation in an NADase site of the TIRB1 domain, or (c) an ortholog of the TIRA polypeptide of SEQ ID NO: 1 (such as the ccRpp2-R1 polypeptide from Cajanus cajun, as provided in SEQ ID NO: 48, or any of the TIRB orthologs provided at Tables 3-4 of WO2022/140257, the contents of which are incorporated by reference herein in their entirety) or (d) an allelic variant of the TIRB polypeptide of SEQ ID NO: 2 (such as any one of the allelic variants encoded by the polynucleotide of SEQ ID NOS: 39, 41 and 43). In particular embodiments, the polypeptide of (i) is linked to the polypeptide of (ii) via any of the linker sequences disclosed herein.

“Fusion proteins” also refer to polypeptide sequences wherein the sequence of a TIRA polypeptide (or active variant or fragment thereof) is linked to the sequence of a TIRB polypeptide (or active variant or fragment thereof) via a cleavable linker. Such fusion proteins undergo cleavage, for example self-cleavage, following expression of the fusion protein, resulting in the separation of the constituent polypeptides.

In example embodiments, the TIRA polypeptide, or active variant or fragment thereof, is at the N-terminus of the fusion protein and the TIRB polypeptide, or active variant or fragment thereof, is at the C-terminus of the fusion protein. In a particular embodiment, the fusion protein comprising the TIRA polypeptide at the N-terminus and the TIRB polypeptide at the C-terminus comprises SEQ ID NO: 12. In other example embodiments, the TIRA polypeptide, or active variant or fragment thereof, is at the C-terminus of the fusion protein and the TIRB polypeptide, or active variant or fragment thereof, is at the N-terminus of the fusion protein. In particular embodiments, the fusion protein comprising the TIRB polypeptide at the N-terminus and the TIRA polypeptide at the C-terminus comprises any one of SEQ ID NOS: 9-11.

In embodiments, the TIRA polypeptide is coupled to the TIRB polypeptide in the fusion protein via a linker sequence comprising one or more amino acids (aa), such as comprising at least 1aa, 2aa, 3aa, 5aa, 10aa, 30aa, or at least 50aa. In particular embodiments, the TIRA polypeptide, or active variant or fragment thereof, is linked to the TIRB polypeptide, or active variant or fragment thereof, via a linker sequence comprising the peptide sequence of any one of SEQ ID NOS: 36-38. In particular embodiments, the TIRA polynucleotide, or active variant or fragment thereof, is linked to the TIRB polynucleotide, or active variant or fragment thereof, via a linker sequence comprising the nucleotide sequence of any one of SEQ ID NOS: 33-35.

Linker sequences may include non-cleavable linker sequences or cleavable linker sequences. In particular embodiments, the TIRA polypeptide, or active variant or fragment thereof, is linked to the TIRB polypeptide, or active variant or fragment thereof, in a fusion protein (e.g., fusion protein of SEQ ID NOS: 9 or 12) via a non-cleavable linker sequence (xLinker-01 peptide; SEQ ID NO: 36) and/or the TIRA polynucleotide, or active variant or fragment thereof, is linked to the TIRB polynucleotide, or active variant or fragment thereof, in the fusion protein (e.g., fusion protein of SEQ ID NOS: 9 or 12) via a non-cleavable linker sequence (xLinker-01; SEQ ID NO: 33). In other particular embodiments, the TIRA polypeptide, or active variant or fragment thereof, is linked to the TIRB polypeptide, or active variant or fragment thereof, in a fusion protein (e.g., fusion protein of SEQ ID NOS: 10 or 11) via a cleavable linker sequence (e.g., the self-cleavable linker sequence of xT2ALinker-03 peptide; SEQ ID NO: 38 or xT2ALinker-04 peptide; SEQ ID NO: 37) and/or the TIRA polynucleotide, or active variant or fragment thereof, is linked to the TIRB polynucleotide, or active variant or fragment thereof, in the fusion protein (e.g., fusion protein of SEQ ID NOS: 10 or 11) via a cleavable linker sequence (e.g., the self-cleavable linker sequence of xT2ALinker-03; SEQ ID NO: 35 or xT2ALinker-04; SEQ ID NO: 34). Still other cleavable or non-cleavable linker sequences may be used.

In embodiments of a TIRATIRB fusion protein comprising a cleavable linker sequence, following expression of the fusion protein (that is, following transcription and translation of a polynucleotide sequence encoding the fusion protein comprising the cleavable linker sequence), the fusion protein is cleaved, e.g., self-cleaved, to release the constituent TIRA and TIRB polypeptides (or active variants or fragments thereof) in independent form, unlinked from each other.

In other embodiments, the fusion protein comprises a tag or marker sequence fused at the N-terminus or the C-terminus of the protein. Polynucleotides encoding fusion proteins comprising a linker sequence having one or more amino acids comprise a linker nucleotide sequence that is a multiple of 3 nucleotides such that the nucleotide sequence encoding the TIRA polypeptide (or active variant or fragment thereof) is translationally in frame with the nucleotide sequence encoding the TIRB polypeptide (or active variant or fragment thereof).

Fusion proteins are made by methods known in the art. In example embodiments, the fusion protein of the present disclosure is made by operably linking a single promoter to a nucleic acid comprising (i) a polynucleotide encoding the TIRA polypeptide, or active variant or fragment thereof, arranged transcriptionally in frame with (ii) a polynucleotide encoding the TIRB polypeptide or active variant or fragment thereof. As a result, a single transcript is created, the translation of which results in a protein comprising the TIRA polypeptide fused to the TIRB polypeptide. In embodiments where the fusion protein comprises a cleavable linker sequence, following translation, the fusion protein is cleaved to form the constituent TIRA and TIRB polypeptides. In particular embodiments, the fusion protein of the present disclosure is made by operably linking a single promoter to a nucleic acid comprising (i) a polynucleotide comprising any of SEQ ID NOS: 3-5 or active variant or fragment thereof, arranged transcriptionally in frame with (ii) a polynucleotide comprising any of SEQ ID NOS: 6-8 or active variant or fragment thereof.

The TIRATIRB polypeptides of any one of SEQ ID NOS: 9-12 encode fusion resistance proteins that comprise the conserved domains of the TIRA and TIRB polypeptides previously disclosed. Particularly, the fusion proteins of SEQ ID NOS: 9-12 comprise a TIRA1 domain, a TIRA2 domain, a TIRB1 domain and a TIRB2 domain. The TIRATIRB fusion polypeptide of any of SEQ ID NOS: 9-12, and active variants and fragments thereof, confer or increase disease resistance to a plant, plant part or seed, such as to a legume plant, legume plant part, or legume seed, when expressed in the plant, plant part, or seed. In particular embodiments, expression of the TIRATIRB fusion protein of SEQ ID NOS: 9-12, or an active fragment or variant of any of SEQ ID NOS: 9-12, confers or increases ASR and/or powdery mildew resistance to a soybean plant, plant part or seed when expressed in the plant, plant part or seed. In other particular embodiments, expression of the RG32RG34 fusion protein, or an active fragment or variant thereof, confers or increases resistance to soybean cyst nematode, root knot nematode, bacterial pathogens such as Pseudomonas syringae, and/or sucking pests such as aphids, stinkbugs, and whiteflies, in a soybean plant, plant part or seed when expressed in the plant, plant part or seed. Various methods by which such an increase in disease resistance can be measured are provided in the Examples and are discussed elsewhere herein.

Active fragments and variants of a TIRATIRB fusion protein (any one of SEQ ID NOS: 9-12) are also provided. Further provided are polynucleotides comprising a nucleotide sequence encoding an active fragment or variant of the fusion protein of any of SEQ ID NOS: 9-12; and polynucleotides comprising any one of SEQ ID NOS: 13-16, or an active variant or fragment thereof.

Fragments of the TIRATIRB fusion protein that increase disease resistance when expressed in a plant, plant part, or seed include TIRATIRB fusion protein fragments that are shorter than the full-length sequence and can comprise a truncation at either the N or C terminus or an internal deletion. An active fragment of a TIRATIRB polypeptide when expressed in a plant can be a polypeptide that is, for example, 10, 25, 50, 100, 150, 200, 250 or more amino acids in length of any one of SEQ ID NOS: 9-12. Such biologically active portions can be prepared by recombinant techniques and evaluated for activity of being able to confer increased resistance when expressed in a plant, plant part, or seed.

Variant TIRATIRB polypeptides comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of any of SEQ ID NOS: 9-12. Such active variants will increase disease resistance in a plant when expressed in a plant, plant part or seed. In some embodiments, a variant polypeptide comprises a deletion and/or addition of one or more amino acids at one or more internal sites within the TIRA polypeptide and/or TIRB polypeptide portion of the fusion protein, and/or a substitution of one or more amino acids at one or more sites in the TIRA and/or TIRB portion of the fusion polypeptide.

Particular embodiments of a variant of the TIRATIRB fusion polypeptide include variants having one or more mutations (e.g., gain of function or loss of function mutations) in one or more

NADase sites of the TIRA polypeptide and/or TIRB polypeptide resulting in a change (e.g., increase or decrease) in NADase activity of the fusion protein or the constituent TIR polypeptides. Example embodiments include variant fusion proteins comprising a loss of function mutation in an NADase site of the TIRA1 domain of the fusion protein, such as at a position corresponding to position 85 of SEQ ID NO: 1 (e.g., corresponding to the E85A mutation), a gain of function mutation in an NADase site of the TIRA2 domain of the fusion protein, such as at a position corresponding to position 251 of SEQ ID NO: 1 (e.g., corresponding to the V251E mutation), a loss of function mutation in an NADase site of the TIRB1 domain of the fusion protein, such as at a position corresponding to position 87 of SEQ ID NO: 2 (e.g., corresponding to the E87A mutation), or any combination thereof, including but not limited to a single mutant (e.g., E85A, E87A, or V251E), a double mutant (e.g., E85A+V251E; E85A+E87A; V251E+E87A) or a triple mutant (e.g., E85A+V251E+E87A). Such variants will have a modified (e.g., reduced) NADase activity while retaining the ability of the fusion protein to (i) mount an immune response and (ii) increase disease resistance when expressed in a plant, plant part or seed. In embodiments where the variant fusion protein comprising the one or more mutations in the NADase sites comprises a cleavable linker sequence, following expression of the fusion protein, the protein is cleaved to the constituent TIR polypeptides comprising the corresponding NADase site mutations. In embodiments where the constituent TIRA polypeptide comprises the E85A mutation (or a corresponding mutation) and/or the constituent TIRB polypeptide comprises the E87A mutation (or a corresponding mutation), the resulting TIR polypeptide has decreased NADase activity relative to an unmutated version. In embodiments where the constituent TIRA polypeptide comprises the V251E mutation (or a corresponding mutation), the resulting TIRA polypeptide has increased NADase activity relative to an unmutated version.

Variant TIRATIRB polypeptides include fusion proteins comprising an ortholog, paralog, or allelic variant of a TIRA polypeptide having an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1, linked to an ortholog, paralog, or allelic variant of a TIRB polypeptide having an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 2.

Such active variants will increase disease resistance in a plant when expressed in a plant, plant part or seed.

Variant polynucleotide sequences encoding for the variant fusion proteins are also provided. In example embodiments, the variant fusion proteins are encoded by variant polynucleotides comprising any one of SEQ ID NOS: 17-19 and 21.

In other instances, the fusion protein variant comprises a tag, such as a His tag. In still other instances, the fusion protein variant comprises a detectable marker.

Fragments and variants of a nucleotide sequence can encode fusion protein fragments that retain the biological activity of the original fusion protein and have the ability to increase disease resistance. Alternatively, nucleotide sequence fragments or variants that can be used as hybridization probes or in recombinant DNA constructs designed for gene editing do not necessarily code protein fragments that maintain biological activity. Thus, the fragment of the nucleotide sequence may be in the range of at least about 15, 50, 100, 150, 200, 250, 300, 350, 400, 450, 500, 550, 600, 650, 700, 750, 800, 750, 900, 950, 1000 nucleotides or less than the full-length nucleotide sequence coding the protein disclosed herein (i.e, any one of SEQ ID NOS: 17-19 and 21).

A variant of the nucleotide sequence has as at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the nucleotide sequence of any one of SEQ ID NOs: 17-19 and 21. In specific embodiments, the variant nucleotide sequence encodes an active fusion protein of the invention. In other embodiments, the variant polynucleotide need not encode an active variant fusion protein and can be used as components of a gene editing construct or as probes or primers or other tools useful in generating the plants and seeds provided herein.

In some embodiments, fragments and variants of the fusion protein disclosed herein each comprise one or more conserved domains of the canonical fusion protein. In some embodiments, the active variant or fragment can comprise a polypeptide comprising at least 40%, 50%, 60%, at least 65%, at least 70%, at least 75%, at least 80%, at least 85%, at least 90%, at least 95%, at least 96%, at least 97%, at least 98%, at least 99% or 100% identity to one or more of the conserved domains in the canonical fusion protein sequence.

In one example, a variant or fragment of the fusion protein (any one of SEQ ID NOS: 9-11) may comprise one or more of the conserved TIR domains. For example, active variants are provided whereby the amino acid sequence shares at least 80%, 85%, 90%, 91%, 92%, 93%, 94%, 95%, 96%, 97%, 98%, 99% sequence identity across the full length of any of SEQ ID NOS: 9-11, and further comprises a region having at least 90%, 91%, 92%, 93%, 94%, 95%, 96%, 97%, 98%, 99%, or 100% identity to the TIR domain (PFAM01582) set forth in Table 1.

The variants and fragments disclosed herein can be altered, for example, by including amino acid substitutions, deletions, truncations, and insertions. Methods for such manipulations are known in the art. For example, amino acid sequence variants and fragments of the fusion protein can be prepared by mutations in the corresponding polynucleotide sequence. Methods for mutagenesis and polynucleotide alterations are known in the art.

The polynucleotide of any one of SEQ ID NO: 13-19 and 21, or active variants and fragments thereof, encoding the fusion protein of any of SEQ ID NOS: 9-12 and active variants and fragments thereof, may be included in an expression cassette operably linked to a heterologous plant active promoter. In example embodiments, expression of the TIRATIRB fusion protein, or active variants or fragments thereof, is driven by a constitutive promoter or an endogenous promoter. In other example embodiments, expression of the TIRATIRB fusion protein, or active variants or fragments thereof, is driven by an inducible promoter, such as a rust-inducible promoter.

3. Expression Cassettes and Regulatory Elements

Polynucleotides provided herein can be provided in expression cassettes for expression (herein also referred to as “DNA constructs”) in an organism of interest. The expression cassette will include 5′ and 3′ regulatory sequences operably linked to a polynucleotide encoding a TIRA polypeptide, a TIRB polypeptide, or a TIRATIRB fusion protein, or active variants or fragments of a TIRA polypeptide, a TIRB polypeptide, or a TIRATIRB fusion protein, that allows for expression of the polynucleotide. The cassette may additionally contain at least one additional gene or genetic element to be co-transformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory elements or regions. The expression cassette may additionally contain a selectable marker gene.

“DNA construct” refers to the genetic elements operably linked to each other making up a recombinant DNA molecule and may comprise elements that provide expression of a DNA polynucleotide molecule in a host cell and elements that provide maintenance of the construct in the host cell. The various genetic elements within the DNA construct can be native to polynucleotide encoding the polypeptide or heterologous to the native polynucleotide encoding the polypeptide.

DNA constructs, vectors, and expression cassettes can be prepared that incorporate the nucleotide sequence encoding the TIRA polypeptide, TIRB polypeptide, or TIRATIRB fusion protein, or an active variant or fragment of the TIRA polypeptide, TIRB polypeptide, or TIRATIRB fusion protein, for use in directing the expression of the sequences directly from the host plant cell. Examples of such constructs suitable for this purpose and methods are generally described, for example, in Svab et al., Proc. Natl. Acad. Sci. USA 87:8526-8530, (1990) and Svab et al., Proc. Natl. Acad. Sci. USA 90:913-917 (1993) and in U.S. Pat. No. 5,693,507.

A plant expression cassette comprises the operable linkage of genetic elements that when transferred into a plant cell provides expression of a desirable gene product. “Plant expression cassette” refers to a DNA construct comprising the regulatory elements that are operably linked to provide the expression of a desired nucleic acid in a plant. Promoters, leaders, introns, transit peptide encoding polynucleic acids, 3′ transcriptional termination regions are all genetic elements that may be operably linked by those skilled in the art of plant molecular biology to provide a desirable level of expression or functionality to a TIRA polypeptide and/or a TIRB polypeptide or a TIRATIRB fusion protein, or an active variant or fragment of any of the aforementioned.

A DNA construct can contain one or more plant expression cassettes expressing the DNA molecules of the present invention or other DNA molecules useful in the genetic engineering of crop plants. One example of a DNA construct that may be used for expressing the TIRA polypeptide or active variant or fragment thereof is a vector with a nucleic acid sequence encoding the TIRA polypeptide or active fragment or variant thereof. One example of a DNA construct that may be used for expressing the TIRB polypeptide or active variant or fragment thereof is a vector with a nucleic acid sequence encoding the TIRB polypeptide or active fragment or variant thereof. One example of a DNA construct that may be used for expressing the TIRATIRB fusion polypeptide or active variant or fragment thereof is a vector with a nucleic acid sequence encoding each of the TIRA and TIRB polypeptides or active fragment or variant of either.

In embodiments, a DNA construct comprising a polynucleotide encoding the TIRA polypeptide of SEQ ID NO: 1, or an active variant or fragment thereof, comprises one or more of the native introns of a genomic sequence (SEQ ID NO: 3) encoding the TIRA polypeptide. As non-limiting examples, the DNA construct may comprise at least 1 or all native introns of the genomic sequence encoding the TIRA polypeptide. In particular embodiments, a DNA construct comprising a polynucleotide encoding the TIRA polypeptide of SEQ ID NO: 1, or an active variant or fragment thereof, comprises one or more of (i) a first native intron (intron_1, position 8021 to 8369 of SEQ ID NO: 71 or position 1231 to 1579 of SEQ ID NO: 72, and (ii) a second native intron (intron_2, position 8883 to 9159 of SEQ ID NO: 71 or position 2093 to 2369 of SEQ ID NO: 72. In still further embodiments, one or more of the native introns may be replaced with other introns. Further still, the DNA construct comprising one or more native introns of the genomic sequence encoding the TIRA polypeptide may comprise one or more additional introns derived from other sources that enhance expression of the polypeptide of interest.

In embodiments, a DNA construct comprising a polynucleotide encoding the TIRB polypeptide of SEQ ID NO: 2, or an active variant or fragment thereof, comprises one or more of the native introns of a genomic sequence (SEQ ID NO: 6) encoding the TIRA polypeptide. As non-limiting examples, the DNA construct may comprise at least 1 or all native introns of the genomic sequence encoding the TIRB polypeptide. In particular embodiments, a DNA construct comprising a polynucleotide encoding the TIRB polypeptide of SEQ ID NO: 2 or 17, or an active variant or fragment thereof, comprises one or more of (i) a first native intron (intron_1, position 8021 to 8369 of SEQ ID NO: 71 or position 1231 to 1579 of SEQ ID NO: 72, and (ii) a second native intron (intron_2, position 8883 to 9159 of SEQ ID NO: 71 or position 2093 to 2369 of SEQ ID NO: 72). In still further embodiments, one or more of the native introns may be replaced with other introns. Further still, the DNA construct comprising one or more native introns of the genomic sequence encoding the TIRB polypeptide may comprise one or more additional introns derived from other sources that enhance expression of the polypeptide of interest.

In some embodiments, a vector may comprise multiple expression cassettes, such as where co-expression of the TIRA polypeptide and TIRB polypeptide, or active fragments or variants thereof are desired. One example of a DNA construct that may be used for co-expressing the TIRA polypeptide or active variant or fragment thereof with the TIRB polypeptide or active variant or fragment thereof is a vector with a nucleic acid sequence comprising (i) a first polynucleotide encoding the TIRA polypeptide or active fragment or variant thereof; and (ii) a second polynucleotide encoding the TIRB polynucleotide or active fragment or variant thereof. In embodiments, the nucleic acid sequence is operably linked to a heterologous regulatory element. In one particular example, the vector comprises a first expression cassette comprising the first polynucleotide encoding the TIRA polypeptide or active fragment or variant thereof operably coupled to a first heterologous regulatory element, such as a first plant active promoter driving expression of the TIRA polypeptide or active fragment or variant thereof in a plant, plant part or seed, the vector further comprising a second expression cassette comprising the second polynucleotide encoding the TIRB polypeptide or active fragment or variant thereof operably coupled to a second, different heterologous regulatory element, such as a second, different plant active promoter driving co-expression of the TIRB polypeptide or active fragment or variant thereof in the plant, plant part or seed.

In another example embodiment, the vector comprises an expression cassette comprising a polynucleotide having the nucleotide sequence of a genomic locus comprising each of the TirA and TirB genes and encoding the TIRA and TIRB polypeptides coupled to a common heterologous regulatory element, such as a single native plant active promoter driving co-expression of the TIRA and TIRB polypeptides. In yet another embodiment, the vector comprises an expression cassette comprising a polynucleotide having the genomic or endogenous sequence of the TirA gene encoding the TIRA polypeptide and a polynucleotide having the genomic or endogenous sequence of the TirB gene encoding the TIRB polypeptide driven by a common bi-directional native promoter. In a particular example embodiment, a polynucleotide having the genomic sequence of the TirA gene (SEQ ID NO: 3) and a polynucleotide having the genomic sequence of the TirB gene (SEQ ID NO: 6) is driven by a common bi-directional native promoter (SEQ ID NO: 49) derived from the genomic locus of the Rg32 and Rg34 genes.

One example of a DNA construct that may be used for expressing the TIRATIRB fusion protein or active variant or fragment thereof is a vector with a nucleic acid sequence encoding the TIRATIRB fusion protein or active fragment or variant thereof. One example of a DNA construct that may be used for expressing the TIRATIRB fusion protein or active variant or fragment thereof is a vector with a nucleic acid sequence comprising (i) a first polynucleotide encoding the TIRA polypeptide or active fragment or variant thereof; and (ii) a second polynucleotide encoding the TIRB polynucleotide or active fragment or variant thereof, wherein the nucleic acid sequence is operably linked to a common heterologous regulatory element. In one particular example, the vector comprises an expression cassette comprising the first polynucleotide encoding the TIRA polypeptide or active fragment or variant thereof and the second polynucleotide encoding the TIRA polypeptide or active fragment or variant thereof, each operably coupled to a common heterologous regulatory element, such as a single plant active promoter driving expression of both polypeptides fused together as a single fusion protein. In another particular example, the vector comprises an expression cassette comprising a polynucleotide comprising a nucleotide sequence of the genomic locus comprising each of the TirA and TIrB genes and encoding the TIRA and TIRB polypeptides coupled to a common heterologous regulatory element, such as a single native plant active promoter driving expression of both polypeptides as a fusion protein (such as the bidirectional promoter of SEQ ID NO: 49).

Expression of the DNA constructs, expression cassettes and vectors of the present disclosure in a plant, plant part or seed confers the plant with disease resistance. In particular embodiments, co-expression of a TIRA polypeptide (or active fragments and variants thereof) and a TIRB polypeptide (or active variants and fragments thereof) via expression of a nucleic acid molecule comprising a nucleotide sequence comprising the first polynucleotide encoding the TIRA polypeptide and the second polynucleotide encoding the TIRB polypeptide confers a legume plant, plant part or seed (e.g., soybean plant, plant part or seed) with disease resistance (e.g., fungal pathogen resistance, ASR resistance, powdery mildew resistance, nematode resistance, SCN resistance, RKN resistance, bacterial pathogen resistance, P. syringae resistance, sucking pest resistance, aphid resistance, stink bug resistance, and/or whitefly resistance).

The translation leader sequence means a DNA molecule located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders, plant virus coat protein leaders, plant rubisco gene leaders among others (Turner and Foster, Molecular Biotechnology 3:225, 1995).

The “3′ non-translated sequences” (or 3′ untranslated sequences or 3′-UTR) means DNA sequences located downstream of a structural polynucleotide sequence and include sequences encoding polyadenylation and other regulatory signals capable of affecting mRNA processing or gene expression. The polyadenylation signal functions in plants to cause the addition of multiple adenylate nucleotides to the 3′ end of the mRNA precursor. The polyadenylation sequence can be derived from the natural gene, from a variety of plant genes, or from T-DNA.

An example of the polyadenylation sequence is the nopaline synthase 3′ sequence (nos 3′; Fraley et al., Proc. Natl. Acad. Sci. USA 80:4803-4807, 1983). The use of different 3′ non-translated sequences is exemplified by Ingelbrecht et al., Plant Cell 1:671-680, 1989.

In particular embodiments, the 3′-UTR of the TirA gene may be included, wherein the 3′-UTR of the TirA gene is derived from the genomic sequence of SEQ ID NO: 3 and may include at least a 500 bp, 1000 bp or 2000 bp region lying immediately downstream of the stop codon. In other particular embodiments, the 3′-UTR of the TirB gene may be included wherein the 3′-UTR of the TirB gene is derived from the genomic sequence of SEQ ID NO: 5 and may include at least a 500 bp, 100 bp or 2000 bp region lying immediately downstream of the stop codon.

In specific embodiments, the various 3′-UTRs disclosed in WO2019103918, WO2021000878, WO2021022022, WO2022173659, WO2021260673, WO2021263249, or U.S. Provisional Applications 63/481,627, 63/426,524, 63/509586, or 63/383609 may be used; each of which is herein incorporated by reference in its entirety, including for example, those disclosed in WO2022173659 as SEQ ID NO: 20.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used. In particular embodiments, the native terminator of the TirA gene (tGcaRG3a; SEQ ID NO: 28) and/or the native terminator of the TirB gene (tGcaRG3b; SEQ ID NO: 29) may be used. Termination regions used in the expression cassettes can be obtained from, e.g., the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In specific embodiments, the various terminators disclosed in WO2019103918, WO2021000878, WO2021022022, WO2022173659, WO2021260673, WO202163249, or U.S. Provisional Applications 63/481,627, 63/426,524 or 63/509586 may be used, including for example, those disclosed in WO2019103918 including SEQ ID NO: 8 (RG1 terminator), SEQ ID NO: 11, SEQ ID NO: 14, SEQ ID NO: 17, SEQ ID NO: 20, SEQ ID NO: 23, SEQ ID NO: 26, SEQ ID NO: 29, or SEQ ID NO: 32; those disclosed in WO2022173659 including SEQ ID NO: 18 (RG30 terminator); or those disclosed in WO2021022022 as SEQ ID NO: 9 (TirA terminator) or SEQ ID NO: 15 (TirB terminator), those disclosed in U.S. Provisional Application No. 63/481,627 as SEQ ID NO: 9 (RG31 terminator) or SEQ ID NO: 11 (RG35 terminator); or those disclosed in U.S. provisional Application Nos. 63/426,524 and 63/509,586 as SEQ ID NO: 9 (RG32 terminator) or SEQ ID NO: 12 or 13 (RG34 terminator); each of which is herein incorporated by reference in its entirety. In particular embodiments, the native terminator of the TirA gene (tGcaRG3a; SEQ ID NO: 28) or the native terminator of the TirB gene (tGcaRG3b; SEQ ID NO: 29) may be used.

The “5′ non-translated sequences” (or 5′ untranslated sequences or 5′-UTR) means DNA sequences located upstream of an initiation codon of structural polynucleotide sequence and include sequences capable of affecting translation of an mRNA sequence. The 5′-UTR sequence is also referred to as a leader sequence. In different organisms, the 5′-UTR may remain untranslated, and form complex secondary structures to regulate translation of the downstream sequence. The leader sequence can be derived from the natural gene or from a variety of plant genes. In particular embodiments, the 5′-UTR of the TirA gene may be included, wherein the 5′-UTR of the TirA gene is derived from the genomic sequence of SEQ ID NO: 3 and may include at least a 500 bp, 1000 bp, or 2000 bp region lying immediately upstream of the start codon or a 500 bp region lying immediately downstream from a transcription start site. In other particular embodiments, the 5′-UTR of the TirB gene may be included wherein the 5′-UTR of the TirB gene is derived from the genomic sequence of SEQ ID NO: 6 and may include at least a 500 bp, 1000 bp, or 2000 bp region lying immediately upstream of the start codon or a 500 bp region lying immediately downstream from a transcription start site.

In specific embodiments, the various 5′-UTRs disclosed in WO2019103918, WO2021000878, WO2021022022, WO2022173659, WO2021260673, WO2021263249, or U.S. Provisional Applications 63/481,627, 63/426,524, 63/509586, or 63/383609 may be used; each of which is herein incorporated by reference in its entirety, including for example, those disclosed in WO2022173659 as SEQ ID NO: 19.

A number of non-translated leader sequences derived from viruses are also known to enhance expression, and these are particularly effective in dicotyledonous cells. The expression cassette may comprise one or more of such leader sequences. Specifically, leader sequences from tobacco mosaic virus (TMV, the “W-sequence”), maize chlorotic mottle virus (MCMV), and alfalfa mosaic virus (AMV) have been shown to be effective in enhancing expression (e.g., Gallie et al. Nucl. Acids Res. 15:8693-8711 (1987); Skuzeski et al. Plant Molec. Biol. 15:65-79 (1990)). Other leader sequences known in the art include but are not limited to: picomavirus leaders, for example, EMCV leader (encephalomyocarditis 5′ noncoding region) (Elroy-Stein, O., Fuerst, T. R., and Moss, B. PNAS USA 86:6126-6130 (1989)); potyvirus leaders, for example, tobacco etch virus (TEV) leader (Allison et al., 1986); maize dwarf mosaic virus (MDMV) leader; Virology 154:9-20); human immunoglobulin heavy-chain binding protein (BiP) leader, (Macejak, D. G., and Samow, P., Nature 353:90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, 237-256 (1989); and maize chlorotic mottle virus leader (MCMV) (Lommel, S. A. et al., Virology 81:382-385 (1991). See also, Della-Cioppa et al., Plant Physiology 84:965-968 (1987).

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al, eds. (1980).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin, 5-enolpyruvylshikimate-3-phosphate synthase (EPSPS) or acetolactate synthase (ALS). Selection markers used routinely in transformation include the nptll gene, which confers resistance to kanamycin and related antibiotics (Messing & Vierra Gene 19:259-268 (1982); Bevan et al., Nature 304:184-187 (1983)), the pat and bar genes, which confer resistance to the herbicide glufosinate (also called phosphinothricin; see White et al., Nucl. Acids Res 18:1062 (1990), Spencer et al. Theor. Appl. Genet 79:625-631 (1990) and U.S. Pat. Nos. 5,561,236 and 5,276,268), the hph gene, which confers resistance to the antibiotic hygromycin (Blochinger & Diggelmann, Mol. Cell Biol. 4:2929-2931), and the dhfr gene, which confers resistance to methatrexate (Bourouis et al., EMBO J. 2 (7): 1099-1104 (1983)), the EPSPS gene, which confers resistance to glyphosate (U.S. Pat. Nos. 4,940,935 and 5,188,642), the glyphosate N-acetyltransferase (GAT) gene, which also confers resistance to glyphosate (Castle et al. (2004) Science, 304:1151-1154; U.S. Patent App. Pub. Nos. 20070004912, 20050246798, and 20050060767); and the mannose-6-phosphate isomerase gene, which provides the ability to metabolize mannose (U.S. Pat. Nos. 5,767,378 and 5,994,629).

### A. Promoters

A number of promoters can be used in the various methods and compositions disclosed herein. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters can be used. Non-limiting examples of constitutive promoters include CaMV 35S promoter (Odell et al. (985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten e/a/. (1984) EMBO J. 3:2723-2730). Inducible promoters include those that drive expression of pathogenesis-related proteins (PR proteins), which are induced following infection by a pathogen. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116; and WO 99/43819, herein incorporated by reference. Promoters that are expressed locally at or near the site of pathogen infection may also be used (Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977; Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; Cordero et al. (1992) Physiol. Mol. Plant Path. 41:189-200; U.S. Pat. No. 5,750,386 (nematode-inducible); and the references cited therein).

In particular embodiments, a constitutive promoter used for expression of a polypeptide of the invention in a plant comprises a soybean ubiquitin promoter (prGmUbi1), a CMV 35S promoter (pr35S), a promoter of a Ubiquitin gene from Glycine argyrea accession PI_599400 contig 000104F (prGaUbiPI599400), a promoter of a Ubiquitin gene from Arabidopsis thaliana (prUBQ3) or a Medicago truncatula promoter (prMt12344 (SEQ ID NO: 22) or prMt51186 (SEQ ID NO: 23) or prMt15303 (SEQ ID NO: 24)) can be used.

In specific embodiments, the various constitutive promoters disclosed in WO2019103918, WO2021000878, WO2021022022, WO2022173659, WO2021260673, WO2021263249, or U.S. Provisional Applications 63/481,627, 63/426,524, 63/509586, or 63/383609 may be used; each of which is herein incorporated by reference in its entirety.

Wound-inducible promoters may be used in the constructions of the invention. Such wound-inducible promoters include pin II promoter (Ryan (1990) Ann. Rev. Phytopath. 28:425-449; Ouan et al. (1996) Nature Biotechnology 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2): 141-150); and the like, herein incorporated by reference).

Still other inducible promoters may be used for the expression of a polypeptide of the invention in the constructs of the invention. In embodiments, the inducible promoter is a rust-reactive or rust-inducible promoter. As used herein, a “rust-inducible promoter” is a plant promoter that is induced or activated in response to rust exposure or rust infection of the plant. In particular embodiments, a rust-reactive or rust-inducible promoter (e.g., prLuFIS1; SEQ ID NO: 25) of the Fis1 gene from flax (Linum usitatissimum) may be used, such as disclosed in WO2021022022 as SEQ ID NO: 22 and in U.S. Provisional App. No. 63/383,609 as SEQ ID NO: 25, the contents of which are incorporated by reference herein in their entirety. In other specific embodiments, a rust reactive promoter (e.g., prGmACO3) derived from the ACO3 gene (Glyma.02G268200) from soybean (Glycine max) may be used, such as disclosed in U.S. Provisional App. No. 63/481,627 as SEQ ID NO: 14; or a rust reactive promoter (e.g., prGmMYB) of a MYB gene (Glyma. 19G164600) from soybean (Glycine max) may be used, such as disclosed in U.S. Provisional App. No. 63/481,627 as SEQ ID NO: 15, the contents of which are incorporated by reference herein in their entirety.

Tissue-preferred promoters for use in the invention include those set forth in Yamamoto et al. (1997) Plant J. 12 (2): 255-265; Kawamata et al. (1997) Plant Cell Physiol. 38 (7): 792-803; Hansen et al. (1997) Mol. Gen Genet. 254 (3): 337-343; Russell et al. (1997) Transgenic Res. 6 (2): 157-168; Rinehart et al. (1996) Plant Physiol. 112 (3): 1331-1341; Van Camp et al. (1996) Plant Physiol. 112 (2): 525-535; Canevascim et al. (1996) Plant Physiol. 112 (2): 513-524; Yamamoto et al. (1994) Plant Cell Physiol. 35 (5): 773-778; Lam (1994) Results Probl. Cell Differ. 20:181-196; Orozco et al. (1993) PlantMolBiol. 23 (6): 1129-1138; Matsuoka et al. (1993) Proc Natl. Acad. Sci. USA 90 (20): 9586-9590; and Guevara-Garcia et al. (1993) Plant J. 4 (3): 495-505.

Leaf-preferred promoters include those set forth in Yamamoto et al. (1997) Plant J. 12 (2): 255-265; Kwon et al. (1994) Plant Physiol. 105:357-67; Yamamoto et al. (1994) Plant Cell Physiol. 35 (5): 773-778; Gotor et al. (1993) Plant J. 3:509-18; Orozco et al. (1993) Plant Mol. Biol. 23 (6): 1129-1138; and Matsuoka et al. (1993) Proc. Natl. Acad. Sci. USA 90 (20): 9586-9590.

Root-preferred promoters are known and include those set forth in Hire et al. (1992) Plant Mol. Biol. 20 (2): 207-218 (soybean root-specific glutamine synthetase gene); Keller and Baumgartner (1991) Plant Cell 3 (10): 1051-1061 (root-specific control element); Sanger et al. (1990) Plant Mol. Biol. 14 (3): 433-443 (mannopine synthase (MAS) gene of Agrobacterium tumefaciens); and Miao et al. (1991) Plant Cell 3 (1): 11-22 (cytosolic glutamine synthetase (GS)); Bogusz et al. (1990) Plant Cell 2 (7): 633-641; Leach and Aoyagi (1991) Plant Science (Limerick) 79 (1): 69-76 (rolC and rolD); Teeri et al. (1989) EMBO J. 8 (2): 343-350; Kuster et al. (1995) Plant Mol. Biol. 29 (4): 759-772 (the VfENOD-GRP3 gene promoter); and, Capana et al. (1994) Plant Mol. Biol. 25 (4): 681-691 (rolB promoter). See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (promoters active during seed development such as promoters of seed storage proteins) as well as “seed-germinating” promoters (promoters active during seed germination). See Thompson et al. (1989) BioEssays 10:108. Seed-preferred promoters include, but are not limited to, Ciml (cytokinin-induced message); cZ19B1 (maize 19 kDa zein); milps (myo-inositol-1-phosphate synthase) (see WO 00/11177 and U.S. Pat. No. 6,225,529). Gamma-zein is an endosperm-specific promoter. Globulin 1 (Gib-1) is a representative embryo-specific promoter. For dicots, seed-specific promoters include, but are not limited to, bean β-phaseolin, napin, beta-conglycinin, soybean lectin, cruciferin, and the like. For Monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed.

In some embodiments, promoters that control expression of resistance genes can be used to express the polynucleotide of interest. Such promoters include, but are not limited to the various native R-gene promoters set forth in WO2019103918, WO2021000878, WO2021022022, WO2022173659, WO2021260673, WO2021263249, or U.S. Provisional Applications 63/481,627, 63/426,524, 63/509586, or 63/383609; each of which is herein incorporated by reference in its entirety, including those disclosed in WO2019103918 as SEQ ID NO: 7 (RG1 promoter), SEQ ID NO: 10, SEQ ID NO: 13, SEQ ID NO: 16, SEQ ID NO: 19, SEQ ID NO: 22, SEQ ID NO: 25, SEQ ID NO: 28, or SEQ ID NO: 31; in WO2021000878 as SEQ ID NO: 7 (Rpp 6907 promoter); in WO2021022022 as SEQ ID NO: 9 (TirA promoter) or SEQ ID NO: 14 (TirB promoter); in WO2022173659 as SEQ ID NO: 15 (RG30 promoter); in WO2021263249 or WO2021260673 as SEQ ID NO: 7 (RG21 promoter); and those disclosed in U.S. Provisional Application 63/481,627 as SEQ ID NO: 7-8 (RG31 promoter) or SEQ ID NO: 10 (RG35 promoter), or in U.S. Provisional Application 63/426,524 and 63/509,586 as SEQ ID NO: 8 (RG32 promoter) or SEQ ID NO: 10 or 11 (RG34 promoter). Still other native promoters include the native TirA promoter (prGcaRG3a as set forth herein at SEQ ID NO: 26) and the native TirB promoter (prGcaRG3b as set forth herein at SEQ ID NO: 27), as well as a modified version of the native promoters.

In further embodiments, the native promoter may be a promoter sequence derived from a genomic locus comprising each of the Rg32 and Rg34 genes, as disclosed in U.S. Provisional Applications 63/426,524 and 63/509,586 as SEQ ID NO: 18. In a particular example embodiment, the native promoter derived from the genomic locus comprises the sequence as set forth herein as SEQ ID NO: 49, which drives bidirectional expression of genes operably coupled thereto (such as the bidirectional expression of the Rg32 and Rg34 genes; or bidirectional expression of the TirA and TirB genes). As elaborated at Example 9, bidirectional expression of the TIR polypeptides is obtained when expression is driven of the Tir genes using the sense strand and the reverse complement of the bidirectional promoter sequence of SEQ ID NO: 49.

For expression in a bacterial host, promoters that function in bacteria are known in the art. Such promoters include any of the known crystal protein gene promoters, including the promoters of any of the proteins of the invention, and promoters specific for B. thuringiensis sigma factors. Alternatively, mutagenized or recombinant crystal protein-encoding gene promoters may be recombinantly engineered and used to promote expression of the novel gene segments disclosed herein.

### B. Native Regulatory Elements

Compositions comprising novel regulatory elements are provided. In one embodiment, a polynucleotide comprising a regulatory element operably linked to a polynucleotide of interest is provided. Such regulatory elements include promoters and comprise the nucleotide sequence set forth in SEQ ID NO: 26 or 27 or an active variant or fragment thereof. An active variant or fragment of the promoter will retain the ability to direct expression of the operably linked polynucleotide sequence. As such, active variants of the promoter sequence comprise at least 80%, 85%, 90%, 91%, 92%, 93%, 94%, 95%, 96%, 97%, 98% or 99% identity to SEQ ID NO: 26 or 27 and retain the ability to direct expression of an operably linked nucleotide sequence. Fragments of such promoter sequences are also provided and can comprise at least 100, 200, 250, 300, 350, 400 or more nucleotides of the sequence set forth in SEQ ID NO: 26 or 27. Fragments of such promoters can be active fragment and retain the ability to direct expression of an operably linked nucleotide sequence.

Regulatory elements comprising terminator sequences set forth in SEQ ID NO: 28 or 29 or an active variant or fragment thereof are provided. An active variant or fragment of the terminator sequence will retain the ability to regulate the expression of an operably linked polynucleotide sequence. As such, active variants of the terminator sequence comprise at least 80%, 85%, 90%, 91%, 92%, 93%, 94%, 95%, 96%, 97%, 98% or 99% identity to SEQ ID NO: 28 or 29 and retain the ability to direct expression of an operably linked nucleotide sequence. Fragments of such terminator sequences are also provided and can comprise at least 100, 200, 250, 300, 350, 400, 450, 500, 550, 600, 650, 700, 750, 800, 850, 900, 950, 1000 or more nucleotides of the sequence set forth in SEQ ID NO: 28 or 29. Fragments of such terminators can be active fragment and retain the ability to regulate expression of an operably linked nucleotide sequence.

Regulatory elements comprising intron sequences as set forth at position 8021 to 8369 (intron_1) of SEQ ID NO: 71; at position 8883 to 9159 (intron_2) of SEQ ID NO: 71; position 1231 to 1579 (intron_1) of SEQ ID NO: 72; and/or at position 2093 to 2369 (intron_2) of SEQ ID NO: 72; or an active variant or fragment thereof are provided. An active variant or fragment of the intron sequence will retain the ability to regulate the expression of an operably linked polynucleotide sequence. As such, active variants of the intron sequence comprise at least 80%, 85%, 90%, 91%, 92%, 93%, 94%, 95%, 96%, 97%, 98% or 99% identity to the above-listed intron sequences and retain the ability to direct expression of an operably linked nucleotide sequence. Fragments of such introns sequences are also provided and can comprise at least 100, 200, 250, 300, 350, 400 or more nucleotides of the sequence set forth as any of the following: position 8021 to 8369 (intron_1) of SEQ ID NO: 71; position 8883 to 9159 (intron_2) of SEQ ID NO: 71; position 1231 to 1579 (intron_1) of SEQ ID NO: 72 and position 2093 to 2369 (intron_2) of SEQ ID NO: 72. Fragments of such introns can be an active fragment and retain the ability to regulate expression of an operably linked nucleotide sequence.

In some aspects, the disclosure provides an expression cassette. In some embodiments, the expression cassette comprises a nucleotide sequence comprising any one of SEQ ID NOS: 3-5 and/or any one of SEQ ID NOS: 6-8 wherein the nucleotide sequence is operably linked to a heterologous nucleotide sequence. In some embodiments, the expression cassette further comprises a selectable marker.

In some embodiments, the expression cassette is an endogenous expression cassette and comprises a nucleotide sequence comprising any one of SEQ ID NOS: 73-74.

In some embodiments, the heterologous sequence of interest is a nucleic acid of interest that encodes an RNA or protein of interest. In some embodiments, the RNA or protein of interest is capable of conferring upon a plant a desired characteristic such as antibiotic resistance, virus resistance, insect resistance, disease resistance, resistance to other pests, herbicide tolerance, improved nutritional value, improved performance in an industrial process or altered reproductive capability. In some embodiments, the RNA or protein of interest comprises a genome editing agent, e.g., a CRISPR/Cas agent (such as a Cas protein and/or guide RNA), a TALEN, a DNA-guided nuclease, a meganuclease, a recombinase, or a zinc finger nuclease. In some embodiments, the heterologous nucleotide sequence encodes a selectable marker.

The heterologous nucleotide sequence of interest can comprise a sequence encoding a polypeptide of interest and in more specific embodiments, the heterologous nucleotide sequence of interest encodes a protein that increases disease resistance (e.g., increases fungal pathogen resistance such as ASR resistance or powdery mildew resistance, increases nematode pathogen resistance, increases bacterial pathogen resistance, and/or sucking pest resistance) in plant. Such sequences include but are not limited to polynucleotides encoding proteins that confer increased ASR resistance as described in US Patent publication Nos. US20200354739, and PCT Publications Nos. WO2019103918, WO2021000878, WO2021154632A1, WO2021022022, WO2021022026, WO2021022101, WO2022173659, WO2021260673, WO2021263249, and U.S. Provisional Applications 63/481,627, 63/426,524, 63/509586, and 63/383609, each of which is incorporated by reference in its entirety.

In some embodiments, the expression cassette in a vector, such as a plasmid, virus, or Agrobacterium. In some embodiments, the expression cassette is in a plant cell as discussed elsewhere herein.

### 4. Plants, Plant Cells and Plant Parts

Plants, plant parts, plant cells and seed are provided which comprise in their genome a nucleic acid sequence operably linked to a promoter active in the plant, wherein the nucleic acid sequence comprises polynucleotides encoding (i) a TIRA polypeptide as set forth in SEQ ID NO: 1 or an active variant or fragment thereof and (ii) a TIRB polypeptide as set forth in SEQ ID NO: 2 or an active variant or fragment thereof. In particular embodiments, the plants, plant parts, plant cells and seed co-express in their genome, the TIRA and TIRB polypeptides, or their active variants and fragments. In other particular embodiments, the plants, plant parts, plant cells or seeds express in their genome a nucleic acid sequence operably linked to a promoter active in the plant, wherein the nucleic acid sequence comprises polynucleotides encoding the TIRA and TIRB polynucleotides as a TIRATIRB fusion protein as set forth in any one of SEQ ID NOS: 9-12, or an active variant or fragment of the fusion protein. In still further embodiments, plants, plant parts, plant cells and seed are provided which comprise in their genome a heterologous nucleic acid sequence comprising polynucleotides set forth in any one of SEQ ID NOS: 3-5, 6-8, 13-19 and 21, or active variants and fragments thereof. Such heterologous polynucleotides can be transiently expressed or stably integrated into the genome.

Although soybean plants are used to exemplify the composition and methods throughout the application, a polynucleotide as provided herein may be introduced to any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

In specific embodiments, the plant is a legume. Examples of legumes include, but are not limited to, the genus Phaseolus (e.g., French bean, dwarf bean, climbing bean (Phaseolus vulgaris), Lima bean (Phaseolus lunatus), Tepary bean (Phaseolus acutifolius), runner bean (Phaseolus coccineus)); the genus Glycine (e.g., Glycine soja, soybeans (Glycine max (L.))); pea (Pisum) (e.g., shelling peas (sometime called smooth or round-seeded peas; Pisum sativum); marrowfat pea (Pisum sativum), sugar pea (Pisum sativum), also called snow pea, edible-podded pea or mangetout (Pisum granda); peanut (Arachis hypogaea), clover (Trifolium spp.), medick (Medicago), kudzu vine (Pueraria lobata), common lucerne, alfalfa (Medicago sativa), chickpea (Cicer), lentils (Lens culinaris), lupins (Lupinus); vetches (Vicia), field bean, broad bean (Vicia faba), vetchling (Lathyrus) (e.g., chickling pea (Lathyrus sativus), heath pea (Lathyrus tuberosus)); genus Vigna (e.g., moth bean (Vigna aconiti folia), adzuki bean (Vigna angularis), urad bean (Vigna mungo), mung bean (Vigna radiata), bambara groundnut (Vigna subterrane), rice bean (Vigna umbellata), Vigna vexillata, Vigna unguiculata (also known as asparagus bean, cowpea); pigeon pea (Cajanus cajari; Cajanus cajan), the genus Macrotyloma (e.g., geocarpa groundnut (Macrotyloma geocarpum), horse bean (Macrotyloma uniflorum); goa bean (Psophocarpus tetragonolobus), African yam bean (Sphenostylis stenocarpa), Egyptian black bean, lablab bean (Lablab purpureus), yam bean (Pachyrhizus erosus), guar bean (Cyamopsis tetragonolobus); and/or the genus Canavalia (e.g., jack bean (Canavalia ensiformis)), sword bean (Canavalia gladiata).

In one embodiment, the legume plant is soybean, and more particularly Glycine max.

Glycine (soybean or soya bean) is a genus in the bean family Fabaceae. The Glycine plants provided herein can be Glycine arenaria, Glycine argyrea, Glycine cyrtoloba, Glycine canescens, Glycine clandestine, Glycine curvata, Glycinefalcata, Glycine latifolia, Glycine microphylla, Glycine pescadrensis, Glycine stenophita, Glycine syndetica, Glycine soja Seib. Et Zucc., Glycine max (L.) Merrill., Glycine tabacina, or Glycine tomentella.

In some embodiments, the plants provided herein (legumes or soybeans plants) are elite plants, elite germplasm or are derived from an elite line or an elite germplasm. Numerous elite lines are available and known to those of skill in the art of soybean breeding and are discussed in further detail elsewhere herein.

In some embodiments, the plants provided herein can comprise one or more additional polynucleotides that encode an additional polypeptide that increases disease resistance of the plant. Such combinations are described in further detail elsewhere herein.

In specific embodiments, the plants, plant parts or seeds having the heterologous polynucleotide or polypeptide disclosed herein or active variants and fragments thereof can have an increased expression of the polynucleotide or polypeptide. In other embodiments, the plants, plant parts or seeds having the heterologous polynucleotide or polypeptide disclosed herein or active variants and fragments thereof can have an increased level of activity of the polypeptide. Methods to generate such increased levels of expression or activity are disclosed elsewhere herein and include, but are not limited to, breeding, gene editing, and transgenic techniques.

Plants produced as described above can be propagated to produce progeny plants, and the progeny plants that have stably incorporated into its genome a polynucleotide conferring the increased protein content and/or altered seed composition can be selected and can be further propagated if desired.

In some embodiments, a plant cell, seed, or plant part or harvest product can be obtained from the plant produced as above and the plant cell, seed, or plant part can be screened using methods disclosed above for the evidence of stable incorporation of the polynucleotide. The term “stable incorporation” refers to the integration of a nucleic acid sequence into the genome of a plant and the nucleic acid sequence is capable of being inherited by the progeny thereof.

In some embodiments, plant products can be harvested from the plant disclosed above and processed to produce processed products, such as flour, soy meal, oil, starch, and the like. These processed products are also within the scope of this invention provided that they comprise a polynucleotide or polypeptide or variant or fragment thereof disclosed herein. Other soybean plant products include but are not limited to protein concentrate, protein isolate, soybean hulls, meal, flower, oil and the whole soybean itself.

Seed lots are provided comprising populations of seeds that comprise in their genome a heterologous nucleic acid sequence comprising polynucleotides encoding a TIRA polypeptide set forth in SEQ ID NO: 1, or an active variant or fragment thereof, and a TIRB polypeptide set forth in SEQ ID NO: 2, or active variant or fragment thereof (e.g., polynucleotides encoding the TIRA polypeptide co-expressed with the TIRB polypeptide); or encoding a TIRBTIRB fusion protein as set forth in any one of SEQ ID NOS: 9-12; and having an increased disease resistance. In other embodiments, the seed lots comprises populations of seed, which comprise in their genome a heterologous nucleic acid sequence comprising polynucleotides set forth in any one of SEQ ID NOS: 8-13 and 15-22 or variants and fragments thereof.

Such seed can be from any plant, including but not limited to dicotyledonous crop plants, legumes, or soybean. Methods of making a seed lot comprise harvesting seed from a plant having the increase resistance to a plant pathogen. Such seed lots can comprise at least 50, 100, 1000, 100000 seeds or more of the invention.

Further provided is an ensemble of plants that produce seeds having an increased resistance to a plant pathogen as described herein. Such an ensemble of plants have stably integrated into their genomes a heterologous nucleic acid sequence comprising polynucleotides encoding (i) a TIRA polypeptide as set forth in SEQ ID NO: 1 or an active variant or fragment thereof, and (ii) a TIRB polypeptide as set forth in SEQ ID NO: 2 or an active variant or fragment thereof and have an increased disease resistance. In other embodiments, the ensemble of plants have stably integrated into their genomes a heterologous nucleic acid sequence comprising polynucleotides encoding a fusion protein comprising (i) a TIRA polypeptide as set forth in SEQ ID NO: 1 or an active variant or fragment thereof, and (ii) a TIRB polypeptide as set forth in SEQ ID NO: 2 or an active variant or fragment thereof and have an increased disease resistance, such as the fusion protein of any of SEQ ID NOS: 9-12 or an active variant or fragment thereof. In other embodiments, the seed lots comprises populations of seed, which comprise in their genome a heterologous nucleic acid sequence comprising polynucleotides set forth in any one of SEQ ID NOS: 3-8, 13-19 and 21 or variants and fragments thereof. The term ensemble encompasses any collection of plants linked together by proximity, such as plants in a field, a greenhouse or a tray. The ensemble of plants comprises at least 50, 100, 1000, 10000, 100000 or more plants of the invention.

I. Plants with a Rebalanced Relative Expression Level of TIRA and TIRB Polypeptides

In embodiments, the plants, plant parts, plant cells and seed disclosed herein which comprise in their genome a polynucleotide encoding a TIRA polypeptide, co-expressed with a TIRB polypeptide (or active variant or fragments of either TIR polypeptide), or a fusion protein thereof, have an expression level of the TIRA polypeptide relative to the TIRB polypeptide modified as compared to the relative expression levels of the polypeptides in a control plant. In particular embodiments, plants, plant parts and seed which comprise, stably integrated in their genome, (i) a polynucleotide encoding a TIRA polypeptide as set forth in SEQ ID NO: 1, or an active variant or fragment thereof, and a polynucleotide encoding a TIRB polypeptide as set forth in SEQ ID NO: 2, or active variant or fragments thereof, or (ii) a polynucleotide encoding a fusion protein comprising the TIRA polypeptide as set forth in SEQ ID NO: 1, or an active variant or fragment thereof, and a polynucleotide encoding a TIRB polypeptide as set forth in SEQ ID NO: 2, (e.g., the fusion protein as set forth in any one of SEQ ID NOS: 9-12, or an active variant or fragment thereof, or the polynucleotide of any of SEQ ID NOS: 13-19 and 21), the polynucleotide coupled to a heterologous promoter, have an expression level of the TIRA polypeptide or active variant or fragment thereof, that is rebalanced relative to the expression level of the TIRB polypeptide, or active variant or fragment thereof, in the plant, plant part or seed, as compared to the relative expression levels of the polypeptides in a control plant.

As used herein, “expression level” or “relative expression level” refers to an amount of TIRA transcript (e.g., primary transcript, or mRNA) that is available for translation, or an amount of TIRA polypeptide resulting from the translation of the transcript, relative to an amount of TIRB transcript (e.g., primary transcript, or mRNA) that is available for translation, or an amount of TIRB polypeptide resulting from the translation of the transcript.

As used herein, “unbalanced expression level” refers to the presence, in a plant cell, of an excess amount of one TIR transcript or polypeptide, relative to the other TIR transcript or polypeptide. In example embodiments, the unbalanced expression level refers to an excess, in a plant cell, of an amount of a TIRA transcript (that is, a primary transcript or mRNA encoding a TIRA polypeptide, or active variant or fragment thereof) relative to a TIRB transcript (that is, a primary transcript or mRNA encoding a TIRB polypeptide, or active variant or fragment thereof). In other embodiments, the unbalanced expression level refers to an excess, in a plant cell, of an amount of TIRB transcript (that is, a primary transcript or mRNA encoding a TIRB polypeptide, or active variant or fragment thereof) relative to a TIRA transcript (that is, a primary transcript or mRNA encoding a TIRA polypeptide, or active variant or fragment thereof). In particular embodiments, the unbalanced expression level of the TIRB polypeptide relative to the TIRA polypeptide in a control plant, or plant cell, is in a ratio of 5:1 to 30:1, such as a ratio of 20:1.

As used herein, “rebalanced expression level” refers to the presence, in a plant cell, upon introduction of a polynucleotide of the invention encoding a TIRA polypeptide and a TIRB polypeptide, an amount of a TIRA transcript (that is, a primary transcript or mRNA encoding a TIRA polypeptide, or active variant or fragment thereof) that is comparable to the amount of a TIRB transcript (that is, a primary transcript or mRNA encoding a TIRB polypeptide, or active variant or fragment thereof). In particular embodiments, introduction of a polynucleotide of the invention encoding a TIRA polypeptide and a TIRB polypeptide results in an expression level of the TIRB polypeptide relative to the TIRA polypeptide that is in a ratio of 0.5:1 to 2:1, such as a ratio of 1:1 or 1.5:1. In other particular embodiments, introduction of a polynucleotide of the invention encoding a TIRA polypeptide and a TIRB polypeptide results in an expression level of the TIRA polypeptide relative to the TIRB polypeptide that is in a ratio of 0.5:1 to 2:1, such as a ratio of 1:1 or 1.5:1. In particular embodiments, introduction of a polynucleotide of the invention results in the transcript levels of the TIRB polypeptide being reduced from being ~20-fold higher than the TIRA polypeptide to being ~1.5-fold higher than the TIRA polypeptide.

Plants, plant parts, plant cells and seeds comprising an unbalanced expression level of TIRA polypeptide relative to TIRB polypeptide are able to increase disease resistance in the plant. However, in some embodiments, such plants may display reduced agronomic performance and/or adverse growth phenotypes, including but not limited to delayed germination, smaller stature, stunted growth, and premature death, as disclosed in the Examples herein. In comparison, embodiments of plants, plant parts, plant cells and seeds comprising a rebalanced expression level of TIRA polypeptide relative to TIRB polypeptide show a reduction in negative growth phenotypes, including but not limited to improved germination, improved stature, and higher vigor, and/or improved agronomic performance, as disclosed in the Examples herein.

In embodiments, a plant, plant part, plant cell or seed having a rebalanced expression level of the TIRA polypeptide relative to the TIRB polypeptide and improved agronomic performance and increased disease resistance has differential expression of the TIR polypeptides in the plant, plant part, plant cell or seed via distinct regulatory elements, such as distinct promoters and/or terminators. In example embodiments, rebalancing is achieved by driving constitutive expression of the TIRA polypeptide and/or the TIRB polypeptide via a native promoter and optionally a native terminator. In a particular embodiment, a plant, plant part, plant cell or seed having a rebalanced expression level has constitutive expression of the TIRA polypeptide via a native promoter of the TIRA gene (prGcaRG3a; SEQ ID NO: 26) and optionally the corresponding native terminator (such as prGcaRG3a; SEQ ID NO: 28); and constitutive expression of the TIRB polypeptide via a native promoter of the TIRB gene (prGcaRG3b; SEQ ID NO: 27) and optionally the corresponding terminator (tGcaRG3b; SEQ ID NO: 29).

In another embodiment, a plant, plant part, plant cell or seed having a rebalanced expression level of the TIRA polypeptide relative to the TIRB polypeptide and improved agronomic performance and increased disease resistance has constitutive expression of the TIRA polypeptide driven in the plant, plant part, plant cell or seed via a constitutive plant active promoter and selective expression of the TIRB polypeptide driven in the plant, plant part, plant cell or seed via an inducible promoter. In a particular embodiment, constitutive expression of the TIRA polypeptide is driven by a constitutive plant-active promoter (e.g., prMt12344; SEQ ID NO: 22, or prMt51186; SEQ ID NO: 23; or prMt15303; SEQ ID NO: 24, all from Medicago truntula) while expression of the TIRB polypeptide is driven by an inducible promoter (e.g., rust-inducible promoter prLuFIS1; SEQ ID NO: 25) in the plant, plant part, plant cell or seed.

In other embodiments, a plant, plant part, plant cell or seed having a rebalanced expression level of the TIRA polypeptide relative to the TIRB polypeptide and improved agronomic performance and increased disease resistance comprises a nucleic acid having a nucleotide sequence expressing the TIRA polypeptide transcriptionally upstream of the TIRB polypeptide. In a particular embodiment, a plant, plant part, plant cell or seed having a rebalanced expression level of the TIR polypeptides comprises a polynucleotide comprising (i) a first expression cassette having a nucleotide sequence driving the expression of the TIRA polypeptide or active variant or fragment thereof via a first promoter and (ii) a second expression cassette having a nucleotide sequence driving the expression of the TIRB polypeptide or active variant or fragment thereof via a second, different promoter, wherein the first expression cassette is positioned transcriptionally upstream of the second expression cassette on the polynucleotide.

In yet another particular embodiments, a plant, plant part, plant cell or seed having a rebalanced expression level of the TIRA polypeptide relative to the TIRB polypeptide and improved agronomic performance and increased disease resistance comprises a nucleic acid having a nucleotide sequence expressing the TIRA polypeptide and a nucleotide sequence expressing the TIRB polypeptide each operably coupled to a bidirectional promoter. In a particular embodiment, the bidirectional promoter is the promoter of SEQ ID NO: 49. In a specific embodiment, the nucleotide sequence expressing the TIRA polypeptide is operably coupled upstream of the bidirectional promoter while the nucleotide sequence expressing the TIRB polypeptide is operably coupled downstream of the bidirectional promoter. As a result, expression of one TIR polypeptide is driven by the sense strand sequence of the bidirectional promoter while expression of the other TIR polypeptide is driven by the antisense or reverse complement sequence of the bidirectional promoter. In another specific embodiment, the nucleotide sequence expressing the TIRB polypeptide is operably coupled upstream of the bidirectional promoter while the nucleotide sequence expressing the TIRA polypeptide is operably coupled downstream of the bidirectional promoter.

In still other embodiments, rebalancing of the expression level of the TIRA polypeptide relative to the TIRB polypeptide in a plant, plant part, plant cell or seed and improved agronomic performance and increased disease resistance is achieved by expressing the TIRA polypeptide as a fusion protein with the TIRB polypeptide (e.g., with the TIRA polypeptide at the N-terminus or the C-terminus of the fusion protein). In a particular embodiment, a plant, plant part, plant cell or seed having a rebalanced expression level of the TIR polypeptides comprises a polynucleotide having a single expression cassette, the polynucleotide comprising a nucleotide sequence encoding a fusion protein of TIRA polypeptide, or active variant or fragment thereof linked to the TIRB polypeptide or active variant or fragment thereof, expression of the fusion protein driven by a common promoter. In specific embodiments, the plant comprises the fusion protein of any of SEQ ID NOs: 9-12 or an active variant or fragment thereof, or the plant comprises a polynucleotide encoding the fusion protein of any of SEQ ID NOS: 9-12 or an active variant or fragment thereof, or the plant comprises the polynucleotide of any of SEQ ID NOS: 13-19 and 21. In particular embodiments, the fusion protein comprises a cleavable linker sequence between the TIRA polypeptide and TIRB polypeptide of the fusion protein, wherein following expression of the fusion, cleavage of the fusion protein at the linker sequence results in the release of the individual constituent TIR polypeptides at a balanced level.

II. Plants with a Modified NADase Activity Level of TIRA and TIRB Polypeptides

In embodiments, the plants, plant parts, plant cells and seed disclosed herein which comprise in their genome a polynucleotide encoding a TIRA polypeptide co-expressed with a TIRB polypeptide (or active variant or fragments of either), or a fusion protein thereof, may have a modified NADase activity level. In particular embodiments, the NADase activity of the TIRA and/or TIRB polypeptide is decreased by the inclusion of a loss of function mutation in the TIRA1 and/or TIRBA2 and/or TIRB1 domains. In other particular embodiments, the NADase activity of the TIRA polypeptide is increased by the inclusion of a gain of function mutation in the TIRA1 domain and/or TIRB1 domains. In example embodiments, plants, plant parts and seed are provided which comprise in their genome a polynucleotide encoding a TIRA polypeptide as set forth in SEQ ID NO: 1, or an active variant or fragment thereof, the polynucleotide further comprising a loss of function mutation that decreases an NADase activity of the TIRA polypeptide, or active variant or fragment thereof, while maintaining the ability of the mutated TIRA polypeptide to confer disease resistance in the plant when co-expressed with a TIRB polypeptide, or active variant or fragment thereof. In particular embodiments, the plant comprises a polynucleotide encoding a TIRA polypeptide having a loss of function mutation that decreases an NADase activity of the TIRA polypeptide, the polynucleotide including a mutation in a TIRA1 domain of the TIRA polypeptide, such as a Glutamic acid (E) to Alanine (A) mutation (or a conservative variant thereof) at a position corresponding to position 85 of SEQ ID NO: 1. In particular embodiments, the plant additionally or alternatively comprises a polynucleotide encoding a TIRA polypeptide having a gain of function mutation that increases an NADase activity of the TIRA polypeptide, the polynucleotide including a mutation in a TIRA2 domain of the TIRA polypeptide, such as a Valine (V) to Glutamic acid (E) mutation (or a conservative variant thereof) at a position corresponding to position 251 of SEQ ID NO: 1.

In particular embodiments, plants comprising a fusion protein comprising a TIRA polypeptide having a modified NADase activity comprise the polynucleotide of any one of SEQ ID NOS: 17, 18 and 21.

In other example embodiments, plants, plant parts and seed are provided which comprise in their genome a polynucleotide encoding a TIRB polypeptide as set forth in SEQ ID NO: 2, or an active variant or fragment thereof, the polynucleotide further comprising a loss of function mutation that decreases an NADase activity of the TIRB polypeptide, or active variant or fragment thereof, while maintaining the ability of the mutated TIRB polypeptide to confer disease resistance in the plant when co-expressed with a TIRA polypeptide, or active variant or fragment thereof. In particular embodiments, the plant comprises a polynucleotide encoding a TIRB polypeptide having a loss of function mutation that decreases an NADase activity of the TIRB polypeptide, wherein the polynucleotide comprises a mutation in a TIRB1 domain of the TIRB polypeptide, such as a Glutamic acid (E) to Alanine (A) mutation (or a conservative variant thereof) at a position corresponding to position 87 of SEQ ID NO: 2 and no mutations in a TIRB2 domain of the TIRB polypeptide.

In particular embodiments, plants comprising a fusion protein comprising a TIRB polypeptide having a modified NADase activity comprise the polynucleotide of any one of SEQ ID NOS: 19 and 21.

In still other example embodiments, plants, plant parts and seed are provided which comprise in their genome a polynucleotide encoding a TIRA polypeptide as set forth in SEQ ID NO: 1 and a TIRB polypeptide as set forth in SEQ ID NO: 2, or an active variant or fragment of either, the polynucleotide further comprising a loss of function mutation that decreases an NADase activity of each of the TIRA and TIRB polypeptide, or active variant or fragment thereof, while maintaining the ability of the mutated TIR polypeptides to confer disease resistance in the plant when co-expressed with each other. In particular embodiments, the plant comprises a polynucleotide encoding mutated TIRA and TIRB polypeptides, each having a loss of function mutation that decreases an NADase activity of the polypeptides, wherein the polynucleotide comprises a mutation in a TIRB1 domain of the TIRB polypeptide, such as a Glutamic acid (E) to Alanine (A) mutation (or a conservative variant thereof) at a position corresponding to position 87 of SEQ ID NO: 2 and no mutations in a TIRB2 domain of the TIRB polypeptide; and a mutation in a TIRA1 domain of the TIRA polypeptide, such as a Glutamic acid (E) to Alanine (A) mutation (or a conservative variant thereof) at a position corresponding to position 85 of SEQ ID NO: 1. In particular embodiments, the plant additionally or optionally comprises a gain of function mutation that increases the NADase activity of the TIRA polypeptide, wherein the polynucleotide comprises the gain of function mutation in the TIRA2 domain of the TIRA polypeptide, such as a Valine (V) to Glutamic acid (E) mutation (or a conservative variant thereof) at a position corresponding to position 251 of SEQ ID NO: 1.

In particular embodiments, plants comprising a fusion protein comprising a TIRA and a TIRB polypeptide having a modified NADase activity comprise the polynucleotide of SEQ ID NO: 21.

5. Methods for Producing a Plant that has Increased Disease Resistance

Provided herein are methods of producing a plant, plant part or a seed that has increased disease resistance by introducing into the plant, plant part, or plant cell, a nucleic acid sequence comprising polynucleotides encoding a TIRA polypeptide set forth in SEQ ID NO: 1 or an active variant or fragment thereof, and a TIRB polypeptide set forth in SEQ ID NO: 2 or an active variant or fragment thereof, wherein co-expression of the TIRA polypeptide with the TIRB polypeptide increases disease resistance of the plant. In other embodiments, the method comprises introducing into the plant, plant cell or plant part a nucleic acid sequence comprising a first polynucleotide set forth in any of SEQ ID NOS: 3-5 and a second polynucleotide set forth in any one of SEQ ID NOS: 6-8, or variants and fragments thereof, wherein co-expression of the first and second polypeptide increases disease resistance of the plant.

Also provided herein are methods of producing a plant, plant part or a seed that has increased disease resistance by introducing into the plant, plant part, or plant cell, a nucleic acid sequence comprising polynucleotides encoding a TIRATIRB fusion protein set forth in any of SEQ ID NOS: 9-12 or an active variant or fragment thereof, wherein expression of the TIRATIRB fusion protein increases disease resistance of the plant. In other embodiments, the method comprises introducing into the plant, plant cell or plant part a nucleic acid sequence comprising a polynucleotide set forth in any of SEQ ID NOS: 13-19 and 21, wherein expression of the fusion polypeptide encoded by the polynucleotide increases disease resistance of the plant. In particular embodiments, the fusion polypeptide comprises a cleavable linker sequence such that following expression of the fusion protein in the plant, plant part or cell, the fusion protein is cleaved to the constituent TIRA and TIRB polypeptides, thereby expressing the TIR polypeptides at a relative level that results in the increased disease resistance.

A nucleic acid sequence may be introduced into a plant cell by various ways, for example, by transformation, by genome modification techniques (such as by genome editing or targeted integration), or by breeding. In one aspect, the plant can be produced by transforming the nucleic acid sequence encoding the TIRA and TIRB polypeptides or TIR fusion proteins disclosed above into a recipient plant. In one aspect, the method can comprise editing the genome of the recipient plant so that the resulting plant comprises a polynucleotide encoding the TIRA and TIRB polypeptides or fusion protein disclosed herein or an active variant or fragment thereof. In another aspect, the method can comprise breeding a donor plant comprising a polynucleotide encoding the TIRA and TIRB polypeptides provided herein or an active variant or fragment thereof with a recipient plant and selecting for incorporation of the polynucleotide into the recipient plant genome.

a. Methods and Compositions to Increase Expression and/or Activity of a Polypeptide of Interest

Methods and compositions are provided that increase disease resistance in a plant by increasing expression and/or activity of the TIRA polypeptide or active variant or fragment thereof, and the TIRB polypeptide or active variant or fragment thereof.

In particular embodiments, the methods and compositions provided herein increase co-expression of the TIRA and TIRB polypeptides, or their fragments and variants. As used herein, “increasing the expression”, “increasing the co-expression”, “increased expression”, or “increased co-expression” of a TIRA and a TIRB polypeptide, or an active variant or fragment of the TIRA and/or TIRB polypeptide, means the level of the TIRA and TIRB polypeptides (including absolute levels of each polypeptide and relative levels of the TIRA polypeptide compared to the TIRB polypeptide), or active variants or fragments of either polypeptide, produced by the given plant, plant cell, plant part, or seed is statistically higher than the expression level compared against an appropriate control plant, plant part, plant cell or seed. In specific embodiments, the increase in expression or co-expression can comprise any statistically significant increase in the concentration of the polypeptides (e.g., increase in the concentration of the TIRA polypeptide, increase in the concentration of the TIRB polypeptide, or increase in concentration of both polypeptides) by at least 1%, 2%, 3%, 4%, 5%, 6%, 7%, 8%, 9%, 10%, 15%, 20%, 30%, 40%, 50%, 60%, 70%, 80%, 90%, 100% when compared to an appropriate control. In other embodiments, the increase in expression can comprise an increase in the level of the target protein concentration by at least 1-fold, 10 fold, 20 fold, 30 fold, 40 fold, 50 fold, 60 fold, 70 fold, 80 fold, 90 fold, 100 fold, 150 fold, 175 fold, 200 fold or more when compared to an appropriate control plant. Methods to assay for an increase in the level or expression of a target polypeptide are known and include, for example, the detection of the protein via antibodies or an increase in the level of expression of the mRNA encoding the protein.

In some embodiments, the increase in expression of the TIRA polypeptide, or active variant or fragment thereof, in a plant, plant part, plant cell or seed, is comparable to the increase in expression of the TIRB polypeptide, or active variant or fragment thereof, such as where the increase in expression of both the polypeptides is by at least 1%, between 1% to 10%, between 10% to 50%, between 25% to 75%, between 50% to 100%, by at least 1-fold, between 1 fold to 10 fold, between 10 fold to 50 fold, between 25 fold to 75 fold, between 50 fold to 100 fold or high, as compared to an appropriate control.

In other embodiments, the increase in expression of the TIRA polypeptide, or active variant or fragment thereof, in a plant, plant part, plant cell or seed, is different from the increase in expression of the TIRB polypeptide, or active variant or fragment thereof. In particular embodiments, the increase in expression of the TIRA polypeptide (or active variant or fragment thereof) is more than the increase in expression of the TIRB polypeptide (or active variant or fragment thereof), for example, in embodiments where expression of the TIRA polypeptide or active variant or fragment thereof is driven by a different promoter than the expression of the TIRB polypeptide or active variant or fragment thereof. In example embodiments, a higher increase in expression level of the TIRA polypeptide relative to the increase in expression level of the TIRB polypeptide is achieved in embodiments where expression of the TIRA and TIRB polypeptides are driven by different native promoters (e.g., expression of TIRA polypeptide driven by prGCaRG3a (SEQ ID NO: 26) and expression of TIRB polypeptide driven by prGCaRG3b (SEQ ID NO: 27)), or where expression of the TIRA polypeptide is driven by a constitutive promoter while expression of the TIRB polypeptide is driven by an inducible promoter (e.g., prLuFIS (SEQ ID NO: 25)). In other embodiments, the increase in expression of the TIRB polypeptide (or active variant or fragment thereof) is less than the increase in expression of the TIRA polypeptide (or active variant or fragment thereof). In example embodiments, the increase in expression of the TIRA polypeptide is 1-5 fold times the increase in expression of the TIRB polypeptide in the plant, plant part, plant cell or seed. In other example embodiments, the increase in expression of the TIRB polypeptide is 0.5 fold times the increase in expression of the TIRA polypeptide. In specific embodiments, the higher increase in expression of the TIRA polypeptide relative to the increase in expression level of the TIRB polypeptide results in a rebalanced expression of the TIRA polypeptide relative to the TIRB polypeptide as compared to an appropriate control plant.

In other particular embodiments, the increase in expression of the TIRA polypeptide or active variant or fragment thereof, and the increase in expression of the TIRB polypeptide or active variant or fragment thereof, in a plant, plant cell or plant part is driven by expression of both polypeptides by a common bidirectional promoter (e.g., bidirectional promoter of SEQ ID NO: 49) wherein a nucleotide sequence encoding the TIRA polypeptide is operably coupled upstream of the bidirectional promoter while the nucleotide sequence encoding the TIRB polypeptide is operably coupled downstream of the promoter, or a nucleotide sequence encoding the TIRA polypeptide is operably coupled downstream of the bidirectional promoter while the nucleotide sequence encoding the TIRB polypeptide is operably coupled upstream of the promoter.

In other embodiments, methods and compositions are provided that increase the activity of the TIRA polypeptide or active variant or fragment thereof, and the activity of the TIRB polypeptide or active variant or fragment thereof, in a plant, plant cell or plant part. As used herein, “increasing the activity” or “increased activity” of the TIRA and/or TIRB polypeptide, or active variants or fragments thereof, means the level of the enzyme activity or protein functionality of the TIRA and/or TIRB polypeptides is statistically higher when compared to an appropriate control. In particular embodiments, the increased activity of the TIRA polypeptide or active variants or fragments thereof means one or more of: (i) the ability of the TIRA polypeptide (or active variant or fragment thereof) to mount an immune response in a plant cell is increased, (ii) the ability of the TIRA polypeptide (or active variant or fragment thereof) to confer disease resistance to a plant when co-expressed with a TIRB polypeptide (or active variant or fragment thereof) is increased, and/or (iii) an NADase activity of the polypeptide is increased. In other particular embodiments, the increased activity of the TIRB polypeptide or active variants or fragments thereof means one or more of: (i) the ability of the TIRB polypeptide (or active variant or fragment thereof) to mount an immune response in a plant cell is increased, (ii) the ability of the TIRB polypeptide (or active variant or fragment thereof) to confer disease resistance to a plant when co-expressed with a TIRA polypeptide (or active variant or fragment thereof) is increased, and/or (iii) an NADase activity of the polypeptide is increased. In some embodiments, the increased activity means an increase in the ability of the TIRA and TIRB polypeptides (or active variant or fragment thereof) to confer disease resistance to a plant when co-expressed while an NADase activity of the TIRA and/or TIRB polypeptide (or active variant or fragment thereof) is decreased.

In specific embodiments, the increase in activity can comprise an increase in the level of enzyme activity or protein functionality of the TIRA and/or TIRB polypeptide or active variant or fragment thereof by at least 1%, 2%, 3%, 4%, 5%, 6%, 7%, 8%, 9%, 10%, 15%, 20%, 30%, 40%, 50%, 60%, 70%, 80%, 90%, 100% when compared to an appropriate control plant, plant part, plant cell or seed. In other embodiments, the increase in the activity of the TIRA and/or TIRB polypeptide or active variant or fragment thereof can comprise an increase in the level of enzymatic activity or protein functionality by at least 1-fold, 10 fold, 20 fold, 30 fold, 40 fold, 50 fold, 60 fold, 70 fold, 80 fold, 90 fold, 100 fold, 150 fold, 175 fold, 200 fold or more when compared to an appropriate control plant, plant part, plant cell or seed. Methods to assay for an increase in the enzyme activity or protein functionality include direct assays for the activity of the specific protein and as well as indirect assays.

In still other embodiments, the increase in activity and/or expression level of TIRA and TIRB polypeptide comprises an increase in expression level of a fusion protein comprising a TIRA polypeptide and a TIRB polypeptide, or active variants or fragments thereof, such as the fusion protein of any of SEQ ID NOS: 9-12 or an active variant or fragment thereof. In particular embodiments, the fusion protein comprises a cleavable linker sequence such that following expression of the fusion protein in the plant, plant part or cell, the fusion protein is cleaved to the constituent TIRA and TIRB polypeptides, thereby increasing the activity and/or relative expression level of the TIRA and TIRB polypeptides.

In some embodiments, increasing the activity and/or expression level of a TIRA and TIRB polypeptide includes the introduction of a nucleic acid construct into a plant that results in the increased expression and/or activity of the TIRA and TIRB polypeptides or active variants or fragments thereof. In other embodiments, increasing the activity and/or expression level of a TIRA and TIRB polypeptide includes the introduction of a nucleic acid construct into a plant that results in the increased expression and/or activity of a fusion protein comprising the TIRA and TIRB polypeptides or active variants or fragments thereof. The nucleic acid construct can be stably integrated in the genome or be provided transiently. For example, the nucleic acid construct can comprise the nucleic acid sequence encoding the TIRA polypeptide of SEQ ID NO: 1 and the TIRB polypeptide of SEQ ID NO: 2, or active variants or fragments thereof. In another example, the nucleic acid construct can comprise a nucleic acid sequence encoding the fusion protein of any of SEQ ID NOS: 9-12, or active variants or fragments thereof.

As such, plants, plant parts, seeds and plant cells are provided having stably incorporated into their genome a polynucleotide operably linked to a promoter active in the plant, wherein the polynucleotide encodes (i) a polypeptide having an amino acid sequence having at least 80%, 85%, 90%, 91%, 92%, 93%, 94%, 95%, 96%, 97%, 98%, 99% or 100% sequence identity to any one of SEQ ID NO: 1, or an active variant or active fragment thereof, and (ii) a polypeptide having an amino acid sequence having at least 80%, 85%, 90%, 91%, 92%, 93%, 94%, 95%, 96%, 97%, 98%, 99% or 100% sequence identity to any one of SEQ ID NO: 2, or an active variant or active fragment thereof, wherein the increase in expression or activity of the polypeptides increases the disease resistance and/or the pathogen resistance of the plant. In some embodiments, plants, plant parts, seeds and plant cells are provided having stably incorporated into their genome a polynucleotide operably linked to a promoter active in the plant, wherein the polynucleotide encodes a fusion protein having an amino acid sequence having at least 80%, 85%, 90%, 91%, 92%, 93%, 94%, 95%, 96%, 97%, 98%, 99% or 100% sequence identity to any one of SEQ ID NOS: 9-12, or an active variant or active fragment thereof, wherein the increase in expression or activity of the fusion protein increases the disease resistance and/or the pathogen resistance of the plant.

Upon transformation, the target polypeptides are integrated into the genome and expressed in a manner that increases the activity or expression level of the TIRA and TIRB polypeptides in the plant.

Methods are therefore provided whereby the pathogen resistance of a plant is increased by introducing into the genome of the plant a nucleic acid construct that results in the increased expression and/or activity of (i) a polypeptide having an amino acid sequence having at least 80%, 85%, 90%, 91%, 92%, 93%, 94%, 95%, 96%, 97%, 98%, 99% or 100% sequence identity to any one of SEQ ID NO: 1, or an active variant or active fragment thereof, and (ii) a polypeptide having an amino acid sequence having at least 80%, 85%, 90%, 91%, 92%, 93%, 94%, 95%, 96%, 97%, 98%, 99% or 100% sequence identity to any one of SEQ ID NO: 2, or an active variant or active fragment thereof, wherein the modification increases the pathogen resistance and/or the disease resistance of the plant. In one embodiment, the method comprises introducing into the genome of a plant a nucleic acid sequence encoding (i) a polypeptide having an amino acid sequence having at least 80%, 85%, 90%, 91%, 92%, 93%, 94%, 95%, 96%, 97%, 98%, 99% or 100% sequence identity to any one of SEQ ID NO: 1, or an active variant or active fragment thereof, and (ii) a polypeptide having an amino acid sequence having at least 80%, 85%, 90%, 91%, 92%, 93%, 94%, 95%, 96%, 97%, 98%, 99% or 100% sequence identity to any one of SEQ ID NO: 2, or an active variant or active fragment thereof, where the increased activity or expression of the polypeptides increases the pathogen resistance and/or the disease resistance of the plant. Methods are also provided whereby the pathogen resistance of a plant is increased by introducing into the genome of the plant a nucleic acid construct that results in the increased expression and/or activity of a fusion protein having an amino acid sequence having at least 80%, 85%, 90%, 91%, 92%, 93%, 94%, 95%, 96%, 97%, 98%, 99% or 100% sequence identity to any one of SEQ ID NOS: 9-12, or an active variant or active fragment thereof, wherein the modification increases the pathogen resistance and/or the disease resistance of the plant. In some embodiments, an expression cassette comprising a promoter active in the plant is operably linked to the polynucleotides of interest encoding the TIRA and TIRB polypeptides, or their active variants or fragments thereof, is introduced into the genome of the plant (e.g., an expression cassette comprising a promoter active in the plant operably linked to a polynucleotide encoding a fusion protein comprising the TIRA and TIRB polypeptides, or a first expression cassette comprising a first promoter active in the plant operably linked to a first polynucleotide encoding a TIRA polypeptide and a second expression cassette comprising a second promoter active in the plant operably linked to a second polynucleotide encoding a TIRB polypeptide). In other embodiments, the polynucleotides of interest can be introduced into the genome of the plant and integrated at a genomic location (via for example, targeted integration) that allows for the expression of the polypeptide.

In other embodiments, the methods comprise introducing a nucleic acid construct that produces a modification in the genome of the plant that results in an increased expression or an increased activity of (i) a TIRA polypeptide having an amino acid sequence with at least 80%, 85%, 90%, 91%, 92%, 93%, 94%, 95%, 96%, 97%, 98%, 99%, or 100% identity to any one of SEQ ID NO: 1 or an active variant or fragment thereof, and (ii) a TIRB polypeptide having an amino acid sequence with at least 80%, 85%, 90%, 91%, 92%, 93%, 94%, 95%, 96%, 97%, 98%, 99%, or 100% identity to any one of SEQ ID NO: 2, or an active variant or fragment thereof, where the modification increases the pathogen resistance of the plant.

In embodiments, methods are also provided for altering the relative expression level of a TIRA polypeptide and a TIRB polypeptide, or active fragments or variants thereof, in a plant to thereby rebalance their relative expression level. In embodiments, the method rebalances the expression level from an unbalanced expression level where expression of the TIRB polypeptide exceeds expression of the TIRA polypeptide (e.g., a ratio in the range of 5:1 to 20:1), or wherein expression of the TIRA polypeptide exceeds expression of the TIRB polypeptide (e.g., a ratio in the range of 5:1 to 20:1), to a balanced expression level of 0.5:1 to 2:1. In embodiments, the method of rebalancing comprises, introducing into the genome of a plant, such as a legume plant, a first heterologous nucleotide sequence encoding a TIRA polypeptide, or an active variant thereof, having at least 80%, 85%, 90%, 91%, 92%, 93%, 94%, 95%, 96%, 97%, 98% 99% or 100% identity to SEQ ID NO: 1; a second heterologous nucleotide sequence encoding a TIRB polypeptide, or an active variant thereof, having at least 80%, 85%, 90%, 91%, 92%, 93%, 94%, 95%, 96%, 97%, 98% 99% or 100% identity to SEQ ID NO: 2; and a heterologous promoter operably coupled to the first and/or the second heterologous nucleotide sequence, wherein an expression level of the TIRA polypeptide, or active variant thereof, relative to the TIRB polypeptide, or active variant thereof, is rebalanced in the plant, plant part or seed as compared to the expression level in a control plant, plant part or seed, wherein the rebalanced expression level of the TIRA polypeptide, or active variant thereof, relative to the TIRB polypeptide, or active variant thereof, comprises a ratio of about 0.5:1 to about 2:1, and wherein the TIRA polypeptide, or active variant thereof, confers disease resistance to the legume plant, plant or seed when co-expressed with the TIRB polypeptide, or active variant thereof, and wherein the TIRA polypeptide, or active variant thereof, confers disease resistance to the legume plant, plant or seed when co-expressed with the TIRA polypeptide, or active variant thereof.

b. Screening Methods

In some embodiments, methods are provided for screening plants that comprise polynucleotides encoding TIRA and TIRB polypeptides and wherein the TIR polypeptides are expressed in a balanced level so as to confer increased disease resistance while also improving agronomic performance.

In particular embodiments, the method of screening for a plant having increased disease resistance (e.g., increased ASR resistance or increased powdery mildew resistance or bacterial pathogen resistance or nematode resistance or sucking pest resistance) and increased agronomic performance, comprises, introducing into the genome of the plant, a nucleic acid molecule comprising a heterologous promoter operably coupled to (i) a first polynucleotide encoding a encoding a TIRA polypeptide, or active variant thereof, comprising an amino acid sequence having at least 40%, 45%, 50%, 55%, 60%, 65%, 70%, 75%, 80%, 85%, 90%, 91%, 92%, 93%, 94%, 95%, 96%, 97%, 98% 99% or 100% identity to SEQ ID NO: 1; and (ii) a second polynucleotide encoding a TIRB polypeptide, or active variant thereof, comprising an amino acid sequence having at least 40%, 45%, 50%, 55%, 60%, 65%, 70%, 75%, 80%, 85%, 90%, 91%, 92%, 93%, 94%, 95%, 96%, 97%, 98% 99% or 100% identity to SEQ ID NO: 2. In embodiments, the first and second polynucleotides may be introduced via distinct expression cassettes or via a single expression cassette. In embodiments, the first and second polynucleotide may be linked so as to express the TIRA and TIRB polypeptides, or fragments or variants thereof, as a fusion protein, as discussed earlier herein. In embodiments, the fusion protein comprises a self-cleavable linker that is cleaved following expression of the fusion protein, thereby releasing the constituent TIR polypeptides into distinct polypeptides.

The method of screening further comprises assaying an expression level of the TIRA polypeptide, or active variant thereof, and the expression level of the TIRB polypeptide, or active variant thereof, in the plant. Any known methods of assaying for expression levels may be used including but not limited to estimating transcript levels (e.g., via qRT-PCR, RNA-seq, Northern blotting, etc.). The method of screening further comprises, after estimating the expression levels of the TIR polypeptides, comparing the expression level of the TIRA polypeptide or active variant thereof relative to the expression level of the TIRB polypeptide, or active variant thereof, in the plant to determine if the relative expression level is balanced. In particular embodiments, it is determined if the relative expression level of the TIRA polypeptide or active variant thereof relative to the expression level of the TIRB polypeptide, or active variant thereof, in the plant, is between 0.5:1 to 2:1, thereby determining a balanced expression level of the TIRA polypeptide, or active variant thereof, relative to the expression level of the TIRB polypeptide in the plant. The method of screening further comprises selecting a plant having a balanced expression level of TIRA polypeptide, or active variant thereof, relative to the TIRB polypeptide, or active variant thereof. Such plants having a balanced expression level of the TIR polypeptides have increased disease resistance and improved agronomic performance (e.g., reduced growth defects and higher yield).

In particular embodiments, a method of producing a plant having increased disease resistance and increased agronomic performance comprises: (a) introducing into the genome of the plant, a nucleic acid molecule comprising a heterologous promoter operably coupled to (i) a first polynucleotide encoding a encoding a TIRA polypeptide, or active variant thereof, comprising an amino acid sequence having at least 40%, 45%, 50%, 55%, 60%, 65%, 70%, 75%, 80%, 85%, 90%, 91%, 92%, 93%, 94%, 95%, 96%, 97%, 98% 99% or 100% identity to SEQ ID NO: 1 and further comprising one or more mutations at positions corresponding to position 85 and/or position 271 of SEQ ID NO: 1, wherein the one or more mutations alter an NADase activity of the TIRA polypeptide or active variant thereof; and (ii) a second polynucleotide encoding a TIRB polypeptide, or active variant thereof, comprising an amino acid sequence having at least 40%, 45%, 50%, 55%, 60%, 65%, 70%, 75%, 80%, 85%, 90%, 91%, 92%, 93%, 94%, 95%, 96%, 97%, 98% 99% or 100% identity to SEQ ID NO: 2 and further comprising a mutation at a position corresponding to position 87 of SEQ ID NO: 2, wherein the mutation reduces an NADase activity of the TIRB polypeptide or active variant thereof. The method further comprises assaying an expression level of the TIRA polypeptide, or active variant thereof, and the expression level of the TIRB polypeptide, or active variant thereof, in the plant; and comparing the expression level of the TIRA polypeptide or active variant thereof relative to the expression level of the TIRB polypeptide, or active variant thereof, in the plant to determine if the relative expression level is between 0.5:1 to 2:1, thereby determining a balanced expression level of the TIRA polypeptide, or active variant thereof, relative to the expression level of the TIRB polypeptide in the plant. A plant is thereby selected having a balanced expression level of TIRA polypeptide, or active variant thereof, relative to the TIRB polypeptide, or active variant thereof.

In embodiments, methods are also provided for screening and selection of polynucleotides, nucleic acid molecules, and compositions that confer increased disease resistance and also confer increased agronomic performance when expressed in a plant.

In a particular embodiment, a method is provided for producing a TirA-TirB composition capable of conferring increased disease resistance and improved agronomic performance when introduced into a plant. The method comprises generating a first polynucleotide encoding a encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1 and a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2.

The screening method further comprises mutating the first polynucleotide to introduce one or more mutations at positions corresponding to position 85 and/or position 271 of SEQ ID NO: 1, wherein the one or more mutations alter an NADase activity of the TIRA polypeptide or active variant thereof. In particular embodiments, the first polynucleotide is mutated to introduce a loss of function mutation at positions corresponding to position 85 and/or 271 of SEQ ID NO: 1. In one particular embodiment, where the TIRA polypeptide or variant thereof comprises a Glu residue at a position corresponding to position 85 of SEQ ID NO: 1, wherein the Glu residue is predicted to have an NADase cleavage activity, a loss of function E85A mutation is introduced that reduces the NADase activity of the TIRA polypeptide. In another example embodiment, where the TIRA polypeptide or variant thereof comprises a Glu residue at a position corresponding to position 271 of SEQ ID NO: 1, wherein the Glu residue is predicted to have an NADase cleavage activity, a loss of function E271A mutation may be introduced that reduces the NADase activity of the TIRA polypeptide. In yet another example embodiment, where the TIRA polypeptide or variant thereof comprises a non-Glu residue at a position corresponding to position 271 of SEQ ID NO: 1, such as a Val residue, wherein the Val residue is not predicted to have an NADase cleavage activity, a gain of function V271E mutation is introduced that increases the NADase activity of the TIRA polypeptide.

The screening method further comprises mutating the second polynucleotide to introduce a mutation at a position corresponding to position 87 of SEQ ID NO: 2, wherein the mutation reduces an NADase activity of the TIRB polypeptide or active variant thereof. In one particular embodiment, where the TIRB polypeptide or variant thereof comprises a Glu residue at a position corresponding to position 87 of SEQ ID NO: 2, wherein the Glu residue is predicted to have an NADase cleavage activity, a loss of function E87A mutation is introduced that reduces the NADase activity of the TIRB polypeptide.

The screening method further comprises expressing the first polynucleotide comprising the one or more mutations and the second polynucleotide comprising the mutation in a plant via an operably-linked heterologous promoter. In embodiments, the first and second polynucleotide are expressed as distinct expression cassettes or via a single expression cassette. In other embodiments, the first and second polynucleotide are linked to express the TIR polypeptides as a fusion protein. In embodiments, the fusion protein comprises a self-cleavable linker that is cleaved following expression of the fusion protein, thereby releasing the constituent TIR polypeptides into distinct polypeptides.

The screening method further comprises, in embodiments, screening for a plant having a balanced expression level of the TIRA polypeptide or active variant thereof relative to the TIRB polypeptide or active variant thereof in the plant, wherein the balanced expression level comprises a ratio of 0.5:1 to 2:1. By selecting for a composition comprising polynucleotides encoding for the TIR polypeptides that, when expressed in a plant, result in a balanced relative expression level of the TIR polypeptides, plants may be produced having the composition introduced into their genome, whereby the plants have increased disease resistance and improved agronomic performance.

c. Transformation Methods

In some embodiments, methods of introducing a polynucleotide into a plant comprises transforming a polynucleotide disclosed herein or an active variant or fragment thereof into a recipient plant to obtain a transgenic plant and said transgenic plant has increased pathogen resistance and/or disease resistance. Expression cassettes comprising polynucleotides encoding the polypeptides as described above can be used to transform plants of interest.

Transformation results in the introduction of a heterologous nucleic acid into a plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. In some embodiments, the stable transformation occurs via a random integration event. In other embodiments, the stable transformation occurs via a targeted integration into the genome of the sequence of interest employing genome modification machinery such as, for example, CRISPRs or TALENs. “Transient transformation” is intended to mean that a polynucleotide is introduced into the host cell and does not integrate into the genome of the host cell.

Methods for transformation typically involve introducing a nucleotide construct into a plant. In some embodiments, the transformation method is an Agrobacterium-mediated transformation. In some embodiments, the transformation method is a biolistic-mediated transformation. Transformation may also be performed by infection, transfection, microinjection, electroporation, microprojection, biolistics or particle bombardment, electroporation, silica/carbon fibers, ultrasound mediated, PEG mediated, calcium phosphate co-precipitation, poly cation DMSO technique, DEAE dextran procedure, Agrobacterium and viral mediated (e.g., Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; and 7,541,517; each of which is herein incorporated by reference. See, also, Rakoczy-Trojanowska, M. (2002) Cell Mol Biol Lett. 7:849-858; Jones et al. (2005) Plant Methods, Vol. 1, Article 5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:57 Sup′/Sup5-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047.

Methods of transformation of plant cells or tissues include but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3 (7): 637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

Methods for transformation of chloroplasts are known in the art. See, for example, Svab et al. (1990) Proc. Natl. Acad. Sci. USA 87 (21): 8526-8530; Svab and Maliga (1993) Proc. Natl. Acad. Sci. USA 90 (3): 913-917; Staub and Maliga (1993) EMBO J. 12 (2): 601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91 (15): 7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

“Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical See, for example, Ammirato et al., Handbook of Plant Cell Culture Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

d. Crossing

In some embodiments, the method comprises crossing a donor plant comprising a polynucleotide encoding a (i) a TIRA polypeptide or active variant or fragment thereof and a TIRB polypeptide or active variant or fragment thereof or (ii) a TIRATIRB fusion protein with a recipient plant, and the polypeptide or fusion protein is able to confer increased pathogen resistance in the recipient plant. As used herein, the terms “crossing” and “breeding” refer to the fusion of gametes to produce progeny (e.g., by fertilization, such as to produce seed by pollination in plants). In some embodiments, a “cross,” “breeding,” or “cross-fertilization” is fertilization of one individual by another (e.g., cross-pollination in plants). The plant disclosed herein may be a whole plant, or may be a plant cell, seed, or tissue, or a plant part such as leaf, stem, pollen, or cell that can be cultivated into a whole plant.

In some embodiments, a progeny plant created by the crossing or breeding process is repeatedly crossed back to one of its parents through a process referred to herein as “backcrossing”. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in Techniques et Utilisations des Marqueurs Moleculaires Les Colloques, Vol. 72, pp. 45-56 (1995); and Openshaw et al., Marker-assisted Selection in Backcross Breeding, in Proceedings of the Symposium “Analysis of Molecular Marker Data,” pp. 41-43 (1994). The initial cross gives rise to the F1 generation. The term “BC1” refers to the second use of the recurrent parent, “BC2” refers to the third use of the recurrent parent, and so on.

In some embodiments, the donor soybean plant is a Glycine max plant. In some embodiments, the donor soybean plant is a Glycine soja plant. In some embodiments, the recipient soybean plant is an elite Glycine max plant or an elite Glycine soja plant.

e. Gene Editing

Further provided are plants, plant cells and seeds having a genomic modification created through gene editing. Such methods include, but are not limited to, meganucleases designed against the plant genomic sequence of interest, CRISPR-Cas9, TALENs, and other technologies for precise editing of genomes (Feng, et al. Cell Research 23:1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol: 51-65).

Various embodiments of the methods described herein use gene editing. In some embodiments, gene editing is used to mutagenize the genome of a plant to produce plants having one or more of the polypeptides that are able to increase disease resistance in a plant. In other instances, the gene editing is used to allow for targeted insertion into the genome of a nucleotide sequence encoding the TIRA and TIRB polypeptides, or the TIRATIRB fusion protein, or active variants or fragments thereof.

“Target site,” “target sequence,” “target DNA,” “target locus,” “genomic target site,” “genomic target sequence,” and “genomic target locus” are used interchangeably herein and refer to a polynucleotide sequence, for example in the genome (including chloroplastic and mitochondrial DNA) of a cell, to which an endonuclease is recruited, and optionally nicks or cleaves the DNA of the target site. The target site can be an endogenous site in the plant genome, or alternatively, the target site can be heterologous to the plant and thereby not be naturally occurring in the genome, or the target site can be found in a heterologous genomic location compared to where it occurs in nature

In some embodiments, provided herein are plants transformed with and expressing gene-editing machinery as described above, which, when crossed with a target plant, result in gene editing in the target plant.

The term “polynucleotide modification template” includes a polynucleotide that comprises at least one nucleotide modification when compared to the nucleotide sequence to be edited. A nucleotide modification can be at least one nucleotide substitution, addition, or deletion. The polynucleotide modification template can further comprise homologous nucleotide sequences flanking at least one nucleotide modification, wherein flanking homologous nucleotide sequences provides sufficient homology to the desired nucleotide sequence to be edited.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., increased disease resistance. The recombination template can be introduced into the plant either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest, e.g., any one of SEQ ID NOS: 8-13 and 15-22 or variants or fragments thereof may be provided on/as a template. By designing the system such that one or more single strand or double strand breaks are introduced within, upstream, and/or downstream of the corresponding region in the genome of a plant not comprising the gene sequence of interest, this region can be replaced with the template.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is affected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is affected by a (modified) meganuclease, which are endodeoxyribonucleases characterized by a large recognition site (double-stranded DNA sequences of 12 to 40 base pairs). Exemplary methods for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is affected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as, but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. Nos. 10,519,456 and 10,285,348 82, the entire contents of which are herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In another aspect, provided herein is a method of editing plant genomic DNA. In some embodiments, the method comprises using a first soybean plant expressing a DNA modification enzyme and at least one optional guide nucleic acid as described above to pollinate a target plant comprising genomic DNA to be edited.

### 6. Stacking

The polynucleotides encoding the TIRA polypeptide, TIRB polypeptide, and/or TIRATIRB fusion protein, and variants and fragments of the TIRA polypeptide, TIRB polypeptide, and/or TIRATIRB fusion protein, as provided herein, can be stacked with one or more polynucleotides encoding a desirable trait such as a polynucleotide that confers, for example, insect, disease or herbicide resistance or other desirable agronomic traits of interest including, but not limited to, traits associated with high oil content; traits associated with increase protein content, increased digestibility; balanced amino acid content; improved drought resistance, modified maturity and/or flowering time, and high energy content. Such traits may refer to properties of both seed and non-seed plant tissues, or to food or feed prepared from plants or seeds having such traits.

As used herein, gene or trait “stacking” comprises combining desired genes or traits into one transgenic plant line. The additional polynucleotide can be introduced by a variety of approaches including by transgenic means, by breeding, or by genome editing. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. In embodiments, the two or more genes may be transferred via distinct expression cassettes or via a common expression cassette. Another way to stack genes is by re-transforming a transgenic plant comprising a desired trait with another gene of interest conferring another desired trait to thereby provide a progeny transgenic plant comprising the combination of traits. Such methods can include, for example, random integration techniques or targeted integration via a gene editing system such as Crispr or meganucleases. For example, gene stacking can be used to combine two different insect resistance traits, two different herbicide resistance traits, two different agronomic performance traits, an insect resistance trait with a disease resistance trait a herbicide resistance trait (such as, for example, Bt11), or an agronomic performance trait, etc. The use of a selectable marker in addition to a gene of interest would also be considered gene stacking. In embodiments, the offspring or progeny plant having the desired combination of traits is identified through the use of genetic markers or molecular markers including but not limited to SNPs, QTLs, primers or probes directed to desired trait-associated genes or transgenes, promoters, microRNAs, siRNAs, mRNAs, ds RNAs, transcriptional profiles, and methylation patterns.

In some embodiments, a nucleic acid molecule or vector of the disclosure can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), disease resistance, virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

In an example embodiment, polynucleotides may be stacked wherein their co-expression is required to increase disease resistance within a plant. In a particular embodiment, a TIRA polypeptide, or active variant or fragment thereof, is stacked with a TIRB polypeptide, or active variant or fragment thereof, to increase resistance in a plant to ASR and/or powdery mildew and/or a bacterial pathogen (e.g., P. syringae) and/or a sucking pest (e.g., aphid or stinkbug or white fly) and/or a nematode (e.g., SCN or RKN). In another particular embodiment, a TIRA polypeptide, or active variant or fragment thereof, is stacked with a TIRB polypeptide, or active variant or fragment thereof, wherein an expression level of the TIRA polypeptide is rebalanced relative to the TIRB polypeptide, as compared to their expression levels in a control plant, so as to increase resistance in the plant to ASR and/or powdery mildew with a concomitant reduction in negative growth phenotypes associated with an unbalanced relative expression level of the polypeptides.

In another particular embodiment, polynucleotides may be stacked (or, alternatively, multiple expression cassettes may be stacked on a single polynucleotide) so as to express more than one polypeptide that increase disease resistance within a plant. This is a particular advantage where, for example, one polypeptide is particularly suitable for providing resistance to one class of plant pathogens (e.g., a first rust isolate) while the other provides resistance to a different class of plant pathogens (or a different result isolate). For example, a first polypeptide encoding a TIRATIRB fusion protein is stacked with a second polypeptide encoding another disease resistance protein. In other example embodiments, a first polypeptide is provided that provides resistance via a first mode of action against a plant pathogen (e.g., against ASR) while the other provides resistance to the same plant pathogen (e.g., also against ASR) via a second, different mode of action. Stacking polypeptides encoded by different polypeptides is also an advantage where one polypeptide expresses inherent pathogen-resistance but is somewhat labile.

Such additional disease resistance proteins include, but are not limited to, one or more of the proteins encoded by various resistance genes as set forth in: WO2019103918 (including, for example, but not limited to, RG1 (SEQ ID NO: 47 and active variants or fragments thereof) or SEQ ID NO: 42, 43, 44, 45, 46, 48, 49 or 50 disclosed therein); WO2021000878 (including for example Rpp 6907 (SEQ ID NO: 1 of WO202100878) and active variants or fragments thereof); WO2021022022 (for example, TirA or Tir B (SEQ ID NOS: 11 or 16 of WO2021022022) or active variants or fragments thereof); WO2021260673 (for example, but not limited to, RG21 and/or RG22 (SEQ ID NOS: 1 or 12 of WO2021260673) or active variants or fragments thereof); WO2022173659 (for example, but not limited to, RG30 (SEQ ID NO: 5 of WO2022173659) or active variants or fragments thereof); WO2022159341 (for example but not limited to SEQ ID NOS: 1 and 148 of WO2022159341 or active variants or fragments thereof); WO2021154632A1, WO2021022026, WO2021022101, US20220135997 (for example, but not limited to, FIT1 (SEQ ID NO: 2 of US20220135997), an active variant or fragment thereof or any of the FIT1 paralogs or orthologs disclosed therein (such as SEQ ID NOS: 4, 6, 8, 10, 12, 14, 16, 18 or 20 of US20220135997); U.S. Pat. No. 10,842,097 (for example, but not limited to, CeRpp1 or active variants or fragments thereof or any other resistance genes disclosed therein); WO2022140257 (for example, CcRpp2-RI and/or CcRpp2-R3 (SEQ ID NOS: 2 or 4 of WO2022140257) or an active variant or fragment thereof); and/or or the genes encoding the resistance proteins disclosed in U.S. Provisional Application 63/481,627 as RG31 (SEQ ID NO: 1) or RG35 (SEQ ID NO: 2); or disclosed in U.S. Provisional application 63/509,586 and 63/426,524 as RG32 (SEQ ID NO: 1) and RG34 (SEQ ID NO: 2 or 17). each of which is incorporated by reference in their entirety.

In other embodiments, the nucleic acid sequence encoding the TIRA polypeptide, the TIRB polypeptide and/or the TIRATIRB fusion protein, or an active variant or fragment of any of these polypeptides, is stacked with a native trait that confers disease resistance. In specific embodiments, the native trait that confers disease resistance is a protein that confers increased resistance to ASR or to pathogens from the genus Phakopsora, including the species Phakopsora pachyrhizi and Phakopsora meibomiae. For example, the various intervals, locus or resistance genes as set forth in WO2009079729, U.S. Pat. No. 9,091,681, WO2010009404, WO2017222827, WO2021000878, WO2021022026, WO2021022101, WO2021154632, WO2022173659, (each of which is incorporated by reference in their entirety) can be bred into a glycine max plant comprising the TIRA polypeptide and/or TIRB polypeptide, or the TIRATIRB fusion protein, or active variant or fragment of any thereof.

As such, in some embodiments, the TIRA protein and TIRB protein, or fusions thereof, can be deployed as a “native construct” or as a “native stack.” As used herein, a native construct comprises the native gene (that is, the native regulatory region and the native coding region) as found in nature, unmodified by man. Such native constructs are integrated into the genome in a heterologous location (that is, a location different from the native location in the genome). In such instances, the native constructs or native stacks of TIRA and TIRB (e.g., SEQ ID NO: 3 and SEQ ID NO: 6, or native variants or fragments thereof) are stably integrated into the genome via any method, including for example, TIN or random integration. In some embodiments, a native construct employed comprises at least the native gene of TIRA and TIRB or native variants of fragments thereof with at least one additional native gene of interest. The additional native gene of interest can include for example, the native gene, or native variant thereof, of any of the R-genes disclosed herein or any other native gene of interest including native genes related to any other pathogen resistance or agronomic performance.

The native construct or the native stack when integrated via targeted insertion can be deployed to neighbor either other native traits of interest and/or other heterologous traits of interest, such as herbicide tolerance traits or insect control traits as disclosed herein. See for example, the stacks of WO2022040134, US20220056470, and WO2023164453, the contents of which are incorporated by reference herein in their entirety.

Polynucleotides conferring resistance/tolerance to a herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional herbicide tolerant traits include, PPO tolerant traits including, for example, one or more PPO trait set forth in US20190062777, U.S. Pat. Nos. 10,370,677, 11,124,803, WO2017217793, WO2020251313, U.S. Pat. Nos. 10,392,630, 10,378,023, WO2016099153, WO2019117579, WO2019117578, and U.S. Pat. No. 10,100,329, each of which is herein incorporated by reference in their entirety. HPPD tolerant traits include: WO2009144079, U.S. Pat. No. 8,642,748, EP2453012, WO2013026740, U.S. Pat. Nos. 9,078,446, 10,793,872, 10,508,089, 10,400,249, 10,597,674, WO2018119364, WO2018119361, U.S. Pat. No. 11,180,770, US20200157086, US20210147866, U.S. Pat. No. 11,279,944, US202000331866, WO2019227036, WO2019227028, WO2022115296, and WO2011068567, each of which is herein incorporated by reference in their entirety. ACCase tolerant traits include: US20120284812, US20120284853, US20160108423, US20160244780, US20160264990, US20170275645, US20210153448, U.S. Pat. No. 10,696,975B2, U.S. Pat. No. 10,370,678, CN109082416, U.S. Pat. No. 10,694,694, US20170265469, US20170231225, each of which is herein incorporated by reference. Dicamba tolerant traits include, for example, RE45048 or U.S. Pat. No. 7,884,262. Various traits the confer tolerance to AOPP herbicides, phenoxy acid herbicides and/or pyridinyloxy acid herbicides include, for example, U.S. Pat. Nos. 10,174,337, 8,278,505, WO05107437, WO11022469, U.S. Pat. No. 10,023,874, and US2019241903 (and other traits therein), each of which is herein incorporated by reference. Additional herbicides tolerant traits of interest for stacking include glucosyl transferase polypeptides as set forth in 2018213022 or Solanesyl Diphosphate Synthase polypeptides as set forth in WO2020236790, a BIO3-BIO1 and/or BioA enzyme as described in European patent application EP23154964.3, each of which is herein incorporated by reference in their entirety.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

In certain embodiments, the additional polypeptide is a resistance protein conferring enhanced pathogen resistance, such as enhanced resistance to any one of the following pathogens: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, Fusarium virguliforme, sucking and piercing pests such as aphid, stinkbug, and whitefly, or bacterial pathogens including Pseudomonas syringae. Exemplary polynucleotides encoding proteins that confer increased pathogen resistance that may be stacked with polynucleotides of the invention include polynucleotides encoding proteins that confer increased ASR resistance as described in US Patent publication Nos. US20200354739 and PCT Publications Nos. WO2019103918, WO2021154632A1, WO2021022022, WO2021022026, WO2021022101, WO2021260673, and WO2021263249, each of which is incorporated by reference in its entirety.

Disease resistance proteins that increase resistance to various plant disease including rust, include, but are not limited to, one or more of the various resistance genes set forth in: WO2019103918; WO202100878; WO2021022022; WO2021260673; WO2022173659; WO2022159341; WO2021154632A1, WO2021022026, WO2021022101, US20220135997; U.S. Pat. No. 10,842,097; or WO2022140257; each of which is incorporated by reference in their entirety. In other embodiments, the nucleic acid sequence encoding the RG32 and/or RG34 polypeptide or active variant or fragments thereof is stacked with a native trait that confers disease resistance. For example, the various intervals, loci, or resistance genes as set forth in WO2009079729, U.S. Pat. No. 9,091,681, WO2010009404, WO2017222827, WO2021000878, WO2021022026, WO2021022101, WO2021154632, WO2022173659, (each of which is incorporated by reference in their entirety) can used to introduce the trait of interest into Glycine max. Disease resistance proteins and/or native traits that increase resistance to various plant diseases including Northern Corn Leaf Blight (NCLB) include, for example, U.S. Pat. No. 8,921,646, US2021000059, U.S. Pat. No. 10,858,668, US20200199610, WO2022/013268, WO2022/013268, U.S. Pat. Nos. 9,040,772, 10,897,862, EP3839073, each of which is herein incorporated by reference.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

In other embodiments, the polynucleotides provided herein are stacked with polynucleotides that increase protein content, and/or alter seed composition and/or fatty acid content. Such sequences include, but are not limited to, sequences disclosed in PCT Appl. No. PCT/CN2022/075977 and PCT Appl. No. PCT/CN2022/075982 both filed on Feb. 11, 2022, WO2021/044027 (which discloses various amino acid permease (AAP) polypeptides from soybean and a variety of other plants including, for example KJN37208 (a soybean AAP8 polypeptide), XP XP_003526513 (a soybean AAP8 polypeptide), NP_001242816 LOC100777963 (a soybean AAP polypeptide) and AA XP_028228300 (a soybean AAP6-like polypeptide)); US2020/0131524 which discloses various UPL3 polypeptides from a variety of plants including, for example, SEQ ID NO: 28 and a variety of UPL3 polypeptide homologs listed in Table 1; and US2021/0403933 which discloses various HECT E3 ligases, including HEL1 and HEL2, and other polypeptides and gene edits of interest, each of which is incorporated by reference in its entirety.

Double stranded RNA molecules useful with the disclosure include but are not limited to those that suppress target genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. In some examples, the trait can be identified through visual observation, such as by comparing the plant height, plant vigor, or plant flowering time of a plant with the desired combination of traits with a control plant (e.g., a plant not comprising the combination of traits or a parent plant from which the selected plant was derived).

### 7. Assay, Kits and Primers

Also provided are the kits, probes, primers and antibodies that can be used to introduce a polynucleotide sequence as described in this disclosure into a recipient plant or to detect a polynucleotide or polypeptide sequence as described in this disclosure in a plant. The polypeptide and the polynucleotide or variant and fragments thereof provided herein can be packaged as components of a kit with instructions for completing the assay described herein. A DNA detection kit is provided for use in detecting the nucleotides sequences encoding (i) the TIRA polypeptide or variants and fragments thereof, (ii) the TIRB polypeptide or variants and fragments thereof and/or (ii) a TIRATIRB fusion protein or variants or fragments thereof. A DNA detection kit is additionally or alternatively provided for detecting a transgenic event or a gene edit comprising a TIRA polypeptide, a TIRB polypeptide and/or a TIRATIRB fusion protein in a plant.

In some embodiments, the kit may comprise one or more probes having a sequence corresponding to or complementary to a sequence having 80%, 85%, 86%, 87%, 88%, 89%, 90%, 91%, 92%, 93%, 94%, 95%, 96%, 97%, 98%, 99% or 100% sequence identity with a specific region of the nucleotides sequence set forth in any one of SEQ ID NOS: 3-8, 13-19 and 21, which allows for the detection of the sequence. In some embodiments, the kit may comprise any reagent and material required to perform the assay or detection method. In specific embodiments, the probes can be used to specifically hybridize to target polynucleotide and thereby detect the nucleotide sequence set forth in any of SEQ ID NOS: 3-8, 13-19 and 21, or variants or fragments thereof.

Further provided are antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of toxin polypeptides. Thus, this disclosure provides kits comprising antibodies that specifically bind to the polypeptides described herein, including, for example, polypeptides having the sequence of any one of SEQ ID NOS: 1, 2, and 9-12 or variants or fragments thereof.

### 8. Methods of Use of Plants and Seeds

Methods of preventing pathogen damage or disease damage or ASR damage or powdery mildew damage to a plant are provided. Methods of controlling disease, or ASR, or powdery mildew, in an area of cultivation are also provided. Such methods comprise planting in an area of cultivation, a plant or seed having stably integrated into its genome a nucleotide encoding the TIRA and TIRB polypeptides, or an active variant or fragment thereof, and growing said plant or said seed, wherein co-expression of said TIRA and TIRB polypeptides, or an active variant or fragment of either, increases the resistance of the plant to a plant disease and/or plant pathogen. Such methods can alternatively comprise planting in an area of cultivation, a plant or seed having stably integrated into its genome a nucleotide encoding the TIRATIRB fusion protein, or an active variant or fragment thereof, and growing said plant or said seed, wherein expression of said TIRATIRB fusion protein, or an active variant or fragment thereof, increases the resistance of the plant to a plant disease and/or plant pathogen.

The various compositions and methods disclosed herein can provide an increased or enhanced resistance to various plant pathogens and/or plant diseases. Exemplary plant pathogens and/or plant diseases include, but are not limited to, soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme, or sucking and piercing pests such as aphid, stinkbug, and whitefly, or bacterial pathogens such as Pseudomonas syringae.

In specific embodiments the plant disease is Asian Soybean rust. In other embodiments, the plant pathogen is from the genus Phakopsora, including the species Phakopsora pachyrhizi and Phakopsora meibomiae, known to cause ASR in plants. In other specific embodiments, the plant disease is Powdery Mildew. In other embodiments, the plant pathogen is from the genus, including the species Podosphaera xanthii, known to cause Powdery mildew in plants.

In particular embodiments, co-expression of the TIRA and TIRB polypeptides, or expression of the fusion protein, in a plant, increases the resistance of the plant to ASR and/or powdery mildew.

In other embodiments, the plants co-expressing the TIRA and TIRB polypeptides, or an active variant or fragment of either, or the plants expressing the TIRATIRB fusion protein, are contacted with one or more fungicides to further prevent ASR or powdery mildew associated damage or any other plant disease of interest to the plant (i.e., the legume or the soybean). Such fungicides can be applied to any part of the plant, including for example, the seed or the leaf or to the area of cultivation. Such fungicidal compounds can be formulated or tank mixed with other fungicides or applied sequentially with other fungicides. Such fungicides may include a fungicide from one or more of the following chemical classes: Benzimidazoles, Dicarboximides, Azoles, Pyrimidines, Phenylamides, Morpholines, Carboxamides, Anilinopyrimidine, Strobilurins, Carboxylic acid amides, Inorganics, Dithiocarbamates, or Phthalimides. See, Morton, V. and Staub, T. 2008 A Short History of Fungicides. Online, APSnet Features. doi: 10.1094/APSnetFeature-2008-0308, herein incorporated by reference.

Methods are provided for screening or assaying a plant (a legume plant or a soybean plant) for resistance, to determine immunity or susceptibility of the plant to a plant disease. Such methods include, but are not limited to, screening a plant (a legume plant or a soybean plant), assaying a plant for immunity, resistance or susceptibility to a plant disease by contacting a plant cell, tissue or organ to a pathogen (e.g., Phakopsora pachyrhizi, Phakopsora meibomiae, or Podosphaera xanthii) and measuring the resistance, immunity or susceptibility of the plant or plant part to a plant disease (e.g., ASR or powdery mildew) caused by the pathogen. Any observable phenotype of the plant pathogen and/or disease can be measured. These include, but are not limited to, number, size, and/or density of disease related lesions, color of lesions (e.g., colors ranging anywhere from a tan coloration to a reddish-brown coloration), number and density of pustule formation, cyst formation, sporulation, defoliation, yield loss, or any combination thereof. Further embodiments include change in any of the aforementioned phenotypes. Still further embodiments include measured delays or expediting of proliferation of a pathogen (e.g., fungus).

9. Methods and Compositions to Generate a Resistance and/or Virulence Profile for a Plant Pathogen in an Area of Cultivation.

Resistance management of fungi in crops currently involves applying a combination of fungicides with a different mode of action at specific time points throughout the crop growing season based on product availability for the control of a specific pathogen and/or employing ASR genetic resistance traits within the crop. Approximate knowledge of the presence of resistance of a pathogen in a region or country and general recommendations are today provided, however, this only offers general information on the presence of resistance and recommendations on how to deal with it. Examples of the present disclosure provide methods of how resistance management can be improved and tailored to a specific situation. This may be achieved by determining a resistance and/or virulence profile for a plant pathogen through qualitative and quantitative pesticide (e.g., fungicide) sensitivity of the plant pathogen to a specific pesticide and/or qualitative and quantitative virulence of a plant pathogen to a crop in a specific location. This can be determined rapidly, for example close to real-time or within 48 hours as is explained in more detail below. By knowing the genetic variances of a plant pathogen population and their respective quantity or frequency at a specific location and at a given time, a more tailored treatment program or disease control measure can be provided within season that also considers resistance development of the plant pathogen. It may also be combined with a recommendation of an optimal or particular resistant crop. This will allow for a more sustainable use of the tools available for pathogen management and avoid non-effective solutions for that particular location. In addition, it can help to improve agronomic practice in order to deliver the best control of the pathogen and a sounder resistance management strategy.

Methods and compositions are provided to generate a resistance and/or virulence profile for a plant pathogen in an area of cultivation. The resistance and/or virulence profile of the area of cultivation can be employed to determine a recommended pesticide application protocol for controlling the development, reproduction, and/or viability of the ASR plant pathogen population either within the area of cultivation and/or within neighboring areas of cultivation.

a. Resistance and/or Virulence Profile

Methods and compositions are provided to generate a resistance and/or virulence profile for a plant pathogen, such as a fungal plant pathogen, a bacterial plant pathogen, an oomycete plant pathogen, a nematode plant pathogen, and/or a sucking pest plant pathogen in an area of cultivation. In particular embodiments, methods and compositions are provided to generate a resistance and/or virulence profile for an ASR pathogen. In other particular embodiments, methods and compositions are provided to generate a resistance and/or virulence profile for soybean cyst nematode, root knot nematode, pathogens causing powdery mildew, Pseudomonas syringae, and/or stinkbugs, aphids, and white flies.

As used herein, “sensitivity” refers to the susceptibility of a plant pathogen to a corresponding pesticide such as a fungicide or to a genetic resistance trait. Variations of sensitivity to pesticides and/or genetic resistance traits can result from a range of different mechanisms. In one example embodiment, “sensitivity” refers to the susceptibility of a ASR plant pathogen to an ASR pesticide such as an ASR fungicide or to an ASR genetic resistance trait. Variations of sensitivity to ASR pesticides and/or ASR genetic resistance traits can result from a range of different mechanisms.

“Resistance” refers to the ability of a plant pathogen to survive the exposure to corresponding pesticides designed to control it, including fungicides or through exposure to a genetic resistance trait. In specific embodiments, “ASR Resistance” refers to the ability of an ASR plant pathogen to survive the exposure to ASR pesticides designed to control it, including fungicides or through exposure to an ASR genetic resistance trait. Several mechanisms can shape the resistance or adaptation level. In contrast to sensitivity, resistance of an ASR plant pathogen means that the ASR plant pathogen or the ASR plant pathogen population becomes less sensitive to such an extent that a pesticide such as a fungicide or an ASR genetic resistance trait is no longer effective in controlling the development, reproduction and viability of the ASR plant pathogen and/or the ASR plant pathogen population.

Resistance development refers to a plant pathogen and/or plant pathogen population which develops a lower sensitivity to a corresponding pesticide and/or a genetic resistance trait. In one example embodiment, resistance development refers to an ASR plant pathogen and/or ASR plant pathogen population which develops a lower sensitivity to an ASR pesticide and/or an ASR genetic resistance trait. Lower sensitivity can result in complete resistance or gradual resistance to a fungicide or an ASR genetic resistance trait. A single mechanism or a combination of mechanisms can shape resistance development. The resistance development depends on how fast these mechanisms are selected in a population of time and space.

A resistance management program used herein should be understood as measures that can be adopted to minimize development of resistance of a plant pathogen and/or plant pathogen population to a plant protection product, such as measured adopted to minimize development of resistance of an ASR pathogen and/or ASR pathogen population to a plant protection product, such as a pesticide or an ASR genetic resistance trait.

As used herein, resistance profile refers to the ability of a plant pathogen or a population of plant pathogens to overcome and survive the exposure to a genetic resistance trait. In particular embodiments, resistance profile refers to the ability of an ASR plant pathogen or a population of ASR plant pathogens to overcome and survive the exposure to an ASR genetic resistance trait, a pesticide, a fungicide, a pesticide class or fungicide class as defined by FRAC, or other chemical or biological agent used for controlling development, viability and reproduction of the ASR plant pathogen.

As used herein, virulence profile refers to the ability of a plant pathogen or a population of a plant pathogen to infect and cause damage to a host, wherein the host may have a type of built-in resistance to the pathogen (i.e, a corresponding genetic resistance trait). In particular embodiments, virulence profile refers to the ability of an ASR plant pathogen or a population of an ASR plant pathogen to infect and cause damage to a host, wherein the host may have a type of built-in resistance to the pathogen (i.e, ASR genetic resistance trait).

b. Methods of Generating a Resistance and/or Virulence Profile for an ASR Plant Pathogen

A variety of methods can be used to obtain a resistance and/or virulence profile for a population of ASR plant pathogens within an area of cultivation. In one non-limiting embodiment, the frequency of the presence of one or more ASR plant pathogen effectors from an ASR plant pathogen sample is obtained from a location within the area of cultivation. In other embodiments, the frequency of the presence of one or more ASR plant pathogen effectors is obtained from a plurality of locations within the area of cultivation and a map of the frequencies throughout the area of cultivation is generated. In other cases, an average frequency of the presence of one or more of the ASR plant pathogen effectors is determined for the area of cultivation as a whole.

It will be appreciated that while the disclosed methods are with reference to obtain a resistance and/or virulence profile for a population of ASR plant pathogens within an area of cultivation, similar methods may be used to obtain the resistance and/or virulence profile for another population of plant pathogens within the area of cultivation, including, as non-limiting embodiments, profiles for a population of SCN pathogens, RKN pathogens, bacterial pathogens such as P. syringae, sucking pests such as aphids and stinkbugs and whiteflies, oomycete pathogens, as well as pathogens responsible for causing powdery mildew.

i. Obtaining Samples and Information Indicating ASR Plant Pathogen Susceptibility of the Plants in the Area of Cultivation

The frequency of an ASR effector of interest with the ASR plant pathogen population can be obtained from an ASR plant pathogen sample taken from a single location within the area of cultivation, or the information can be obtained from a plurality of locations within the area of cultivation, such that least 1, 2, 3, 4, 5, 6, 7, 8, 9, 10, 20, 30, 50 or more samples within the area of cultivation. The geographical location, the size of the area of cultivation, and the disease pressure in the area can impact the exact number of samples take and the exact location from which the sample are obtained within the area of cultivation. In addition, the selection of locations within the area of cultivation where ASR plant pathogens are collected can be influenced by previously acquired disease pressure data. In specific embodiments, the resistance profile and/or the virulence profile is generated from an ASR plant pathogen sample obtained from a single location within an area of cultivation. In other embodiments, the resistance profile and/or the virulence profile is generated from information obtained from multiple ASR plant pathogen samples taken from more than one location within the area of cultivation.

The area of cultivation can be any area location where a plant is grown. The area of cultivation includes agricultural fields, cultivated fields, and protected areas such as greenhouses.

A “sample” can be obtained by collecting at least some plant material or at least one environmental sample (aerial or soil sample) from the area of cultivation. Therefore, a sample may be a field sample such as a leaf, several leaves and/or other part of the crop that is collected from a crop or a field or alternative host plants (e.g., crop wilt type forms or volunteer crops). Additionally, or alternatively, a sample may be an environmental sample comprising soil and/or aerial material collected from where a single crop grows or collected from several points across an area of cultivation. In one example, a “sample” is to be understood as a sample of a plurality of plants, a spore trap or soil. The sample comprises at least one ASR plant pathogen and should be understood as a representation of a local population of the at least one plant pathogen.

In some embodiments, the area of cultivation can comprise a plant and/or the sample collected is from a plant or any part of a plant. The plants within the area of cultivation comprise any plant type including a legume plant. Examples are listed elsewhere herein. The frequency of the presence of an ASR plant pathogen effector within an ASR plant pathogen sample can be obtained via a variety of methods, including through an analysis of proteomic information (protein expression/amino acid sequence), transcriptional information, or genomic DNA information. Such information can be obtained through various biological materials and methods. As used herein, “biological material” means any material from an ASR plant pathogen that allows for the determination of the frequency of the one or more ASR plant pathogen effector of interest. For example, the biological material can comprise a polynucleotide. The polynucleotide can comprise DNA (genomic DNA, regulatory regions or variants or fragments thereof that influence the expression of the ASR effector of interest, introns or coding regions or variants or fragments thereof of the ASR effector(s) of interest, or a SNP or a haplotype associated with the ASR effector of interest) or RNA encoding the ASR effector of interest. Polynucleotides within the biological material can be amplified and detected using a variety of techniques employed in the art. In other embodiments, the biological material comprises a polypeptide encoding the ASR effector of interest or an active variant or fragment thereof.

For example, the amino acid sequence of given ASR effector or an active variant or fragment thereof can be detected through an antibody assay which specifically detects the ASR effector polypeptide of interest. The use of proteomics or expression of genes to produce a particular protein can employ detection of a single protein of interest or multiple differentially expressed proteins to determine the frequency of a given protein within the ASR pathogen population.

Genotypic information can be obtained by isolating, amplifying, and/or sequencing the genomic DNA of the ASR plant pathogen population with the sample, followed by the detection of the genomic DNA (including, for example, a haplotype or SNP) corresponding to the ASR effector sequence of interest. Alternatively, the transcriptome can be assayed to determine the frequency of the ASR effector within the ASR plant pathogen sample.

Test kits are provided that provide for detection of the specific set of ASR effector proteins of interest via a variety of different diagnostic methods, including DNA detection, RNA detection and protein detection. Such kits may comprise a set of nucleic acid probes and/or primers, each comprising a nucleotides sequence that specifically hybridizes to a nucleotide sequence encoding the ASR effector protein of interest. In other embodiments, the kit comprises an antibody that specifically recognizes an ASR effector polypeptide of interest. In some embodiments, these kits are employed in a field setting. See, for example, Shin et al. (2015) Vector Ecology 41:1, 63-71 detecting particular markers for resistance and Tian et al. (2018) Scientific Rep 8, no. 12587, identifying pest resistance related to increase expression of detoxifying genes.

Examples of nucleotide sequences and antibodies that can detect the various ASR effector proteins or DNA or RNA sequences encoding the ASR effector proteins can be found, for example in Krasileva et al. (2010) Plant Cell 22:2444-2458, Activation of an Arabidopsis resistance protein is specified by the in planta association of its leucine-rich repeat domain with the cognate oomycete effector, and Gupta et al. (2023) Nature Communications 14, Article 1835, Major proliferation of transposable elements shaped the genome of the soybean rust pathogen Phakopsora pachyrhizi.

ii. ASR Genetic Resistance Traits; ASR Plant Pathogen Effectors; and Methods of Detection

### 1. ASR Plant Pathogen Effectors

An ASR plant pathogen effector comprises a polypeptide produced by the ASR pathogen that can suppress the plant host defence mechanisms and facilitate infection of the plant by the ASR pathogen. The frequency of any ASR plant pathogen effector can be detected in the methods provided herein. In some embodiments, the frequency within the ASR plant pathogen population of one or more of the following ASR effector proteins is determined: SEQ ID NO: 51 of effector protein SPE335, or SEQ ID NO: 52 of effector protein SPE087, SEQ ID NO: 53 of effector protein SPE130, or SEQ ID NO: 54 of effector protein SPE196, or SEQ ID NO: 55 of effector protein SPE248, or active variants or fragments thereof. The SEQ ID NOS of each of the nucleotide (NT) sequence of each of these ASR effector proteins is set forth in Table 1B.

TABLE 1B
Effector sequences

| Effector | SEQ ID |
|---|---|
| SPE-335 | 51 |
| SPE-087 | 52 |
| SPE-130 | 53 |
| SPE-196 | 54 |
| SPE-248 | 55 |

### 2. ASR Genetic Resistance Traits

ASR genetic resistance traits comprise any trait (native trait, gene edit, or transgenic trait) stably integrated into the genome of a plant, in particular a legume plant or a soybean plant, whereby expression of the trait increases plant disease resistance against the ASR plant pathogen. ASR genetic resistance traits include R genes or disease resistance polypeptides which encode for proteins that recognize (either directly or indirectly) ASR effector proteins. A given protein encoded by an R-gene or a disease resistance polypeptide will recognize (either directly or indirectly) a specific ASR effector protein or a specific combination of ASR effector proteins.

In specific embodiments of the methods provided herein, the frequency of the ASR pathogen effector determined will vary depending on the plant planted in the area of cultivation. For example, the frequency of the presence of a given ASR effector within the ASR pathogen population can be determined based the ASR genetic resistance trait within the legume, such a soybean plant, within the area of cultivation. Table 1C provides a summary of the ASR genetic resistance trait and the trait's corresponding ASR effector(s). As used herein, a “corresponding” ASR effector protein or active variant or fragment thereof comprises an ASR effector protein which interacts (directly or indirectly) with the ASR genetic resistance trait, whereby the interaction (direct or indirect) increases the tolerance of the plant (soybean plant or legume plant) to the ASR pathogen.

TABLE 1C
Summary ASR genetic resistance trait
and corresponding ASR effector

| ASR Resistance Gene | Corresponding Effector Protein |
|---|---|
| TIRA and TIRB | SPE-335 (SEQ ID NO: 51) |
| TIRA and TIRB | SPE-087 (SEQ ID NO: 52) |
| TIRA and TIRB | SPE-130 (SEQ ID NO: 53) |
| TIRA and TIRB | SPE-196 (SEQ ID NO: 54) |
| TIRA and TIRB | SPE-248 (SEQ ID NO: 55) |

A given legume or soybean plant or seed within the area of cultivation can comprise one or more of the ASR resistance proteins or active variants or fragments thereof set forth in Table C. As such, the frequency of one or more of the corresponding ASR effector proteins or active variants or fragments thereof to a given ASR genetic resistance trait will be determined in order to generate a resistance and/or virulence profile of the area of cultivation.

In one embodiment, where an area of cultivation comprising a plant comprising a polynucleotide encoding a polypeptide comprising the amino acid sequence set forth in SEQ ID NO: 1 (TIRA) and SEQ ID NO: 2 (TIRB), or an active variant or active fragment or fusion of either, wherein expression of said polypeptide or active variant or fragment thereof increases tolerance of the plant to an ASR plant pathogen; the frequency of the presence of one or more of the corresponding: SEQ ID NO: 51 of effector protein SPE335, or SEQ ID NO: 52 of effector protein SPE087, SEQ ID NO: 53 of effector protein SPE130, or SEQ ID NO: 54 of effector protein SPE196, or SEQ ID NO: 55 of effector protein SPE248, or active variants or fragments thereof, can be determined within the sample and/or within the area of cultivation. As discussed elsewhere herein, the frequency of the effector can be determined at the polynucleotide or polypeptide level.

In specific embodiments, the polynucleotide encoding the ASR genetic resistance trait is heterologous to the plant.

### 3. Methods of Detection

Determining the frequency of an ASR effector polypeptide or active variant or fragment thereof within a sample from an area of cultivation can be performed via detection of polypeptides or polypeptides as discussed elsewhere herein, including through the use of primers, probes, PCR, ect.

In one embodiment, a PCR reaction is employed to determine the frequency of the ASR effector polypeptide or active variant for fragment thereof within the ASR plant pathogen population in the area of cultivation. In other embodiments, when detecting a polynucleotide of interest (ie. a polynucleotide of an ASR effective protein), stringent hybridization conditions can be used. employed to determine the frequency of the ASR effector polypeptide or active variant for fragment thereof within the ASR plant pathogen population in the area of cultivation. Such methods are described elsewhere herein. Such methods are described elsewhere herein.

In other embodiments, the biological material is subjected to DNA sequencing for determining if a given ASR effector is present. As used herein, a genetic variance, gene variance or a variance of a plant pathogen is to be understood as a mutation in the DNA sequence different from the wild type sequence. For example, in a genetic variance at least one nucleotide in a DNA sequence has been permanently changed, deleted or inserted.

Genotype refers to the unique combination of multiple genetic variances, gene variances or variances of a plant pathogen. If multiple independent genetic variations can be brought together into different unique combinations, then each of these unique combinations might express a different sensitivity or virulence profile.

In embodiments where the biological material is subjected to DNA sequencing for determining the nucleic acid sequence in the sample, this operation may comprise using a sequencer capable of sequencing at least 200, 300, 400 or 500 base pairs in a single read. For example, Oxford Nanopore sequencing Technologies such as MinION, GridION or PromethION or PacBio Sequel Systems implementing single-molecule real-time sequencing as provided by PACBIO may offer the required capability. These technologies may be referred to as third generation sequencer and they provide a high throughput combined with larger sequenced genetic regions from a few hundred base pairs up to 10,000 base pairs. The technology provided by Oxford Nanopore Technologies comprises flow cells which contain an array of tiny holes referred to as nanopores that are embedded in an electro-resistant membrane. Each nanopore corresponds to its own electrode connected to a channel and sensor chip, which measures the electric current that flows through the nanopore. When a molecule passes through a nanopore, the current is disrupted to produce a characteristic ‘squiggle’ or a current intensity value. The squiggle is then decoded using base calling algorithms to determine the DNA. Base calling is a computational process of translating the squiggle into DNA sequences. Specific bioinformatics pipelines may be combined to enable quantification of one or more ASR effector polypeptides in a given ASR pathogen populations.

Before subjecting a sample to DNA sequence, DNA may be extracted from the received sample. This DNA sample may include a diversity of individuals from a plant pathogenic species representative of genetic variation of the disease. In one example, specific panels of multiplex PCR are designed to co-amplify multiple targets in a single PCR reaction. This optimized step requires the identification of non-self-cross-hybridizing primers in conserved regions to cope with natural variability between individuals composing a population. Through this optimization genetic loci comprising one or more of the ASR effector polypeptides of interest are amplified from the largest fraction of strains in a natural population. Primers may also be checked to ensure that other plant pathogens or other ASR effector proteins that could occur in the same area are not unspecific amplified. Thereafter, DNA barcoding may be performed to identify a specific sample if multiple samples are bulked before sequencing the plant pathogen. Thereafter, sequencing preparation may be performed followed by the operation of DNA sequencing using a third-generation sequencer as described above.

It should be understood that the present disclosure is not limited to the specific third generation sequencers as described herein but that any sequencing technology can be applied that generates data within a timeframe giving a current insight into a disease of a plant.

Referring now again to the operation determining the presence of genetic polymorphisms based on the DNA sequencing. This operation may comprise comparing the DNA sequence of the sample with a reference DNA sequence of a plant pathogen and identifying genetic polymorphisms that are unique to a given ASR effector of interest.

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### 4. Assay, Kits and Primers

Also provided are the kits, probes, primers and antibodies that can be used to determine the frequency of an ASR plant pathogen effector in an area of cultivation. The polypeptide and the polynucleotide or variant and fragments thereof of the ASR plant pathogen effector can be packaged as components of a kit with instructions for completing the assay described herein. A DNA or RNA detection kit is provided for use in detecting the nucleotides sequences encoding the ASR plant pathogen effector polypeptide or variants and fragments thereof.

In some embodiments, the kit may comprise one or more probes having a sequence corresponding to or complementary to a sequence having 80%, 85%, 86%, 87%, 88%, 89%, 90%, 91%, 92%, 93%, 94%, 95%, 96%, 97%, 98%, 99% or 100% sequence identity with a specific region of the nucleotides sequence, which allows for the detection of the sequence of interest. In some embodiments, the kit may comprise any reagent and material required to perform the assay or detection method. In specific embodiments, the probes can be used to specifically hybridize to target polynucleotide and thereby detect the nucleotide sequence set forth in SEQ ID NO: 21 or 22 or variants or fragments thereof.

Further provided are antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of toxin polypeptides. Thus, this disclosure provides kits comprising antibodies that specifically bind to the polypeptides described herein, including, for example, polypeptides having the sequence of SEQ ID NO: 19 or 20 or variants or fragments thereof.

c. Determining the Frequency of the Presence of an ASR Plant Pathogen Effector in the Biological Material and Generating Recommended Pesticide Application

Quantification of one or more of an ASR effector of interest (SPE335, SPE087, SPE130, SPE196, or SPE248, or an active variant thereof) can be expressed as a frequency within the ASR plant pathogen population within the area of cultivation. The frequency of occurrence can be determined as a percentage of the whole population of the ASR plant pathogen present in the sample. As an example, one or more ASR effector of interest can be determined to be present at a certain frequency within the ASR population. Such a frequency within the ASR population may be at least about 1%, 2%, 5%, 10%, 15%, 20%, 25%, 30%, 35%, 40%, 45%, 50%, 55%, 60%, 65%, 70%, 75%, 80%, 85%, 90%, 95% or 100% of the ASR plant pathogen population from the sample and/or within the area of cultivation; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within a sample and/or within the ASR plant pathogen population in the area of cultivation. The frequency of the ASR effector polypeptides can also be expressed in alternative ways, for example, heatmaps, boxplots, or pies associated with or without maps.

The legume plant or soybean plant in the area of cultivation can comprise one or more ASR genetic resistance traits, such as TIRA or TIRB or an active variant or fragment thereof. As explained elsewhere herein, depending on the ASR genetic resistance trait within the crop planted in the area of cultivation, the ASR effector(s) detected within the population will change. For example, when the area of cultivation comprises a legume or soybean plant expressing TIRA and TIRB or an active variant or fragment thereof, any one of the corresponding ASR effectors of TIRA and/or TIRB can be detected in the ASR plant pathogen population. For example, any one or combination of SPE335, SPE087, SPE130, SPE196, or SPE248, or active variants or fragments thereof can be detected. A resistance profile in such an area of cultivation where the frequency of any one of or any combination of SPE335, SPE087, SPE130, SPE196, or SPE248, or an active variant thereof, is present at a high frequency within the population indicates that a pesticide application protocol with a greater ability to control the ASR pathogen will be needed. A resistance profile in such an area of cultivation where the frequency of any one or combination of SPE335, SPE087, SPE130, SPE196, or SPE248, or an active variant thereof is present at a low frequency within the population indicates that a pesticide application protocol with a decreased ability to control the ASR pathogen will be needed, as the ASR genetic resistance within the plants will be able to control the ASR pathogen population. Such methods allow for improved ASR pathogen resistance management in a given area of cultivation. While plants having TIRA and TIRB were used in the example above, a similar protocol can be used for any of the ASR genetic resistance traits disclosed herein with their corresponding ASR effector proteins or any combination thereof. See, for example, Table 1C herein for the corresponding ASR effector protein for a given ASR genetic resistance trait.

As used herein, “high frequency within the population” of the corresponding ASR effector protein for the ASR genetic resistance trait will result in a more susceptible plant population. A high effector protein frequency within the population occurs when the one or more of the ASR effectors, either alone or in combination, are present within the ASR plant pathogen population at least a frequency of about 1%, 2%, 5%, 10%, 15%, 20%, 25%, 30%, 35%, 40%, 45%, 50%, 55%, 60%, 65%, 70%, 75%, 80%, 85%, 90%, 95% or 100% frequency; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within the ASR plant pathogen population.

As used herein, “low frequency within the population” of the corresponding ASR effector protein for the ASR genetic resistance trait will result in a less susceptible plant population. A low ASR effector protein frequency within the population occurs when the one or more of the ASR effectors, either alone or in combination, are present within the ASR plant pathogen population at least a frequency of about 1%, 2%, 5%, 10%, 15%, 20%, 25%, 30%, 35%, 40%, 45%, 50%, 55%, 60%, 65%, 70%, 75%, 80%, 85%, 90%, 95% or 100% frequency; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within the ASR plant pathogen population.

The frequency of an ASR plant pathogen effector of interest at the sampling locations can be used to generate the frequency of the various ASR plant pathogen effectors within the area of cultivation. By combining data from different sampling locations within the area of cultivation the predicted local frequency of a given ASR plant pathogen effector within the ASR plant pathogen population can be determined for the local situation. A resistance map and/or a virulence map for that given area of cultivation can then be determined.

The resistance profile map and/or virulence profile map may then be employed to identify candidate pesticides for use in a pesticide application protocol to increase the resistance of the legume plants or soybean plants to the ASR plant pathogens within the area of cultivation. A recommended pesticide application protocol for the area of cultivation comprising at least one disease control measure for controlling the development, reproduction and/or viability of the ASR plant pathogen based on the resistance profile and/or virulence profile can then be generated.

Disease control measure or treatment program is to be understood as management of the ASR plant pathogen, for example, by application of a plant protection product such as a pesticide, timing or intervals of application of a plant protection product or selection of an optimal or particular resistant crop in order to control the development, reproduction and/or viability of the pathogen whilst minimizing resistance development. As discussed elsewhere herein, the disease control measure assembled will take into consideration the resistance profile and/or virulence profile of the ASR plant pathogens detected in the area of cultivation.

A disease control measure can include the application of a fungicide that can increase the development, reproduction and/or viability of the ASR plant pathogen population within the area of cultivation. Such fungicide applications include, but are not limited to, demethylation inhibitors (DMI's—tebuconazole, cyproconazole, protioconazole (triazolintione), epoxiconazole, flutriafol and others); quinone oxidase inhibitors (Qol's—azoxystrobin, trifloxystrobin, picoxystrobin and pyraclostrobin), succinate dehydrogenase inhibitors (SDHI's—fluxpyroxade, bixafen and benzovindiflupyr), cupric (oxychloride), nitriles (e 3), strobilurins, and mancozeb. Additional fungicidal application include fungicides belonging to the Carboxamide group, which have a specific mode of action, inhibiting fungal respiration, of complex II-succinate dehydrogenase (SDHI), to control ASR. Members of the carboxamide group include bixafen, fluxopyiraxade and benzovindiflupyr. Fungicides benzovindiflupyr, bixafen, fluxapyroxad, furametpyr, isopyrazam, penflufen, penthiopyrad and silkxane; or, multi-site fungicides such as mancozeb, chlorothalonil, and/or metiram.

In other embodiments, the method further comprises determining at least one disease control measure for applying to a neighbouring location based on the resistance and/or virulence profile.

Exemplary methods for determining a resistance profile and/or virulence profile in an ASR plant pathogen population present in an area of cultivation according to the present disclosure are as follows. The method comprises collecting samples from the area of cultivation wherein the samples could be a field sample or an environmental sample, for example an aerial sampling by spore traps, plant material or soil material as discussed above. Biological material from the sample is obtained, including for example, polynucleotides or polypeptides. In some embodiments, polynucleotides from the biological material are sequenced and/or amplified and/or detected or polypeptides are detected in order to detect an ASR plant pathogen effector of interest that possibly confer a higher virulence to a resistant plant or cultivar. The method further comprises determining the qualitative presence of the ASR plant pathogen effector in the plant pathogen or pathogen population and quantifying the frequency of the one or more ASR plant pathogen effector of interest. Thereafter, the method further comprises generating a resistance profile and/or virulence profile of the ASR plant pathogen or ASR pathogen population by combining the results with information or knowledge obtained from dedicated studies assessing the impact of a given frequency of the one or more ASR plant pathogen effector on crop productivity.

Such methods enable the quantitative and qualitative description of the resistance profile and/or the virulence profile of a population of a ASR plant pathogen present in a location to be determined rapidly such as close to real-time, within 48 hours or a few days. This provides opportunities to have a clear and more detailed understanding of the possible resistance profiles present in a location and to help determine a more tailored disease control measure and/or resistance management program for the current season and/or future seasons. As such, the most effective type of pesticide(s) for controlling the pathogen and/or the best crop variety can be selected such that pesticides with high presence of resistance can be avoided.

Referring now again to the operation of collecting a sample of a ASR plant pathogen, this operation may comprise receiving a sample from a farmer who has collected the sample from a field or receiving a sample from another source, e.g. a wild population of spontaneous crops or flowers, where the profile of resistance of a plant pathogen population is to be determined. The sample may be specimens that the farmer has collected from various plants across a field. Alternatively, or additionally, a sample may be environmental (e.g., soil and/or aerial samples).

Referring now again to the operation of generating a resistance profile and/or virulence profile of the plant pathogen. This operation may comprise interpreting the data determined by polynucleotide sequencing, polynucleotide amplification and detection and/or by polypeptide detection in order to detect the ASR effectors within the biological material. By applying knowledge of the susceptibility of the legume and/or soybean plants in the area or cultivation or to be planted in the area of cultivation in combination with the frequency of the different ASR effectors present in the sample(s) from the area of cultivation can be determined. More specifically, generating a resistance profile may comprise associating the presence of one or more ASR effector and corresponding frequency in the ASR plant pathogen population of the sample as determined in the preceding operation 103 with a level of susceptibility of the legumes and/or soybean plants in the area of cultivation or to be planted in the area of cultivation. The resistance level can be considered to be an index, which condenses complex information of frequencies and resistance factors of each ASR effector or a combination of ASR effectors to a single value comparable between different samples. The resistance level of a plant pathogen population of a sample requires an understanding of the effect of each ASR effector detected to the resistance of the plant (legumes and/or soybeans) within the area of cultivation.

Further provided is a method comprising determining at least one pesticide or other disease control measure for controlling the development, reproduction and/or viability of the crop pathogen based on the resistance profile and/or virulence profile. For example, a resistance profile and/or virulence profile may be determined before any use of pesticides to control the ASR pathogen. The resistance profile of the plants in the area of cultivation to specific ASR effectors (e.g., SPE335, SPE087, SPE130, SPE196, or SPE248, or an active variant thereof) of the pathogen population can be generated or measured as described above in example method by monitoring the presence of one or more of the ASR effectors in the sample and quantify their frequency. Similarly, a virulence profile can be generated as described above. Based on the resistance profile and/or virulence profile a pesticide or other disease control measure can be determined or recommended. The method may further comprise an operation of collecting or receiving a subsequent or additional sample from the same location, crop or plant as the initial sample, wherein the subsequent sample has been treated with the pesticide or other disease control measure determined in operation. The subsequent sample is subjected to the operations described above and thereafter a resistance profile and/or virulence profile is generated similar to operation. Finally, the generated resistance profile and/or virulence profile is compared with that of the initial sample. For example, if a resistance profile was generated for the initial sample then it is compared with a resistance profile generated for the subsequent sample, similarly if a virulence profile was generated for the initial sample then it is compared with a virulence profile of the subsequent sample. From the comparison of the two resistance profiles, it is possible to determine if the population resistance to a fungicide class improved or deteriorated as a consequence of the agronomic decisions taken to control the disease. For example, the ratio between a sensitivity index generated before and after a pesticide treatment provides information about the sustainability of the disease control measure applied and can be used as a tool to identify and inform about suboptimal disease control measures and resistance management. The sharing of information about the disease control measures such as date of application, plant protection product applied, rate applied and other information will enable alerts about a risk decision and propose alternatives for the following seasons.

In one example, based on the comparison, the method may further comprise determining at least one pesticide or other disease control measure for controlling the plant pathogen. This pesticide or other disease control measure may be the same or different to the one(s) applied before the subsequent sample was collected and received.

The first or initial sample described herein may be collected at the beginning of the season and the subsequent or additional sample may be collected during or at the end of the season (middle or end). The beginning and the middle/end of the season may be a period of a day, several days, a week or several weeks, a month or several months, a year or several years depending on the crop, pathogen or sampling frequency needed for the particular situation. In the event that a more frequent analysis of a resistance profile is required, other samplings could be done for example after each application or over a time series.

Although the samples have been described to be collected at different time points relative to a season, it should be understood that the samples may alternatively or additionally be collected when the relevant plant is in a particular growth stage.

Furthermore, the present disclosure is not limited to an initial sample and a single subsequent sample. Several subsequent samples may be collected at different time points and then processed so as to generate a resistance profile and/or virulence profile.

It is also envisaged that the methods described herein for determining at least one pesticide or other disease control measure (as described above) for a location can be applied to neighboring locations. This is based on the assumption that the variant(s) of the plant pathogen present in the location are also present in the neighboring locations. This may be the case for environmental aerial samples wherein the plants or crops are untreated with pesticides. It is well known that different situations can be found in neighboring locations, often due to different resistant plants or crops being cultivated and/or due to different treatment programs of pesticides, such as fungicides, having been adopted in previous years. In such cases, additional considerations need to be taken before applying disease control measures to a location that has been determined for an adjacent location.

A method for determining a resistance profile and/or virulence profile for a plant pathogen in a location is provided. The method comprises receiving a sample of a plant pathogen from the location wherein the samples could be a field sample or an environmental sample. The method further comprises subjecting the sample to polynucleotide sequencing, polynucleotide amplification, polynucleotide detection and/or polypeptide detection to determine the presence of one or more ASR effectors withing the ASR population. The presence of at least one ASR effector within the ASR plant pathogen population is determined and said at least one ASR effector is quantified. Thereafter, the method further comprises generating a resistance profile and/or virulence profile of the plant pathogen or pathogen population.

### 5. Other Plant Pathogen Effectors

Plant pathogen effectors comprise a polypeptide produced by the pathogen that can suppress the plant host defence mechanisms and facilitate infection of the plant by the pathogen. It will be appreciated that while the methods of section 9a-c are described with reference to determining the frequency of ASR plant pathogen effectors, generating a resistance or virulence profile of ASR plant pathogens, and generating recommended pesticide application, similar methods may be adapted for other plant pathogen effectors. In particular embodiments, the frequency for a bacterial pathogen (such as P. syringae), a nematode pathogen (such as Soybean cyst nematode (SCN), or Root Knot nematode (RKN)), or oomycete plant pathogen populations, or one or more corresponding effector proteins is determined. The SEQ ID NOS of each of the nucleotide (NT) sequence and/or amino acid (AA) sequence of each of these effector proteins and the corresponding pathogen that can be assayed for is set forth in Table 1D and elaborated in Example 10.

TABLE 1D
Effector sequences

| Effector | SEQ ID | Pathogen/Disease |
|---|---|---|
| SNE-05 NT | 56 | SCN |
| SNE-18 NT | 57 | SCN |
| SNE-28 NT | 58 | SCN |
| HopT1-1 NT | 59 | Pseudomonas syringae pathovars tomato strain |
| | | DC3000 |
| HopT1-1 AA | 61 | Pseudomonas syringae pathovars tomato strain |
| | | DC3000 |
| HopT1-2 NT | 60 | Pseudomonas syringae pathovars tomato strain |
| | | DC3000 |
| HopT1-2 AA | 62 | Pseudomonas syringae pathovars tomato strain |
| | | DC3000 |
| EPCSEP-066 | 63 | Powdery Mildew |
| (NT) | | |
| EPCSEP-066 | 65 | Powdery Mildew |
| (AA) | | |
| EPCSEP-099 | 64 | Powdery Mildew |
| (NT) | | |
| EPCSEP-099 | 66 | Powdery Mildew |
| (AA) | | |
| DMCEP-046 | 67 | Oomycetes |
| (NT) | | |
| DMCEP-046 | 69 | Oomycetes |
| (NT) | | |
| DMCEP-084 | 68 | Oomycetes |
| (NT) | | |
| DMCEP-084 | 70 | Oomycetes |
| (NT) | | |

### 10. Methods for Identifying Variant Polypeptides Encoding an R Gene or Other Plant Pathogen Genetic Resistance Trait

Variants of a polypeptide encoding an R gene or other plant pathogen genetic resistance trait (e.g., ASR genetic resistance trait), including sequences from other organisms, can be identified based on their sequence identity and/or functional identity with the given TIRA and/or TIRB polypeptide and genes disclosed herein. In one example, variant polypeptides and polynucleotides of the TIRA and/or TIRB polypeptides, and polynucleotide sequences encoding such polypeptides, comprise proteins that interact with the same set of effector proteins (see Tables 1B, 1C and 1D) to generate a localized hypersensitive response via a common mode of action when expressed in a plant (see also Examples 8 and 10). Variant polypeptide and polynucleotides including orthologs and allelic variants of TIRA and/or TIRB polypeptides of SEQ ID NOS: 1 and 2 and/or RG32 and/or RG34 genes of SEQ ID NOS: 3-8 are expected to interact with effector proteins to generate a localized hypersensitive cell death response.

Accordingly, in some embodiments, methods are disclosed of identifying novel disease resistance polypeptides, the method comprising: identifying a set of plant pathogen effector proteins that interact with the TIRA polypeptide of SEQ ID NO: 1; assaying for interaction of the identified set of plant pathogen effector proteins with a putative TIRA polypeptide or an TIRA polypeptide having at least 40%, at least 50%, at least 60%, at least 70%, at least 80%, at least 90% or at least 95% sequence identity to SEQ ID NO: 1; and in response to detection of interaction between the putative TIRA polypeptide and the identified effector(s), such as via the presence of a hypersensitive response or localized cell death, indicating that the putative TIRA polypeptide has the same mode of action as the TIRA polypeptide. In other embodiments, methods are disclosed of identifying novel disease resistance polypeptides, the method comprising: identifying a set of plant pathogen effector proteins that interact with the TIRB polypeptide of SEQ ID NO: 2; assaying for interaction of the identified set of plant pathogen effector proteins with a putative TIRB polypeptide or an TIRB polypeptide having at least 40%, at least 50%, at least 60%, at least 70%, at least 80%, at least 90% or at least 95% sequence identity to SEQ ID NO: 2; and in response to detection of interaction between the putative TIRB polypeptide and the identified effector(s), such as via the presence of a hypersensitive response or localized cell death, indicating that the putative TIRB polypeptide has the same mode of action as the TIRB polypeptide. In still other embodiments, methods are disclosed of identifying novel disease resistance polypeptides, the method comprising: identifying a set of plant pathogen effector proteins that interact with the TIRB and TIRB polypeptides, co-expressed; assaying for interaction of the identified set of plant pathogen effector proteins with a putative TIRA polypeptide and a putative TIRB polypeptide, co-expressed, wherein the putative TIRA polypeptide has at least 40%, at least 50%, at least 60%, at least 70%, at least 80%, at least 90% or at least 95% sequence identity to SEQ ID NO: 1, and the putative TIRB polypeptide has at least 40%, at least 50%, at least 60%, at least 70%, at least 80%, at least 90% or at least 95% sequence identity to SEQ ID NO: 2; and in response to detection of interaction between the co-expressed putative TIRA and TIRB polypeptides and the identified effector(s), such as via the presence of a hypersensitive response or localized cell death, indicating that the putative TIRA and TIRB polypeptides have the same mode of action as the TIRA and TIRB polypeptides.

In some embodiments, the effector proteins are expressed in a cell, purified, and infiltrated into a plant cell for an interaction assay with the putative TIRA or TIRB polypeptide. In other embodiments, a transient expression system, such as an Agrobacterium-mediated transient expression system in a plant can be used to observe the hypersensitive response cell death phenotype (HR) triggered by co-expression of a putative TIRA or TIRB gene system comprising (a) a TIRA or TIRB variant (e.g., orthologs or homologs or allelic variants of TIRA or TIRB) and their putative cognate effector (e.g., an effector protein recognized by the TIRA polypeptide or TIRB polypeptide of SEQ ID NOS: 1 or 2). Presence of an interaction, e.g., presence of an HR response and localized cell death (such as shown in Examples 8 and 10), indicates that the protein encoded by the putative TirA or TirB genes have functional identity and a common mode of action as the TIRA or TIRB, respectively, of the present disclosure, even if the sequence identity is low (e.g., lower than 60% sequence identity, such as 50% identity or 40% identity or lower).

### NON-LIMITING EMBODIMENTS INCLUDE

### Example Embodiments of Methods for Controlling Disease Resistance

A1. A method of controlling disease resistance in an area of cultivation comprising the step of planting in the area of cultivation a legume plant, a plant part or a seed having stably incorporated into its genome:
(a) a first heterologous nucleotide sequence encoding a TIRA polypeptide, or an active variant thereof, having at least 80%, 85%, 90%, 91%, 92%, 93%, 94%, 95%, 96%, 97%, 98% 99% or 100% identity to SEQ ID NO: 1;
(b) a second heterologous nucleotide sequence encoding a TIRB polypeptide, or an active variant thereof, having at least 80%, 85%, 90%, 91%, 92%, 93%, 94%, 95%, 96%, 97%, 98% 99% or 100% identity to SEQ ID NO: 2; and
(c) a heterologous promoter operably coupled to the first and/or the second heterologous nucleotide sequence,
wherein an expression level of the TIRA polypeptide, or active variant thereof, relative to the TIRB polypeptide, or active variant thereof, is rebalanced in the plant, plant part or seed as compared to the expression level in a control plant, plant part or seed, wherein the rebalanced expression level of the TIRA polypeptide, or active variant thereof, relative to the TIRB polypeptide, or active variant thereof, comprises a ratio of about 0.5:1 to about 2:1, and wherein the TIRA polypeptide, or active variant thereof, confers disease resistance to the legume plant, plant or seed when co-expressed with the TIRB polypeptide, or active variant thereof, and wherein the TIRA polypeptide, or active variant thereof, confers disease resistance to the legume plant, plant or seed when co-expressed with the TIRA polypeptide, or active variant thereof.

A2. The method of embodiment A1, wherein one or more of the TIRA polypeptide, TIRB polypeptide, or active variants thereof, are tagged with a detectable marker.

A3. The method of embodiment A2, wherein the TIRA polypeptide, or active variant thereof, and the TIRB polypeptide, or active variant thereof, are expressed in the legume plant, plant part or seed as a fusion protein.

A4. The method of embodiment A3, wherein the TIRA polypeptide or active variant thereof is at the N-terminus of the fusion protein and is fused to the TIRB polypeptide or active variant thereof via a linker sequence.

A5. The method of embodiment A3, wherein the TIRB polypeptide or active variant thereof is at the N-terminus of the fusion protein and is fused to the TIRA polypeptide or active variant thereof via a linker sequence.

A6. The method of any one of embodiments A3-A5, wherein the first heterologous nucleotide sequence encoding the TIRA polypeptide or active variant thereof, and the second heterologous nucleotide sequence encoding the TIRB polypeptide or active variant thereof, are both operably coupled to, and transcribed by, the heterologous promoter, and wherein the heterologous promoter is an endogenous promoter, a rust inducible promoter, or a constitutive promoter active in said plant.

A7. The method of any one of embodiments A3-A6, wherein the heterologous promoter comprises a nucleotide sequence having at least 95% sequence identity to any one of SEQ ID NOS: 23-28 or wherein the promoter comprises the sequence of any one of SEQ ID NOS: 22-27.

A8. The method of any one of embodiments A3-A7, wherein the fusion protein comprises one or more or a loss of function mutation that reduces an NADase activity of the TIRA polypeptide or active variant thereof and a gain of function mutation that increases an NADase activity of the TIRA polypeptide or active variant thereof, wherein the TIRA polypeptide or active variant thereof with the loss of function mutation that decreases the NADase activity or the gain of function mutation that increases the NADase activity is able to confer disease resistance to the legume plant, plant or seed when co-expressed with the TIRB polypeptide or active variant thereof.

A9. The method of any one of embodiments A3-A8, wherein the fusion protein comprises a loss of function mutation that reduces an NADase activity of the TIRB polypeptide or active variant thereof, wherein the TIRB polypeptide or active variant thereof with the loss of function mutation that decreases the NADase activity is able to confer disease resistance to the legume plant, plant or seed when co-expressed with the TIRA polypeptide or active variant thereof.

A10. The method of any one of embodiments A8 and A9, wherein the TIRA polypeptide or active variant thereof has a dumbbell structure comprising a TIRA1 region and a TIRA2 region, each of the TIRA1 and TIRA2 region comprising an NADase site, wherein the TIRB polypeptide or active variant thereof has a dumbbell structure comprising a TIRB1 region and a TIRB2 region, each of the TIRB1 and TIRB2 region comprising an NADase site, and wherein the loss of function mutation that reduces an NADase activity of the TIRA polypeptide or active variant thereof comprises a loss of function mutation in the NADase site of the TIRA1 region or the TIRA2 region;
wherein the gain of function mutation that increases an NADase activity of the TIRA polypeptide or active variant thereof comprises a gain of function mutation in the NADase site of the TIRA2 region,
wherein the loss of function mutation that reduces an NADase activity of the TIRB polypeptide or active variant thereof comprises a loss of function mutation in the NADase site of the TIRB1 region; and
wherein the fusion protein does not comprise a loss of function mutation in the NADase site of the TIRB2 region of the TIRB polypeptide or active variant thereof.

A11. The method of any one of embodiments A8-A10 wherein:
(a) the loss of function mutation that reduces the NADase activity of the TIRA polypeptide or active variant thereof comprises at least one mutation at a position corresponding to position 85 of SEQ ID NO: 1;
(b) the gain of function mutation that increases the NADase activity of the TIRA polypeptide or active variant thereof comprises at least one mutation at a position corresponding to position 251 of SEQ ID NO: 1
(c) the loss of function mutation that reduces the NADase activity of the TIRB polypeptide or active variant thereof comprises at least one mutation at a position corresponding to position 87 of SEQ ID NO: 2.

A12. The method of embodiment A1, wherein the first heterologous nucleotide sequence encoding the TIRA polypeptide, or active variant thereof, is operably coupled to a first heterologous promoter in a first expression cassette, and wherein the second heterologous nucleotide sequence encoding the TIRB polypeptide, or active variant thereof, is operably coupled to a second, different heterologous promoter in a second expression cassette, and wherein the first expression cassette is positioned transcriptionally upstream of the second expression cassette.

A13. The method of embodiment A1, wherein the first heterologous nucleotide sequence encoding the TIRA polypeptide, or active variant thereof, is operably coupled to a first heterologous promoter in a first expression cassette, and wherein the second heterologous nucleotide sequence encoding the TIRB polypeptide, or active variant thereof, is operably coupled to a second, different heterologous promoter in a second expression cassette, and wherein the first expression cassette is positioned transcriptionally downstream of the second expression cassette.

A14. The method of embodiment A12 or A13, wherein the first heterologous nucleotide sequence comprises:
(i) a nucleotide sequence encoding a polypeptide having at least 80%, at least 85%, at least 90%, at least 91%, at least 92%, at least 93%, at least 94%, at least 95%, at least 96%, at least 97%, at least 98%, or at least 99% sequence identity to SEQ NO: 1; or
(ii) a nucleotide sequence encoding the polypeptide of SEQ NO: 1; or
(iii) the nucleotide sequence of (i) or (ii) and further comprising a loss of function mutation that decreases an NADase activity of the TIRA polypeptide or active variant thereof while maintaining an ability of the TIRA polypeptide or active variant thereof to confer disease resistance to the legume plant, plant or seed when co-expressed with the TIRB polypeptide, or active variant thereof; or
(iv) the nucleotide sequence of (i) or (ii) and further comprising a gain of function mutation that increases an NADase activity of the TIRA polypeptide or active variant thereof while maintaining an ability of the TIRA polypeptide or active variant thereof to confer disease resistance to the legume plant, plant or seed when co-expressed with the TIRB polypeptide, or active variant thereof.

A15. The method of embodiment A12 or A13 or A14, wherein the second heterologous nucleotide sequence comprises:
(v) a nucleotide sequence encoding a polypeptide having at least 80%, at least 85%, at least 90%, at least 91%, at least 92%, at least 93%, at least 94%, at least 95%, at least 96%, at least 97%, at least 98%, or at least 99% sequence identity to SEQ ID NO: 2; or
(vi) a nucleotide sequence encoding the polypeptide of SEQ ID NO: 2; or
(vii) the nucleotide sequence of (v) or (vi) and further comprising a loss of function mutation that decreases an NADase activity of the TIRB polypeptide or active variant thereof while maintaining an ability of the TIRB polypeptide or active variant thereof to confer disease resistance to the legume plant, plant or seed when co-expressed with the TIRA polypeptide, or active variant thereof.

A16. The method of embodiment A15, wherein the TIRA polypeptide or active variant thereof has a dumbbell structure comprising a TIRA1 region and a TIRA2 region, each of the TIRA1 and TIRA2 region comprising an NADase site, wherein the TIRB polypeptide or active variant thereof has a dumbbell structure comprising a TIRB1 region and a TIRB2 region, each of the TIRB1 and TIRB2 region comprising an NADase site, and
wherein the loss of function mutation that reduces an NADase activity of the TIRA polypeptide or active variant thereof comprises a loss of function mutation in the NADase domain of the TIRA1 region and/or TIRA2 region,
wherein the gain of function mutation that reduces an NADase activity of the TIRA polypeptide or active variant thereof comprises a loss of function mutation in the NADase domain of the TIRA2 region, and
wherein the loss of function mutation that reduces an NADase activity of the TIRB polypeptide or active variant thereof comprises a loss of function mutation in the NADase domain of the TIRB1 region and does not comprise a loss of function mutation in the NADase domain of the TIRB2 region of the TIRB polypeptide or active variant thereof.

A17. The method of embodiment A16, wherein
(a) the loss of function mutation in the NADase domain of the TIRA1 region of the TIRA polypeptide or active variant thereof comprises at least one mutation at a position corresponding to position 85 of SEQ ID NO: 1;
(b) the gain of function mutation in the NADase domain of the TIRA2 region of the TIRA polypeptide or active variant thereof comprises at least one mutation at a position corresponding to position 251 of SEQ ID NO: 1; and/or
(c) the loss of function mutation in the NADase domain of the TIRB1 region of the TIRB polypeptide or active variant thereof comprises at least one mutation at a position corresponding to position 87 of SEQ ID NO: 87.

A18. The method of any one of embodiments A13-A17, wherein the first heterologous promoter is an endogenous promoter, a rust inducible promoter, or a constitutive promoter active in said plant; and wherein the second promoter is an endogenous promoter, an inducible promoter, optionally a rust inducible promoter, or a constitutive promoter active in said plant.

A19. The method of any one of embodiments A13-A18, wherein the first heterologous promoter is an endogenous promoter or a constitutive promoter active in said plant; and wherein the second promoter is a rust inducible promoter active in said plant.

A20. The method of any one of embodiments A13-A19, wherein:
(i) the endogenous promoter comprises a nucleotide sequence having at least 95% sequence identity to any one of SEQ ID NOS: 26-27, or wherein the endogenous promoter comprises any one of SEQ ID NO: 26-27
(ii) the rust inducible promoter comprises a nucleotide sequence having at least 95% sequence identity to SEQ ID NO: 25, or wherein the endogenous promoter comprises SEQ ID NO: 25; and/or
(iii) the constitutive promoter comprises a nucleotide sequence having at least 95% sequence identity to any one of SEQ ID NOS: 22-24, or wherein the constitutive promoter comprises any one of SEQ ID NOS: 22-24.

A21. A legume plant produced by the method of any one of embodiments A1-A20.

A22. The legume plant of embodiment A20, wherein the legume plant is a legume crop plant, and optionally wherein the legume crop plant is alfalfa, clover, pea, bean, lentil, lupin, mesquite, carob, soybean, pigeon pea, peanut or tamarind.

A23. The legume plant of embodiment A20, wherein the legume crop plant is a soybean plant.

A23. The legume plant of embodiment A21, wherein the soybean plant is an elite soybean plant.

A24. The legume plant of any one of embodiments A21-A23, wherein the plant has resistance to one or more of the following: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum or Myzus persicae.

A25. The legume plant of embodiment A24, wherein the plant has resistance to Asian Soybean Rust.

A26. The legume plant of embodiment A24, wherein the plant has resistance to powdery mildew.

A27. The legume plant of any one of embodiments A21-A26, wherein the rebalanced expression level confers the plant with improved agronomic performance as compared to the control plant.

B1. A method of controlling disease resistance in a field comprising the step of planting in an area of cultivation, a legume plant, a plant part or a seed having stably incorporated into its genome:
(a) a first heterologous nucleotide sequence encoding a TIRA polypeptide, or an active variant thereof, having at least 80%, 85%, 90%, 91%, 92%, 93%, 94%, 95%, 96%, 97%, 98% 99% or 100% identity to SEQ ID NO: 1;
(b) a second heterologous nucleotide sequence encoding a TIRB polypeptide, or an active variant thereof, having at least 80%, 85%, 90%, 91%, 92%, 93%, 94%, 95%, 96%, 97%, 98% 99% or 100% identity to SEQ ID NO: 1; and
(c) a heterologous promoter operably coupled to the first and/or the second heterologous nucleotide sequence,
   wherein at least one of the first heterologous nucleotide sequence and the second heterologous nucleotide sequence further comprises a loss of function mutation that reduces an NADase activity of the encoded polypeptide, or active variant thereof, and wherein expression of the first heterologous nucleotide sequence and the second heterologous nucleotide sequence in the plant confers disease resistance.

B2. The method of embodiment B1, wherein the TIRA polypeptide or active variant thereof has a dumbbell structure comprising a TIRA1 region and a TIRA2 region, wherein the TIRB polypeptide or active variant thereof has a dumbbell structure comprising a TIRB1 region and a TIRB2 region, and wherein each of the TIRA1, TIRA2, TIRB1 and TIRB2 regions comprise an NADase activity, and
wherein the loss of function mutation in the first heterologous nucleotide sequence that reduces the NADase activity of the TIRA polypeptide or active variant thereof comprises a loss of function mutation in the TIRA1 region and/or TIRA2 region, and
wherein the loss of function mutation in the second heterologous nucleotide sequence that reduces the NADase activity of the TIRB polypeptide or active variant thereof comprises a loss of function mutation in the TIRB1 region.

B3. The method of embodiment B2, wherein the second heterologous nucleotide sequence does not comprise a loss of function mutation in the TIRB2 region.

B4. The method of any one of embodiments B2-B3 wherein:
the loss of function mutation in the first heterologous nucleotide sequence that reduces the NADase activity of the TIRA polypeptide or active variant thereof comprises at least one mutation at a position corresponding to position 85 or position 251 of SEQ ID NO: 1; and
the loss of function mutation in the second heterologous nucleotide sequence that that reduces the NADase activity of the TIRB polypeptide or active variant thereof comprises at least one mutation at a position corresponding to position 87 of SEQ ID NO: 87.

B5. The method of any one of embodiments B1-B4, wherein the TIRA polypeptide, or active variant thereof, and the TIRB polypeptide, or active variant thereof, are expressed in the legume plant, plant part or seed as a fusion protein.

B6. The method of embodiment B5, wherein the TIRA polypeptide or active variant thereof is at the N-terminus of the fusion protein and is fused to the TIRB polypeptide or active variant thereof via a linker sequence.

B7. The method of embodiment B5, wherein the TIRB polypeptide or active variant thereof is at the N-terminus of the fusion protein and is fused to the TIRA polypeptide or active variant thereof via a linker sequence.

B8. The method of any one of embodiments B5-B7, wherein the first heterologous nucleotide sequence and the second heterologous nucleotide sequence are both operably coupled to, and transcribed by, the heterologous promoter, and wherein the heterologous promoter is an endogenous promoter, an inducible promoter, optionally a rust inducible promoter, or a constitutive promoter active in said plant.

B9. The method of any one of embodiments B5-B8, wherein the heterologous promoter comprises a nucleotide sequence having at least 95% sequence identity to any one of SEQ NOS: 23-28 or wherein the promoter comprises the sequence of any one of SEQ ID NOS: 23-28.

B10. The method of any one of embodiments B1-B4, wherein the first heterologous nucleotide sequence encoding the TIRA polypeptide, or active variant thereof, is operably coupled to a first heterologous promoter in a first expression cassette, and wherein the second heterologous nucleotide sequence encoding the TIRB polypeptide, or active variant thereof, is operably coupled to a second, different heterologous promoter in a second expression cassette, and wherein the first expression cassette is positioned transcriptionally upstream of the second expression cassette.

B11. The method of embodiment B10, wherein the first heterologous promoter is an endogenous promoter, an inducible promoter, or a constitutive promoter active in said plant; and wherein the second promoter is an endogenous promoter, an inducible promoter, or a constitutive promoter active in said plant, wherein the inducible promoter is optionally a rust inducible promoter active in said plant.

B12. The method of embodiment B11, wherein the first heterologous promoter is an endogenous promoter or a constitutive promoter active in said plant; and wherein the second promoter is a rust inducible promoter active in said plant.

B13. The method of any one of embodiments B11-B12, wherein:
(i) the endogenous promoter comprises a nucleotide sequence having at least 95% sequence identity to any one of SEQ ID NOS: 26-27, or wherein the endogenous promoter comprises any one of SEQ ID NOS: 26-27;
(ii) the rust inducible promoter comprises a nucleotide sequence having at least 95% sequence identity to SEQ ID NO: 25, or wherein the endogenous promoter comprises SEQ ID NO: 25; and/or
(iii) the constitutive promoter comprises a nucleotide sequence having at least 95% sequence identity to any one of SEQ ID NOS: 22-24, or wherein the endogenous promoter comprises any one of SEQ ID NOS: 22-24.

B14. A legume plant produced by the method of any one of embodiments B1-B13.

B15. The legume plant of embodiment B14, wherein the legume plant is a legume crop plant, and optionally wherein the legume crop plant is alfalfa, clover, pea, bean, lentil, lupin, mesquite, carob, soybean, pigeon pea, peanut or tamarind.

B16. The legume plant of embodiment B15, wherein the legume crop plant is a soybean plant.

B17. The legume plant of embodiment B16, wherein the soybean plant is an elite soybean plant.

B18. The legume plant of any one of embodiments B14-B17, wherein the plant has resistance to one or more of the following: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum or Myzus persicae.

B19. The legume plant of embodiment B18, wherein the plant has resistance to Asian Soybean Rust.

B20. The legume plant of embodiment B18, wherein the plant has resistance to powdery mildew.

B21. The legume plant of any one of embodiments B14-B20, wherein the reduced NADase activity of the TIRA polypeptide and/or TIRB polypeptide, or active variants thereof, confers the plant with improved agronomic performance as compared to a control plant.

C1. A method of controlling disease resistance in a legume plant, or a method of producing a legume plant having disease resistance comprising the step of introducing a genetic modification into a genome of the plant, wherein the genetic modification
(a) rebalances an expression level of a TIRA polypeptide, or active variant thereof, having at least 80%, 85%, 90%, 91%, 92%, 93%, 94%, 95%, 96%, 97%, 98% 99% or 100% sequence identity to SEQ ID NO: 1, relative to a TIRB polypeptide, or active variant thereof, in the legume plant, having at least 80%, 85%, 90%, 91%, 92%, 93%, 94%, 95%, 96%, 97%, 98% 99% or 100% sequence identity to SEQ ID NO: 2, wherein the expression level in the plant is rebalanced relative to the expression level in a control plant not comprising the genetic modification, and wherein the rebalanced expression level of the TIRA polypeptide or active variant thereof relative to the TIRB polypeptide or active variant thereof in the plant comprises a ratio of about 0.5:1 to about 2:1; or
(b) alters an NADase activity of the TIRA polypeptide, or active variant thereof, and/or decreases an NADase activity of the TIRB polypeptide, or active variant thereof.

C2. The method of embodiment C1, wherein introducing the genetic modification of (a) that rebalances the expression level comprises introducing into the genome of the legume plant, a nucleic acid molecule comprising a heterologous promoter active in the legume plant operably coupled to a first polynucleotide encoding a TIRA polypeptide, or active variant thereof, having at least 80% sequence identity to SEQ ID NO: 1 and a second polynucleotide encoding a TIRB polypeptide, or active variant thereof, having at least 80% sequence identity to SEQ ID NO: 2, wherein the TIRA polypeptide or active variant thereof confers disease resistance to the legume plant when co-expressed with the TIRB polypeptide or active variant thereof, and wherein the TIRB polypeptide or active variant thereof confers disease resistance to the legume plant when co-expressed with the TIRA polypeptide or active variant thereof.

C3. The method of embodiment C1, wherein introducing the genetic modification of (b) that alters the NADase activity level of the TIRA polypeptide or active variant thereof comprises introducing into the genome of the legume plant, a nucleic acid molecule comprising a heterologous promoter active in the legume plant operably coupled to a first polynucleotide encoding a TIRA polypeptide having at least 80% sequence identity to SEQ ID NO: 1 and further comprising one or more of a loss of function mutation that decreases the NADase activity of the TIRA polypeptide and a gain of function mutation that increases the NADase activity of the TIRA polypeptide; and/or introducing into the genome of the legume plant, operably coupled to the heterologous promoter of the nucleic acid molecule, a second polynucleotide encoding a TIRB polypeptide having at least 80% sequence identity to SEQ ID NO: 2 and further comprising a loss of function mutation that decreases the NADase activity of the TIRB polypeptide, wherein the mutated TIRA polypeptide confers disease resistance to the legume plant when co-expressed with the mutated or unmutated TIRB polypeptide, and wherein the mutated TIRB polypeptide confers disease resistance to the legume plant when co-expressed with the mutated or unmutated TIRA polypeptide.

C4. The method of embodiment C3, wherein the loss of function mutation that decreases the NADase activity of the TIRA polypeptide comprises at least one mutation at a position corresponding to position 85 of SEQ ID NO: 1; wherein the gain of function mutation that increases the NADase activity of the TIRA polypeptide comprises at least one mutation at a position corresponding to position 251 of SEQ ID NO: 1; and wherein the loss of function mutation that decreases the NADase activity of the TIRB polypeptide comprises at least one mutation at a position corresponding to position 87 of SEQ ID NO: 2.

C5. The method of any one of embodiments C2-C4, wherein the genetic modification comprises expressing the TIRA polypeptide or active variant thereof and the TIRB polypeptide or active variant thereof as a fusion protein.

C6. The method of embodiment C5, wherein the TIRA polypeptide or active variant thereof is at the N-terminus of the fusion protein.

C7. The method of embodiment C5, wherein the TIRA polypeptide or active variant thereof is at the C-terminus of the fusion protein.

C8. The method of any one of embodiments C5-C7, wherein the first polynucleotide is separated from the second polynucleotide in the nucleic acid molecule via a linker sequence.

C9. The method of any one of embodiments C2-C8, wherein the heterologous promoter is an endogenous promoter, a rust inducible promoter, or a constitutive promoter.

C10. The method of embodiment C9, wherein the heterologous promoter comprises a nucleotide sequence having at least 95% identity to one of SEQ ID NOS: 22-27.

C11. The method of any one of embodiments C2-C4, wherein the genetic modification comprises expressing the TIRA polypeptide or active variant thereof, via a first expression cassette comprising the first polynucleotide operably coupled to a first heterologous promoter active in said plant, and expressing the TIRB polypeptide or active variant thereof via a second expression cassette comprising the second polynucleotide operably coupled to a second heterologous promoter active in said plant.

C12. The method of embodiment C11, wherein the first and/or the second heterologous promoter is an endogenous promoter, a rust inducible promoter, or a constitutive promoter. C13. The method of embodiment C12, wherein the first and/or the second heterologous promoter comprises a nucleotide sequence having at least 95% identity to one of SEQ ID NOS: 22-27.

C14. The method of any one of embodiments C11-C13, wherein the first heterologous promoter is an endogenous promoter or a constitutive promoter comprising any one of SEQ ID NOS 22-24 or 26-27; and wherein the second promoter is a rust inducible promoter comprising SEQ ID NO: 25.

C15. The method of any one of embodiments C1-C₁₅, wherein the genetic modification further confers the plant with improved agronomic performance compared to the control plant.

C16. The method of any one of embodiments C2-C₁₅, wherein introducing the genetic modification comprises:
(i) transforming a legume plant cell with said nucleic acid molecule and regenerating a transgenic plant from the transformed legume plant cell; or
(ii) crossing a first legume plant comprising said nucleic acid molecule with a second legume plant not comprising the nucleic acid molecule, optionally wherein one of the first and the second legume plant is an elite legume plant.

C17. A legume plant produced by the method of any one of embodiments C1-C₁₆, wherein the legume plant is a legume crop plant, and optionally wherein the legume crop plant is alfalfa, clover, pea, bean, lentil, lupin, mesquite, carob, soybean, pigeon pea, peanut or tamarind.

C18. The legume plant of embodiment C17, wherein the legume crop plant is a soybean plant, and optionally an elite soybean plant.

C19. The legume plant of any one of embodiments C16-C18, wherein the plant has resistance to one or more of the following: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum or Myzus persicae.

C20. The legume plant of embodiment C19, wherein the plant has resistance to Asian Soybean Rust.

C21. The legume plant of embodiment C19, wherein the plant has resistance to powdery mildew.

### Example Embodiments of Nucleic Acids and Polypeptides for Controlling Disease Resistance

D0. A nucleic acid molecule comprising a nucleotide sequence operably linked to a heterologous regulatory element, wherein the nucleotide sequence comprises:
(a) a first polynucleotide encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
   wherein the TIRA polypeptide or active variant thereof and the TIRB polypeptide or active variant thereof are expressed as a fusion protein; and
   wherein expression of said nucleic acid molecule in plant confers disease resistance when expressed in a plant.

D0.1 The nucleic acid molecule of embodiment DO, wherein the TIRA polypeptide or active variant thereof is at the N-terminus of the fusion protein.

D0.2. The nucleic acid molecule of embodiment DO, wherein the TIRB polypeptide or active variant thereof is at the N-terminus of the fusion protein.

D0.3. The nucleic acid molecule of embodiment DO, wherein expression of said nucleic acid molecule in plant confers Asian Soy Rust resistance when expressed in the plant.

D0.4. The nucleic acid molecule of embodiment DO, wherein expression of said nucleic acid molecule in plant confers Powdery mildew resistance when expressed in the plant.

D1. A nucleic acid molecule comprising a nucleotide sequence operably linked to a heterologous regulatory element, wherein the nucleotide sequence comprises:
(c) a first polynucleotide encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(d) a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
   wherein introduction of the nucleic acid molecule in a plant causes an expression level of the TIRA polypeptide, or active variant thereof, relative to the TIRB polypeptide, or active variant thereof, to be rebalanced in the plant as compared to the expression level in a control plant, wherein the rebalanced expression level of the TIRA polypeptide, or active variant thereof, relative to the TIRB polypeptide, or active variant thereof, comprises a ratio of about 0.5:1 to about 2:1, and
   wherein the nucleic acid molecule confers disease resistance when expressed in a plant.

D2. The nucleic acid molecule of embodiment D1,
wherein the first polynucleotide has a nucleotide sequence comprising:
(i) a nucleotide sequence encoding a TIRA polypeptide having at least 80% sequence identity to SEQ ID NO: 1;
(ii) a nucleotide sequence encoding the TIRA polypeptide of SEQ ID NO: 1; or
(iii) the nucleotide sequence of (i) or (ii) and further comprising a mutation that alters an NADase activity of the TIRA polypeptide; and
wherein the second polynucleotide has a nucleotide sequence comprising:
(iv) a nucleotide sequence encoding a TIRB polypeptide having at least 80% sequence identity to SEQ ID NO: 2;
(v) a nucleotide sequence encoding the TIRB polypeptide of SEQ ID NO: 2; or
(vi) the nucleotide sequence of (iv) or (v) and further comprising a loss of function mutation that decreases an NADase activity of the TIRB polypeptide.

D3. The nucleic acid molecule of embodiment D2, wherein the TIRA polypeptide, or active variant thereof, confers the plant with disease resistance when co-expressed with the TIRB polypeptide or active variant thereof, and wherein the TIRB polypeptide, or active variant thereof, confers the plant with disease resistance when co-expressed with the TIRA polypeptide or active variant thereof.

D4. The nucleic acid molecule of any one of embodiments D2-D3, wherein the TIRA polypeptide or active variant thereof and the TIRB polypeptide or active variant thereof are expressed as a fusion protein.

D5. The nucleic acid molecule of embodiment D4, wherein the TIRA polypeptide or active variant thereof is at the N-terminus of the fusion protein or wherein the TIRB polypeptide or active variant thereof is at the N-terminus of the fusion protein.

D6. The nucleic acid molecule of any of embodiments D1-D5, wherein the heterologous regulatory element is a heterologous promoter active in said plant, and wherein the heterologous promoter is a constitutive promoter, an inducible promoter, or an endogenous promoter, and wherein the inducible promoter is optionally a rust inducible promoter.

D7. The nucleic acid molecule of embodiment D6, wherein the heterologous promoter is selected from the group comprising SEQ ID NOS: 22-27.

D8. The nucleic acid molecule of any of embodiments D2-D3, wherein the heterologous regulatory element comprises a first heterologous promoter active in said plant operably coupled to the first polynucleotide in a first expression cassette; and a second heterologous promoter active in said plant operably coupled to the second polynucleotide in a second expression cassette.

D9. The nucleic acid molecule of embodiment D8, wherein the second expression cassette is positioned transcriptionally upstream of the first expression cassette.

D9-1. The nucleic acid molecule of embodiment D8, wherein the first expression cassette is positioned transcriptionally upstream of the second expression cassette

D10. The nucleic acid molecule of any of embodiments D8, D9, and D9-1, wherein the first heterologous promoter is a constitutive promoter, an inducible promoter, or an endogenous promoter; wherein the second heterologous promoter is a constitutive promoter, an inducible promoter, or an endogenous promoter; and wherein the inducible promoter is optionally a rust inducible promoter.

D11. The nucleic acid molecule of embodiment D10, wherein the first heterologous promoter is selected from the group comprising SEQ ID NOS: 22-24 and 26-27 and wherein the second heterologous promoter is SEQ ID NO: 25 or 27.

D12. The nucleic acid molecule of any one of embodiments D2-D11, wherein the mutation that alters an NADase activity of the TIRA polypeptide or active variant thereof comprises one or more of (i) a loss of function mutation at an NADase site of a TIRA1 region and/or a TIRA2 region of the TIRA polypeptide or active variant thereof and (ii) a gain of function mutation at an NADase site of a TIRA2 region of the TIRA polypeptide or active variant thereof; and wherein the loss of function mutation that decreases an NADase activity of the TIRB polypeptide or active variant thereof comprises a loss of function mutation in an NADase site of a TIRB1 region of the TIRB polypeptide or active variant thereof.

D13. The nucleic acid molecule of embodiment D12, wherein the TIRB polypeptide or active variant thereof does not comprise a loss of function mutation in an NADase site of a TIRB2 region of the TIRB polypeptide or active variant thereof.

D14. The nucleic acid molecule of any one of embodiments D1-D13, wherein one or more of the TIRA polypeptide, TIRB polypeptide, or active variants thereof, are tagged with a detectable marker.

D15. A vector comprising the nucleic acid molecule of any one of embodiments DO-D0.2 or D1-D14.

D16. A transgenic cell comprising the nucleic acid of any one of embodiments DO-D0.2 or D1-D14 or the vector of embodiment D15.

D17. The transgenic cell of embodiment D16, wherein the transgenic cell is a transgenic plant cell.

D18. The transgenic cell of embodiment D17, wherein the transgenic plant cell is a transgenic plant cell of a legume plant, optionally wherein the legume plant is selected from the group comprising alfalfa, clover, pea, bean lentil, lupin, mesquite, carob, soybean, pigeon pea, peanut, and tamarind.

D19. The transgenic cell of embodiment D18, wherein the transgenic plant cell is a transgenic soybean plant cell.

D20. A plant, or plant part, derived from the transgenic cell of any one of embodiments D16-D19.

D21. The plant part of embodiment D20, wherein the plant part is a transgenic seed, wherein said transgenic seed has stably incorporated the first polynucleotide and the second polynucleotide into its genome.

D22. A harvested product derived from the transgenic seed of embodiment D21, wherein the harvested product comprises the first and the second polynucleotide.

D23. A processed product derived from the harvested product of embodiment D22, wherein the processed product is a flour, a meal, an oil, a starch, or a product derived from any of the foregoing, wherein the processed product comprises the first polynucleotide and the second polynucleotide.

D24. A DNA construct comprising a polynucleotide operably linked to a heterologous regulatory element, wherein the polynucleotide encodes a polypeptide comprising:
(a) an amino acid sequence having at least 90% identity to SEQ ID NO: 1 or 2, wherein increased expression of the polypeptide in a plant increases disease resistance in the plant;
(b) an amino acid sequence having at least 95% identity to SEQ ID NO: 1 or 2, wherein increased expression of the polypeptide in a plant increases disease resistance in the plant; or,
(c) an amino acid sequence comprising SEQ ID NO: 1 or 2.

D25. The DNA construct of embodiment AD24, wherein the polynucleotide encodes a polypeptide comprising:
(d) an amino acid sequence having at least 90% identity to SEQ ID NO: 1 and an amino acid sequence having at least 90% identity to SEQ ID NO: 2, wherein increased expression of the polypeptide in a plant increases disease resistance in the plant;
(e) an amino acid sequence having at least 95% identity to SEQ ID NO: 1, and an amino acid sequence having at least 95% sequence identity to SEQ ID NO: 2, wherein increased expression of the polypeptide in a plant increases disease resistance in the plant; or,
(f) an amino acid sequence comprising SEQ ID NO: 1 and further comprising one of SEQ ID NO: 2.

D26. The DNA construct of embodiment D24, wherein the polynucleotide that encodes the polypeptide comprises:
(a) a nucleotide sequence having at least 90% sequence identity to any of SEQ ID NOs: 3-8;
(b) a nucleotide sequence having at least 95% identity to any of SEQ ID NOS. 3-8; or
(c) a nucleotide sequence comprising any of SEQ ID NOS: 3-8.

D27. The DNA construct of embodiment D25, wherein the polynucleotide that encodes the polypeptide comprises:
(a) a nucleotide sequence having at least 90% sequence identity to SEQ ID NOs: 3, 4, or 5, and a nucleotide sequence having at least 90% sequence identity to SEQ ID NOS: 6, or 7 or 8;
(b) a nucleotide sequence having at least 95% identity SEQ ID NOS. 3, 4, or 5, and a nucleotide sequence having at least 95% sequence identity to SEQ ID NOS: 6, or 7 or 8; or
(c) a nucleotide sequence comprising any of SEQ ID NOS: 3-5 and a nucleotide sequence comprising any of SEQ ID NOS: 6-8.

D28. The DNA construct of any one of embodiments D24-27, wherein said heterologous regulatory element comprises a promoter active in a plant.

D29. The DNA construct of embodiment D28, wherein said promoter is a tissue-specific promoter or a constitutive promoter.

D30. The DNA construct of embodiment D24, wherein said heterologous regulatory element comprises a terminator sequence, an intron, a 5′ UTR or a 3′ UTR.

D31. The DNA construct of embodiment D30, wherein said polynucleotide encoding the polypeptide is operably linked to a promoter sequence comprising:
(a) a nucleotide sequence having at least 95% identity to any one of SEQ ID NOS: 26, 27 or 49, where said nucleotide sequence is capable of driving expression of the polypeptide of interest in plant cell; or
(b) a nucleotide comprising any one of SEQ ID NOS: 27, 28, or 29.

D32. The DNA construct of any one of embodiments D24-31, wherein the polynucleotide encoding the polypeptide comprises at least 1 native intron or at least one heterologous intron.

D33. A vector comprising the DNA construct of any of embodiments D24-32.

D34. A vector comprising a polynucleotide encoding a polypeptide comprising:
(a) an amino acid sequence having at least 90% identity to SEQ ID NO: 1, or 2, wherein increased expression of the polypeptide in a plant increases disease resistance in the plant;
(b) an amino acid sequence having at least 95% identity to SEQ ID NO: 1 or 2, wherein increased expression of the polypeptide in a plant increases disease resistance in the plant; or,
(c) an amino acid sequence comprising SEQ ID NO: 1, or 2.

D35. The vector of embodiment D34, wherein said polynucleotide comprises
(a) a nucleotide sequence having at least 90% sequence identity to any one of SEQ ID NOS: 3-8;
(b) a nucleotide sequence having at least 95% identity to any one of SEQ ID NOS: 3-8; or
(c) a nucleotide sequence comprising any one of SEQ ID NOS: 3-8.

D36. A cell comprising the DNA construct of any of embodiments D24-32, or the vector of any of embodiments D34-35.

D37. A cell comprising a heterologous polynucleotide comprising encoding a polypeptide comprising:
(a) an amino acid sequence having at least 90% identity to SEQ ID NO: 1, or 2, wherein increased expression of the polypeptide in a plant increases disease resistance in the plant;
(a) an amino acid sequence having at least 95% identity to SEQ ID NO: 1, or 2, wherein increased expression of the polypeptide in a plant increases disease resistance in the plant; or,
(b) an amino acid sequence comprising SEQ ID NO: 1, or 2.

D38. The cell of embodiment D37, wherein the encoded polypeptide comprises:
(d) an amino acid sequence having at least 90% identity to SEQ ID NO: 1, and an amino acid sequence having at least 90% sequence identity to SEQ ID NO: 2, wherein increased expression of the polypeptide in a plant increases disease resistance in the plant;
(e) an amino acid sequence having at least 95% identity to SEQ ID NO: 1, and an amino acid sequence having at least 95% sequence identity to SEQ ID NO: 2, wherein increased expression of the polypeptide in a plant increases disease resistance in the plant; or,
(f) an amino acid sequence comprising SEQ ID NO: 1, and one of SEQ ID NO: 2.

D39. The cell of embodiment D38, wherein said polynucleotide comprises:
(a) a nucleotide sequence having at least 90% sequence identity to any one of SEQ ID NOS: 3-8;
(b) a nucleotide sequence having at least 95% identity to any one of SEQ ID NOS: 3-8; or
(c) a nucleotide sequence comprising any one of SEQ ID NOS: 3-8.

D40. The cell of embodiment D39, wherein said polynucleotide comprises:
(a) a nucleotide sequence having at least 90% sequence identity to any one of SEQ ID NOS: 3-5 and a nucleotide sequence having at least 90% sequence identity to any one of SEQ ID NOS: 6-8;
(b) a nucleotide sequence having at least 95% identity to any one of SEQ ID NOS: 3-5 and a nucleotide sequence having at least 95% sequence identity to any one of SEQ ID NOS: 6-8; or
(c) a nucleotide sequence comprising any one of SEQ ID NOS: 3-5 and a nucleotide sequence comprising any one of SEQ ID NOS: 6-8.

D41. The cell of any one of embodiments D37-40, wherein said cell is a plant cell.

D42. The cell of any one of embodiments D37-41, wherein said polynucleotide is stably integrated into the genome of the cell.

D43. The plant cell of embodiment D41 or D42, wherein the plant cell has an increased level of expression of the polypeptide and the plant cell has increased disease resistance relative to a control plant cell.

D44. The plant cell of any one of embodiments D41-43, wherein the plant cell is (a) a monocot cell, (b) a dicot cell, (c) a legume cell, (d) a soybean cell, (e) a barley cell, a maize cell, an oat cell, a rice cell, a sorghum cell, a sugarcane cell or a wheat cell; or (f) a sunflower cell, a tomato cell, a cotton cell, a sugar beet cell or a tobacco cell.

D45. A plant comprising the plant cell of any of embodiments D43-44.

D46. The plant of embodiment D45, wherein the transgenic plant has increased resistance to Asian Soybean Rust.

D47. The plant of embodiment D46, wherein the transgenic plant is a legume plant.

D48. The plant of embodiment D45, wherein the legume plant is a soybean plant.

D49. The plant of embodiment D48, wherein the soybean plant is an elite soybean plant.

D50. A seed of the plant of any of embodiments D45-49, wherein said seed has stably integrated into its genome the said heterologous polynucleotide.

D51. A harvested product derived from the plant of any of embodiments D45-49 or the transgenic seed of embodiment D50.

D52. A processed product derived from the harvested product of embodiment D51, wherein the processed product is a flour, a meal, an oil, a starch, or a product derived from any of the foregoing.

D53. A method of producing a plant having an increased disease resistance comprising:
(a) introducing into the genome of a plant cell a heterologous polynucleotide encoding a polypeptide comprising:
   i) an amino acid sequence a having at least 90% identity to SEQ ID NO: 1, or 2, wherein increased expression of the polypeptide in a plant increases disease resistance in the plant;
   ii) an amino acid sequence having at least 95% identity to SEQ ID NO: 1 or 2, wherein increased expression of the polypeptide in a plant increases disease resistance in the plant; or,
   iii) an amino acid sequence comprising SEQ ID NO: 1, or 2; and
(b) regenerating the soybean plant cell of (a) into a soybean plant
wherein expression of said polypeptide in the plant increases the disease resistance of the plant.

D54. The method of embodiment D53, wherein said heterologous polynucleotide comprises:
(a) a nucleotide sequence having at least 90% sequence identity to any one of SEQ ID NOs: 3-8;
(b) a nucleotide sequence having at least 95% identity to any one of SEQ ID NOS: 3-8; or
(c) a nucleotide sequence comprising any one of SEQ ID NOS. 3-8.

D55. The method of embodiments D53-54, wherein said plant is a legume plant.

D56. The method of embodiment D55, wherein said legume plant is a soybean plant.

D57. The method of any one of embodiments D53-56, wherein said increase in disease resistance comprises an increased resistance to Asian Soybean Rust.

D58. The method of any one of embodiments D53-56, wherein said increase in disease resistance comprises an increased resistance to the pathogens Phakopsora pachyrhizi and Phakopsora meibomiae.

D59. The method of any one of embodiments D53-56, wherein said increase in disease resistance comprises an increased resistance to a nematode pathogen including soybean cyst nematode or root knot nematode, a bacterial pathogen including Pseudomonas syringae, or a sucking pest including an aphid, a stinkbug and a whitefly.

D60. The method of any one of embodiments D53-59, wherein introducing said heterologous polynucleotide is through gene editing.

D61. The method of any one of embodiments D53-59, wherein introducing said heterologous polynucleotide into the plant genome is through transformation of the plant cell with a DNA construct of any one of embodiments D24-32 or the vector of embodiments D33-35.

D62. The method of embodiments any one of embodiments D53-61, further comprising crossing a first soybean plant comprising the heterologous polynucleotide or vector with a second, different legume plant or soybean plant.

D63. The method of embodiment D62, wherein the method further comprises obtaining a progeny legume plant or soybean plant for one or more generations from the transgenic soybean plant, wherein the progeny soybean plant comprises the heterologous polynucleotide and has increased resistance to the soybean pathogen.

D64. A method of decreasing Asian Soybean Rust damage or controlling an ASR pathogen in an area of cultivation comprising the planting in said area of cultivation a plant of any one of embodiments D45-49 or seed of embodiment D50.

D65. A method of determining the presence of a polypeptide having at least 90%, 95% or 100% sequence identity to SEQ ID NO: 1 or 2, comprising the steps of:
(a) isolating nucleic acid molecules from a legume plant and generating an amplicon comprising at least a fragment of a polynucleotide encoding said polypeptide using a probe and/or primer; or
(b) isolating proteins from said soybean plant and detecting presence of said polypeptide;
thereby determining the presence of the polypeptide in the legume plant.

D66. A method for producing an Asian Soybean Rust (ASR) resistant soybean plant comprising the steps of:
(a) selecting a soybean plant from a plurality of soybean plants by detecting the presence of a polynucleotide encoding a polypeptide comprising the amino acid sequence having at least 90%, 95% or 100% sequence identity to SEQ ID NO: 1, or 2; and
(b) generating an ASR resistant progeny soybean plant from said selected soybean plant in a breeding program.

D67. The method of embodiment D66, wherein said polynucleotide comprises a sequence having at least 90%, 95%, or 100% identity to any one of SEQ ID NOS: 3-8.

D68. An isolated or recombinant polypeptide comprising:
(a) an amino acid sequence having at least 90% identity to SEQ ID NO: 1, or 2, wherein increased expression of the polypeptide in a plant increases disease resistance in the plant;
(b) an amino acid sequence having at least 95% identity to SEQ ID NO: 1, or 2, wherein increased expression of the polypeptide in a plant increases disease resistance in the plant; or,
(c) an amino acid sequence comprising SEQ ID NO: 1, or 2.

D69. The isolated or recombinant polypeptide of embodiment D68, wherein the polypeptide comprises:
(d) an amino acid sequence having at least 90% identity to SEQ ID NO: 1, and an amino acid sequence having at least 90% sequence identity to SEQ ID NO: 2, wherein increased expression of the polypeptide in a plant increases disease resistance in the plant;
(e) an amino acid sequence having at least 95% identity to SEQ ID NO: 1, and an amino acid sequence having at least 95% sequence identity to SEQ ID NO: 2, wherein increased expression of the polypeptide in a plant increases disease resistance in the plant; or,
(f) an amino acid sequence comprising SEQ ID NO: 1, and an amino acid sequence comprising SEQ ID NO: 2.

D70. The isolated or recombinant polypeptide of embodiment D68 or 69, further comprising a heterologous amino acid sequence.

D71. A composition comprising the isolated or recombinant polypeptide of any one of embodiments D68-71.

### Example Embodiments of Fusion Proteins for Controlling Disease Resistance

E1. A fusion protein comprising:
(a) a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2, wherein expression of said fusion protein in a plant confers the plant with increased disease resistance relative to a control plant not expressing the fusion protein.

E2. The fusion protein of embodiment E1, wherein the TIRA polypeptide or active variant thereof is at the N-terminus of the fusion protein.

E2.1. The fusion protein of embodiment E2, wherein the fusion protein comprises an amino acid sequence having at least 80%, 85%, 90%, 91%, 92%, 93%, 94%, 95%, 96%, 97%, 98% 99% or 100% identity to SEQ ID NO: 12.

E3. The fusion protein of embodiment E1, wherein the TIRB polypeptide or active variant thereof is at the N-terminus of the fusion protein.

E3.1 The fusion protein of embodiment E3, wherein the fusion protein comprises an amino acid sequence having at least 80%, 85%, 90%, 91%, 92%, 93%, 94%, 95%, 96%, 97%, 98% 99% or 100% identity to any of SEQ ID NOS: 9-11.

E4. The fusion protein of embodiment E1, wherein expression of the fusion protein in the plant rebalances an expression level of the TIRA polypeptide or active variant thereof, relative to the TIRB polypeptide, or active variant thereof, in the plant as compared to the expression level in a control plant, wherein the rebalanced expression level of the TIRA polypeptide, or active variant thereof, relative to the TIRB polypeptide, or active variant thereof, comprises a ratio of about 0.5:1 to about 2:1.

E4.1. The fusion protein of embodiment E1, further comprising a cleavable linker coupling the TIRA polypeptide or active variant thereof of (a) to the TIRB polypeptide or active variant thereof of (b), wherein cleavage of the fusion protein at the cleavable linker following expression of the fusion protein in the plant rebalances an expression level of the TIRA polypeptide or active variant thereof, relative to the TIRB polypeptide, or active variant thereof, in the plant as compared to the expression level in a control plant, wherein the rebalanced expression level of the TIRA polypeptide, or active variant thereof, relative to the TIRB polypeptide, or active variant thereof, comprises a ratio of about 0.5:1 to about 2:1.

E5. The fusion protein of embodiment E1,
wherein the TIRA polypeptide or active variant thereof has an amino acid sequence comprising:
i. an amino acid sequence having at least 80% sequence identity to SEQ ID NO: 1;
ii. an amino acid sequence of SEQ ID NO: 1; or
iii. the amino acid sequence of (i) or (ii) and further comprising a mutation that alters an NADase activity of the TIRA polypeptide, wherein the mutation comprises a loss of function mutation that reduces the NADase activity of the TIRA polypeptide and/or a gain of function mutation that increases the NADase activity of the TIRA polypeptide; and
wherein the TIRB polypeptide or active variant thereof has an amino acid sequence comprising:
iv. an amino acid sequence having at least 80% sequence identity to SEQ ID NO: 2;
v. an amino acid sequence of SEQ ID NO: 2; or
vi. the amino acid sequence of (iv) or (v) and further comprising a loss of function mutation that decreases an NADase activity of the TIRB polypeptide.

E6. The fusion protein of embodiment E5, wherein the TIRA polypeptide, or active variant thereof, confers the plant with disease resistance when co-expressed with the TIRB polypeptide or active variant thereof, and wherein the TIRB polypeptide, or active variant thereof, confers the plant with disease resistance when co-expressed with the TIRA polypeptide or active variant thereof.

E7. The fusion protein of embodiment E5 or E6, wherein the mutation that alters an NADase activity of the TIRA polypeptide or active variant thereof comprises a loss of function mutation in an NADase site of a TIRA1 region and/or a TIRA2 region of the TIRA polypeptide or active variant thereof, and/or a gain of function mutation in an NADase site of the TIRA2 region of the TIRA polypeptide or active variant thereof; and wherein the loss of function mutation that decreases an NADase activity of the TIRB polypeptide or active variant thereof comprises a loss of function mutation in an NADase site of a TIRB1 region of the TIRB polypeptide or active variant thereof.

E8. The fusion protein of embodiment E57, wherein the TIRB polypeptide or active variant thereof does not comprise a loss of function mutation in an NADase site of a TIRB2 region of the TIRB polypeptide or active variant thereof.

E9. The fusion protein of any one of embodiments E1-E8, wherein one or more of the TIRA polypeptide, TIRB polypeptide, or active variants thereof, are tagged with a detectable marker.

E10. A nucleic acid molecule encoding the fusion protein of any one of embodiments E1-E9.

E11. A transgenic cell comprising the fusion protein of any one of embodiments E1-E9.

E12. The transgenic cell of embodiment E11, wherein the transgenic cell is a transgenic plant cell of a legume plant, optionally wherein the legume plant is selected from the group comprising alfalfa, clover, pea, bean lentil, lupin, mesquite, carob, soybean, pigeon pea, peanut, and tamarind.

E13. The transgenic cell of embodiment E12, wherein the transgenic plant cell is a transgenic soybean plant cell.

E13. A plant, or plant part, derived from the transgenic cell of any one of embodiments E11-E12.

E14. The plant part of embodiment E13, wherein the plant part is a transgenic seed, wherein said transgenic seed comprises the fusion protein.

E15. A harvested product derived from the transgenic seed of embodiment E14, wherein the harvested product comprises the fusion protein.

E16. A processed product derived from the harvested product of embodiment E15, wherein the processed product is a flour, a meal, an oil, a starch, or a product derived from any of the foregoing, wherein the processed product comprises the fusion protein.

E17. A fusion protein comprising:
(a) a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
wherein expression of said fusion protein in a plant confers the plant with increased resistance to Asian soy rust relative to a control plant not expressing the fusion protein.

E18. The fusion protein of claim E17, further comprising a self-cleavable linker sequence linking the TIRA polypeptide, or active variant thereof, to the TIRB polypeptide, or active variant thereof, and wherein self-cleavage of the fusion protein at the linker sequence following expression of the fusion protein results in separation of the TIRA polypeptide or active variant thereof, from the TIRB polypeptide, or active variant thereof.

E19. The fusion protein of claim E17 or E18, wherein:
the TIRA polypeptide or active variant thereof further comprises one or more of (i) a loss of function mutation at a position corresponding to position 85 of SEQ ID NO: 1 that decreases an NADase activity of the TIRA polypeptide and (ii) a gain of function mutation at a position corresponding to position 251 of SEQ ID NO: 1 that increases the NADase activity of the TIRA polypeptide; and
the TIRB polypeptide or active variant thereof further comprises a loss of function mutation at a position corresponding to position 87 of SEQ ID NO: 2 that decreases the NADase activity of the TIRB polypeptide.

E20. A plant comprising the fusion protein of any of claims E17-19, wherein the plant is an ASR resistant soybean plant.

E21. A fusion protein comprising:
(a) a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
wherein expression of said fusion protein in a plant confers the plant with increased resistance to Powdery mildew relative to a control plant not expressing the fusion protein.

E22. A plant comprising the fusion protein of claim E21, wherein the plant is a powdery mildew resistant soybean plant.

### Example Embodiments of Compositions for Controlling Disease Resistance

F1. A composition comprising:
(a) a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
wherein presence of said composition in a plant confers the plant with increased disease resistance relative to a control plant not comprising the composition.

F2. The composition of embodiment F1, wherein the TIRA polypeptide or active variant thereof is at the N-terminus of a fusion protein with the TIRB polypeptide or active variant thereof.

F3. The composition of embodiment F1, wherein the TIRB polypeptide or active variant thereof is at the N-terminus of a fusion protein with the TIRA polypeptide or active variant thereof.

F5. The composition of any one of embodiments F1-F3, wherein an expression level of the TIRA polypeptide or active variant thereof, relative to the TIRB polypeptide, or active variant thereof, is rebalanced in a plant comprising the composition as compared to the expression level in a control plant, wherein the rebalanced expression level of the TIRA polypeptide, or active variant thereof, relative to the TIRB polypeptide, or active variant thereof, comprises a ratio of about 0.5:1 to about 2:1.

F6. The composition of any one of embodiments F1-F5,
wherein the TIRA polypeptide or active variant thereof has an amino acid sequence comprising:
i. an amino acid sequence having at least 80% sequence identity to SEQ ID NO: 1;
ii. an amino acid sequence of SEQ ID NO: 1; or
iii. the amino acid sequence of (i) or (ii) and further comprising a loss of function mutation that decreases an NADase activity of the TIRA polypeptide or
iv. the amino acid sequence of (i) or (ii) and further comprising a gain of function mutation that increases an NADase activity of the TIRA polypeptide; and
wherein the TIRB polypeptide or active variant thereof has an amino acid sequence comprising:
v. an amino acid sequence having at least 80% sequence identity to SEQ ID NO: 2;
vi. an amino acid sequence of SEQ ID NO: 2; or
vii. the amino acid sequence of (iv) or (v) and further comprising a loss of function mutation that decreases an NADase activity of the TIRB polypeptide.

F7. The composition of embodiment F6, wherein the TIRA polypeptide, or active variant thereof, confers the plant with disease resistance when co-expressed with the TIRB polypeptide or active variant thereof, and wherein the TIRB polypeptide, or active variant thereof, confers the plant with disease resistance when co-expressed with the TIRA polypeptide or active variant thereof.

F8. The composition of embodiment F7, wherein the loss of function mutation that decreases an NADase activity of the TIRA polypeptide or active variant thereof comprises a loss of function mutation in an NADase site of a TIRA1 region and/or a TIRA2 region of the TIRA polypeptide or active variant thereof; wherein the gain of function mutation that increases an NADase activity of the TIRA polypeptide or active variant thereof comprises a gain of function mutation in an NADase site of the TIRA2 region of the TIRA polypeptide or active variant thereof; and wherein the loss of function mutation that decreases an NADase activity of the TIRB polypeptide or active variant thereof comprises a loss of function mutation in an NADase site of a TIRB 1 region of the TIRB polypeptide or active variant thereof.

F9. The composition of embodiment F8, wherein the TIRB polypeptide or active variant thereof does not comprise a loss of function mutation in an NADase site of a TIRB2 region of the TIRB polypeptide or active variant thereof.

F10. The composition of any one of embodiments F1-F9, wherein one or more of the TIRA polypeptide, TIRB polypeptide, or active variants thereof, are tagged with a detectable marker.

F11. The composition of any one of embodiments F1-F10, wherein presence of said composition in a plant confers the plant with increased resistance to Asian Soy rust relative to a control plant not comprising the composition.

F12. The composition of any one of embodiments F1-F10, wherein presence of said composition in a plant confers the plant with increased resistance to powdery mildew relative to a control plant not comprising the composition

### Example Embodiments of Plants Having Increased Disease Resistance

G1. A plant comprising in its genome a stably integrated nucleic acid molecule, the nucleic acid molecule comprising a heterologous promoter operably coupled to:
(a) a first polynucleotide encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and
(b) a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2,
wherein presence of said nucleic acid molecule in the plant confers the plant with increased disease resistance relative to a control plant not comprising the nucleic acid molecule.

G2. The plant of claim G1, wherein the first polynucleotide comprises one or more mutations that alter an NADase activity of the TIRA polypeptide, or active variant thereof, and wherein the second polynucleotide comprises one or more mutations that alter an NADase activity of the TIRA polypeptide, or active variant thereof, and wherein presence of the one or more mutations that alter the NADase activity of the TIRA polypeptide, TIRB polypeptide, or active variants thereof, in the plant confer the plant with improved agronomic performance relative to a control plant not comprising the one or more mutations.

G3. The plant of claim G2, wherein the one or more mutations that alter an NADase activity of the TIRA polypeptide, or active variant thereof, comprise one or more mutations at positions corresponding to position 85 and/or 271 of SEQ ID NO: 1 and wherein the one or more mutations that alter an NADase activity of the TIRB polypeptide, or active variant thereof, comprise a mutation at a position corresponding to position 87 of SEQ ID NO: 2.

G4. The plant of any of claims G1-G3, wherein the plant is resistant to Asian Soy Rust.

G5. The plant of claim G1, wherein the plant is resistant to powdery mildew.

### Example Embodiments of Methods for Producing and/or Screening for Plants Having Increased Disease Resistance and Improved Agronomic Performance

H1. A method of screening for a plant having increased disease resistance and increased agronomic performance, comprising:
(a) introducing into the genome of the plant, a nucleic acid molecule comprising a heterologous promoter operably coupled to (i) a first polynucleotide encoding a encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1; and (ii) a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2;
(b) assaying an expression level of the TIRA polypeptide, or active variant thereof, and the expression level of the TIRB polypeptide, or active variant thereof, in the plant;
(c) comparing the expression level of the TIRA polypeptide or active variant thereof relative to the expression level of the TIRB polypeptide, or active variant thereof, in the plant to determine if the relative expression level is between 0.5:1 to 2:1, thereby determining a balanced expression level of the TIRA polypeptide, or active variant thereof, relative to the expression level of the TIRB polypeptide in the plant.

H2. The method of claim H1, further comprising, selecting a plant having a balanced expression level of TIRA polypeptide, or active variant thereof, relative to the TIRB polypeptide, or active variant thereof.

H3. A method of producing a plant having increased disease resistance and increased agronomic performance, comprising:
(a) introducing into the genome of the plant, a nucleic acid molecule comprising a heterologous promoter operably coupled to:
   (i) a first polynucleotide encoding a encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1 and further comprising one or more mutations at positions corresponding to position 85 and/or position 271 of SEQ ID NO: 1, wherein the one or more mutations alter an NADase activity of the TIRA polypeptide or active variant thereof; and
   (ii) a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2 and further comprising a mutation at a position corresponding to position 87 of SEQ ID NO: 2, wherein the mutation reduces an NADase activity of the TIRB polypeptide or active variant thereof; and
(b) assaying an expression level of the TIRA polypeptide, or active variant thereof, and the expression level of the TIRB polypeptide, or active variant thereof, in the plant;
(c) comparing the expression level of the TIRA polypeptide or active variant thereof relative to the expression level of the TIRB polypeptide, or active variant thereof, in the plant to determine if the relative expression level is between 0.5:1 to 2:1, thereby determining a balanced expression level of the TIRA polypeptide, or active variant thereof, relative to the expression level of the TIRB polypeptide in the plant.

H4. The method of claim H3, further comprising, selecting a plant having a balanced expression level of TIRA polypeptide, or active variant thereof, relative to the TIRB polypeptide, or active variant thereof.

H5. A method of producing a TirA-TirB composition capable of conferring increased disease resistance and improved agronomic performance when introduced into a plant, the method comprising:
(a) generating a first polynucleotide encoding a encoding a TIRA polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1 and a second polynucleotide encoding a TIRB polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2;
(b) mutating the first polynucleotide to introduce one or more mutations at positions corresponding to position 85 and/or position 271 of SEQ ID NO: 1, wherein the one or more mutations alter an NADase activity of the TIRA polypeptide or active variant thereof;
(c) mutating the second polynucleotide to introduce a mutation at a position corresponding to position 87 of SEQ ID NO: 2, wherein the mutation reduces an NADase activity of the TIRB polypeptide or active variant thereof;
(d) expressing the first polynucleotide comprising the one or more mutations and the second polynucleotide comprising the mutation in a plant via an operably-linked heterologous promoter;
(e) screening for a plant having a balanced expression level of the TIRA polypeptide or active variant thereof relative to the TIRB polypeptide or active variant thereof in the plant, wherein the balanced expression level comprises a ratio of 0.5:1 to 2:1.

### Example Embodiments of Methods of Identifying Novel Disease Resistance Proteins

I1. A method of identifying novel disease resistance polypeptides comprising:
(a) providing a set of plant pathogen effector proteins that interact with the TIRA polypeptide of SEQ ID NO: 1;
(b) assaying for interaction of the identified set of plant pathogen effector proteins with a putative TIRA polypeptide having at least 40%, at least 50%, at least 60%, at least 70%, at least 80%, at least 90% or at least 95% sequence identity to SEQ ID NO: 1; and
(c) identifying the putative TIRA polypeptide from step (b) and thereby identifying a novel disease resistance polypeptide.

I2. The method of embodiment I1, wherein step (b) comprises assaying for a hypersensitive response or localized cell death in a plant or plant cell or a plant tissue, thereby indicating that the putative TIRA polypeptide has the same mode of action as the TIRA polypeptide.

I3. A method of identifying novel disease resistance polypeptides comprising:
(a) providing a set of plant pathogen effector proteins that interact with the TIRB polypeptide of SEQ ID NO: 2;
(b) assaying for interaction of the identified set of plant pathogen effector proteins with a putative TIRB polypeptide having at least 40%, at least 50%, at least 60%, at least 70%, at least 80%, at least 90% or at least 95% sequence identity to SEQ ID NO: 2; and
(c) identifying the putative TIRB polypeptide from step (b) and thereby identifying a novel disease resistance polypeptide.

I4. The method of embodiment 13, wherein step (b) comprises assaying for a hypersensitive response or localized cell death in a plant or plant cell or a plant tissue, thereby indicating that the putative RG34 polypeptide has the same mode of action as the TIRB polypeptide.

### Example Embodiments of Methods of Generating a Resistance and/or Virulence Profile

J1. A method for generating a resistance and/or virulence profile for an ASR plant pathogen in an area of cultivation, the method comprising:
a. obtaining a biological material from one or more samples comprising ASR plant pathogens or parts thereof, wherein the samples are obtained from one or more locations within the area of cultivation;
b. determining the frequency of the presence of at least one of effectors SPE-87, SPE-130, SPE-196, SPE-248 or SPE-335, or an active variant thereof within the one or more sample; and
c. generating a resistance profile and/or virulence profile of the area of cultivation of the ASR plant pathogens.

J2. The method of embodiment J1, wherein the biological material comprises ASR plant pathogens, polynucleotides from the ASR plant pathogens, and/or polypeptides from the ASR plant pathogens.

J3. The method of embodiment J1 or J2, wherein the biological material is obtained from a plurality of samples comprising the ASR plant pathogens or parts thereof, wherein the samples are obtained from a plurality of locations within the area of cultivation; and wherein the frequency of at least one of effectors SPE-87, SPE-130, SPE-196, SPE-248 or SPE-335 is determined for each of the plurality of locations.

J4. The method of any one of embodiments J1-J3, wherein determining the frequency of the presence of effectors SPE-87, SPE-130, SPE-196, SPE-248 or SPE-335 or an active variant thereof, comprises detecting a nucleotide sequence having at least 90%, 91%, 92%, 93%, 94%, 95%, 96%, 97%, 98%, 99% or 100% identity to one of SEQ ID NO: 51-55, or detecting a polypeptide encoded thereby.

J5. The method of any one of embodiments J1-J4, wherein generating a virulence profile comprises associating the frequency of at least one of effector SPE-87, SPE-130, SPE-196, SPE-248 or SPE-335 or active variant thereof within the ASR plant pathogen population with a level of virulence to the legumes or soybean plants planted or to be planted in the area of cultivation.

J6. The method of any one of embodiments J1-J5, wherein generating a resistance profile comprises associating the frequency of at least one of effector SPE-87, SPE-130, SPE-196, SPE-248 or SPE-335 or active variant thereof within the ASR plant pathogen population with a level of resistance of the legume or soybean plants planted or to be planted in the area of cultivation have toward the ASR pathogen population.

J7. The method of any one of embodiments J1-J6, wherein the area of cultivation comprises legume plants comprising a polynucleotide encoding a polypeptide comprising:
a. an amino acid sequence having at least 90%, 91%, 92%, 93%, 94%, 95%, 96%, 97%, 98%, or 99% sequence identity to SEQ ID NO: 1 or 2, wherein is expression of said polypeptide in the plant increases the tolerance of the legume or soybean plant or plant part to ASR plant pathogens; or,
b. an amino acid sequence set forth in SEQ ID NO: 1, or 2,
wherein the frequency of the corresponding ASR effector is determined.

J8. The method of embodiment J7, wherein the said polynucleotide is stably integrated into the genome of the legume plant and is heterologous to the legume plant, optionally wherein the plant is a soybean plant.

J9. The method of any one of embodiments J1-J8, further comprising
a. identifying candidate pesticides for use in a pesticide application protocol based on the resistance and/or virulence profile map; and,
b. generating a recommended pesticide application protocol for the area of cultivation comprising at least one disease control measure for controlling the development, reproduction and/or viability of the ASR plant pathogen based on the resistance profile and/or virulence profile.

J10. The method according to any one of embodiments J1-J9, wherein generating a resistance and/or virulence profile comprises associating the frequency of one or more effectors SPE-87, SPE-130, SPE-196, SPE-248 or SPE-335 within the sample or within the area of cultivation with a level of resistance and/or virulence to (a) the legume or soybean plants or seeds planted in the area of cultivation, or (b) a pesticide; and/or (c) a group of pesticides.

J12. The method according to any of embodiments J1-J11, wherein the ASR pathogen samples are obtained from a plurality of plants, spore traps and/or soil.

J13. The method according to any one of embodiments J9-J12, further comprising:
a. obtaining biological material from a one or more second ASR plant pathogen sample obtained from the one or more locations within the area of cultivation, where the area of cultivation has been subject to at least one disease control measure;
b. determining the frequency of the presence of at least one effector SPE-87, SPE-130, SPE-196, SPE-248 or SPE-335 or an active variant thereof within the second ASR plant pathogen samples; and,
c. generating a second resistance profile and/or virulence profile of the area of cultivation from the one or more second ASR plant pathogen sample; and,
d. comparing the first resistance profile and/or virulence profile with the second resistance profile and/or virulence profile.

J14. The method according to embodiment J13, wherein the sample and the second ASR plant pathogen samples are obtained from a plurality of plants, spore traps and/or soil within the area of cultivation.

J15. The method according to any one of embodiments J1-J14, wherein the method further comprises determining at least one disease control measure for applying to a neighbouring location based on the resistance and/or virulence profile.

J16. A method for generating a resistance and/or virulence profile for a bacterial plant pathogen in an area of cultivation, the method comprising:
a. obtaining a biological material from one or more samples comprising Pseudomonas syringae plant pathogens or parts thereof, wherein the samples are obtained from one or more locations within the area of cultivation;
b. determining the frequency of the presence of at least one of effectors HopT1-1, HopT1-2 or an active variant thereof within the one or more sample; and
c. generating a resistance profile and/or virulence profile of the area of cultivation of the bacterial plant pathogens.

J17. A method for generating a resistance and/or virulence profile for a Powdery Mildew pathogen in an area of cultivation, the method comprising:
a. obtaining a biological material from one or more samples comprising Powdery mildew plant pathogens or parts thereof, wherein the samples are obtained from one or more locations within the area of cultivation;
b. determining the frequency of the presence of at least one of effectors EPCSEP-66, EPCSEP-99 or an active variant thereof within the one or more sample; and
c. generating a resistance profile and/or virulence profile of the area of cultivation of the powdery mildew plant pathogens.

J18. A method for generating a resistance and/or virulence profile for a Soybean cyst nematode (SCN) pathogen in an area of cultivation, the method comprising:
a. obtaining a biological material from one or more samples comprising Powdery mildew plant pathogens or parts thereof, wherein the samples are obtained from one or more locations within the area of cultivation;
b. determining the frequency of the presence of at least one of effectors DMCEP-46, DMCEP-84 or an active variant thereof within the one or more sample; and
c. generating a resistance profile and/or virulence profile of the area of cultivation of the SCN plant pathogens.

### Examples

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1: Dumbbell TiR Polypeptides, TirA and TirB, are Encoded by TirA and TirB Genes

Three resistant Glycine canescens lines (PI440935, PI595799 and PI483193), exhibiting ASR resistance against multiple rust strains, were crossed to a susceptible Glycine canescens line, PI 505154. Using segregation and marker analysis, the resistance locus in these lines were mapped to the same genetic interval. The associated interval, and the causative genes located thereon, were mapped to soybean chromosome 14 (top arm position). Two resistance genes of interest, termed TirA and TirB, were identified in the mapped interval. These genes were identified based on the presence of conserved Toll-Interleukin Receptor (TIR) motifs, as shown in Table 1. Details regarding the identification and validation of the TIRA, TIRB genes are provided at WIPO Pub. No. WO2021/022022, the contents of which are incorporated herein in their entirety.

Each Tir gene was found to encode a protein comprising a dumbbell Toll-Interleukin Receptor (TIR) motif (pfam01582, Table 1). The dumbbell protein encoded by the TirA gene (SEQ ID NOS: 3-5) is herein referred to as TIRA polypeptide (SEQ ID NO: 1) or TIRAITIRA2 polypeptide. The dumbbell protein encoded by the TirB gene (SEQ ID NOS: 6-8) is herein referred to as a TIRB polypeptide (SEQ ID NO: 2) or TIRBITIRB2 polypeptide.

### Example 2: Expression of Both TIR Proteins TIRA and TIRB is Required for Conferring Rust Resistance to Soybean

I. Cloning of TirA and TirB genes

DNA constructs, including expression cassettes and vectors, were generated comprising the identified genes, in native form (that is, the genomic DNA sequence including one or more introns and exons), operably coupled to a heterologous regulatory element. In particular embodiments, the constructs comprise the resistance genes operably linked to a heterologous promoter active in a plant cell and capable of driving expression of the gene(s) in the plant cell. Transcription of each gene was driven by a Medicago truncatula promoter and terminator sequence. Binary vectors with individual gene cassettes comprising the TirA gene or the TirB gene were created. A molecular stack of two cassettes comprising the two genes was also built. A list of the binary vectors created comprising the TirA and TirB genes, alone or stacked, is provided at Table 2.

Target sequences (including available restriction enzyme sites for construction) were synthesized by GenScript. Then the target sequences and base vectors were cut by restriction enzymes and ligated by T4 ligase to generate the final vector. Positive clones were identified by RDA and Sanger sequencing. Target sequences (including overlap region in the end for HiFi ligation) were synthesized by GenScript. Then the target sequences and base vector were cut by suitable restriction enzymes and ligated by HiFi DNA Assembly kit (NEB) to generate final vector. Positive clones were identified by RDA and Sanger sequencing.

TABLE 2
List of binary vectors created for TirA and TirB genes

| Construct | | | | |
|---|---|---|---|---|
| ID # | Tir gene | Promoter | Terminator | Marker |
| 24192 | TirB (cGcaRG3b-02; SEQ ID NO: 6) | prMt12344 | tMt12344 (SEQ | EPSPS |
| | | (SEQ ID NO: 22) | ID NO: 30) | |
| 24205 | TirA (cGcaRG3a-01; SEQ ID NO: 3) | prMt51186 | tMt51186 (SEQ | EPSPS |
| | | (SEQ ID NO: 23) | ID NO: 31) | |
| 24217 | TirB (cGcaRG3b-02; SEQ ID NO: 6); | prMt12344 | tMt12344 (SEQ | EPSPS |
| | TirA (cGcaRG3a-01; SEQ ID NO: 3) | (SEQ ID NO: 22); | ID NO: 30); | |
| | | prMt51186 | tMt51186 (SEQ | |
| | | (SEQ ID NO: 23) | ID NO: 31) | |

The base vector used for creation of constructs 24192, 24205, and 24217 comprised the following features, listed in Table 3:

TABLE 3
Features of Base vector used in creation of constructs 24192, 24205, and 24217

| | |
|---|---|
| bNLB | Left border repeat region of T-DNA of Agrobacterium tumefaciens nopaline |
| | ti-plasmid |
| bNRB | Right border region of T-DNA of Agrobacterium tumefaciens nopaline ti- |
| | plasmid. |
| cmEPSPSpt | Plastid targeted soybean codon modified class 2 A. tumefaciens EPSPS with a |
| | T273S modification having a glyphosate tolerance Ki higher than dmEPSPS in |
| | vitro. Contains the CTP from the Chlamydomonas P28. |
| cRepA | RepA gene with A to G at nt735 |
| cSpec | Also called aadA; gene encoding the enzyme aminoglycoside |
| | 3′adenyltransferase that confers resistance to spectinomycin and |
| | streptomycin for maintenance of the vector in E. coli and Agrobacterium |
| cVirG | virG (putative) from pAD1289 with TTG start codon. virGN54D came from |
| | pAD1289 described in Hansen et al. 1994, PNAS 91: 7603-7607 |
| iGmEF | The first intron of the soybean elongation factor (EF) gene |
| oCOLE | The ColE1 origin of replication functional in E. coli |
| oVS1 | origin of replication and partitioning region from plasmid pVS1 of |
| | Pseudomonas (Itoh et al. 1984, Plasmid 11: 206-220); similar to GenBank |
| | Accession Number U10487; serves as origin of replication in Agrobacterium |
| | tumefaciens host |
| prGmEF | Translation elongation factor EF-1 alpha/Tu promoter, including the first |
| | intron and neighboring utr, from soybean (williams 82). |
| prVirG | virG promoter (Winans J. Bact. 172: 2433-38 (1990)) composed of two |
| | promoter elements, one responsive to acetosyringone and phosphate- |
| | starvation (bp 45 to 83) and another to medium acidification (86 to 128) |
| tPsE9 tNOS | 3′-UTR of the pea (Pisum sativum) rib-1,5-bisphospate carboxylase (rbcS2) |
| | small subunit E9. |
| u5GmEF-01 | First 5′ UTR of the soybean elongation factor (EF) gene. |
| u5GmEF-02 | Second 5′ UTR of the soybean elongation factor (EF) gene. |
| xPSIp28 | Soybean codon optimized plastid transit peptide derived from the |
| | Chlamydomonas reinhardtii PSI P28 gene |
| xSTOPS | 6-frame stop to minimize unintended ORF read-through |
| xTAG | 40 bp site for plant insert intactness testing and to stop readthrough ORFs. |

### I. Soybean Transformation

Binary vectors 24192, 24205, and 24217 were electroporated into Escherichia coli. Transformants were selected and plasmid DNA was isolated using standard miniprep methods. Transformants were characterized by diagnostic restriction enzyme digestions of the miniprep DNA. Positive clones displaying the expected pattern of digestion bands were selected and used for transformation into Agrobacterium tumefaciens. Transformants were selected and sequence verified for the presence of the appropriate binary construct before being submitted for Agrobacterium-mediated transformation.

Transgenic soybean lines were produced from immature seed cultures following the Agrobacterium-mediated transformation protocol. Briefly, immature seeds were harvested from soybean pods of ASR susceptible plant lines grown in the greenhouse under standard conditions. Seeds were surface sterilized, immature cotyledons were aseptically excised and cultures were maintained in flasks containing liquid media on rotary shakers under cool fluorescent light. Agrobacterium tumefaciens carrying plasmids with genes of interest were used to transform the immature cotyledons of the susceptible plant line. Selection of primary soybean events was achieved using an appropriate selection agent. In addition, Taqman analysis of the primary soybean events was performed to identify events harboring the TirA cassette, the TirB cassette, or both cassettes. Transgenic events were selected and regenerated to maturity. These plants were grown under the same conditions as the wild-type plants but in separate growth chambers.

### II. Characterization of Rust Resistance of Transgenic Events

Soybean transgenic events at TO and T1 generations, created from constructs 24192, 24205, and 24217, were then characterized for their resistance against soybean rust. Leaves from primary events comprising the individual Tir genes and the molecular stack were placed in a petri dish on a moist paper towel and then inoculated with a spore suspension of soybean rust isolates. Leaves from null events served as negative control. After 14 days, these leaves were evaluated for resistance to soybean rust. The rating and evaluation were performed using methods well known in the art. The symptom evaluation is a modified version of a rust rating scale from Burdon and Speer (Euphytica, 33:891-896, 1984; also, T A G 1984). The ratings for all the assessed transgenic events are shown in the table of FIG. 16. The transgenic events were compared to a control (which comprises the same genetic background without the transgene). The results use standard soy rust rating scales with Reddish-brown (RB) types indicative of being resistant while Tan ratings are indicative of being susceptible. Numbers after the RB ratings are based on a combination of density of lesions or size of the lesions with a 1-4 scale from high to intermediate resistance, and indication of no sporulation (NSP) or very little sporulation (SPL). Numbers after Tan ratings are based on a combination of density of pustules and level of sporulation with 1-5 scale from low to high sporulation.

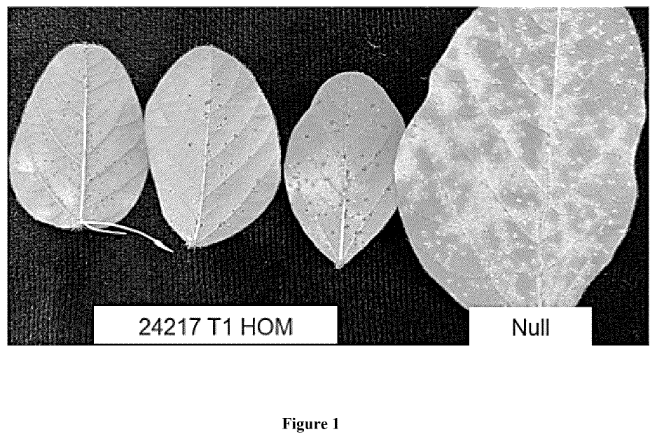

As shown at the table of FIGS. 16, TO and T1 soybean transgenic events expressing either the TIRA polypeptide (that is, events comprising only the TirA gene of construct 24205) or TIRB polypeptide (that is, events comprising only the TirB gene of construct 24192) alone did not confer resistance to soybean rust. However, soybean resistance was observed when both TIRA and TIRB polypeptides were co-expressed (that is, in events comprising the stacked TirA and TirB genes of construct 24217). As further illustrated at FIG. 1, TO events comprising the pair of genes conferred strong rust resistance to rust isolates indicating that both the dumbbell TIR proteins (that is, both a TIRA polypeptide and a TIRB polypeptide) are required for conferring rust resistance in soybean plants. Resistance evaluations of subsequent generations further confirmed that TirA and TirB are the causative genes.

### Example 3: Imbalanced Expression of TIRA and TIRB Polypeptides is Associated with Negative Growth Phenotypes

Individual T1 plants from soybean transgenic events, generated from the constructs of Table 2, expressing only TIRA polypeptide or only TIRB polypeptide were noticed as having mild growth defects and negative growth phenotypes. These included delayed germination, stunted growth, and in some cases, lethality.

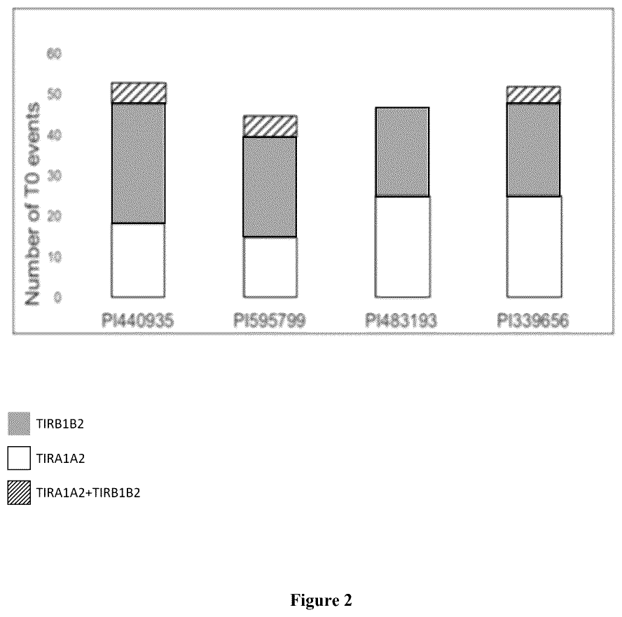

As shown at FIG. 2, fewer events were able to be generated from constructs containing both Tir genes (construct 24217) relative to those generated from constructs comprising only one Tir gene (either only TirA, construct 24205; or only TirB, construct 24192) due to a significantly lower transformation efficiency.

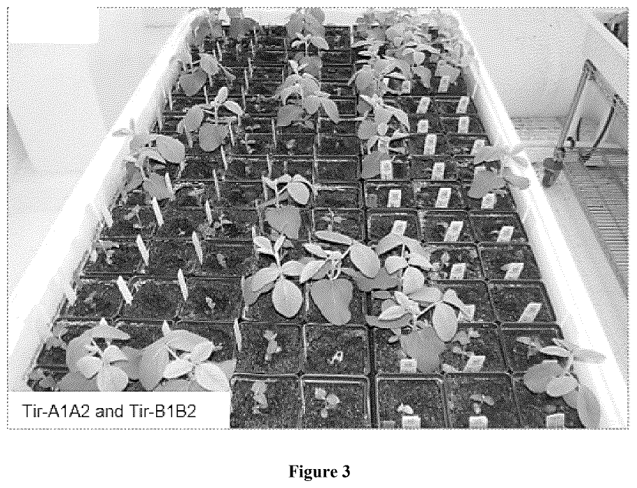

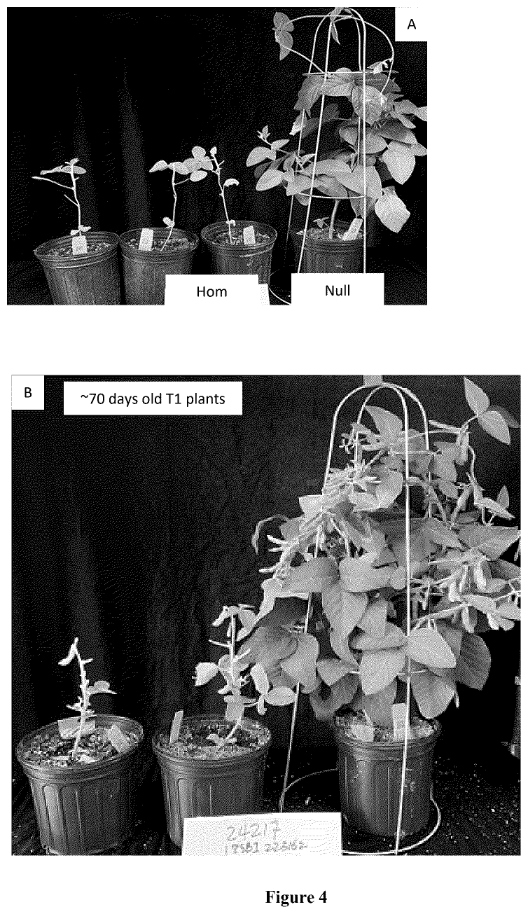

Plants grown from events containing only the TIRB polypeptide had smaller statures while plants from soybean transgenic events expressing only the TIRA polypeptide had fewer visible defects. In comparison, severe growth defects were observed in homozygous and heterozygous T1 plants containing both genes (construct 24217, expressing both the TIRA and TIRB polypeptide) when compared to null segregants (FIGS. 3, 4A-B).

We hypothesized that one of the genes, likely TirB, is the primary cause of the growth defects. Since the observed negative growth phenotypes are not observed in the wild donors from which the Tir genes were derived, we analyzed the wild type donors to ascertain additional information about the expression and/or activity of the Tir genes and their role in causing the observed negative growth phenotypes in the transgenic plants.

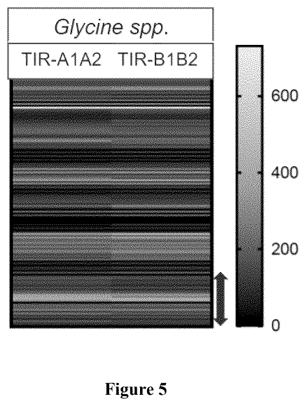

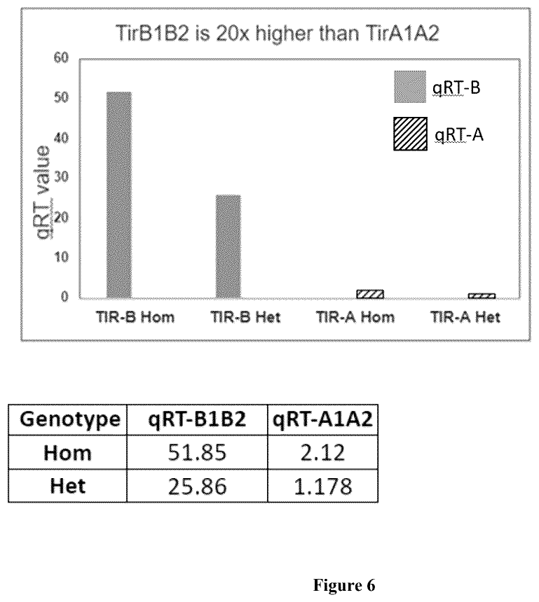

We first examined the transcript levels in the endogenous gene expression of the TIRA and TIRB polypeptides in the wild accession lines. Transcript levels were determined by qRT-PCR. Comparison of transcript levels of the two genes in wild accessions indicated that the genes are expressed at comparable levels (FIG. 5, see arrows). In comparison, transcript levels of the TIRB polypeptide were found to be significantly higher (about 20× higher) than that of the TIRA polypeptide in the transgenic event comprising both the genes (construct 24217) (FIG. 6). Based on these observations, we hypothesized that imbalanced relative expression levels and/or imbalanced relative activity of the TIR proteins in the transgenic plants was the cause of the observed negative growth phenotypes. More specifically, excessive or unbalanced expression levels and/or activity levels of the TIRB polypeptide relative to the expression levels of TIRA polypeptide, in a plant cell comprising the transgene, is the cause of the observed growth defects.

### Example 4: Rebalancing of Expression and/or Activity of TIRA and TIRB Polypeptides in Transgenic Plants Improves Growth Phenotypes

Novel expression constructs comprising the molecular stack of TirA and TirB genes were created to try to rebalance the relative expression levels of the TIR polypeptides and improve the observed growth defects. Rebalancing was performed by using a variety of promoter-terminator combinations, gene orientation combinations, and/or using alternately spliced genomic sequences of TirA and TirB in the novel expression constructs. Table 5 summarizes the construct designs including the promoter/terminator combinations, coding sequence modifications, and orientations used in the various constructs to improve the expression of the two proteins relative to one another.

TABLE 5
Construct designs comprising varying promoter/terminator combinations,
coding sequence modifications, and orientations used to rebalance
expression levels of TIRA and TIRB polypeptides.

| Con- | |
|---|---|
| struct | Design |
| 24217 | TirB (cGcaRG3b-02; SEQ ID NO: 6) driven by prMt12344 |
| | (SEQ ID NO: 22) and tMt12344 (SEQ ID NO: 30) upstream of |
| | TirA (cGcaRG3a-01; SEQ ID NO: 3) driven by prMt51186 |
| | (SEQ ID NO: 23) and tMt51186 (SEQ ID NO: 31) |
| 24916 | TirB (cGcaRG3b-03; SEQ ID NO: 8) driven by prGcaRG3b |
| | (SEQ ID NO: 26) and tMt12344 (SEQ ID: 30) upstream of |
| | TirA (cGcaRG3a-02; SEQ ID NO: 4) driven by prGcaRG3a |
| | (SEQ ID NO: 26) and tMt51186 (SEQ ID NO: 31) |
| 25337 | TirB (cGcaRG3b-03; SEQ ID NO: 8) driven by prLuFIS1 |
| | (SEQ ID NO: 25) and tMt12344 (SEQ ID NO: 30) upstream of |
| | TirA (cGcaRG3a-02; SEQ ID NO: 4) driven by prMt51186 |
| | (SEQ ID NO: 23) and tMt51186 (SEQ ID NO: 31) |
| 24953 | TirA (cGcaRG3a-02; SEQ ID NO: 4) driven by prMt51186 |
| | (SEQ ID NO: 23) and tMt51186 (SEQ ID NO: 31) upstream of |
| | TirB (cGcaRG3b-03; SEQ ID NO: 8) driven by prMt15303 |
| | (SEQ ID NO: 23) and tMt12344 (SEQ ID NO: 30) |
| 24915 | TirB (cGcaRG3b-03; SEQ ID NO: 8) driven by prMt12344 |
| | (SEQ ID NO: 22) and tMt12344 (SEQ ID NO: 30) upstream of |
| | TirA (cGcaRG3a-02; SEQ ID NO: 4) driven by prMt51186 |
| | (SEQ ID NO: 23) and tMt51186 (SEQ ID NO: 31) |

### I. Rebalancing Expression Using Promoters to Enhance TIRA Polypeptide Expression.

Novel expression constructs comprising the molecular stack of TirA and TirB genes were created using a variety of promoter-terminator combinations in an effort to raise the expression level of the TIRA polypeptide relative to the TIRB, thereby attempting to rebalance the transcript levels in resulting transgenic plants. The various promoters used included native promoters and rust-inducible promoters.

In one example, with reference to construct 24916 (Table 5), expression of the TirA gene was driven by the native promoter for TirA derived from G. canescens (prGcaRG3a; SEQ ID NO: 26) and the corresponding native terminator (tGcaRG3a; SEQ ID NO: 28) while expression of the TirB gene was driven by the native promoter for TirB derived from G. canescens (prGcaRG3b; SEQ ID NO: 27) and the corresponding native terminator (tGcaRG3b; SEQ ID NO: 29). The native promoters and terminators were identified as comprising a region upstream and downstream of the genomic coding sequence. Constitutive expression of the TIR polypeptides encoded by the two Tir genes, driven by their corresponding native promoters and terminators, resulted in a significant improvement in transformation efficiency (number of events went from 1 for construct 24217 to 27 events for construct 24916, see Table 6). In addition, the imbalance was improved with the transcript levels of TirB being reduced from being ~20-fold higher than TirA (for events generated from construct 24217) to ~1.5-fold higher than TirA (for events generated from construct 24916).

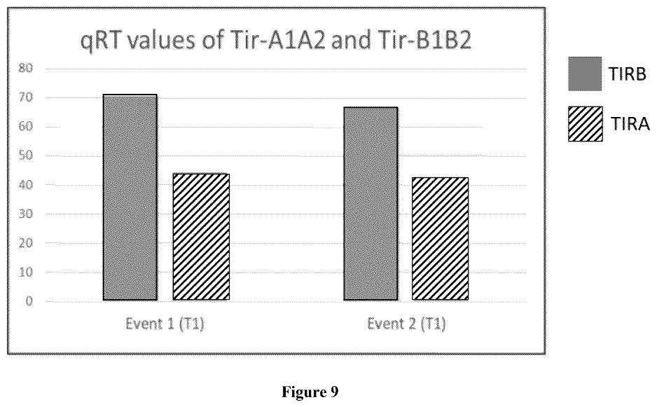

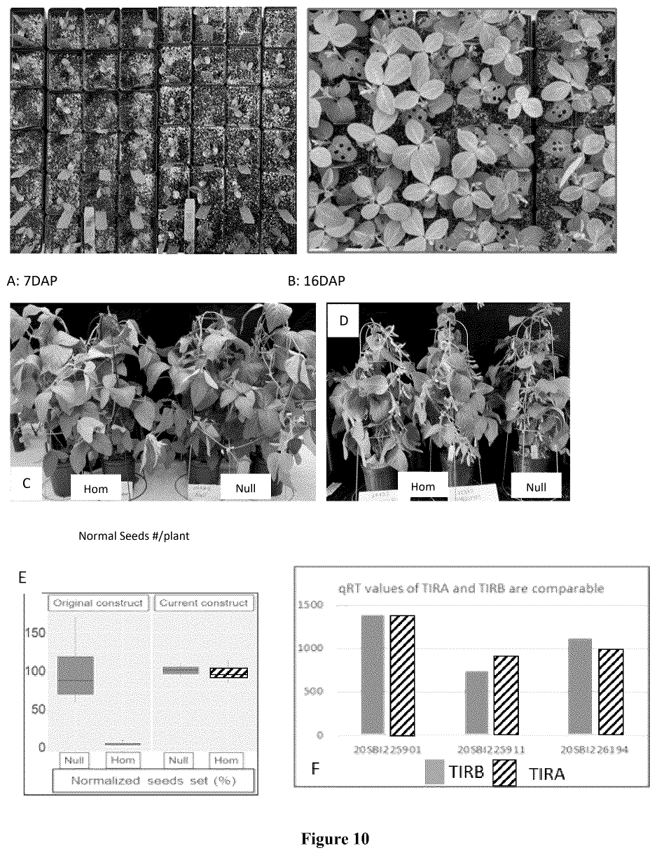

In another example, with reference to construct 25337, expression of the TirA gene was driven by the Medicago promoter prMt51186 (SEQ ID NO: 23) and corresponding terminator (tMt51186; SEQ ID NO: 31) while expression of the TirB gene was driven by a rust-reactive/rust-inducible flax promoter prLuFis1 (SEQ ID NO: 25) and the Medicago terminator tMt12344 (SEQ ID NO: 30). Targeted rust reactive expression of the TirB gene resulted in a significant improvement in transformation efficiency (number of events went from 1 for construct 24217 to 29 events for construct 25337, see Tables 6 and FIG. 20). In addition, the imbalance was improved with the transcript levels of TirB being reduced from being ~20-fold higher than TirA (for events generated from construct 24217) to being substantially equal to TirA (for events generated from construct 25337) (see Tables 6, and FIG. 10F and 20).

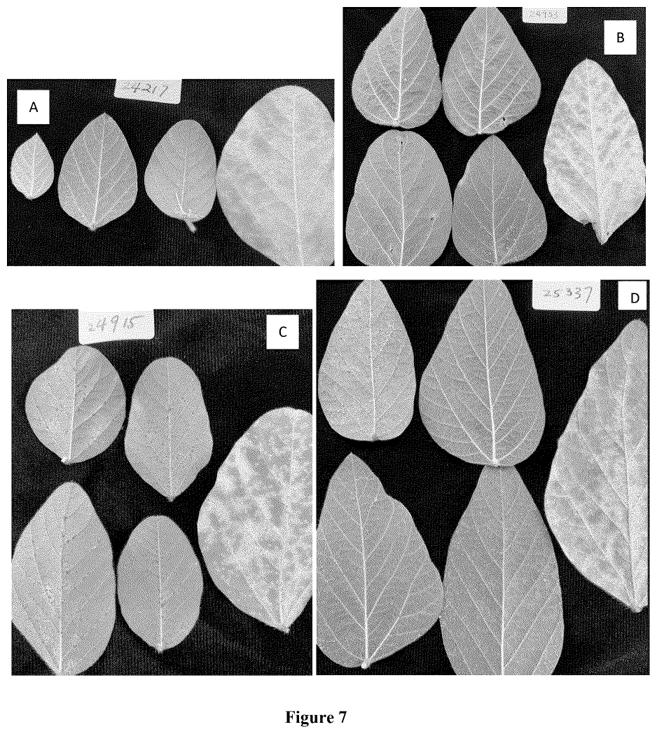

Overall, rebalancing of expression levels using alternate promoter designs improved previously observed growth defects and improved agronomic performance. TO events and their homozygous T1 lines generated from constructs wherein rebalancing was achieved using different promoters showed a substantial improvement in severity of growth defects. The number of events from constructs driven by rust reactive promoters (construct 25337) or native promoters was substantially higher while the events showed similar levels of rust resistance as observed with the control 24217 (FIGS. 7C-D, 10, and 11, FIG. 20, Tables 6). However, the growth defects, while reduced, were still visible.

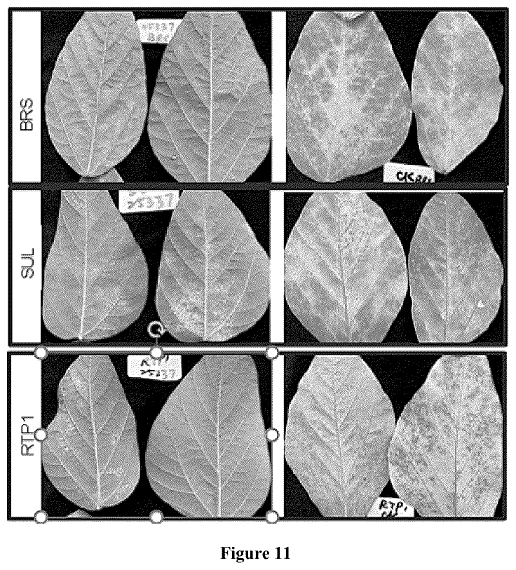

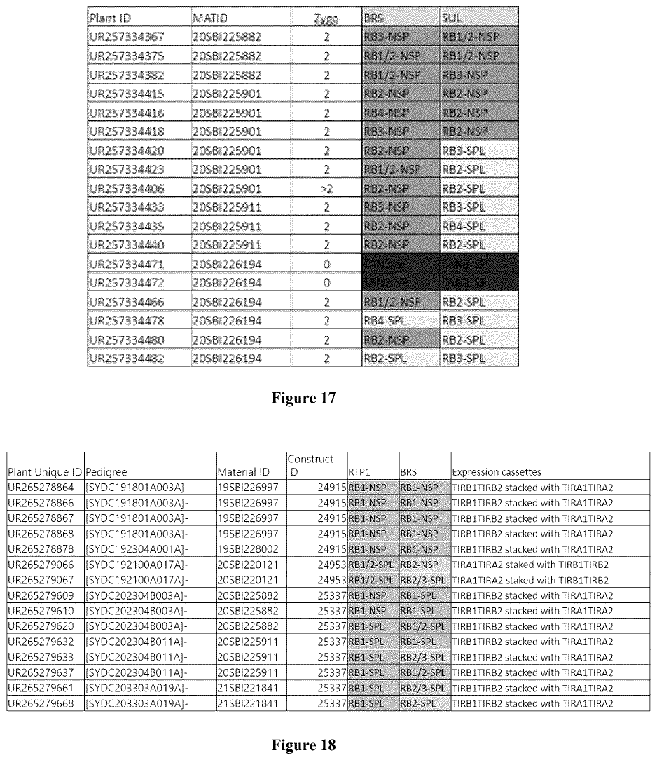

Unexpectedly, homozygous T1 plants from one construct, 25337, which comprises a molecular stack comprising (1) the prLuFis promoter (a potentially rust inducible promoter) to express the TIRB polypeptide, and (2) the prMt51186 promoter to express the TIRA polypeptide showed normal growth with a lack of visible growth defects at all stages (FIG. 10, Panels A-D, and FIG. 20). Further, there was no statistical difference in seeds set between homozygous and null segregants (FIG. 10, Panel E). Supporting our hypothesis, measurements of TIRA polypeptide and TIRB polypeptide transcripts in the homozygous T1 plants were found to be nearly equal between the two genes (FIG. 10, Panel F). We evaluated the rust resistance efficacy of T1 plants generated from construct 25337 and their spectrum. As expected, strong resistance over a broad spectrum was observed when tested with 3 diverse rust isolates (RTP1, BRS and SUL) (FIGS. 11, 17, and 20).

TABLE 6
Promoter/terminator combinations and orientations used to rebalance expression of TIRA polypeptide and TIRB polypeptide.

| Construct | | | | | | | |
|---|---|---|---|---|---|---|---|
| ID | 24217 | 24915 | 24916 | 24953 | 25046 | 25047 | 25337 |
| Promoter- | TirB - | TirB - | TirB - | TirB - | prMt12344 | prMt12344 | TirB - |
| Terminator | prMt12344 | prMt12344 | prGcaRG3b | prMt15303 | tMt12344 | tMt12344 | prLuFIS |
| | tMt12344 | tMt12344 | tMt12344 | tMt12344 | | | tMt12344 |
| | TirA - | TirA - | TirA - | TirA - | | | TirA - |
| | prMt51186 | prMt51186 | prGcaRG3a | prMt51186 | | | prMt51186 |
| | tMt51186 | tMt51186 | tMt51186 | tMt51186 | | | tMt51186 |
| Gene order | B-A | B-A | B-A | A-B | B-A | B-A | B-A |
| CDS | TirB: | TirB: | TirB: | TirB: | Fusion | Fusion | TirB: |
| | cGcaRG3b−02; | cGcaRG3b−03; | cGcaRG3b−03; | cGcaRG3b−03; | cGcaRG3b+a−01; | cGcaRG3b+a−02; | cGcaRG3b−03; |
| | SEQ ID NO: 6 | SEQ ID NO: 8 | SEQ ID NO: 8 | SEQ ID NO: 8 | SEQ ID NO: 13 | SEQ ID NO: 14 | SEQ ID NO: 8 |
| | TirA: | TirA: | TirA: | TirA: | | | TirA: |
| | cGcaRG3a−01; | cGcaRG3a−02; | cGcaRG3a−02; | cGcaRG3a−02; | | | cGcaRG3a−02; |
| | SEQ ID NO: 3 | SEQ ID NO: 4 | SEQ ID NO: 4 | SEQ ID NO: 4 | | | SEQ ID NO: 4 |
| # of T0 | 1 | 16 | 27 | 28 | 16 | 11 | 29 |
| events | | | | | | | |
| Relative | 20:1 | | 1.5:1 | 1.5:1 | 1.5:1 | 1.5:1 | 1:1 |
| expression | | | | | | | |
| of TirB: | | | | | | | |
| TirA | | | | | | | |

### II. Rebalancing Expression Using Change in Relative Orientation of Tir Genes in Molecular Stack.

In one example, with reference to construct 24953, the relative orientation of the genes in the molecular stack was changed. While constructs 24217, 24916, and 25337 comprised the TirB gene positioned upstream (in a transcriptional direction) of the TirA gene, construct 24953 comprised the TirA gene positioned upstream (in a transcriptional direction) of the TirB gene (Tables 5, 6, and 12).

Changing the relative orientation of the Tir genes in the constructs resulted in a significant improvement in transformation efficiency (number of events went from 1 for construct 24217 to 28 events for construct 24953, see Tables 5 and FIG. 20). In addition, the imbalance was improved with the transcript levels of TirB gene being reduced from being ~20-fold higher than TirA (for events generated from construct 24217) to ~1.5-fold higher than TirA (for events generated from construct 24953). In still other examples, the change in orientation can include altering the positioning of the genes and their promoters to drive transcription in opposite directions.

Overall, rebalancing of expression levels using alternate gene orientations improved previously observed growth defects. In addition, rust resistance was conferred. TO events and their homozygous T1 lines generated from constructs wherein rebalancing was achieved using an alternate orientation wherein TirA is transcriptionally upstream of TirB also showed an improvement in severity of growth defects (FIG. 7A, Table 6, and FIGS. 18 and 20 and 21). Rebalancing expression by reducing the number of native introns in the genomic sequence of the genes in the molecular stack.

In still other examples, rebalancing was additionally or optionally achieved by modifying the genomic sequence of one or more of the Tir genes inserted in the constructs using alternative gene modeling (or alternative splicing) to provide shorter coding sequences (Table 8). Particularly, the genomic sequence of the gene was modified to alter (specifically, reduce) the number of native introns.

TABLE 8
Alternate coding sequences for TirA and TirB used to
rebalance expression of TIRA1TIRA2 and TIRB1TIRB2.

| TirA Coding | | |
|---|---|---|
| sequence | Design details | Construct |
| cGcaRG3a-01; SEQ | Genomic sequence with all | 24217 |
| ID NO: 3 | introns | |
| cGcaRG3a-02; SEQ | Genomic sequence without | 24915, 24916, |
| ID NO: 5 | introns | 24953, 25337 |
| cDNA for TirA; | cDNA | |
| SEQ ID NO: 4 | | |
| cGcaRG3b-02; SEQ | Genomic sequence with all | 24217 |
| ID NO: 6 | introns | |
| cGcaRG3b-03; SEQ | Genomic sequence without native | 24915, 24916, |
| ID NO: 8 | introns and including iAtBAF60 | 24953, 25337 |
| | (SEQ ID NO: 33) | |
| cDNA for TirB; | cDNA | |
| SEQ ID NO: 7 | | |

In some examples, the cDNA sequence of TirA is used instead of the genomic sequence wherein all the native introns were removed. In other examples, one or more native introns are removed from the genomic sequence of TirB. In still other examples, one or more of the native introns of TirB are replaced with an alternate intron, such as an intron from Arabidopsis thaliana to reduce expression in E. coli. In particular examples, such as in construct 24915, the TirB sequence used in the cassette comprises the intron iAtBAF60 (cGcaRG3b-03; SEQ ID NO: 8; iAtBAF60; SEQ ID NO: 32), while expression of the TirA cDNA (cGcaRG3a-02; SEQ ID NO: 5) and the TirB gene modified genomic DNA sequence is driven by Medicago trunculata promoters. In another particular example, with reference to construct 24916, the TirB sequence used in the cassette comprises the intron iAtBAF60 (cGcaRG3b-03; SEQ ID NO: 8; iAtBAF60; SEQ ID NO: 32), while expression of the TirA coding sequence and the TirB modified genomic DNA sequence is driven by the corresponding native promoters.

Modifying (specifically, reducing) the number of introns provided in the Tir gene coding sequence resulted in a significant improvement in transformation efficiency (number of events went from 1 for construct 24217 to 16 events for construct 24915 and 27 events for construct 24916, see Tables 5, 8 and FIG. 20). In addition, the imbalance was improved with the transcript levels of TirB being reduced from being ~20-fold higher than TirA (for events generated from construct 24217) to ~1.5-fold higher than TirA (for events generated from construct 24915 and 24916, respectively).

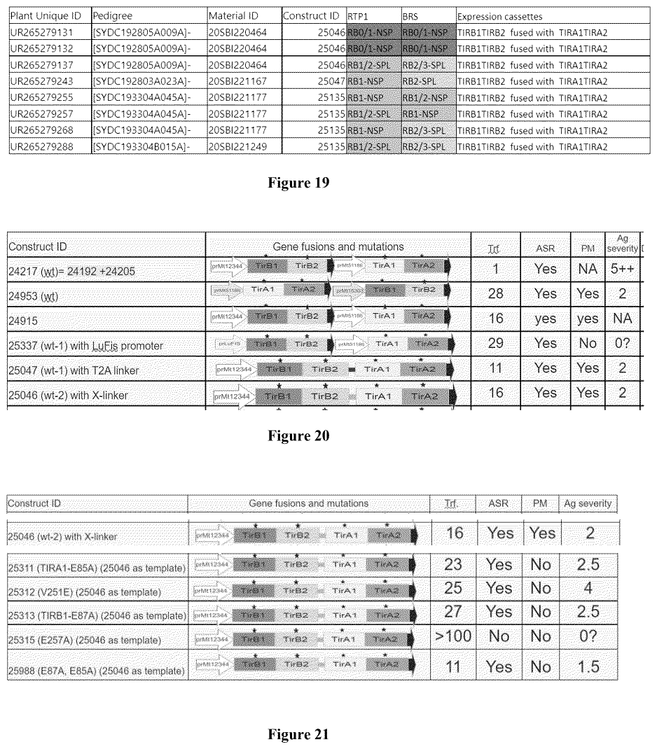

Overall, rebalancing of expression levels using alternately spliced genome sequences improved previously observed growth defects. TO events and their homozygous T1 lines generated from constructs wherein rebalancing was achieved using coding sequences having fewer and/or smaller introns showed an improvement in severity of growth defects. In addition, rust resistance was achieved (Tables 6, and FIGS. 7D and 18-21)

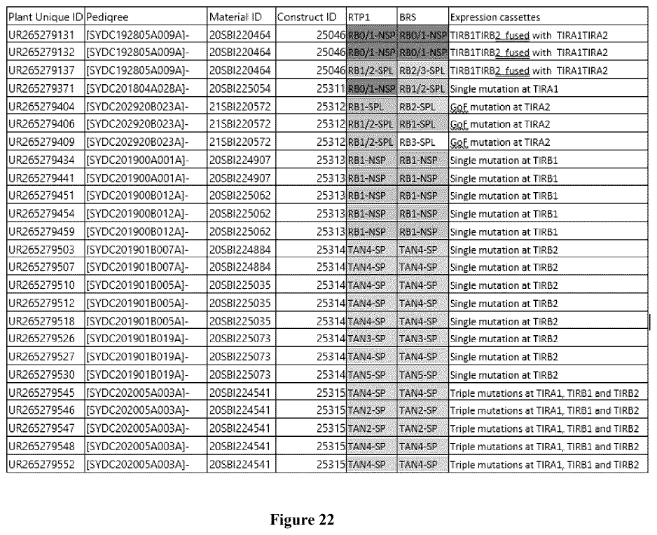

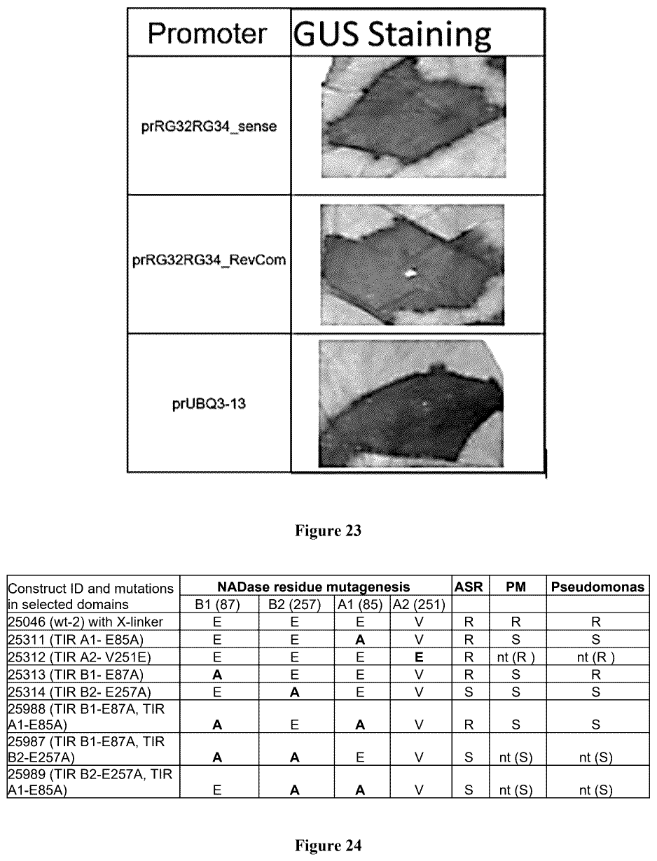

Tables 5, 6, 8 and FIG. 20 summarize the construct designs including the promoter/terminator combinations, coding sequence modifications, and orientations used in the various constructs to improve the expression of the two proteins relative to one another. Tables 6 and FIG. 20 also list the number of TO events created upon transformation of the construct, indicative of transformation efficiency. As shown at Table 6 and FIG. 20, TO events and their homozygous T1 lines from nearly all constructs using the different promoters showed substantial improvement in severity of growth defects. The number of events from these constructs were substantially higher than that of the reference construct 24217. Events from the various constructs also showed similar level of resistance as observed with the reference construct 24217 (see Tables 5-10 and FIGS. 7-11 and 17-22). Even though the severity of the negative growth phenotype was reduced, the growth defect was still visible.

### III. Rebalancing Expression Using Fusion of TIR Proteins

Novel expression constructs were also created comprising the TirA and TirB genes provided on a single, common expression cassette wherein the genes were linked via a linker and expression of both genes was driven using a single, common promoter and terminator (see Table 10). By linking the proteins to create a fusion protein, the expression levels of the TIRA polypeptide and the TIRB polypeptide were expected to be balanced. In particular embodiments, the fusion protein comprised a self-cleavable linker, wherein following expression, the fusion protein cleaves into the constituent TIRA and TIRB polypeptides, thereby rebalancing their relative expression levels.

TABLE 10
Construct designs for fusion proteins comprising
fusions of TIRA and TIRB polypeptides.

| Construct | Design | Insert details |
|---|---|---|
| 25046 | TirAB fusion (cGcaRG3b + a-01; | cGcaRG3b + a-01: native GcaRG3b with |
| | SEQ ID NO: 13) driven by | native introns fused together + native |
| | prMt12344 (SEQ ID NO: 22) and | cGcaRG3a-01 with native introns + |
| | tMt12344 (SEQ ID NO: 30) | synthetic linker, xLinker. |
| 25047 | TirAB fusion (cGcaRG3b + a-02; | cGcaRG3b + a-02: native GcaRG3b with |
| | SEQ ID NO: 14) driven by | native introns fused together + native |
| | prMt12344 (SEQ ID NO: 22) and | cGcaRG3a-01 with native introns + self |
| | tMt12344 (SEQ ID NO: 30) | cleavage peptide signal, xT2ALinker-03 |
| 25135 | TirAB fusion (cGcaRG3b + a-03; | cGcaRG3b + a-03: native GcaRG3b with |
| | SEQ ID NO: 15) driven by | native introns fused together + native |
| | prMt12344 (SEQ ID NO: 22) and | cGcaRG3a-01 with native introns + self |
| | tMt12344 (SEQ ID NO: 30) | cleavage peptide signal, xT2ALinker, |
| | | xT2aLinker-04 |

In one example, with reference to construct 25046, the genomic sequence of the TirA gene comprising the native introns was fused together with the genomic sequence of the TirB gene comprising the native introns by a 38 amino acid long synthetic linker peptide (xLinker-01; SEQ ID NO: 33 and 36). Transcription of the fusion protein was driven by Medicago truncatula promoter prMt12344 (SEQ ID NO: 22) and terminator tMt12344 (SEQ ID NO: 30).

In another example, with reference to construct 25135, the genomic sequence of the TirA gene comprising the native introns was fused together with the genomic sequence of the TirB gene comprising the native introns by a self-cleavable T2a linker peptide derived from the Thosea asigna virus (xT2ALinker-04; SEQ ID NO: 34 and 37). Transcription of the fusion protein in 25135 was driven by Medicago truncatula promoter prMt12344 (SEQ ID NO: 22) and terminator tMt12344 (SEQ ID NO: 30). In yet another example, with reference to construct 25047, the genomic sequence of the TirB gene with the native introns was fused together with the genomic sequence of the TirA gene by the self-cleavable linker T2A (xT2ALinker-03; SEQ ID NO: 35 and 38). Transcription of the fusion protein in 25047 was also driven by Medicago truncatula promoter prMt12344 (SEQ ID NO: 22) and terminator tMt12344 (SEQ ID NO: 30). Inclusion of a self-cleavable linker results in the fusion protein being cleaved into the constituent TIRA and TIRB polypeptides after expression of the fusion protein.

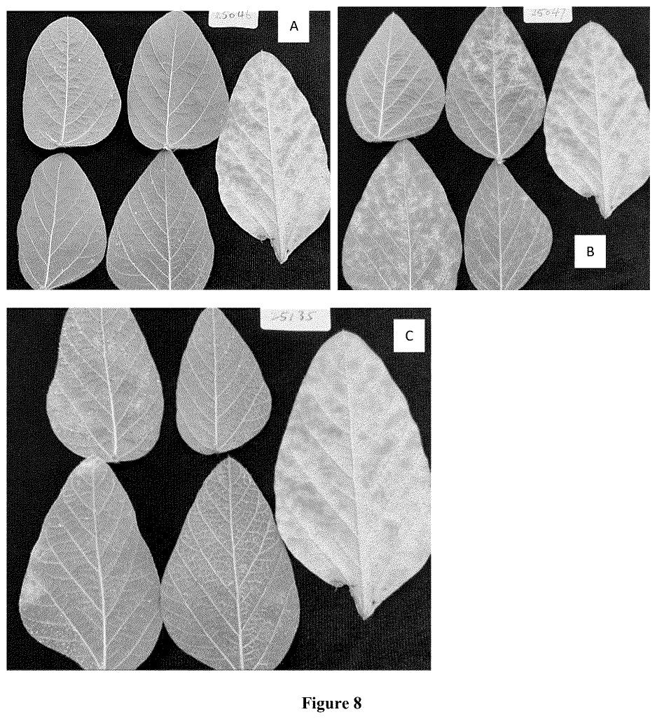

Constructs expressing TIRATIRB Fusion proteins maintained strong rust resistance (FIG. 8, 19, 20 and Table 6). In most events, growth defects were limited. Consistent with our hypothesis, transcript levels of the TIRA polypeptide did not differ many-fold, but rather was about half the level of TIRB polypeptide transcript levels (FIG. 9).

### IV. Rebalancing of TIR Protein Activity Using Targeted Mutagenesis

### A. The Pair of Dumbell TIR Proteins Contains 3 Active NADase Residues

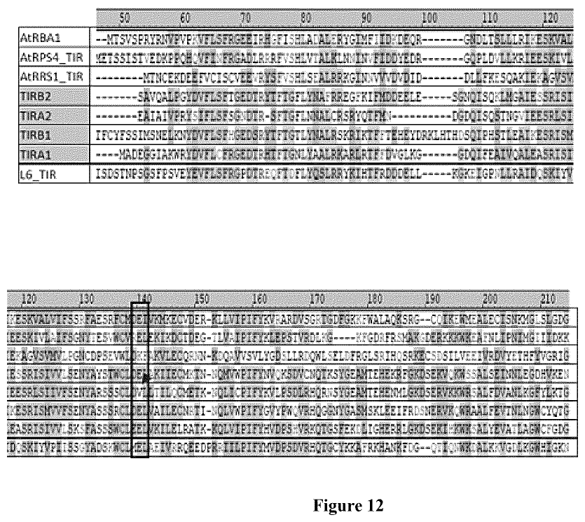

Plant TIR domains of NLR-type resistance proteins (that is, proteins having a nucleotide binding (NB) domain and a leucine rich repeat (LRR) domain) are NADases capable of degrading the oxidized form of nicotinamide adenine dinucleotide (NAD+) which contributes to programmed cell death (PCD) and resistance via EDS1/NRG1 (Jones et al. 2006; Dodds et al 2010; Essuman et al. 2018; Wan et al. 2019; Horsefield et al. 2019). Notably, the glutamatic acid in the BB-loop of the TIR domain is conserved among 70% of TIR containing proteins and has been shown to be critical for NADase activity (Wan et al., 2019, Horsefield et al., 2019; Bayless and Nishimura, 2020). When aligned with other plant TIR domains, we found that the pair of dumbell TIR proteins contain conserved glutamatic acid (E) residues in the TIRA1 domain (at a position corresponding to position 85 of SEQ ID NO: 1), TIRB1 domain (at a position corresponding to position 87 of SEQ ID NO: 2) and TIRB2 domain (at a position corresponding to position 257 of SEQ ID NO: 2), while the TIRA2 domain contains valine (at position 251 of SEQ ID NO: 1), presumably an inactive residue (See FIG. 12, amino acid alignment of L7, AtRBA1, AtRRS1 and AtRRS4). Due to the severity of growth defects and number of potentially active NADase residues in the 4 TIR domains, we hypothesized that one or more of the NADase sites may be contributing to the negative growth phenotype and the severity of the growth defect. Accordingly, we hypothesized that by mutating one or more of the NADase sites across the TIRA1, TIRA2, TIRB1 and TIRB2 domains, activity of the TIR proteins may be modulated (e.g., reduced) and as a result, growth defects may be reduced while resistance function is potentially maintained.

### B. The NADase Residue in the TIRB2 Domain is Required for Rust Resistance.

To understand the functional requirements of the NADase residues in the 4 TIR domains, we conducted site mutagenesis of the putative active residues using construct 25046. We selected construct 25046 as the template for site-directed mutagenesis due to the fusion nature of the construct making it easier to generate mutations. Multiple constructs with single, double and triple loss of function mutations were generated with changes from E to A (alanine) mutations in TIRA1, TIRB1 and TIRB2 domains (constructs 25311, 25313, 25314, 25315, 25988). In addition, one gain of function (GOF) mutation was created by changing the valine to glutamine in TIRA2 (construct 25312). FIG. 21 summarizes the constructs and the corresponding mutations (both their nature and their position).

As used in FIG. 21, the mutations included:
E85A: Glu to Ala mutation at position 85 of the TIRA polypeptide (SEQ ID NO: 1)
V251E: Val to Glu mutation at position 251 of the TIRA polypeptide (SEQ ID NO: 1)
E87A: Glu to Ala mutation at position 87 of the TIRB polypeptide (SEQ ID NO: 2)
E257A: Glue to Ala mutation at position 257 of the TIRA polypeptide (SEQ ID NO: 2)

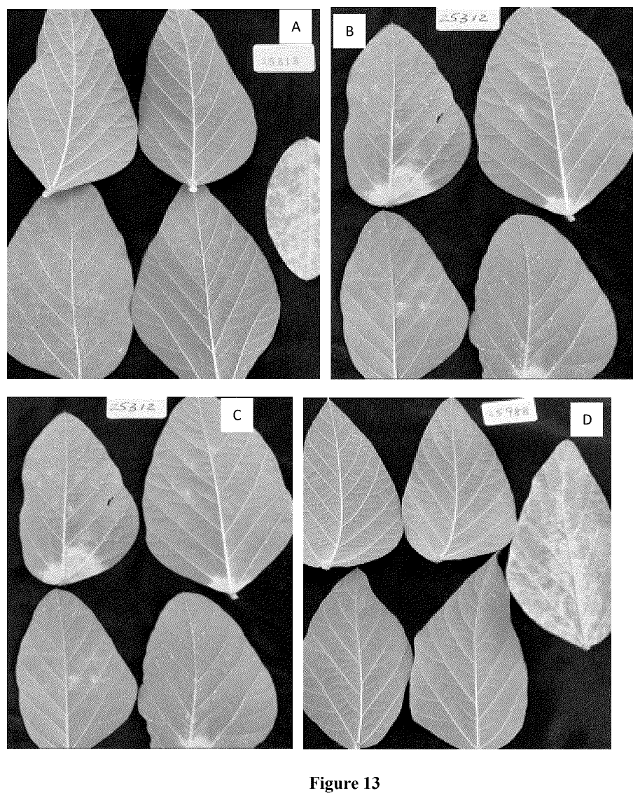

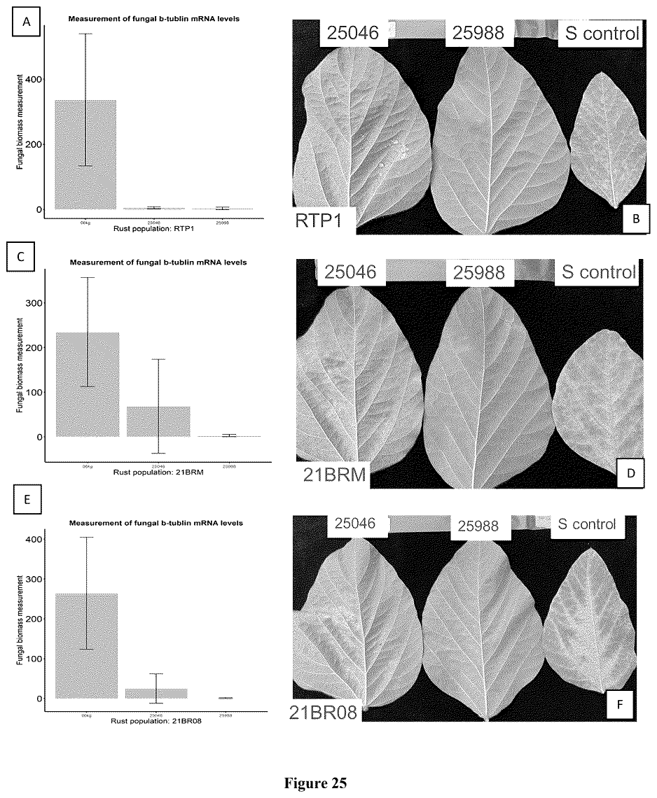

When evaluated in transgenic soybean TO plants, fusion proteins with targeted mutations in the TIRB2 domain abolished soy rust resistance (FIG. 13, panels A-D and FIGS. 21-22, and 24, 25, and 27). Unexpectedly, mutations in TIRA1 and TIRB1 domains did not negatively affect rust resistance. Further, we observed increased levels of rust resistance with double mutations in both the TIRA1 and TIRB1 domains when the E residue in TIRB2 was kept unchanged (FIG. 14, panels A-F). However, constructs with mutation in TIRB2 completely abolished soy rust resistance suggesting that the NADase site in the 2nd TIR domain is critical for soy rust resistance while others are dispensable to this resistance. The gain-of-function mutant did not have substantial impact on level of resistance. Interestingly, we also observed an increased level of resistance with double mutations at TIRA1 and TIRB1 (FIGS. 24, 25A-F and 27). Particularly, the double mutations at TIRA1 and TIRB1displayed significantly higher ASR resistance than wild type controls when tested with 3 different rust isolates (particularly when tested with RTP1, 21BRM and 21BR08) (see FIG. 25A-F).

Growth defects from the mutational reduction in the number of active NADase residues was not significantly improved suggesting that the NADase activities in these domains may have a smaller effect on growth behaviors compared to the balanced expressions of the two proteins.

### C. TIR Proteins Form Hetero-Tetramers to Confer Rust Resistance.

TIR proteins are postulated to form tetramers (Martin, Wang, et al., 2020). We hypothesize that the pair of dumb-bell TIR containing proteins, TIRA polypeptide and TIRB polypeptide, form hetero-tetramers with 4 distinct TIR domains. We hypothesize that the TIRA1 and TIRB1 domains are dispensable for soy rust resistance in that the BB loop in these two domains are closed and not accessible to the substrate after oligomerization. In comparison, the BB loop of the TIRB2 domain is open and sufficient for soy rust resistance.

### D. All NADase Residues are Required to Confer Bacterial Pathogen Resistance, Particularly to Powdery Mildew (PM).

When evaluated in transgenic soybean TO plants, fusion proteins with targeted loss of function mutations in the TIRA1, TIRB1 and TIRB2 domain lost resistance to Powdery mildew (PM; FIG. 24), suggesting that all NADase residues are required in the TIRA and TIRB proteins for conferring soybean plants with increased resistance to Powdery mildew. Surprisingly, the TIRA2 NADase residue appears to be dispensable for Powdery mildew resistance (FIG. 24).

### V. Rebalancing Using a Combination of Promoter Selection, Orientation Selection, Coding Sequence Modification, Fusion of TIR Proteins, and Targeted Mutagenesis in any of the TIR Domains.

It will be appreciated that novel expression constructs may be created using any combination of the rebalancing approaches disclosed herein. This includes rebalancing the expression and/or the activity of the TirA gene with the TirB gene using:
(a) a molecular stack of TirA and TirB genes driven by native promoters, rust inducible promoters, and/or plant active promoters to rebalance relative expression levels; and/or
(b) a molecular stack of TirA and TirB genes wherein one or more of the genes have one or more native introns removed and/or replaced with other plant active introns; and/or
(c) a molecular stack wherein the relative orientation of the genes is varied; transcription of the genes is driven in different directions to rebalance relative expression levels, and/or
(d) linking of the genes via a linker so as to drive the expression of a fusion protein via a common promoter and terminator to rebalance relative expression levels; and/or
(e) targeted site mutagenesis of one or more NADase active sites of the TIRA1, TIRA2 or TIRB 1 domains while maintaining the NADase active site of the TIRB2 domain to rebalance relative activity levels.

In example embodiments, the expression level of the TirB gene (that is, the level of transcripts of the TIRB polypeptide) is balanced with the expression level of the TirA gene (that is, the level of transcripts of the TIRA polypeptide). In particular embodiments, upon balancing, the expression level of the TirA gene (or TIRA polypeptide) is in a range between 0.5 to 2.0 times the expression level of the TirB gene (or TIRB polypeptide). In particular embodiments, upon balancing, the expression level of the TirB gene (or TIRB polypeptide) is in a range between 0.5 to 2.0 times the expression level of the TirA gene (or TIRA polypeptide).

In other example embodiments, the activity level of the TirB gene product (that is, the NADase activity of the TIRB polypeptide) is balanced with the activity level of the TirA gene product (that is, the NADase activity of the TIRA polypeptide). In particular embodiments, upon balancing, the activity of the TIRA polypeptide is in a range between 0.5 times to 2.0 times the activity of the TIRB polypeptide. In particular embodiments, upon balancing, the activity of the TIRB polypeptide is in a range between 0.5 times to 2.0 times the activity of the TIRA polypeptide.

### Example 5: Identification of Homologous, Orthologous and/or Paralogous TIRA1A2 and TIRB1B2 Proteins

Gene identities may be determined by conducting searches using gene alignment tools such as BLAST (Basic Local Alignment Search Tool; Altschul et al. (1993). Therein, searches are conducted under default parameters for similarity to sequences contained in publicly available databases (e.g., the BLAST “nr” database comprising all non-redundant GenBank CDS translations, sequences derived from the 3-dimensional structure Brookhaven Protein Data Bank, the last major release of the 25 SWISS-PROT protein sequence database, EMBL, and DDBJ databases). In addition to public databases, proprietary internal databases were also searched. Certain polynucleotide sequences were analyzed. The identified proteins and their corresponding percent identity values to the TIRA polypeptide (SEQ ID NO: 1) and TIRB polypeptide (SEQ ID NO: 2) is presented in Tables 15 and 16, respectively.

Example orthologs of TIRA and TIRB polypeptides include the polypeptides encoded by the CcRpp2-R1 and CcRpp2-R3 genes from Cajanus cajun (SEQ ID NOS: 45-48). Additional orthologs identifiable based on sequence identity can be found at Tables 3-4 of WO2022/140257, the contents of which are incorporated by reference herein in their entirety.

Sequence data found in Tables 15-16 were derived from https (:) (/) (/) legumeinfo (.) org. The sequences are incorporated by reference herein. The alignment method used here was the same as previously described. Orthologous, homologous and/or paralogous proteins identified were chosen from members of gene families containing the soybean orthologs at the Legume Information System site listed above. Alignments were done using EMBOSS version 6.6.0.0, algorithm needle, with the following parameters: gapopen 10; and gapextend 0.5.

TABLE 15
Homologs, orthologs and/or paralogs of TIRA polypeptide (SEQ ID NO. 1)

| | % Identity | | | |
|---|---|---|---|---|
| | to TIRA | | NT | PRT |
| | (SEQ ID | Source | SEQ | SEQ |
| Protein/Gene Name | NO: 1) | Organism | # | # |
| CcRpp2-R1 | 67.3% | Cajanus cajun | 45 | 46 |
| glyst.G1974.gnm1.ann1.Gst14g036508.1 | 90.60% | Glycine | | |
| | | stenophita | | |
| glyso.F_IGA1003.gnm1.ann1.SoyGsojaF_14G024400.1 | 87.90% | Glycine soja | | |
| glyso.Pl483463.gnm1.ann1.GlysoPl483463.14G021900.1 | 87.70% | Glycine soja | | |
| glyma.Zh35_IGA1004.gnm1.ann1.SoyZH35_14G024400.1 | 87.60% | Glycine max | | |
| glyma.Huaxia3_IGA1007.gnm1.ann1.SoyHX3_14G024400.1 | 87.20% | Glycine max | | |
| glyma.FiskebyIII.gnm1.ann1.GlymaFiskIII.14G023500.1 | 86.40% | Glycine max | | |
| glyma.Lee.gnm1.ann1.GlymaLee.14G022100.1 | 86.40% | Glycine max | | |
| glyma.Wenfeng7_IGA1001.gnm1.ann1.SoyWF7_14G024400.1 | 83.60% | Glycine max | | |
| glyma.Zh13.gnm1.ann1.SoyZH13_14G022300.m1 | 83.50% | Glycine max | | |
| glyma.Wm82.gnm1.ann1.Glyma14g02760.1 | 83.30% | Glycine max | | |
| glyma.Wm82.gnm2.ann1.Glyma.14G024400.1 | 83.30% | Glycine max | | |
| glyma.Wm82.gnm4.ann1.Glyma.14G024400.1 | 83.30% | Glycine max | | |
| glyso.W05.gnm1.ann1.Glysoja.14G037482.1 | 83.30% | Glycine soja | | |
| glyma.Wm82_IGA1008.gnm1.ann1.SoyW82_14G024400.1 | 79.20% | Glycine max | | |
| vigun.Suvita2.gnm1.ann1.VuSuvita2.08G192400.1 | 69.80% | Vigna | | |
| | | unguiculata | | |
| vigun.UCR779.gnm1.ann1.VuUCR779.08G190400.1 | 69.80% | Vigna | | |
| | | unguiculata | | |
| vigan.Shumari.gnm1.ann1.Vigan.03G031000.01 | 69.30% | Vigna | | |
| | | angularis | | |
| phaac.Frijol_Bayo.gnm1.ann1.Phacu.CVR.008G333200.1 | 69.00% | Phaseolus | | |
| | | acutifolius | | |
| phavu.5-593.gnm1.ann1.Pv5-593.08G260300.1 | 69.00% | Phaseolus | | |
| | | vulgaris | | |
| phavu.G19833.gnm1.ann1.Phvul.008G267700.1 | 69.00% | Phaseolus | | |
| | | vulgaris | | |
| phavu.G19833.gnm2.ann1.Phvul.008G267700.1 | 69.00% | Phaseolus | | |
| | | vulgaris | | |
| phaac.Frijol_Bayo.gnm1.ann1.Phacu.CVR.008G333000.1 | 68.90% | Phaseolus | | |
| | | acutifolius | | |
| phaac.W6_15578.gnm2.ann1.Phacu.WLD.008G285700.1 | 68.90% | Phaseolus | | |
| | | acutifolius | | |
| phavu.Ul111.gnm1.ann1.PvUl111.08G265200.1 | 68.90% | Phaseolus | | |
| | | vulgaris | | |
| phavu.LaborOvalle.gnm1.ann1.PvLabOv.08G262800.1 | 68.70% | Phaseolus | | |
| | | vulgaris | | |
| phaac.W6_15578.gnm2.ann1.Phacu.WLD.008G286200.1 | 68.40% | Phaseolus | | |
| | | acutifolius | | |
| phalu.G27455.gnm1.ann1.Pl08G0000412700.1 | 67.00% | Phaseolus | | |
| | | lunatus | | |
| vigun.Sanzi.gnm1.ann1.VuSanzi.08G191300.1 | 67.00% | Vigna | | |
| | | unguiculata | | |
| vigun.CB5-2.gnm1.ann1.VuCB5-2.08G190600.1 | 65.70% | Vigna | | |
| | | unguiculata | | |
| lotja.MG20.gnm3.ann1.Lj0g3v0092229.2 | 61.30% | Lotus | | |
| | | japonicus | | |
| lotja.MG20.gnm3.ann1.Lj0g3v0348599.1 | 61.20% | Lotus | | |
| | | japonicus | | |
| glyso.Pl483463.gnm1.ann1.GlysoPl483463.02G245300.1 | 59.90% | Glycine soja | | |
| glyma.Lee.gnm1.ann1.GlymaLee.02G248900.1 | 59.60% | Glycine max | | |
| vigra.VC1973A.gnm6.ann1.Vradi06g01780.1 | 59.60% | Vigna radiata | | |

TABLE 16
Homologs, orthologs and/or paralogs of TIRB polypeptide (SEQ NO. 2)

| | | % | | |
|---|---|---|---|---|
| | | Identity | | |
| | | to TIRB | | |
| | Source | (SEQ ID | NT | PRT |
| Protein/Gene Name | Organism | NO: 2) | SEQ # | SEQ# |
| CcRpp2-R3 | Cajanus | 62.8 | 47 | 48 |
| | cajun | | | |
| glyst.G1974.gnm1.ann1.Gst14g036507.1 | Glycine | 96.70% | | |
| | stenophita | | | |
| glyfa.G1718.gnm1.ann1.Gfa14g038291.1 | Glycine | 92.90% | | |
| | falcata | | | |
| glyma.Zh13_IGA1005.gnm1.ann1.SoyZH13_14G024500.1 | Glycine max | 92.40% | | |
| glyma.Zh35_IGA1004.gnm1.ann1.SoyZH35_14G024500.1 | Glycine max | 92.40% | | |
| glyso.F_IGA1003.gnm1.ann1.SoyGsojaF_14G024500.1 | Glycine soja | 92.40% | | |
| glyso.Pl483463.gnm1.ann1.GlysoPl483463.14G021800.1 | Glycine soja | 91.60% | | |
| glyma.Huaxia3_IGA1007.gnm1.ann1.SoyHX3_14G024500.1 | Glycine max | 90.50% | | |
| glyma.FiskebyIII.gnm1.ann1.GlymaFiskIII.14G023400.1 | Glycine max | 90.20% | | |
| glyma.Lee.gnm1.ann1.GlymaLee.14G022000.1 | Glycine max | 90.20% | | |
| glycy.G1267.gnm1.ann1.Gcy14g034684.1 | Glycine | 89.60% | | |
| | cyrtoloba | | | |
| glyma.Hefeng25_IGA1002.gnm1.ann1.SoyHF25_14G024500.1 | Glycine max | 87.00% | | |
| glyma.Jinyuan_IGA1006.gnm1.ann1.SoyJY_14G024500.1 | Glycine max | 87.00% | | |
| glyma.Wenfeng7_IGA1001.gnm1.ann1.SoyWF7_14G024500.1 | Glycine max | 87.00% | | |
| glyma.Wm82_IGA1008.gnm1.ann1.SoyW82_14G024500.1 | Glycine max | 87.00% | | |
| glyma.FiskebyIII.gnm1.ann1.GlymaFiskIII.14G023600.1 | Glycine max | 86.80% | | |
| glyma.Lee.gnm1.ann1.GlymaLee.14G022200.1 | Glycine max | 86.80% | | |
| glyma.Wm82.gnm1.ann1.Glyma14g02770.2 | Glycine max | 86.80% | | |
| glyma.Wm82.gnm2.ann1.Glyma.14G024500.1 | Glycine max | 86.80% | | |
| glyma.Wm82.gnm4.ann1.Glyma.14G024500.1 | Glycine max | 86.80% | | |
| glyso.W05.gnm1.ann1.Glysoja.14G037483.1 | Glycine soja | 86.50% | | |
| glyma.Zh13.gnm1.ann1.SoyZH13_14G022200.m1 | Glycine max | 84.70% | | |
| glyso.W05.gnm1.ann1.Glysoja.14G037483.2 | Glycine soja | 79.20% | | |
| vigun.CB5-2.gnm1.ann1.VuCB5-2.08G190500.1 | Vigna | 71.30% | | |
| | unguiculata | | | |
| vigun.IT97K-499-35.gnm1.ann2.Vigun08g199000.1 | Vigna | 71.30% | | |
| | unguiculata | | | |
| vigun.Sanzi.gnm1.ann1.VuSanzi.08G191200.1 | Vigna | 71.30% | | |
| | unguiculata | | | |
| vigun.Suvita2.gnm1.ann1.VuSuvita2.08G192300.1 | Vigna | 71.30% | | |
| | unguiculata | | | |
| vigun.TZ30.gnm1.ann2.VuTZ30.08G186200.1 | Vigna | 71.30% | | |
| | unguiculata | | | |
| vigun.UCR779.gnm1.ann1.VuUCR779.08G190300.1 | Vigna | 71.30% | | |
| | unguiculata | | | |
| vigun.ZN016.gnm1.ann2.VuZN016.08G187500.1 | Vigna | 71.30% | | |
| | unguiculata | | | |
| vigra.VC1973A.gnm6.ann1.Vradi06g01790.1 | Vigna radiata | 70.70% | | |
| phalu.G27455.gnm1.ann1.Pl08G0000412600.1 | Phaseolus | 70.50% | | |
| | lunatus | | | |
| phaac.W6_15578.gnm2.ann1.Phacu.WLD.008G286100.1 | Phaseolus | 69.60% | | |
| | acutifolius | | | |
| phavu.5-593.gnm1.ann1.Pv5-593.08G260200.1 | Phaseolus | 68.80% | | |
| | vulgaris | | | |
| phavu.G19833.gnm1.ann1.Phvul.008G267600.1 | Phaseolus | 68.70% | | |
| | vulgaris | | | |
| phavu.G19833.gnm2.ann1.Phvul.008G267600.1 | Phaseolus | 68.70% | | |
| | vulgaris | | | |
| vigun.IT97K-499-35.gnm1.ann2.Vigun08g199000.2 | Vigna | 68.10% | | |
| | unguiculata | | | |
| phavu.Ul111.gnm1.ann1.PvUl111.08G265100.1 | Phaseolus | 67.90% | | |
| | vulgaris | | | |
| phaac.W6_15578.gnm2.ann1.Phacu.WLD.008G285600.1 | Phaseolus | 64.60% | | |
| | acutifolius | | | |
| phavu.LaborOvalle.gnm1.ann1.PvLabOv.08G262700.1 | Phaseolus | 64.00% | | |
| | vulgaris | | | |
| lotja.MG20.gnm3.ann1.Lj0g3v0132719.1 | Lotus | 61.30% | | |
| | japonicus | | | |
| trisu.Daliak.gnm2.ann1.Ts_23132.1 | Trifolium | 60.80% | | |
| | subterraneum | | | |

Alignment of one or more of the identified homologs, orthologs and/or paralogs of Table 14 with TIRA polypeptide (SEQ ID NO: 1) enables the identification of the corresponding NADase sites. For example, when aligned with other plant TIR domains, a conserved, presumably active, glutamic acid (E) residue can be found in the TIRA1 and/or TIRA2 domain of TIRA polypeptides from various orthologs. Likewise, alignment of one or more of the identified homologs, orthologs and/or paralogs of Table 16 with the TIRB polypeptide (SEQ ID NO: 2) enables the identification of the corresponding NADase sites. For example, when aligned with other plant TIR domains, a conserved, presumably active, glutamic acid (E) residue can be found in the TIRB1 and TIRB2 domains of TIRB proteins from various orthologs.

As discussed with reference to the TIRA and TIRB polypeptides from G. canescens, mutations in one or more of the NADase sites across the TIRA1, TIRA2, and TIRB1 domains may be used to modulate the relative activity of the TIR proteins, thereby conferring increased resistance function with reduced agronomic and growth issues. In particular embodiments, constructs comprising orthologous TirA and TirB genes and further comprising mutations corresponding to the E85A mutation in TIRA (SEQ ID NO: 1) and/or the E87A mutation in TIRB (SEQ ID NO: 2) can be introduced into a plant to confer the plant with increased disease resistance and improved agronomic performance. As a non-limiting example, constructs may be constructed comprising the TirA ortholog ccRpp-R1 (SEQ ID NO: 46) comprising a Glu to Ala mutation at position 86 and/or a Glu to Ala mutation at position 252 of SEQ ID NO: 46; and the TirB ortholog ccRpp-R3 (SEQ ID NO: 48) comprising a Glu to Ala mutation at position 84 of SEQ ID NO: 48. Still other Glu to non-Glu mutations may be inserted without departing from the scope of the invention.

Further still, polynucleotides encoding fusion proteins comprising a TIRA ortholog (e.g., SEQ ID NO: 46) linked to the corresponding TIRB ortholog (e.g., SEQ ID NO: 48) and optionally further comprising mutations corresponding to the E85A mutation in TIRA (SEQ ID NO: 1) and/or the E87A mutation in TIRB (SEQ ID NO: 2), such as an E to A mutation at positions 86 and/or 252 of SEQ ID NO: 46 and a E to A mutation at position 84 of SEQ ID NO: 48, can be introduced into a plant to confer the plant with increased disease resistance and improved agronomic performance. Still other Glu to non-Glu mutations may be introduced at the corresponding NADase sites without departing from the scope of the invention.

### Example 6: Identification of Allelic Variants of TIRA1A2 and TIRB1B2 Proteins

Allele identities were determined by conducting searches of the G. clandestina genome using gene alignment tools such as BLAST (Basic Local Alignment Search Tool; Altschul et al. (1993). Therein, searches were conducted under default parameters for sequences having similarity to the identified TirA and TirB genes (or their gene products). This led to the identification of three allelic variants of TirA in G. clandestina termed RG6a (SEQ ID NO: 39); RG7a (SEQ ID NO: 41), and RG8a (SEQ ID NO: 43), as well as RG6b (SEQ ID NO: 40); RG7b (SEQ ID NO: 42), and RG8b (SEQ ID NO: 44). The percent (%) identity values of the proteins encoded by the allelic variants to the TIRA and TIRB polypeptide sequences is presented in Table 17. Alignments were done using EMBOSS version 6.6.0.0, algorithm needle, with the following parameters: gapopen 10; and gapextend 0.5.

TABLE 17
Percentage identity of protein encoded by allelic
variants to TIRA (SEQ NO. 1) and TirB (SEQ NO. 2)

| | | | % Identity to | % Identity to |
|---|---|---|---|---|
| Allele | NT | | TIRA (SEQ | TIRB (SEQ |
| name | SEQ# | Source organism | NO: 1) | ID NO: 2) |
| RG6a | 39 | G. clandestina | 93.2 | — |
| RG6b | 40 | G. clandestina | — | 98.4 |
| RG7a | 41 | G. canescens | 99.7 | — |
| RG7b | 42 | G. canescens | — | 99.7 |
| RG8a | 43 | G. canescens | 98.5 | — |
| RG8b | 44 | G. canescens | — | 100 |

Alignment of one or more of the identified allelic variants of Table 17 with TirA and/or TirB genes enables the identification of the corresponding NADase sites. For example, when aligned with the other plant TIR domains, a conserved, presumably active, glutamatic acid (E) residue may be found in the TIRA1 and/or TIRA2 domain of TIRA allelic variants, and in the TIRB1 and/or TIRB2 domain of TIRB allelic variants.

Mutations in one or more of the NADase sites across the TIRA1, TIRA2, TIRB1 and TIRB2 domains of the allelic variants, identified for example based on alignments with the TIR polypeptide of the present disclosure, may be used to modulate the relative activity of the TIR proteins, thereby conferring resistance function with reduced agronomic and growth issues. In particular embodiments, constructs comprising RG6a (SEQ ID No: 39) or RG7a (SEQ ID No: 41) or RG8a (SEQ ID No: 43) co-expressed with the corresponding RG6g (SEQ ID No: 40) or RG7b (SEQ ID NO: 42) or RG8b (SEQ ID NO: 44) and further comprising mutations corresponding to the E85A mutation in TIRA (SEQ ID NO: 1) and/or the E87A mutation in TIRB (SEQ ID NO: 2) can be introduced into a plant to confer the plant with increased disease resistance and improved agronomic performance.

Further still, polynucleotides encoding fusion proteins comprising a TIRA allelic variant (e.g., RG6a (SEQ ID No: 39) or RG7a (SEQ ID No: 41) or RG8a (SEQ ID No: 43)) linked to the corresponding TIRB allelic variant (e.g., RG6b (SEQ ID No: 40) or RG7b (SEQ ID NO: 42) or RG8b (SEQ ID NO: 44)) and optionally further comprising mutations corresponding to the E85A mutation in TIRA (SEQ ID NO: 1) and/or the E87A mutation in TIRB (SEQ ID NO: 2) can be introduced into a plant to confer the plant with increased disease resistance and improved agronomic performance.

### Example 7: TIRA Polypeptides and TIRB Polypeptides Confer Broad Spectrum Resistance to Fungal and Bacterial Pathogens

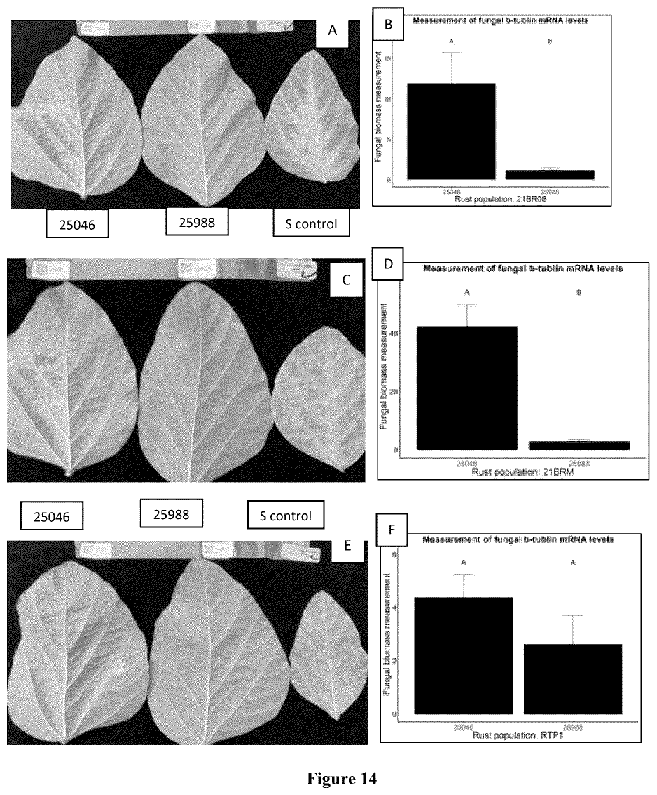

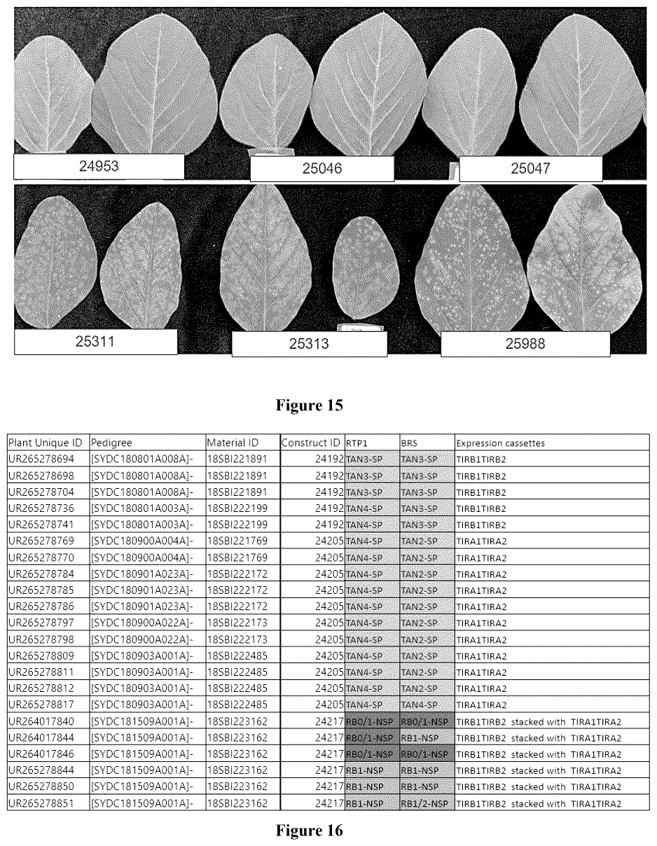

When various transgenic events described herein (at Examples 1-4) expressing wild-type and mutant TIR proteins were grown in greenhouse conditions, we observed that some events were infected with soybean powdery mildew while others were free from mildew. In particular, transgenic events expressing wild-type proteins (constructs 25046, 25047 and 24953) were resistant to powdery mildew while transgenic events expressing mutant proteins (constructs 25311, 25313, and 25988) were susceptible to powdery mildew. Detailed evaluation using detached leaves from these events confirmed this finding (FIG. 15). This led us to conclude that the TIRA polypeptides and TIRB polypeptides, working together, confer broad spectrum resistance, across two significantly different fungal phyla, to soybean rust and powdery mildew. Given the phylogenetic differences between the fungal phyla responsible for soybean rust and powdery mildew, we propose that the TIRA and TIRB polypeptides confer resistance to soybean rust and powdery mildew via two potentially different resistance mechanisms including distinct effector proteins. We further propose that active NADase sites are required at each of the TIRA1, TIRA2, TIRB1 and TIRB2 domains for conferring powdery mildew resistance.

### Example 8: Assaying Variant TIR Polypeptides and Tir Genes for Mode/Site of Action

Variants of the TIR polypeptides and genes, including sequences from other organisms, can be identified based on their sequence identity and/or functional identity with the TIRA and TIRB polypeptides and genes disclosed herein. In one example, variant polypeptides and polynucleotides of the TIRA and TIRB polypeptides, and polynucleotide sequences encoding such polypeptides, comprise proteins that interact with the same set of effector proteins to generate a localized hypersensitive response via a common mode of action when expressed in a plant. Variant polypeptide and polynucleotides including orthologs and allelic variants of TIRA and TIRB polypeptides of SEQ ID NOS: 1-2 and/or TirA and TirB genes of SEQ ID NOS: 3-8 are expected to interact with effector proteins that interact with the TIRA and TIRB polypeptides of SEQ ID NOS: 1-2 to generate a localized hypersensitive cell death response.

Accordingly, in some embodiments, methods are disclosed of identifying novel disease resistance polypeptides having the dumbbell TIR motifs of the TIR polypeptides of SEQ ID NOS: 1 and 2, the method comprising: identifying a set of plant pathogen effector proteins that interact with the TIRA polypeptide of SEQ ID NO: 1 and TIRB polypeptide of SEQ ID NO: 2, assaying for interaction of the identified set of plant pathogen effector proteins with a putative TIR polypeptide having at least 40%, at least 50%, at least 60%, at least 70%, at least 80%, at least 90% or at least 95% sequence identity to SEQ ID NO: 1 or SEQ ID NO: 2; and in response to detection of interaction between the putative TIR polypeptide and the identified effector(s), such as via the presence of a hypersensitive response or localized cell death, indicating that the putative TIR polypeptide has the same mode of action as the TIRA or TIRB polypeptide. In some embodiments, the effector proteins are expressed in a cell, purified, and infiltrated into a plant cell for an interaction assay with the putative TIR polypeptide. In other embodiments, a transient expression system, such as an Agrobacterium-mediated transient expression system in a plant can be used to observe the hypersensitive response cell death phenotype (HR) triggered by co-expression of a putative Tir gene system comprising a TirA and a TirB variant (e.g., TirA and TirB orthologs or homologs or allelic variants) and their putative cognate effector (e.g., an effector protein recognized by the TIR polypeptides of SEQ ID NOS: 1 and 2). Presence of an interaction, e.g., presence of an HR response and localized cell death, indicates that the protein encoded by the putative Tir genes have functional identity and a common mode of action as the TIR polypeptides of the present disclosure, even if the sequence identity is low (e.g., lower than 60% sequence identity, such as 50% identity or 40% identity or lower).

### Example 9: Native Promoter Derived from a Genomic Locus Comprising Rg32 and RG34 Genes Drives Bidirectional Expression

DNA constructs, including expression cassettes and vectors, were generated comprising a native promoter sequence (SEQ ID NO: 49) derived from a genomic locus comprising each of the Rg32 and Rg34 genes (disclosed in U.S. Provisional applications 63/426,524 and 63/509,586 as SEQ ID NO: 7 (genomic locus) and SEQ ID NO: 18 (bidirectional promoter), the contents of which are incorporated herein in their entirety). In view of the opposite relative orientation of the Rg32 gene and Rg34 genes in the genomic locus, it was postulated that the promoter derived from the genomic locus drives bidirectional expression. GUS expression of the promoter was evaluated in both orientations by comparing GUS expression driven via a first construct comprising the sense strand sequence of the native promoter sequence (indicated as prRG32Rg34_sense) relative to a second construct comprising the reverse complement sequence of the native promoter sequence (indicated as prRG32Rg34_RevCom). GUS expression via the native promoter in both orientations was further compared to a control construct comprising a plant active constitutive promoter, prUBQ3-13 (SEQ ID NO: 49). As shown in FIG. 23, GUS expression was assessed via GUS staining. GUS expression driven via the native promoter constructs in either orientation (that is, in sense and reverse complement orientation) displayed equivalent GUS staining compared to the control. This data strongly indicates the promoter of SEQ ID NO: 49 derived from the genomic locus between the Rg32 and Rg34 genes has bidirectional promoter activity.

In particular embodiments, vector constructs, expression constructs or nucleic acid sequences can be generated comprising the native bidirectional promoter of SEQ ID NO: 49 operably coupled to each of the TirA and TirB genes (or polynucleotides encoding TIRA polypeptides and TIRB polypeptides) such that the bidirectional promoter is able to drive expression of the genes in both directions (that is, via the promoter sequence in both the sense orientation and the promoter sequence in the antisense orientation or via the reverse complement sequence) to thereby confer increased disease resistance (e.g., increased ASR resistance). In a particular embodiment, the bidirectional promoter is operably coupled to each of a nucleic acid sequence encoding the TIRA polypeptide and a nucleic acid sequence encoding the TIRB polypeptide. In one specific example embodiment, the nucleic acid sequence encoding the TIRA polypeptide is operably coupled downstream of the bidirectional promoter while the nucleic acid sequence encoding the TIRB polypeptide is operably coupled upstream of the bidirectional promoter, when viewing the sequence in the sense orientation. In another specific example embodiment, the nucleic acid sequence encoding the TIRA polypeptide is operably coupled upstream of the bidirectional promoter while the nucleic acid sequence encoding the TIRB polypeptide is operably coupled downstream of the bidirectional promoter, when viewing the sequence in the sense orientation. Irrespective of the relative orientation of the TirA and TirB genes relative to the bidirectional promoter, significant expression of each of the TIRA polypeptide and the TIRB polypeptide via the bidirectional promoter is expected resulting in increased disease resistance.

### Example 10: Cyclic Nucleotide Monophosphates (cNMPs) Synthetase Residue in TIRA1 Domain Impacts Soybean Rust Resistance

Besides the NADase activity, TIR proteins also function as non-canonical cyclic nucleotide monophosphates (cNMPs) synthetases and this activity is essential for a TIR-mediated hypersensitive response (Yu et al., 2022; Tian and Li, 2022). All four TIR domains, that is TIRA1, TIRA2, TIRB1 and TIRB2 contain a putative cysteine (C) residue located 3 amino acids upstream of the putative NADase residues (FIG. 12). We made single, double, triple and quadruple mutations at these conserved cysteine residues by changing Cysteine to Alanine using site mutagenesis of the putative cysteine residues using construct 25046 (FIGS. 21 and 24).

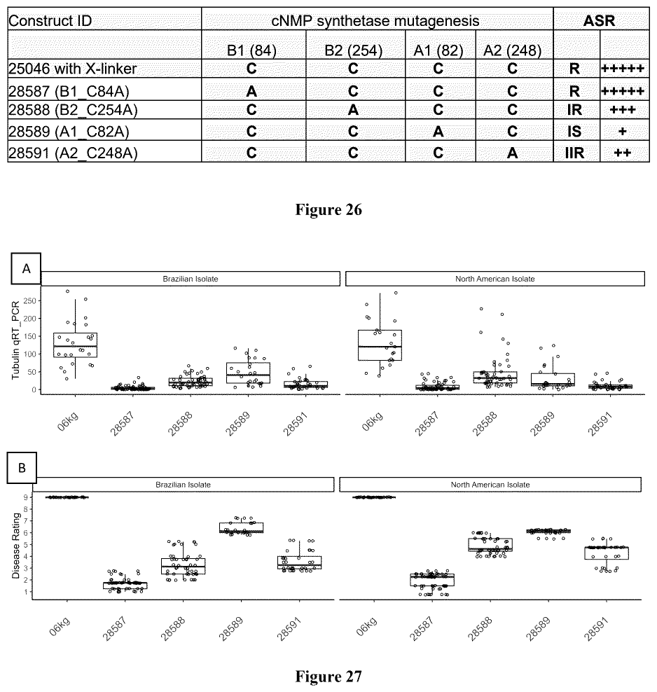

Multiple constructs with single, double, triple, and quadruple loss of function mutations were generated with changes from C to A (alanine) mutations in TIRA1, TIRA2, TIRB1 and TIRB2 domains (constructs 28587, 28588, 28589, and 28591). FIG. 26 summarizes the constructs and the corresponding mutations.

As used in FIG. 26, the mutations included:
C82A: Cys to Ala mutation at position 82 of the TIRA polypeptide (SEQ ID NO: 1) in the TIRA1 domain.
C248A: Cys to Ala mutation at position 248 of the TIRA polypeptide (SEQ ID NO: 1) in the TIRA2 domain.
C84A: Cys to Ala mutation at position 84 of the TIRB polypeptide (SEQ ID NO: 2) in TIRB1 domain.
C254A: Cys to Ala mutation at position 254 of the TIRB polypeptide (SEQ ID NO: 2) in TIRB2 domain.

We first selected 4 mutants with a single C to A mutation at each one of the 4 putative cNMP synthetase residuse to generate soybean stable transgenic events. As shown at FIGS. 26-27, all four single-mutants maintained resistance to ASR, albeit at variable levels. Notably, C to A mutation in the TIRB1 domain (C84A) maintained resistance at comparable level as wild type protein, while corresponding mutations in TIRB2 (C254A), TIRA1 (C82A) and TIRA2 (C248A) were efficacious but levels of resistance were substantially decreased. Inconsistent with NADase site mutagenesis results, we observed that the C residue mutation at TIRA1 had a stronger impact on TIRA and TIRB mediated resistance. Particularly, the C to A mutation in the TIRA1 domain showed the lowest level of resistance among all 4 mutants (FIG. 26-27). We then generated double, triple, and quadruple mutants. It is expected that the double, triple and quadruple mutations at these residues will have a larger impact on the resistance function than the single mutants.

### Example 11: TIRA and TIRB Recognize Effectors for a Variety of Different Plant Pathogens

Variants of the TIRA and TIRB polypeptides and their genes, including ortholog sequences from other organisms, paralog sequences from the same organism, as well as allelic variants can be identified based on their sequence identity and/or functional identity with the TIRA and TIRB polypeptides and genes disclosed herein. In one example, variant polypeptides and polynucleotides of the TIRA and TIRB polypeptides, and polynucleotide sequences encoding such polypeptides, comprise proteins that interact with the same set of effector proteins to generate a localized hypersensitive response via a common mode of action when expressed in a plant. Variant polypeptide and polynucleotides including orthologs, paralogs, annotation variants, splice variants, and allelic variants of the TIRA polypeptide of SEQ ID NO: 1 and/or the TIRB polypeptide of SEQ ID NO: 2 and/or the TirA gene of any of SEQ ID NOS: 3-5 and/or the TirB gene of any of SEQ ID NOS: 6-8 are expected to interact with effector proteins that interact with the TIRA and/or TIRB polypeptides of SEQ ID NOS: 1 and/or 2 to generate a localized hypersensitive cell death response.

Accordingly, in some embodiments, methods are disclosed of identifying novel disease resistance polypeptides having the same mode and/or site of action as the TIRA polypeptide of SEQ ID NO: 1 and/or the TIRB polypeptide of SEQ ID NO: 2, the method comprising: identifying a set of plant pathogen effector proteins that interact with the TIRA polypeptide of SEQ ID NO: 1 and/or the TIRB polypeptide of SEQ ID NO: 2, assaying for interaction of the identified set of plant pathogen effector proteins with a putative TIRA or putative TIRB polypeptide having at least 40%, at least 50%, at least 60%, at least 70%, at least 80%, at least 90% or at least 95% sequence identity to SEQ ID NO: 1 or SEQ ID NO: 2; and in response to detection of interaction between the putative TIRA or TIRB polypeptide and the identified effector(s), such as via the presence of a hypersensitive response or localized cell death, indicating that the putative TIRA or TIRB polypeptide has the same mode of action as the corresponding TIRA or TIRB polypeptide. In some embodiments, the effector proteins are expressed in a cell, purified, and infiltrated into a plant cell for an interaction assay with the putative TIRA and TIRA polypeptide. In other embodiments, a transient expression system, such as an Agrobacterium-mediated transient expression system in a plant can be used to observe the hypersensitive response cell death phenotype (HR) triggered by co-expression of a putative binary TIRA-TIRB gene system comprising a TIRA variant and a TIRB variant (e.g., TIRA and/or TIRB orthologs or homologs or allelic variants) and their putative cognate effector (e.g., an effector protein recognized by the TIRA polypeptide of SEQ ID NO: 1 and the TIRB polypeptide of SEQ ID NO: 2). Presence of an interaction, e.g., presence of an HR response and localized cell death, indicates that the protein encoded by the putative TirA or TrB genes have functional identity and a common mode of action as the corresponding TIRA or TIRB polypeptides, respectively, of the present disclosure, even if the sequence identity is low (e.g., lower than 60% sequence identity, such as 50% identity or 40% identity or lower).

I. Identification of Asian Soybean Rust Effector Proteins that Interact with TIRA and TIRB

Plant pathogens secrete various effectors to subvert host immunity. When these effectors are recognized by plant immunity proteins, this recognition often triggers hypersensitive response in a heterologous system like Nicotiana species. In particular embodiments, TIRA and TIRB polypeptides were found to recognize ASR effector proteins.

Three Soybean rust genome were recently assembled and annotated with over 400 common secreted effectors identified (Gupta et al., 2022). More than 300 curated soy rust effectors were assessed for interaction with TIRA and/or TIRB.

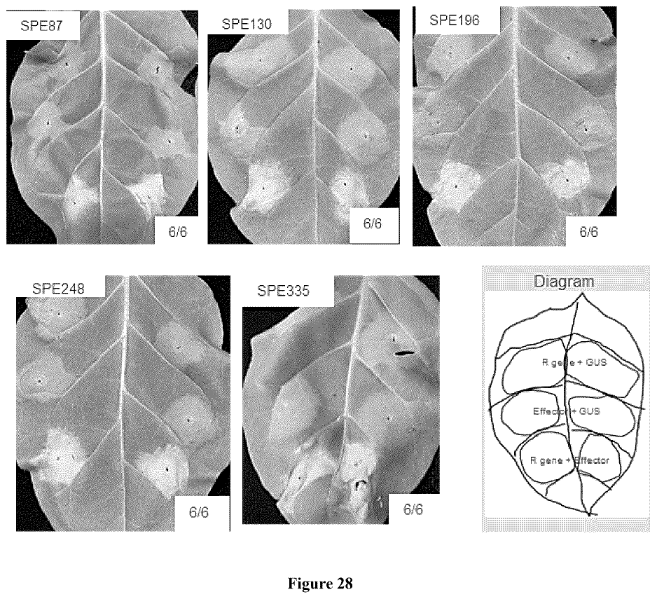

Of the more than 300 effectors, five effectors, effector protein SPE335 (SEQ ID NO: 51), or effector protein SPE087 (SEQ ID NO: 52), or effector protein SPE130 (SEQ ID NO: 53), or effector protein SPE196 (SEQ ID NO: 54), or effector protein SPE248 (SEQ ID NO: 55), were recognized when assessed using heterologous Nicotiana spp. These 5 effectors triggered a specific hypersensitive response in N. tabacum 3-4 days post infiltration, while other annotated effectors, when co-infiltrated with TIRA and TIRB, had no hypersensitive response (HR) up to 5 days after infiltration (FIG. 28).

Agrobacterium strains containing R-genes TIRA and/or TIRB or a given soy rust effector were grown in Luria-Bertani agar plates, supplemented with 50 μg/mL spectinomycin and 50 g/mL kanamycin, overnight at 22 °C in a dark chamber. The cultures were scooped with a toothpick and diluted in induction medium (5 mM MgSO₄, 2 mg/ml MES, and 10 μM acetosyringone, adjusted to pH 5.6 with HCl). Bacterial concentrations were measured and adjusted with induction medium to OD₆₀₀=0.5. Resulting cultures were pre-induced for 2 to 3 h at room temperature. For co-infiltrations, cultures carrying individual constructs were induced separately and mixed in a 1:1 ratio just before infiltration. Young tobacco leaves were inoculated with Agrobacterium cultures using a blunt syringe. The Control construct expressed GUS (Beta-glucuronidase). Cell death was observed 3-5 h after infiltration.

As shown in FIG. 28, each of soy rust effectors SPE-87, 130, 196, 248, and 335, when expressed in N. tabacum via Agrobacterium mediated transient assay, specifically triggered a localized cell death response when co-infiltrated with construct 25046 (fusion construct expressing both TIRA and TIRB). A localized cell death response was not observed when any of the soy rust effectors were co-infiltrated with the control construct, or when construct 25046 expressing TIRA and TIRB was co-infiltrated with the control construct. This indicates that TIRA and TIRB interact with SPE-87, 130, 196, 248, and 335 selectively.

II. Identification of Variant Proteins that have the Same Mode of Action as TIRA and TIRB

Putative TIRA and TIRB variants can be identified by co-expression of the putative TIRA variant and the corresponding putative TIRB variant polypeptide in N. tabacum via Agrobacterium mediated transient assay. Triggering of a localized cell death response when co-infiltrated with any of soy rust effectors SPE-87, 130, 196, 248, and 335, as shown in FIG. 28, will indicate that the putative TIRA variant has the same mode of action as TIRA, and that the putative TIRB variant has the same mode of action as TIRB. In particular embodiments, triggering of a localized cell death response when co-infiltrated with any of soy rust effectors SPE-87, 130, 196, 248, and 335, will indicate that the putative TIRA variant has the same mode of action as TIRA in conferring increased ASR resistance, and that the putative TIRB variant has the same mode of action as TIRB in conferring increased ASR resistance.

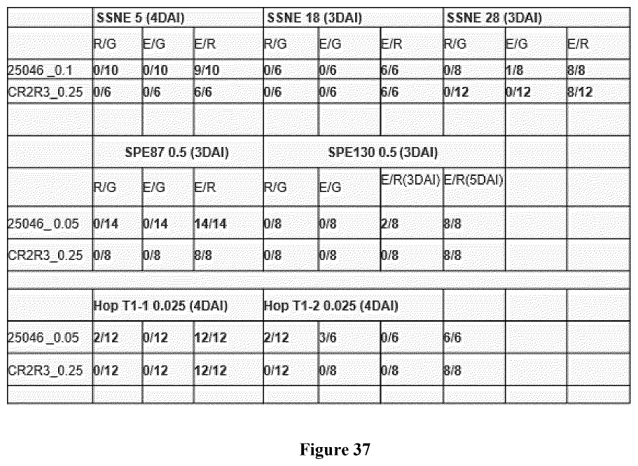

In one particular example, Agrobacterium strains containing TIRA and TIRB homologs from Cajanus cajun (ccRpp2-R1, SEQ ID NO: 46; and ccRpp2-R3, SEQ ID NO: 48) or a soy rust effector or a bacterial effector or a powdery mildew effector or a nematode effector (such as any of the effectors of Tables 1B and 1D) were grown. For co-infiltrations, cultures carrying individual constructs were induced separately and mixed in a 1:1 ratio just before infiltration. Young tobacco leaves were inoculated with Agrobacterium cultures using a blunt syringe. The Control construct expressed GUS (Beta-glucuronidase). Cell death was observed 3-5 h after infiltration. We found that several effectors from soybean rust, cyst nematodes and Pseudomonas syringae that are recognized by TIRA and TIRB are also recognized by their distant homolog in Cajanus cajun. As shown in FIG. 37, below shows an almost identical spectra of recognitions between TIRA and TIRB and their distant homologs with all effectors tested across 3 pathogens, specifically when tested with bacterial effectors HopT1-1 and HopT1-2, nematode effectors SSNE-5, 18, and 28, and rust effectors SPE 87 and 130.

III. Identification of Pseudomonas Effector Proteins that Interact with TIRA and TIRB

Besides resistance to soybean rust, we found that several Pseudomonas syringae pathovars, when infiltrated on transgenic soybean leaves expressing TIRA and TIRB, trigger a hypersensitive response. In particular embodiments, Pseudomonas syringae pathovars tomato strain DC3000 were found to trigger a HR. Bacterial effectors for Pseudomonas have been annotated are available in the art (see for example, Laflamme et al., 2020). As with the assessment of ASR effectors, more than 30 curated Pseudomonas effectors mined from the genome of Pseudomonas syringae pathovars tomato strain DC3000 were assessed for interaction with TIRA and/or TIRB.

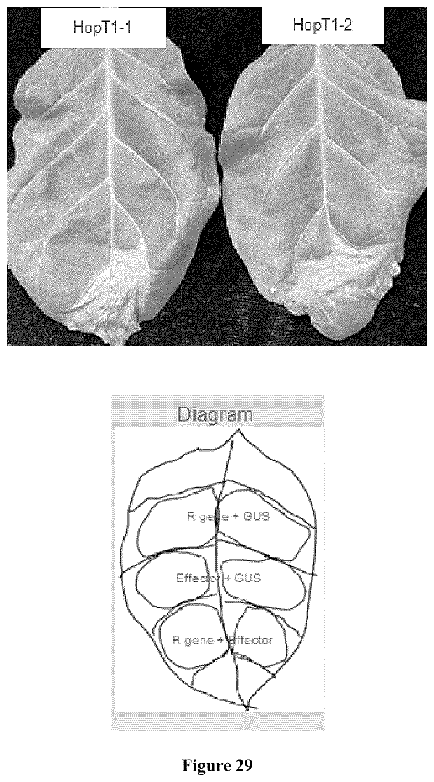

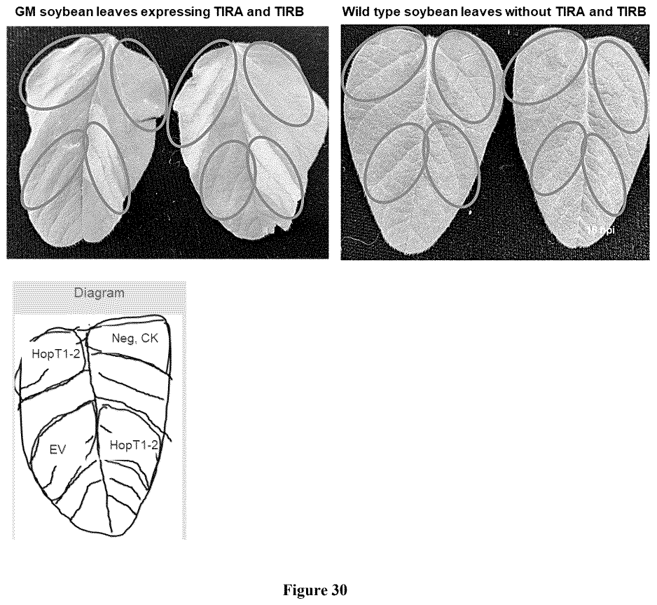

Of the assessed effectors, effectors from the HopT1family of effectors, including HopT1-1 (SEQ ID NO: 61 (protein sequence); SEQ ID NO: 59 (nucleotide sequence)), and HopT1-2 (SEQ ID NO: 60 (nucleotide sequence); SEQ ID NO: 62 (protein sequence)) were recognized when assessed using heterologous Nicotiana spp. The family of HopT1 effectors triggered a specific hypersensitive response in N. tabacum 3-4 days post infiltration, while other annotated effectors, when co-infiltrated with TIRA and TIRB, had no hypersensitive response (HR) up to 5 days after infiltration (FIG. 29).

Pseudomonas syringae pv. glycines does not naturally contain HopT1 effectors. Therefore, when infiltrated into transgenic soybean leaves expressing TIRA and TIRB, no HR was observed. However, when HopT1 effectors were conjugated into Pseudomonas syringae pv. Glycines, the bacteria were able to cause HR on the transgenic soybean leaves expressing TIRA and TIRB (FIG. 30).

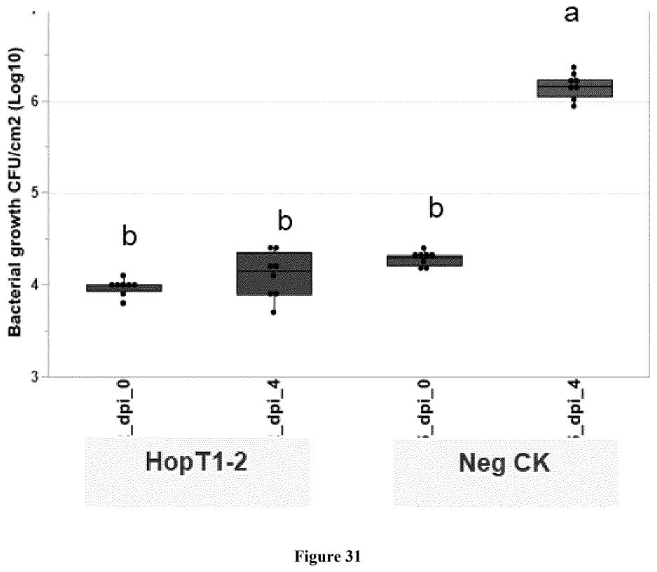

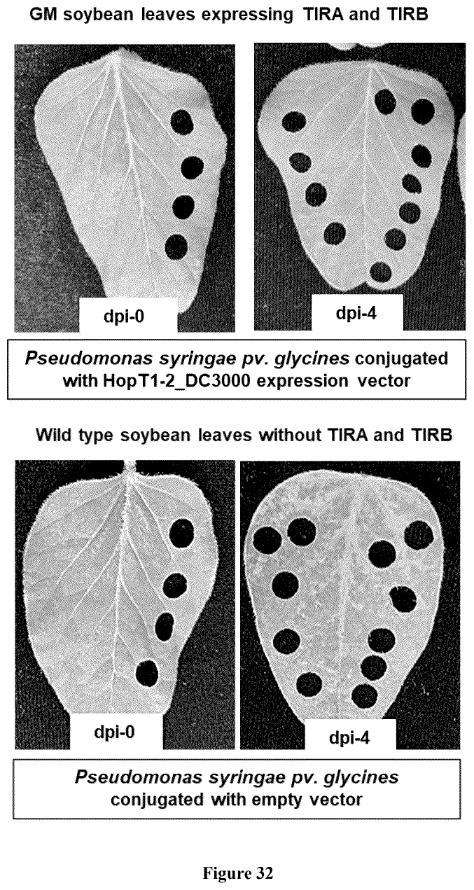

Consistently, little to no bacterial growth was observed when Pseudomonas syringae pv. glycines carrying HopT1 family effectors were infiltrated on transgenic soybean leaves expressing TIRA and TIRB proteins. In contrast, a nearly 100-fold growth of Pseudomonas syringae pv. glycines was observed on wild type soybeans that were not expressing TIRA and TIRB or Pseudomonas syringae pv. glycines not conjugated with HopT1 family effectors on GM soybeans expressing TIRA and TIRB (FIG. 31). Subsequent observations of disease progress, as shown in FIG. 32, support the conclusion that TIRA and TIRB confer HopT1 effector family dependent cell death and resistance.

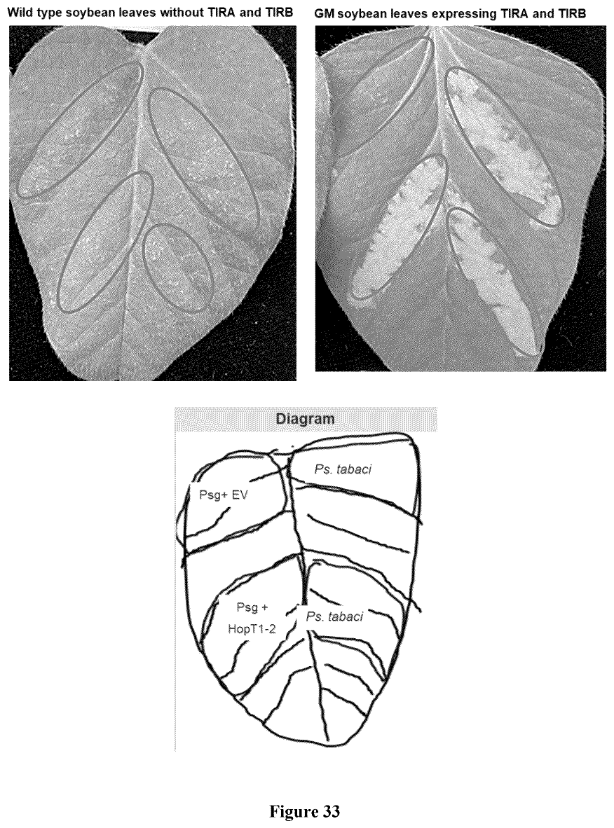

Pseudomonas syringae pv. tabaci (Ps tabaci) causes wildfire disease in soybean, as first reported in 1945. As such, this is one of the most common bacterial diseases globally in soybean (Lay et al., Evaluation of Soybean Wildfire Prediction via Hyperspectral Imaging. Plants. February 16;12 (4): 901 (2023)). Pseudomonas syringae pv. tabaci does contain HopT1-1 and HopT1-2 effectors (Laflamme et al., The pan-genome effector-triggered immunity landscape of a host-pathogen interaction. Science. 2020 Feb. 14;367 (6479): 763-768). We assessed to see if Pseudomonas syringae pv. tabaci triggers TIRA and TIRB dependent resistance. Indeed, as expected, and as shown in FIG. 33, transgenic soybean events expressing TIRA and TIRB developed a strong hypersensitive response within 16 hours after infiltration similar to Pseudomonas syringae pv. glycines carrying HopT1-2, while wild-type soybean had little to no visible response to Pseudomonas syringae pv. tabaci. HopT1-2 proteins in PSt DC3000 share high homology with HopT1-2 in Pseudomonas syringae pv. tabaci with only a 6 amino acid difference. HopT1 effectors are widespread in Pseuodomonas species. Consequently, we believe that any bacterial pathogen known to express a HopT1 effector, or any bacterial pathogen comprising in its genome a nucleotide sequence encoding a HopT1 effector, or a variant thereof, or any bacterial pathogen comprising its genome a sequence encoding a protein having significant sequence similarity (e.g., at least 60%, at least 70%, at least 80%, or at least 90% sequence similarity) or structural similarity to a HopT1 effector, could trigger a TIRA and TIRB dependent hypersensitive response and resistance.

Putative TIRA and TIRB variants can be identified by co-expression of the putative TIRA variant and the corresponding putative TIRB variant polypeptide in N. tabacum via Agrobacterium mediated transient assay. Triggering of a localized cell death response when co-infiltrated with any of Pseudomonas effectors, such as HopT1-1, HopT1-2, or other members of the HopT family, as shown in FIGS. 29-33, will indicate that the putative TIRA variant has the same mode of action as TIRA, and that the putative TIRB variant has the same mode of action as TIRB. In particular embodiments, triggering of a localized cell death response when co-infiltrated with any HopT effector will indicate that the putative TIRA variant has the same mode of action as TIRA in conferring increased bacterial resistance to Pseudomonas, and that the putative TIRB variant has the same mode of action as TIRB in conferring increased bacterial resistance to Pseudomonas.

IV. Identification of Powdery Mildew Effector Proteins that Interact with TIRA and TIRB

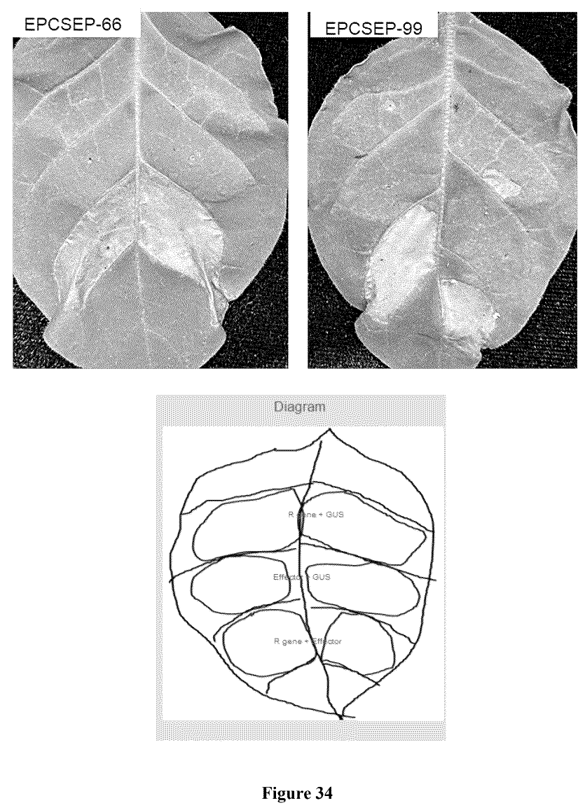

We have already demonstrated that wild-type TIRA and TIRB, not comprising any NADase mutations in their TIRA1, TIRA2, TIRB1, or TIRB2 domains, confer an immune response to powdery mildew (FIGS. 15 and 24, and Example 7). We proceeded to identity which effector proteins from the genome of the pathogen responsible for causing powdery mildew are recognized by TIRA and TIRB. Given that there is no soybean powdery mildew genome or annotated effectors reported, we turned to a related species, pea powdery mildew, Erysiphe pisi (Sharma et al., 2019). About 167 effectors were annotated and curated in Sharma et al., 2019. We synthesized these 167 effectors in the same construct expressing soybean rust effectors. These effectors were then co-infiltrated with TIRA and TIRB in Nicotiana species. Of the assessed effectors, two effectors, EPCSEP-66 (SEQ ID NO: 63 (nucleotide sequence); and SEQ ID NO: 65 (protein sequence)) and EPCSEP-99 (SEQ ID NO: 64 (nucleotide sequence); and SEQ ID NO: 66 (protein sequence)), were recognized when assessed using heterologous Nicotiana spp. The identified effectors triggered a specific hypersensitive response in N. tabacum 3-4 days post infiltration, while other annotated effectors, when co-infiltrated with TIRA and TIRB, had no hypersensitive response (HR) up to 5 days after infiltration (FIG. 34).

Putative TIRA and TIRB variants can be identified by co-expression of the putative TIRA variant and the corresponding putative TIRB variant polypeptide in N. tabacum via Agrobacterium mediated transient assay. Triggering of a localized cell death response when co-infiltrated with any of Powdery mildew effectors, such as EPCSEP-66 and-99, as shown in FIG. 34, will indicate that the putative TIRA variant has the same mode of action as TIRA, and that the putative TIRB variant has the same mode of action as TIRB. In particular embodiments, triggering of a localized cell death response when co-infiltrated with effector EPCSEP-66 or-99 will indicate that the putative TIRA variant has the same mode of action as TIRA in conferring increased resistance to powdery mildew, and that the putative TIRB variant has the same mode of action as TIRB in conferring increased resistance to powdery mildew.

V. Identification of Oomycete Effector Proteins that Interact with TIRA and TIRB

After observing TIRA and TIRB wild spectrum and specific recognitions of effectors from soybean rust, bacteria, and powdery mildew, we determined if TIRA and TIRB recognizes other effector proteins associated with other soybean diseases or pathogens. Two groups of pathogens were evaluated, oomycetes and nematodes. Effector repertoire for Phytophthora sojae is not as complete as for sunflower downy mildew caused by Plasmopara halstedii with about 262 effectors annotated (Sharma et al., 2015). We synthesized and cloned about 262 effectors into the same T-DNA vector previously used and screened each one of them by co-infiltrating with Agrobacterium containing TIRA and TIRB.

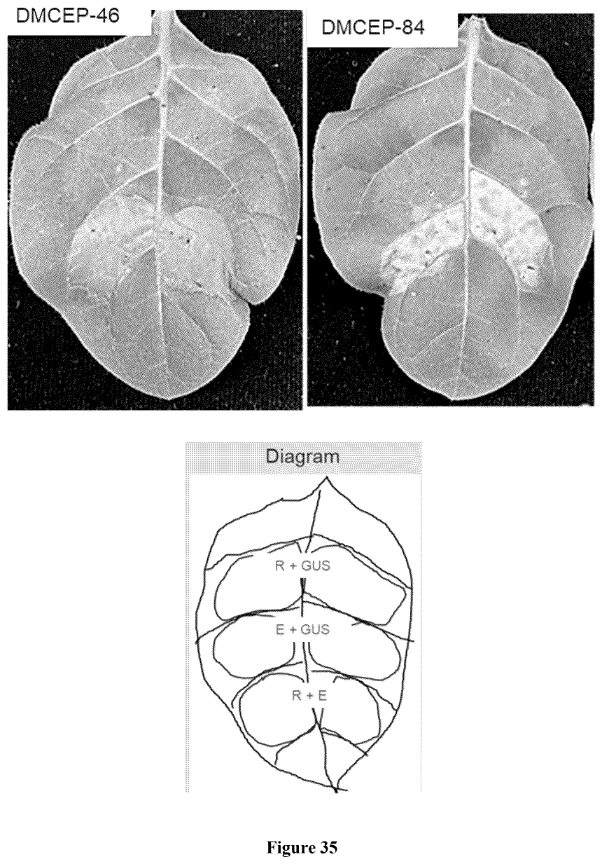

Of the assessed effectors, two effectors comprising a RxLR motif, DMCEP-46 and 84, triggered hypersensitive response (FIG. 35). These were: oomycete effector DMCEP-46 (SEQ ID NO: 67 (nucleotide sequence); and SEQ ID NO: 69 (protein sequence)) and DMCEP-86 (SEQ ID NO: 68 (nucleotide sequence); and SEQ ID NO: 70 (protein sequence)), both encoding RXLR effectors. We believe that this effector is conserved in oomycetes, such as Phytophthora species, including pathogens causing potato late blight (P. infestans), Sudden oak death (Phytophthora ramorum), and soybean Phytophthora root rot (Phytophthora sojae and P. sansomeana).

Putative TIRA and TIRB variants can be identified by co-expression of the putative TIRA variant and the corresponding putative TIRB variant polypeptide in N. tabacum via Agrobacterium mediated transient assay. Triggering of a localized cell death response when co-infiltrated with any effector comprising a RxLR motif, such as DMCEP-46 or 84, as shown in FIG. 35, will indicate that the putative TIRA variant has the same mode of action as TIRA, and that the putative TIRB variant has the same mode of action as TIRB. In particular embodiments, triggering of a localized cell death response when co-infiltrated with effector DMCEP-46 and 84 will indicate that the putative TIRA variant has the same mode of action as TIRA in conferring increased resistance to oomycetes, particularly Phytophthora species pathogens, and that the putative TIRB variant has the same mode of action as TIRB in conferring increased resistance to oomycetes, particularly Phytophthora species pathogens.

VI. Identification of Plant Parasitic Nematode Effector Proteins that Interact with TIRA and TIRB

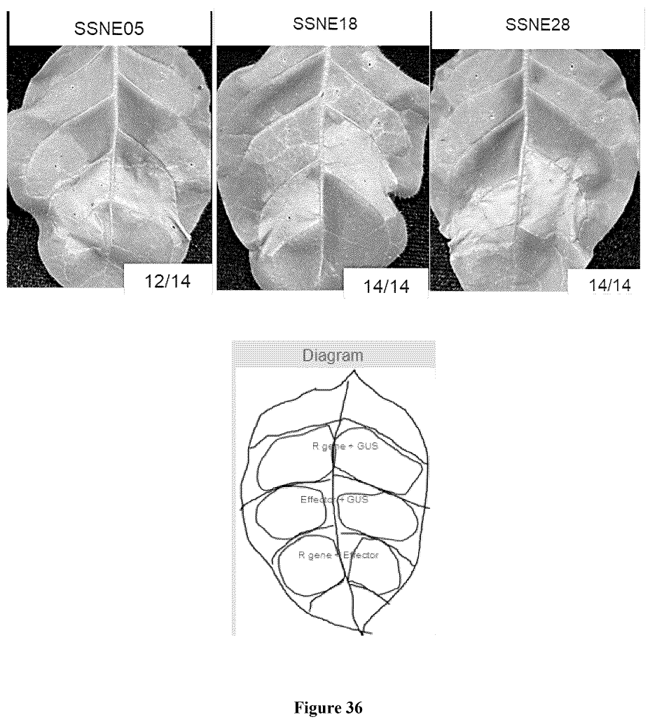

Effector candidates were also identified in soybean cyst nematode secreted by stylet (Gao et al., 2003, Noon et al., 2015 and Gardner et al., 2018). We synthesized and cloned about 65 effectors into the same T-DNA vector previously used and screened each one of them by co-infiltrating with Agrobacterium containing TIRA and TIRB. Of the assessed effectors, three effectors, SSNE-05, 18 and 28, triggered a hypersensitive response (FIG. 36). These were effector SSNE-05 (SEQ ID NO: 56 (nucleotide sequence)); SSNE-18 (SEQ ID NO: 57 (nucleotide sequence); and SSNE-28 (SEQ ID NO: 58 (nucleotide sequence)). Based on this observation, we expect TIRA and TIRB containing events of soybean or other plant species may confer effective resistance to nematodes including cyst nematodes and root knot nematode.

Putative TIRA and TIRB variants can be identified by co-expression of the putative TIRA variant and the corresponding putative TIRB variant polypeptide in N. tabacum via Agrobacterium mediated transient assay. Triggering of a localized cell death response when co-infiltrated with any nematode effector, such as SSNE-05, 18 or 28, as shown in FIG. 36, will indicate that the putative TIRA variant has the same mode of action as TIRA, and that the putative TIRB variant has the same mode of action as TIRB. In particular embodiments, triggering of a localized cell death response when co-infiltrated with effector SSNE-05, 18 or 28 will indicate that the putative TIRA variant has the same mode of action as TIRA in conferring increased resistance to nematodes, particularly soybean cyst nematode or root knot nematode, and that the putative TIRB variant has the same mode of action as TIRB in conferring increased resistance to nematodes, particularly soybean cyst nematode or root knot nematodes.

VII. Identification of Sucking or Piercing Pest Effector Proteins that Interact with TIRA and TIRB

Effector candidates will be identified from the genome of sucking and piercing pests, such as stinkbugs and aphids (e.g., from genome annotations). Effector candidates will be synthesized and cloned into the same T-DNA vector previously used and each one will be screened by co-infiltrating with Agrobacterium containing TIRA and TIRB. Of the assessed effectors, effectors that trigger a hypersensitive response will be identified as effectors that interact with TIRA-TIRB in providing a TIRA-TIRB mediated HR response and resistance.

### Example 12: Endogenous Expression Cassettes Comprising TIRA and TIRB Confer Plant Pathogen Resistance

Expression of TIRA and TIRB will be driven in endogenous or native expression cassettes comprising SEQ ID NO: 71 or 72. Expression is expected to confer resistance against soybean rust and other plant pathogens in a manner similar to construct 25046 expressing both TIRA and TIRB. In embodiments, the endogenous expression cassette of SEQ ID NO: 71 driving the expression of TIRA and TIRB comprises one or more native regulatory elements. Positions of the native regulatory elements with reference to SEQ ID NO: 71 is provided in Table 18 below.

TABLE 18
Position of elements within endogenous
expression cassette of SEQ ID NO: 71

| Name of regulatory | Type of regulatory | Position with reference to |
|---|---|---|
| element | element | SEQ ID NO: 71 |
| prGCaRG3b-03 | Native promoter | 1-7541 |
| 5′UTR | 5′UTR | 7394-7541 |
| TSS | TSS | 7394 |
| cGCaRG3b-02 | Coding sequence | 7542-9271 |
| Intron1 | Intron | 8021-8369 |
| Intron2 | Intron | 8883-9159 |
| RG3b-3′UTR | 3′UTR | 9272-9534 |
| tGCaRG3b-02 | Terminator | 9272-10434 |
| TES | TES | 9534 |
| Stop | Stop codon | 9474-9476 |
| prGCaRG3a-02 | Promoter | 10435-11549 |
| 5′UTR | 5′UTR | 11397-11549 |
| TSS | TSS | 11397 |
| cGCaRG3a-01 | Coding sequence | 11550-14109 |
| RG3a-3′UTR | 3′UTR | 14110-14647 |
| tGCaRG3a-02 | Terminator | 14110-15767 |
| Stop | Stop codon | 14556-14558 |
| TES | TES | 14647 |

In embodiments, the endogenous expression cassette of SEQ ID NO: 72 driving the expression of TIRA and TIRB comprises one or more native regulatory elements. Positions of the native regulatory elements with reference to SEQ ID NO: 72 is provided in Table 19 below.

TABLE 19
Position of elements within endogenous
expression cassette of SEQ ID NO: 72

| Name of regulatory | Type of regulatory | Position with reference to |
|---|---|---|
| element | element | SEQ ID NO: 72 |
| prGCaRG3b-05 | Native promoter | 1-751 |
| 5′UTR | 5′UTR | 604-751 |
| TSS | TSS | 604 |
| cGCaRG3b-02 | Coding sequence | 752-2481 |
| Intron1 | Intron | 1231-1579 |
| Intron2 | Intron | 2093-2369 |
| RG3b-3′UTR | 3′UTR | 2482-2744 |
| tGCaRG3b-02 | Terminator | 2482-3644 |
| TES | TES | 2744 |
| gGCaRG3a | loci | 3645-8857 |
| prGCaRG3a-02 | Promoter | 3645-4759 |
| 5′UTR | 5′UTR | 4607-4759 |
| TSS | TSS | 4607 |
| Start | Start codon | 4701-4703 |
| cGCaRG3a-01 | Coding sequence | 4760-7319 |
| RG3a-3′UTR | 3′UTR | 7320-7857 |
| tGCaRG3a-02 | Terminator | 7320-8977 |
| Stop | Stop codon | 7766-7768 |
| TES | TES | 7857 |

The above examples clearly illustrate the advantages of various embodiments of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

1. A nucleic acid molecule comprising a nucleotide sequence operably linked to a heterologous regulatory element, wherein the nucleotide sequence comprises:
(a) a first polynucleotide encoding a TIRA polypeptide comprising an amino acid sequence having at least 90% identity to SEQ ID NO: 1; and
(b) a second polynucleotide encoding a TIRB polypeptide comprising an amino acid sequence having at least 90%, identity to SEQ ID NO: 2,
wherein introduction of the nucleic acid molecule in a plant causes an expression level of the TIRA polypeptide relative to the TIRB polypeptide to be rebalanced in the plant as compared to the expression level in a control plant, wherein the rebalanced expression level of the TIRA polypeptide relative to the TIRB polypeptide, or active variant thereof, comprises a ratio of about 0.5:1 to about 2:1, and wherein expression of the nucleic acid molecule confers the plant with increased disease resistance.

2. The nucleic acid molecule of claim 1, wherein the first polynucleotide comprises:
(i) a nucleotide sequence having at least 95% sequence identity to any one of SEQ ID NOS: 3-5;
or
(ii) the nucleotide sequence of (i) further comprising a mutation that decreases or increases an NADase activity of the TIRA polypeptide; and wherein the second polynucleotide comprises:
(iii) a nucleotide sequence having at least 95% sequence identity to any one of SEQ ID NOS: 4-6;
or
(iv) the nucleotide sequence of (iii) further comprising a mutation that decreases an NADase activity of the TIRB polypeptide.

3. (canceled)

4. The nucleic acid molecule of any one of claims 1-2, wherein the nucleic acid molecule expresses a fusion protein comprising the TIRA polypeptide and the TIRB polypeptide.

5. (canceled)

6. The nucleic acid molecule of any of claims 1-2, and 4, wherein the heterologous regulatory element is a heterologous promoter active in said plant, and wherein the heterologous promoter is a constitutive promoter, an inducible promoter, or an endogenous promoter, and wherein the inducible promoter is optionally a rust inducible promoter.

7. The nucleic acid molecule of claim 6, wherein the heterologous promoter is selected from the group comprising SEQ ID NOS: 22-27.

8. The nucleic acid molecule of claim 6, wherein the heterologous regulatory element comprises a first heterologous promoter active in said plant operably coupled to the first polynucleotide in a first expression cassette; and a second heterologous promoter active in said plant operably coupled to the second polynucleotide in a second expression cassette.

9. The nucleic acid molecule of claim 8, wherein the second expression cassette is positioned transcriptionally upstream of the first expression cassette.

10. (canceled)

11. The nucleic acid molecule of claim 8 or 9, wherein the first heterologous promoter comprises any one of SEQ ID NOS: 22-24 and 26-27 and wherein the second heterologous promoter comprises SEQ ID NO: 25 or 27.

12. The nucleic acid molecule of claim 2, wherein the mutation of (ii) comprises a mutation at a position corresponding to position 85 or 251 of SEQ ID NO: 1, and wherein the mutation of (iv) comprises a mutation at a position corresponding to position 87 of SEQ ID NO: 2.

13. (canceled)

14. A vector comprising the nucleic acid molecule of any one of claim 1, 2, 4, 6-9, or 11-12.

15. A transgenic cell comprising the nucleic acid of any one of claim 1, 2, 4, 6-9, or 11-12 or the vector of claim 14, wherein the transgenic cell is a transgenic plant cell, wherein the transgenic plant cell is a transgenic plant cell of a legume plant, optionally wherein the legume plant is selected from the group comprising alfalfa, clover, pea, bean lentil, lupin, mesquite, carob, soybean, pigeon pea, peanut, and tamarind.

16. A plant, or plant part, derived from the transgenic cell of claim 15, wherein the plant part is a transgenic seed, wherein said transgenic seed has stably incorporated the first polynucleotide and the second polynucleotide into its genome.

17. A harvested product derived from the transgenic seed of claim 16, wherein the harvested product comprises the first and the second polynucleotide.

18. A processed product derived from the harvested product of claim 17, wherein the processed product is a flour, a meal, an oil, a starch, or a product derived from any of the foregoing, wherein the processed product comprises the first polynucleotide and the second polynucleotide.

19. A fusion protein comprising:
(a) a TIRA polypeptide comprising an amino acid sequence having at least identity to SEQ ID NO: 1; and
(b) a TIRB polypeptide comprising an amino acid sequence having at least identity to SEQ ID NO: 2,
wherein expression of said fusion protein in a plant confers the plant with increased disease resistance relative to a control plant not expressing the fusion protein.

20. The fusion protein of claim 19, wherein (i) the TIRA polypeptide is at the N-terminus of the fusion protein or (ii) the TIRB polypeptide is at the N-terminus of the fusion protein.

21. The fusion protein of claim 20, wherein the fusion protein of (i) comprises an amino acid sequence having at least identity to SEQ ID NO: 12 and wherein the fusion protein of (ii) comprises an amino acid sequence having at least 90% identity to any of SEQ ID NOS: 9-11.

22-24. (canceled)

25. The fusion protein of any of claims 19-21, further comprising a cleavable linker coupling the TIRA polypeptide to the TIRB polypeptide, wherein cleavage of the fusion protein at the cleavable linker following expression of the fusion protein in the plant rebalances an expression level of the TIRA polypeptide relative to the TIRB polypeptide in the plant as compared to the expression level in a control plant, wherein the rebalanced expression level of the TIRA polypeptide relative to the TIRB polypeptide comprises a ratio of about 0.5:1 to about 2:1.

26. The fusion protein of any of claims 19-21 or 25, wherein the TIRA polypeptide
further comprises a mutation that reduces or increases an NADase activity of the TIRA polypeptide, and
wherein the TIRB polypeptide
further comprises a mutation that decreases an NADase activity of the TIRB polypeptide.

27-29. (canceled)

30. A soybean plant, plant part, or seed comprising the fusion protein of any of claim 19-21, or 25-26, wherein the plant is resistant to Asian Soy Rust and/or powdery mildew.

31-32. (canceled)

33. A method of controlling disease in an area of cultivation comprising the step of planting in the area of cultivation a plant having stably incorporated into its genome:
(a) a first heterologous nucleotide sequence encoding a TIRA polypeptide having at least 90% identity to SEQ ID NO: 1;
(b) a second heterologous nucleotide sequence encoding a TIRB polypeptide having at least 90% identity to SEQ ID NO: 2; and
(c) a heterologous promoter operably coupled to the first and/or the second heterologous nucleotide sequence,
wherein an expression level of the TIRA polypeptide relative to the TIRB polypeptide is rebalanced in the plant, as compared to the expression level in a control plant, wherein the rebalanced expression level of comprises a ratio of about 0.5:1 to about 2:1, and wherein co-expression of the TIRA polypeptide with the TIRB polypeptide confers the plant with increased disease resistance.

34. The method of claim 33, wherein one or more of the TIRA polypeptide, or TIRB polypeptide are tagged with a detectable marker, or wherein the TIRA polypeptide and TIRB polypeptide are expressed as a fusion protein.

35-36. (canceled)

37. The method of claim 33 or 34, wherein the heterologous promoter comprises a nucleotide sequence having at least 95% sequence identity to any one of SEQ ID NOS: 23-28 or wherein the promoter comprises the sequence of any one of SEQ ID NOS: 22-27.

38. The method of claim 34, wherein the TIRA polypeptide has a dumbbell structure comprising a TIRA1 region and a TIRA2 region, each of the TIRA1 and TIRA2 region comprising an NADase site, wherein the TIRB polypeptide has a dumbbell structure comprising a TIRB1 region and a TIRB2 region, each of the TIRB1 and TIRB2 region comprising an NADase site and wherein the fusion protein further comprises;
(i) a loss of function mutation in the TIRA1 or TIRA2 region that reduces an NADase activity of the TIRA polypeptide or a gain of function mutation in the TIRA2 region that increases an NADase activity of the TIRA polypeptide; and
(ii) a loss of function mutation in the TIRB1 region that reduces an NADase activity of the TIRB polypeptide

39-40. (canceled)

41. The method of claim 38, wherein:
the loss of function mutation that reduces the NADase activity of the TIRA polypeptide comprises a mutation at a position corresponding to position 85 of SEQ ID NO: 1;
the gain of function mutation that increases the NADase activity of the TIRA polypeptide comprises a mutation at a position corresponding to position 251 of SEQ ID NO: 1; and
the loss of function mutation that reduces the NADase activity of the TIRB polypeptide comprises a mutation at a position corresponding to position 87 of SEQ ID NO: 2.

42. A soybean plant produced by the method of any one of claim 33, 34, 37, or 38-41, wherein the soybean plant has resistance to one or more of Asian Soybean Rust, powdery mildew, Pseudomonas syringae, or soybean cyst nematode, optionally wherein the soybean plant is an elite soybean plant.

43. The method of claim 33, wherein controlling disease comprises controlling the spread of the Asian Soybean Rust and/or powdery mildew and/or Pseudomonas syringae and/or soybean cyst nematode in the area of cultivation.

44-51. (canceled)

52. A method of screening for a plant having increased disease resistance and increased agronomic performance, comprising:
(a) introducing into the genome of the plant, a nucleic acid molecule comprising a heterologous promoter operably coupled to (i) a first polynucleotide encoding a encoding a TIRA polypeptide having at least identity to SEQ ID NO: 1; and (ii) a second polynucleotide encoding a TIRB polypeptide having at least identity to SEQ ID NO: 2;
(b) assaying an expression level of each of the TIRA polypeptide and the TIRB polypeptide in the plant or a progeny plant thereof;
(c) comparing the expression level of the TIRA polypeptide relative to the expression level of the TIRB polypeptide, to determine if the relative expression level is between 0.5:1 to 2:1, thereby determining a balanced expression level of the TIRA polypeptide and the TIRB polypeptide in the plant or progeny plant.

53. The method of claim 52, further comprising, selecting a plant or progeny plant having a balanced expression level of TIRA polypeptide relative to the TIRB polypeptide.

54-60. (canceled)


SEQUENCE LISTING (72 sequences)

SEQ ID NO: 1 - protein, 340 aa, Glycine canescens
MADEGGIAKWRYDVFLCFRGEDTRHTFTGNLYAALRKARLRTFFDVGLKGGDQIFEAIVQALEASRISIVVLSKSFASSSWCLEELVKILELRATKKQLVIPIFYHVDPSHVRKQTGSFGEYLIRHESRLGKDSEKIHKWKSALYEVATLAGWCFGDGSCRYQYEYEFIENIVREAIAIVPRYSIFLSFSGNDTRSFTGFLNNALCRSRYQTFMNDGDQISQSTNGVIEESRLSIIVFSENYARSSSCLDVLLTILQCMETKNQLICPIFYKVLPSDLRHQRNSYGEAMTEHENMLGKDSERVKKWRSALFDVANLKGFYLKTGYEYEFIDKIVEMASKI

SEQ ID NO: 2 - protein, 367 aa, Glycine canescens
MSNELKNYDVFLSFHGEDSRYTFTGTLYNALRSKRIKTFFTEHEYDRKLHTHDSQIPHSTLEAIKESRISMVVFSENYASSSRCLDELVAILECNRTINQLVWPIFYGVYPWQVRHQGGNYGASMSKLEEIFRDSNERVKQWRAALFEVTNLNGWCYQTGRYQYEYEFIERIVESAVQALPGYDVFLSFTGEDTRYTFTGFLYNAFRREGFKIFMDDEELESGNQISQKLMGAIESSRISIVVLSENYAYSTWCLDELAKIIECMKTNNQMVWPIFYNVQKSDVCNQTKSYGEAMTEHEKRFGKDSEKVQKWSSALSEINNLEGDHVKENEYQHEFIERIVQKVIKLEGSKHIANPFLLSNDSYEEE

SEQ ID NO: 3 - DNA, 2560 bp, Glycine canescens
  source 1..2560: mol_type = other DNA
atggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 4 - DNA, 1023 bp, Glycine canescens
  source 1..1023: mol_type = other DNA
atggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacgaatatgaatttattgacaaaatcgtggaaatggccagtaaaatttaa

SEQ ID NO: 5 - DNA, 1023 bp, Glycine canescens; identical to SEQ ID NO: 4
  source 1..1023: mol_type = other DNA

SEQ ID NO: 6 - DNA, 1730 bp, Glycine canescens
  source 1..1730: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaatga

SEQ ID NO: 7 - DNA, 1104 bp, Glycine canescens
  source 1..1104: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtaccaacatgaatttatcgaaaggattgtacaaaaggtcattaaacttgaaggtagcaagcatatagcaaatcctttccttttatccaatgacagctacgaggaagaatga

SEQ ID NO: 8 - DNA, 1513 bp, Glycine canescens
  source 1..1513: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtaccaacatgaatttatcgaaaggattgtacaaaaggtcattaaacttgaaggtagcaagcatatagcaaatcctttccttttatccaatgacagctacgaggaagaatga

SEQ ID NO: 9 - protein, 743 aa, Glycine canescens
MSNELKNYDVFLSFHGEDSRYTFTGTLYNALRSKRIKTFFTEHEYDRKLHTHDSQIPHSTLEAIKESRISMVVFSENYASSSRCLDELVAILECNRTINQLVWPIFYGVYPWQVRHQGGNYGASMSKLEEIFRDSNERVKQWRAALFEVTNLNGWCYQTGRYQYEYEFIERIVESAVQALPGYDVFLSFTGEDTRYTFTGFLYNAFRREGFKIFMDDEELESGNQISQKLMGAIESSRISIVVLSENYAYSTWCLDELAKIIECMKTNNQMVWPIFYNVQKSDVCNQTKSYGEAMTEHEKRFGKDSEKVQKWSSALSEINNLEGDHVKENEYQHEFIERIVQKVIKLEGSKHIANPFLLSNDSYEEEPGSVGGLSPSQASSAASSASSSPGSGISEALRAGAGSGMADEGGIAKWRYDVFLCFRGEDTRHTFTGNLYAALRKARLRTFFDVGLKGGDQIFEAIVQALEASRISIVVLSKSFASSSWCLEELVKILELRATKKQLVIPIFYHVDPSHVRKQTGSFGEYLIRHESRLGKDSEKIHKWKSALYEVATLAGWCFGDGSCYQYEYEFIENIVREAIAIVPRYSIFLSFSGNDTRSFTGFLNNALCRSRYQTFMNDGDQISQSTNGVIEESRLSIIVFSENYARSSSCLDVLLTILQCMETKNQLICPIFYKVLPSDLRHQRNSYGEAMTEHENMLGKDSERVKKWRSALFDVANLKGFYLKTYEYEFIDKIVEMASKI

SEQ ID NO: 10 - protein, 725 aa, Glycine canescens
MSNELKNYDVFLSFHGEDSRYTFTGTLYNALRSKRIKTFFTEHEYDRKLHTHDSQIPHSTLEAIKESRISMVVFSENYASSSRCLDELVAILECNRTINQLVWPIFYGVYPWQVRHQGGNYGASMSKLEEIFRDSNERVKQWRAALFEVTNLNGWCYQTGRYQYEYEFIERIVESAVQALPGYDVFLSFTGEDTRYTFTGFLYNAFRREGFKIFMDDEELESGNQISQKLMGAIESSRISIVVLSENYAYSTWCLDELAKIIECMKTNNQMVWPIFYNVQKSDVCNQTKSYGEAMTEHEKRFGKDSEKVQKWSSALSEINNLEGDHVKENEYQHEFIERIVQKVIKLEGSKHIANPFLLSNDSYEEEGEGRGSLLTCGDVEENPGMADEGGIAKWRYDVFLCFRGEDTRHTFTGNLYAALRKARLRTFFDVGLKGGDQIFEAIVQALEASRISIVVLSKSFASSSWCLEELVKILELRATKKQLVIPIFYHVDPSHVRKQTGSFGEYLIRHESRLGKDSEKIHKWKSALYEVATLAGWCFGDGSCRYQYEYEFIENIVREAIAIVPRYSIFLSFSGNDTRSFTGFLNNALCRSRYQTFMNDGDQISQSTNGVIEESRLSIIVFSENYARSSSCLDVLLTILQCMETKNQLICPIFYKVLPSDLRHQRNSYGEAMTEHENMLGKDSERVKKWRSALFDVANLKGFYLKTGYEYEFIDKIVEMASKI

SEQ ID NO: 11 - protein, 724 aa, Glycine canescens
MSNELKNYDVFLSFHGEDSRYTFTGTLYNALRSKRIKTFFTEHEYDRKLHTHDSQIPHSTLEAIKESRISMVVFSENYASSSRCLDELVAILECNRTINQLVWPIFYGVYPWQVRHQGGNYGASMSKLEEIFRDSNERVKQWRAALFEVTNLNGWCYQTGRYQYEYEFIERIVESAVQALPGYDVFLSFTGEDTRYTFTGFLYNAFRREGFKIFMDDEELESGNQISQKLMGAIESSRISIVVLSENYAYSTWCLDELAKIIECMKTNNQMVWPIFYNVQKSDVCNQTKSYGEAMTEHEKRFGKDSEKVQKWSSALSEINNLEGDHVKENEYQHEFIERIVQKVIKLEGSKHIANPFLLSNDSYEEEGEGRGSLLTCGDVEENPGPMADEGGIAKWRYDVFLCFRGEDTRHTFTGNLYAALRKARLRTFFDVGLKGGDQIFEAIVQALEASRISIVVLSKSFASSSWCLEELVKILELRATKKQLVIPIFYHVDPSHVRKQTGSFGEYLIRHESRLGKDSEKIHKWKSALYEVATLAGWCFGDGSRYQYEYEFIENIVREAIAIVPRYSIFLSFSGNDTRSFTGFLNNALCRSRYQTFMNDGDQISQSTNGVIEESRLSIIVFSENYARSSSCLDVLLTILQCMETKNQLICPIFYKVLPSDLRHQRNSYGEAMTEHENMLGKDSERVKKWRSALFDVANLKGFYLKRYEYEFIDKIVEMASKI

SEQ ID NO: 12 - protein, 745 aa, Glycine canescens
MADEGGIAKWRYDVFLCFRGEDTRHTFTGNLYAALRKARLRTFFDVGLKGGDQIFEAIVQALEASRISIVVLSKSFASSSWCLEELVKILELRATKKQLVIPIFYHVDPSHVRKQTGSFGEYLIRHESRLGKDSEKIHKWKSALYEVATLAGWCFGDGSCRYQYEYEFIENIVREAIAIVPRYSIFLSFSGNDTRSFTGFLNNALCRSRYQTFMNDGDQISQSTNGVIEESRLSIIVFSENYARSSSCLDVLLTILQCMETKNQLICPIFYKVLPSDLRHQRNSYGEAMTEHENMLGKDSERVKKWRSALFDVANLKGFYLKTGYEYEFIDKIVEMASKIPGSVGGLSPSQASSAASSASSSPGSGISEALRAGAGSGMSNELKNYDVFLSFHGEDSRYTFTGTLYNALRSKRIKTFFTEHEYDRKLHTHDSQIPHSTLEAIKESRISMVVFSENYASSSRCLDELVAILECNRTINQLVWPIFYGVYPWQVRHQGGNYGASMSKLEEIFRDSNERVKQWRAALFEVTNLNGWCYQTGRYQYEYEFIERIVESAVQALPGYDVFLSFTGEDTRYTFTGFLYNAFRREGFKIFMDDEELESGNQISQKLMGAIESSRISIVVLSENYAYSTWCLDELAKIIECMKTNNQMVWPIFYNVQKSDVCNQTKSYGEAMTEHEKRFGKDSEKVQKWSSALSEINNLEGDHVKENEYQHEFIERIVQKVIKLEGSKHIANPFLLSNDSYEEE

SEQ ID NO: 13 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 14 - DNA, 4341 bp, Glycine canescens
  source 1..4341: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaaggggaggggaggggctcactgctcacttgcggcgacgtggaggagaacccagggatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 15 - DNA, 4341 bp, Glycine canescens
  source 1..4341: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaaggggaggggaggggctcactgctcacttgcggcgacgtggaggagaacccagggccaatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatt

SEQ ID NO: 16 - DNA, 2238 bp, Glycine canescens
  source 1..2238: mol_type = other DNA
atggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacgaatatgaatttattgacaaaatcgtggaaatggccagtaaaattcctggctctgtgggcggcctgagtccttctcaggcttctagcgccgccagcagcgcctcttcttctcctggctccggaatcagcgaggccctgagagctggcgccggatctggcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtaccaacatgaatttatcgaaaggattgtacaaaaggtcattaaacttgaaggtagcaagcatatagcaaatcctttccttttatccaatgacagctacgaggaagaatga

SEQ ID NO: 17 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagcacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 18 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgagcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 19 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgcacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 20 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgcacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgcacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagcacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 21 - DNA, 4401 bp, Glycine canescens
  source 1..4401: mol_type = other DNA
atgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgcacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaacctggctctgtgggcggcctgagtccttctcaggcttctagcgccgccagcagcgcctcttcttctcctggctccggaatcagcgaggccctgagagctggcgccggatctggcatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagcacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaa

SEQ ID NO: 22 - DNA, 2002 bp, Medicago truncatula
  source 1..2002: mol_type = other DNA
ccttaggaaatatggtggctgtactcattttgatgttgatgaatggtttaactgaattccgtttattatcatttttttttttttttgtattatataaaatgaaatgtatagagttgtacaaaaaaaatgaaaccagagtttagagtttatcaacatagagccaaaagttcacgcgataggggtcaaaatcgaaatatagtgtgaacaaataataaaacataatgaaataaagcaaaatcataaaagaaaaagataaaataatgtttgtacaaaaaaaaaagataaaataatgattgtagtatctaccaaaaaaataataataataatgatggtagtctctcatctgtttgactgaattaaatttatttgacaactttaaatgagttttttctttgataaaaactttaaattatagtataaataatatattataatacctatacacatatatttaaaaagttaagaaaatgggtagaccatgtcatggtttaaaaataaaaaataatttgacaaaaatcataaaaaattaaataaccaagttaataataacaagaatatttaatcatgattttattatattgttgttgacctattgactaatacatactaaaaataaacacacaagaaggagtatggagacctaaaatcgaactcaggattcaacataaaattgttatagtcaaaccattgggttataacaatggggacaataatgaaaatataaaaatgaaccaaaattataaaacaaaaaagataaaataatgatgacaatttctcatttgtttgattggtttggttttatttgacaattttaaatgagttaaataatatttctaatacatacatattaaaaatttaagaaaattgggtagaccatagatccttccgctttatatcaatataattttctcgatgtaaatgggcattttccctcttttgcacaaaaaaaaaaatctcgatgttacattgatatttatgtgcgcctaagactttacattgatatatttatttggtgcgccttcattttttctatattatatcataaattttaaaaacaaagttacaaacacaaagcgcgccttcatttttctatattatatcataaattagaattacgttctcgaagagtcaaaccaacctactgattacgttctcggagagacaaaccaacctagtgatttactactagctaattttggttattcctcgaaagtttactttataatcgtgaaatgttattctgattttcttttgaggccaaaatcgtatccgactcacgagagattgctagtttttcttttgcagccaaattcctttacattttaacaaaagaattttttttttttggtcaagtagccacctagtggctagagcacacacaatttaattgtggagaagtggagtgtctggggttcgaaccccggcccctgcatataatatgcaatatccctaccaactgagctaagctcacgagaattaaaaaaaaagaatattaatctaactaaatatacagttaagcgcaatagaaaaacagacgaacaaaagaaagaaacgttgatatataattattggagaatgctaaccattgttagtattgaaaaatggtgaaaaatgataaatttaactttttaaaagtcaaaatattattgaaatcaatataaacatactacttttgacttctttaaccattaccagagggcaatggttaacgagacccataattattctaaggagctactttgcagataggggatgtccttttttttttttgttgaaagaaattaggggatgtccttatctctttatccctgccaattttcctatttataaataaatatcgagtttatcgctctccacctcaaacgctttctttcttgttcttggtttagggttttcttcacccattagggttagggttcaattttccccttttcaatccccaaatcaaaattcaatcaaaacctcaaatttctcaccttttctcactcaatcatcaatcaag

SEQ ID NO: 23 - DNA, 2505 bp, Medicago truncatula
  source 1..2505: mol_type = other DNA
gttgccatcgtttcatcctagggttttattgttactgttattggcaattagatatctgaaatttggtggcatgcttcagagttagaggatggagttttcaagtttttttggtttttgtttgatgatgttctaagttttttggtttagcttttgctggtctattaatggcatgatgcttcactgatgacaaataacaatgtaatgaagtgtcagttttgattttaagccatattcgtgtattgttaaagcatccaattttgtcaatcaattgatgcttcaagtatttcttttattaatgcaatgaattttcgttttaccttataatccatatgagaactttctttaattttccagttatgtgtatattgattttgtattttgttgtaaagaattaatcatagtgcactgaacctagcaaaacttgggaatttgttgatattgaagctaatttaatcaatagtcttgcttaaatggaataagaatggcgtggtactcttcaaaatatcatgattgcacatttattttccctttaccgcttcacaacatttaattcagcactactatcaattattctctatcaatgcatattcccatgcaccagcaaaagtatgtcatgtaactataaatataggtgcggtgctatcgaaaggcttgccattctcattctaaactcacttagcatcacatgtttgtatcaaaaaatggctttacgtgagtttgagagcttacttgctttgatttgcttgaaaggtcccataatctttgtttattatgtttatggtttatttttatgtgaatatgaaaaccatccagcatcaaaatctaaacgaaggaccgcatattatttaccatcatttttaccaattttcattctcaacaacaccattatttacccaagaaaataattaaacaacaacatattattttccgccaaccgttgctttttctacggacacccaaatgtaaaacacccttaattccaaagatttccaagatataaataaatacaccataaacattcttcttctaacaaagaagacctaaccattgttctaacagtaacacctaaaattcagttacctaacatgtcaaagtgaaaactgagcacaaaaactaaaatcaaaacaccagttaagaaaataattaaacaacaacacaaaattttgcattttgatgtatgaaaagctttaaaaaaagtgactatttctaaaatcacttttaaaaaaagaaacatccaaagtcatgagtgagtcaaaggggttataaacttctgcattaccctaatctttctattcttgaaattccatgagaaaataaacaacagcacataaatgggttaagctttaaaaaaagggataatttgtaaaatcactttttcagacatttttgtttatgcatgtggtaaaagtcatctaaagaacatccaatggaagaatcgtacaaaaaatcattgatctaacagaatggaccaaaacgagattaaaaaaaaacttaaatactcaaacaatcctttttttagttaagaaattaaaacaataccaattaaatatttaaggaaccaaaataacatttaagcctaaattattttgactaacataaatacataattaaaaactattttaaaatttcaatacaacgacacaccttgagagactaaaatgattattattcatattatccattaaaaaggaaaaggagtgcgtgtgaaaagcaggccacacgtatatgggccttgaactcgaattcgaattcgaatcttgtggccacctaaccacttcactaaactcacgaataattggtgaacaattctttacaacgaaactaacgaatatcctaataccagatcctaatgacatttttacccctaaactcttaccctcaacactttcaaaagaatcatcactaaattgtcattttaacacactctagaacctactttgataacgaaaaagtagaaagaaacccgtgctaggtcccacccaatagacaacgcaaaccacaattaccagcgcagtatcacgggtcatgcaccggtcacgtagcgtagataaaacccggaaccaaacttgacctacccccacaaacaagtcattattttattatatttatccttaaaaccctatttctcatttcatttcttctacaaccgtctactctagctcgttagttccgctatcactctactcttctactacttcacttcatcctctttgtttttcagcctagggtggtggtaattattcataactgtttcattatttttccttaatttctcattaattttccgttaatttttcaattagtgtaagctgatctgatttttttcctttgtttttgtttcgaattttgatggatcagccaagatcaagatcgtaatcaacggtaaatattatagttccttcgatctctgttcatttatgcgtcgatcggatttatattttttaatttagtttattttttgttaattttgtttgtaactgataggatttggaagaatcggaaacc

SEQ ID NO: 24 - DNA, 2501 bp, Medicago truncatula
  source 1..2501: mol_type = other DNA
ggatattgaacctacaactagttagcctttagcaacacatgatgtaaaagaatgttggtgtttttatttaacttttctttttcttttttcctttcttttttgtacttagattgagagcttcctatgcaaccaaggagcaaatgtggagctgttaagttttctaattctgtttttaatgctgatcctttttcttgcactagttcttttggtagatgaagaagtatatagctttgatgaatatgcacttgaactgacatgtaatgttactgcatcatgtagcagtaaatatgtttaacttggtttgtaagttgcaattgcaaaggtacatttatttcaaaagaactctagttaatttgagtccaaaatccaaatgcacccaaatatgtgatggttggttagtttgcaaattgcattcaaatgtgtccaactgtactaatattcagatatgtttggtttctcatggagaatgtcccaaatcactttatgtgatttgagaatattttcaacagaaaactaaacataccgtacaatggccaagggttaccttgttttaattttttacaccgttagtggttagctaattattttcactctaattaacaataataaataatgtgttgcttaaaatacaattctaaataatattatatatctaaaatctaaattaaaatacaacatgagtacaactaaaaatgttgcagtaaaactaaaaacaacacaatcataaatttgcaagtccttaccttcctagtgatatgtttatggtgattttaacttatggctaggtttagtaggaagtaaactaacttatatcatacgagtaaataaattaacaaattgagtttgtaatatttgataaaattaatagtcgaactaactcatagacatgaagtgacataaaattatataattgaaagatatcaataaaattaacagaaaaataacaaactataagtaataagctccaatgctactcggagtagttataaaaaaaaaaaattataagttcgtgagaataaattattacgaaacatgtctactatccactaaaaacttaaaaattggtaggtgttagtacttggatgtctttggctttggtgcctttgtctctatctctcttttaacaataattctatttcatcgaaataatttattttaatcatcactctctctcatctaaagtcaccaaagtcagtcaacacaaagtcaatgaagtgcaacaggtgcattacaaattacctcacttttatatttatttttttgtttgaaagaaacctcacttttatatcttcttaatagtgtcctaaaataaaatatctagctaatacttatctacattatttaaaatgaattatttaattttttgaaaaatagagtgttgcatgtatccacaaagaccaaaaaaggaacagatgaaacgaatgctaatcggacggtgaggatcagttgaatgattaggtttagaataaaaagagagttagggaccaccgcaccaaaaaaatctcttaactcatctttctcctcacacataacacattacccacgcgcgcagagaaagcatcaatcaacacctagtctgacgaagaacatcactttgaacccgctgccgtagccggagcatccaaaacctatcctcaacaggccggtactattcgcaagataggttacatcgtcatcaagtccaggccttgcaaggttttctccttttccctttccccttctagattttttttttttttttttttttttttttaattccttattcgatctcactctgattttgatttatgctattctatgattacgatttgtattaattaacatgcttccgtttatattggtaactggggatgtttgaatcgaatataaattggaacatgatttcgttgttttgatttgattgttttgaacggagacatagacatatggtgctattttacatctagatctaacattggttgcaataaataattgtgaaaaaaaataataattaagttccatgttgaaaagagattaacttaatttacagcttatatagcataagcacttatgtatgaactttttataaaataaagtttattagtttttttctatactctttttaagagagcttatgaaaataagcagaaaataacttatagctatctcattaaccgttttcacaagttctttcaaacaatttcgcaaatgctcctgtatatagttaagctcaaataagtcaatccaaacaggccctaggttttgaaacaatttttaaagacggacacctagtcgacatcgttaggatgagttaggtttaatttaaaaacataatacaactggaattatcaattgtgctgaaacctatattccttgactacagggacattggaattttagttttgctgtaatatttctttgtttttatgattttcattttcaattgggctaacttgtcttttgggtgccatatttgtttctcaatacatcttggtaggttgttgaggtttctacttgaaacc

SEQ ID NO: 25 - DNA, 636 bp, Linum usitatissimum
  source 1..636: mol_type = other DNA
ttttgtatcacacgcgttactcatcataaataaccaggagtccaggatctcataacaactgcagtattgacttgtacacactacagtcgaaaaaagttacaatttgaagtttcaacacatccggacgatgatcggataaaaggatgaacaactttctttgaactaccaactaagaattagaaagacacagcagtaagaatcaagatagatgaaggacacagaggaaacaagaaattgatcacagatggcttccttttacatctttacatagatataaacaaagatataattgattcaaagcgcacactgagattggattgggggattgtctgctatcttgatcctattcactctgtgacgcggctaccactcagacaacctcacagctggacaaatcgacaaactcttgaataataattggcctgtagattcagtggaaccatcactacctctaaagctgcacggctgacaccgcaacacaaataacttgccacattttctctctatcaaatcaaaatcgacgtctctttctcctcctcatcactgagtttgttcatacttgcccaaccaaatagcttggtacttttagccaatcagggtgccatagagatcatttccactccaatcaagccctta

SEQ ID NO: 26 - DNA, 1056 bp, Glycine canescens
  source 1..1056: mol_type = other DNA
cacctctaaccgcatggaagagaaggcccactccctcggcatccccctctctatcctcgaccacaatccccacctcgatctcgccatcgacagcatcgacgaggtcgatgaggcgatctgtaatccataagattttacgaatttgcaatccataagattcttacggatttttcaatttgtatgaaccttacggattgtcaatccgtatgaatcttatggaagaatattttttatttttcgttgtctttggtgggtgtatgaagaaatttgtctagtgcatgaaaaaaaatgcttatggatttctgttattggaggttcattttgagctatgactcttgcactttatttttattttgaatagaatatacaattaataatttctatatgttcaaagtaattggatccacatttagacaattatattttttttctctctatttggcgaaaatattgtgtaagtttgtaattaattagaatttaggttgaaactaattattttatttgatgtggttacttattattatatattttaatgatttcatcgtattattaattgcttgtgatacaaagttaattaatagttgtgatgtaaagttaattatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgatatttttttcttgtgacaattataattagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaa

SEQ ID NO: 27 - DNA, 1649 bp, Glycine canescens
  source 1..1649: mol_type = other DNA
caggatagctgacacacaaagtggcaaagaaatgctgctcgatccaggtattccatacttttcaactgcaaactgaagctgaaggtgaaattgttgcttgtacatgtgtatacgtgtcatttcgttccttgttttcatagttttgtgaaaataataaaaaatgttttaaaatgttaaattatagctacacacttgtttaccaattagaaaacccttttgtattttctgcaatcatcattgaatttgagaacctggctagatctcattctcatcagatcactatataccattttggtcttcagagcattgaagtagctaacttacatgtatgttcttacttcataagcatggactatttgtacattttgccacatcatctttatgtcttgatgcagaacaaaaagctgaatactatagaaggatgcgagatgatttgaagaaggctgacaaggaagacaagcttatagagcgtcagcggcttagagaaaaaagaatcaagcagaagttgaaatggaaggctgggaatgcggaagaggacgaccaagatgatatatctgggtcagaagtagatgagactgtagatagacggcataaaaagagtaaaatatactttgatagtgacagtgatgagggtgaaagaaatgaagtttcagggaatgcaggtataactaccggtgcggttacgttggaggagcaagaagctctggctttgaaattgttaaattccatgcactcataggatatgcgtaattcagctggacattttgtgacagcaaaaactggtgaaaagagagcaattcctgaaacattttaggagttctgttaacaaaaaattttgatctcccaatttttttctgtcatctctttcaagaaacgtatacatagaatcaatttactgcttttatttattttaactaaaaaaaatgggactaaaggacatccttgtgtgtaccaaacacggtattaaatattatttggagatattcataatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgttttggaattaagacaacaataaaaggatatggacataatttcaattatttatgagaaacaagaactatcaacgggagcttttttggtttgaaaaaaaaaaacttttggatttctgtttattacaactattataaataagaacttaattattacaactattaaattaaattaagggtttagattaaatacttaattacaaatcttgtcattgataatattttttacaaattttgtgtattttcttttaaatggagatgatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaattt

SEQ ID NO: 28 - DNA, 1089 bp, Glycine canescens
  source 1..1089: mol_type = other DNA
gacacaccaaacatatccttgcatgataacttttgattttggggtgtatcccgccgggaagatattcaagattgtgcagtgtaacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcatagaagaactttaaaataaaagagtattgacgaaaataaagtgcgcaaaaaaaaagatacagaaaattaaaggagtaaaaagtttggagaagaaattgagattgaaaaaagatgaagatttaaagggaagtataacaaatgtaaaagaaaacgataaaatgaataagataaagtgctgaaattaaaatgtttgagagtattgtaataattcattgatccacaagtgtcacgatgagacaagaggatatgtgagtgtttacaagtttcaactcttattacaaatttttaaatcttcctcatcgataactttaatattcaatgtcaatcattaatgtcctttgtctttggtattcgtttttaatatattcttcaagaggtatttgtagatttttgttcctctaatggttaaactcaaatgctttatgaaattattgcttaaaaacatgttattttcccgtgtcaggtgacctctaaaaaagatcaagctcaatactgggctgattcttcaaagccatataaatttatttcagtgccggagattgcataagcctttaaaaattccagatttggaaagtcggtagaatccatgtgtactgctccctttaataaattaaagagtcatccttcagctttgcccagaactagatttgttgtgccaaaatgggagctctttaaggcgtgtttctcgcgcgagagaccttgatcaacaggcataagtt

SEQ ID NO: 29 - DNA, 950 bp, Glycine canescens
  source 1..950: mol_type = other DNA
ttagataaaaaaaacaagtggtactattttgttttgtcatattggcattaggtaaggcaccgtgaaacaaaatttaaacacatcgccttctactcacaaaaataaaaacagagtatacaaaatctatggtaaggttaaataagttttcatcattcatctatttcagaattccatttttaattctctcaatcttctttatccaattttagtctctattttttgaagtaagtttagtctcccacgttaaataaaaactatcaaatacttatatgtccaacaaaattacatgtcacaacacacatgatgtaatcatgtgacaccgatgtgggtgatactaacatgtttagtcacatggtatcatgttttctatgtctttagaaatttttgaaatttaaattttagttcctaaaaaattatcatctttcaattttagtcaatataaatttgttttatttaatttaagtcctttaaaaaaatttagtttttaatccttaattaatttttttaatcttagtatagttatgctttagtgatttgttggtcatgagttcgaatatgtaaaaagtttatttacatatgcaagagtaaggttgtgtacaatatctttttccttatttttgtataacaaggagcctatgaacaatgtgatacataatttttttttctagtttaggtatttctagaatatttaatctcttcattattcaccttggtatcacaaatatcatcacaagtatgttcaaaccattatttggttaggtcctacattttgtcccaattttggattggatttttcttcctacaccagctagtgttcccatacacccaacaataaaacgaaaaactgaaaatacccttccataaggttcatacatattggccaatccgtatcctttgttgtcgtcaagctcagtgccctcccagcctttggccaactcaccgacatcgtc

SEQ ID NO: 30 - DNA, 1007 bp, Medicago truncatula
  source 1..1007: mol_type = other DNA
gagctctctttatcatcatcaacatcatcgtgtgactttaatcgacccttcattcatggcttttactatatgttctattttgtaatctttttcttttcaagttgtctttttttagagggtttttgaataatttgtcaaatgggtatgcttaggtacttttatgtctttttaatatcaaaatgatgcattttggatatgaatgttgttatgatgctgtctatgaagtggtgctgaaaagggttgttgatgggaattggattcagttgtgctgttattgatttggcccttcttaaaaccttgaatgtaatatattgttccctggatggaggttaatatgatggtgctactagttactactatatcttgttctgtgctctgatttgttgttctgtgatcaaataacttgtttaatccttggatcataagctggcttgagttgttggtttagtttattttttggtatgtttggttttgcggcaatttttccgcgatagctcaaaacctatgctagtttttcacgtctatatagacacgcaacaacacaactgaatgtgagttcaggctcaaacaccaaacagaacatatgctccgtgattgtcagtattaaatctggggcaatgaggttttgtaaaatttctatccggtggcggttaagttcagtattttagtttttgaaagtatatattgaaatctggattgtccgaatgtaacagtcaccagtgtagctcacagtgtaaccttagatgattgtgacccacaaggccacaatccaaatttgttggttttatgatggatgtgtaataatgcaacttttgccctgcatttggtttttatactaaattagtttagaaggataaccaaaagctactaggcagtgatacaatctagacatacggatgttattgatgatatattttacattctggattttatataatctgaatctctattctggagaaatttagccataaagcattttgagtgatcctctgttccttttccttaga

SEQ ID NO: 31 - DNA, 1000 bp, Medicago truncatula
  source 1..1000: mol_type = other DNA
gctgtttcattactgtggtagtgctataaattggcctactttttgtttcttacagtctattagcctagcagccgttaggcctcttttttaaatgtaataagttgaattcctgaactcgtgaagcttgccccttattgaaccgtagtgtttttggttgcttaataccattaagcaatgagttgttttggatttatatgcatttaccgaattttatttggatgatagtaagcgttttttccatgttcacaaatatgccatgaaaaaagcagtaaaatttcaataaaccaacatcatgataaaacaaaagtatgcatcgtatgaaaaaaagtgggtgatgaaatgataagtctaattttggtcgaatttggtcgttgttatttttgcatgaacatctcttttgtacaacctaacaacttatttgctatttttaatatttttgtactgcttttgatttgcttgctcttcacaatattatagtgttgcattagttgttcaattttggttgtacaaataatacaacttttaagcagttatcgtgaatgttagaaaaatcatgtgacttatctctgttattgggtgattgcatttgtttcgtgacttgtctttaatcggttacttattttcaataataaataacaatatagcattattttgtgtcactttcttacttttgattattgaaaagccgtgctcctttgcctgtctcttgagccatgtgattcgtcgtgaattgtgaggacgagatcatatttgagaaagaaaatgtgagctagacgtttctatagctttattacgtagcccttgcaattcaattcagcagggaacatatcaaaatgatttgtattgaaaaaattgttctacctacaaaaatctactttattatttgtactgaacgagaaagtgccacaatacttaattagataagtagacattggagctaatctcaaaattcttgcctgctttttctatcatatatctccatatcactaatgaattcatt

SEQ ID NO: 32 - DNA, 409 bp, Arabidopsis thaliana
  source 1..409: mol_type = other DNA
gtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcag

SEQ ID NO: 33 - DNA, 114 bp, synthetic construct
  source 1..114: mol_type = other DNA
cctggctctgtgggcggcctgagtccttctcaggcttctagcgccgccagcagcgcctcttcttctcctggctccggaatcagcgaggccctgagagctggcgccggatctggc

SEQ ID NO: 34 - DNA, 57 bp, synthetic construct
  source 1..57: mol_type = other DNA
ggggaggggaggggctcactgctcacttgcggcgacgtggaggagaacccagggcca

SEQ ID NO: 35 - DNA, 54 bp, synthetic construct
  source 1..54: mol_type = other DNA
ggggaggggaggggctcactgctcacttgcggcgacgtggaggagaacccaggg

SEQ ID NO: 36 - protein, 38 aa, synthetic construct
PGSVGGLSPSQASSAASSASSSPGSGISEALRAGAGSG

SEQ ID NO: 37 - protein, 19 aa, synthetic construct
GEGRGSLLTCGDVEENPGP

SEQ ID NO: 38 - protein, 18 aa, synthetic construct
GEGRGSLLTCGDVEENPG

SEQ ID NO: 39 - DNA, 5209 bp, Glycine clandestina
  source 1..5209: mol_type = other DNA
cacctctaaccgcatggaagagaaggcccactccctcggcatccccctctctatcctcgaccacaatccccacctcaatctcgccatcgacagcatcgacgaggtcggtgaggcgatatgtaattcataagattttacgaatttgcaatccataagattcttacggatttttcaatttgtatgaaccttacggattgccaatccgtatgaatcttatggaagaatattttttatttttcgttgtctttggtgggtgtatgaagaaatttgtctagtgcatgaagaaaaatgcttgtggatttctgttattggaggttcattttgagctatgactcttgcactttatttttattttgaatagaatatacaattaataatttctatatattcaaagtaattggatccacatttagacaataatcttttttttctctctatttggcaaaaatattgtgtaagtttgtaattaattagaatttaggttgaaactaattattttatttcatgtggttagttattattatatattttaatgatttcatcgtattattaattgcttgtgatacaaagttaattaatagttgtgatgtaaagttaattatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgagatttttttcttgtgacaattataatgagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaaatgatagtgtcagcacatcatataatatcttatgttatctgcttaatagtttcattgcgatggcggatgaagggggaattgcgaagtggcgctacgatgtatttctgtgttttagaggggaagacacgcgccacaccttcactggtaatctctatgctgctttgcggaaggcgagattgaggaccttcttcgatggtgggttgaagggtggcgacctaatcgcagcagctattcttgaagcattagaaggatccaggatttcaatcgttgttctctccgaaaacttcgcgtcttcctcgtggtgccttaaagaacttgtcaagatccttgagttaagggcaacaaagaaccaactagttattccaatcttttaccacgtggacccctcccatgtaaggaaacagactggtggtttcggagagtgcctgattaaacatgaaagtcgcttgggaaaggactccgagaaaatacacaagtggaagtcagctttgtatgaagttgccaacttgtccggatggtgtttcagaagcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatcaaatgaatttttagcctaggatggtgtttatgtatcattgttgaaattaaaatttcatatactaagttaagtaactcatgccgattattattgctgaagtcatgtaaacttgcattttaatcctcagagaccaaaaccacttgtgatatttgcacgaagcttttttccttgatttataccttattgaatgttctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggaaattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaattaaggctttcgatcattgtattttctgaaaaatatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatcataataatcacttaatatgtgcatcgttcatgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgctgatttactttgataattttagaaagaaaatgaaaattgtgtatgaatgaactaaaaggccttgtatttttgtaggtacgaatatgaatttattgacaaaatcgtggaaatggccagtaaaatttaaaatggcttaaatcgatcaagccctcgctcaagtgtcaactagaaggcagaactgatcaattcaacagattacagatggttctatgagcagtcattagctcacaaattgatagtaatagtcgtaatgcttacaatcagcactgaggaattgatttggaagtctgaattttacttagaacattagcaatttctagaaaggtgaatatatatgtcctgagggccatggccagaggcctgtcatcttgaaattgtgacacacactctcgtttaagtcatatttactttgatttcccaaatgtcatgcatatacaaataattgtatgagttagattaaagaagctttagcattaaacagattgaagacaattacccagttaagatgctgaatgatgtttttgttgagaatcaaccgacatagttattcatgatgtatataatcgaatttcgtaagacacaccaaacatatccttgcatgataacttttgattttggggtgtatcccgccgggaagatattcaagattgtgcagtgtcacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcacagaacaactttaaaataaaagagtattgacgaaaataaagtgggcaaaaaaaaaaagatacagaaaattaaaggagtaaaaagtttggagaagaaattgagattgaaaaaagatgaagatttaaagggaagtataacaaatgtaaaagaaagcgataaaatgaataagataaagtgctgaaattaaaatgtttgagagtattgtaataattcattgatccacaagtgtcacgatgagacaagaggatatgtgagtgtttacaagtttcaactcttattacaaatttttaaatctttctcatcgataactttaatattcaatgtcaatcattgatgtcttttgtctttggtattcgtttttaatatattcttcaagaggtatttgtagatttttgttcctctaatggttaaactcaaatgctttatgaaattattgcttaaaaacatgttattttccccgtgtcaggtgacctctaaaaaagatcaagctcaatactgggctgattcttcaaagccatataaatttatttcactgccggagattgcataagcctttaaaaattccagatttggaaagtcggtagaatccatgtgtactgctccctttgataaattaaagagtcatccttcagctttccccagaactagatttgctgtgccaaaatgggagctctttaaggcgtgtttctcgcacgagaccttgatcaacaggcataagtt

SEQ ID NO: 40 - DNA, 4618 bp, Glycine clandestina
  source 1..4618: mol_type = other DNA
caggatagctgacacacaaagtggcaaagaaatgctgctcgatccaggtattccatacttttcaactgcaaactgaagctgaaggtgaaattgttgcttgtacatgtgtatacgtgtcatttctttccttgttttcatagttttgtgaaaataataaaaaatgttttaaaatgttaaattatagctacacacttgtttaccaattagaaaacccttttgtattttctgcaatcatcattgaatttgagaacctggctagatctcattctcatcagatcactatataccattttggtcttcagagcattgaagtagctaacttacatgtatgttcttacttcataagcatgggctatttgtacattttgccacatcatctttatgtcttgatgcagaacaaaaagctgaatactatagaaggatgcgagatgatttgaagaaggctgacaaggaagacaagcttatagagcgtcagcggcttagagaaaaaagaatcaagcagaagttgaaatggaaggctgggaatgcggaagaggacgaccaagatgatatatctgggtcagaagtagatgagactgttgatagacggcataaaaagagtaaaatatactttgatagtgacagtgacgagggtgaaagaaatgaagtttcagggaatgctggtataactaccggtgcggttacgttggaggagcaagaagctctggctttgaaattgttaaattccatgcactcataggatatgcgtaattcagctggacattttgtgacagcaaaaactagtgaaaagagagcaattcctgaaacattttaggagttctgttaacaaaaaattttgatctcccaatttttttctgtcatctctttcaagaaacgtatacataagaatcaatttactgcttttatttattttaactaaaaaaaatgggactaaaggacatccttgtgtgtaccaaacacgatattaaatattattaggagatattcataatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgtttttggaattaagacaacgataaaaggatatggacagaatttcaattatttatgagaaacaagaactatcaacgggagcttttttggtttgaaaaaaaaaacttttggatttctgtttattacaactattataaataagaacttaattattacaactattaaattaaattaagggtttagattaaatacttaattacaaatcttgtcattgataattttttttacaaattttgtgtattttttttttaaatggagattatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaatttatgaaagagagaaagagaagtagggaggcattggccattaggaggagtcaacgagacaagcaagccaccttctgctacttctcatccatcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcagggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaactaattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctacactttcacaggttttctctataatgcctttcgccgagagggattcaaagtcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagcgtacgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttaatgacataaagtctgattaggagaaataaagtgaacacagcggatgaattcttaaaagaccttgcacttttgtaggtaccaacatgaatttatcgaaaggattgtacaaaaggtcattaaacttgaaggtagcaagcatatagcaaatcatttccttttatccaatgacagctacgagaaagaatgagtgaaagtcggtcaagtatatgctgtggagctgtgtgactaactgattgtaacatcaataactgcccttgaattcagttgtgcctatcaatgttgtttttacatccagttcaattcctaaggttttttgtatttcttctaaatcttgttcaattctcgaggtctgttgtgttttttctacatcttgtttcaatgttttgtgttgattagataaaaaaaacaagtggtactattttgttttgtcatattggcattaggtaaggcaccgtgaaacaaaatttaaacacatcgccttctactcacaaaaataaaaacagagtatacacaatctatggtaaggttaaataagttttcatcattcatctatttcagaattccatttttaattctctcaattttctttatccaaagttagtctctattttttaagtaagtttagtctcccacattaaataaaaactatcaaatacttatatgtccaacaaaattacatgtcacaacacacatgatgtaatcatgtgacacccatgtgggtgatactaacatgtttagtcacatggtatcatgtgtctttagaaatttttgaaatttaaattttagttcctaaaaaattatcatctttcaattttagtcaataaaaatttgttttatttaatttaagtcctttaaaaaaaattaaattttagtttttaatccttaattaattttttaaatcttagtatagttatgctttagtgatttgttggtcatgagtttgaatatgtaaaaagtttatttgcatatacaagagtaaggttgtgtacaatatctctttccttatctttgtataacaaggagcctatgaacaatgtgatacataattttttttttctagtttaggtatttctagaatatttaatctcttcattattcaccttggtatcacaaatatcatcacaagtatgttcaaaccatttggttaagtcctacattttgtcccaattttggattggatttttcttcctacactagctagtgttcccatacacccagcaataaaatgaaaaactgaaaatacccttccgtaaggttcatacatattggccaatccgtatcctttgttgtcgtcaagctcagtgccctcccagcctttggccaactcaccgacatcgt

SEQ ID NO: 41 - DNA, 5223 bp, Glycine canescens
  source 1..5223: mol_type = other DNA
cacctctaaccgcatggaagagaaggcccactccctcggcatccccctctctatcctcgaccacaatccccacctcgatctcgccatcgacagcatcgacgaggtcgatgaggcgatctgtaatccataagattttacgaatttgcaatccataagattcttacggatttttcaatttgtatgaaccttacggattgtcaatccgtatgaatcttatggaagaatattttttatttttcgttgtctttggtgggtgtatgaagaaatttgtctagtgcatgaaaaaaaatgcttatggatttctgttattggaggttcattttgagctatgactcttgcactttatttttattttgaatagaatatacaattaataatttctatatgttcaaagtaattggatccacatttagacaattatattttttttctctctatttggcgaaaatattgtgtaagtttataattaattagaatttaggttgaaactaattattttatttgatgtggttacttattattatatattttaatgatttcatcgtattattaattgcttgtgatacaaagttaattaatagttgtgatgtaaagttaattatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgatatttttttcttgtgacaattataattagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaaatgatagtgtcagcacatcatataatatcttatgttatctgcttaatagtttcattgcgatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggaaagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaataaaggtgtaaatatgtttttaatagtttagtgtttttttttttaatttttgtaatttttttatttttagtctttataaatatatatatatatatatatatatattttacttttagttttaaaattactttaaatagtactctagatagtaaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaaaatggcttaaatcgatcaagccctcgctcaagtgtcaactagaaggcagaactgatcaattcaacagattacagatggttctatgagcagtcattagctcacaaattgatagtaatagtcgtaatgcttacaatcagcactgaggaattgatttggaagtctgaattttacttagaacattagcaatttctagaaaggtgaatatatatgtcctgagggccatggccagaggcctgtcatcttgaaattgtgacacacactctcgtttaagtcatatttactttgatttcccacatgtcatgcatatacaaataattgtatgagttagattaaagaagctttagcattaaacagattgaagacaattacccagttaagatgctgaatgatgtttttgttgagaatcaaccgacatagttattcgtgatgtatataatcgaatttcgtaagacacaccaaacatatccttgcatgataacttttgattttggggtgtatcccgccgggaagatattcaagattgtgcagtgtaacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcatagaagaactttaaaataaaagagtattgacgaaaataaagtgcgcaaaaaaaaagatacaaaaaattaaaggagtaaaaagtttggagaagaaattgagattgaaaaaagatgaagatttaaagggaagtataacaaatgtaaaagaaaacgataaaatgaataagataaagtgctgaaattaaaatgtttgagagtattgtaataattcattgatccacaagtgtcacgatgagacaagaggatatgtgagtgtttacaagtttcaactcttattacaaatttttaaatcttcctcatcgataactttaatattcaatgtcaatcattaatgtcctttgtctttggtattcgtttttaatatattcttcaagaggtatttgtagatttttgttcctctaatggttaaactcaaatgctttatgaaattattgcttaaaaacatgttattttcccgtgtcaggtgacctctaaaaaagatcaagctcaatactgggctgattcttcaaagccatataaatttatttcagtgccggagattgcataagcctttaaaaattccagatttggaaagtcggtagaatccatgtgtactgctccctttaataaattaaagagtcatccttcagctttgcccagaactagatttgttgtgccaaaatgggagctctttaaggcgtgtttctcgcgcgagagaccttgatcaacaggcataagtt

SEQ ID NO: 42 - DNA, 4604 bp, Glycine canescens
  source 1..4604: mol_type = other DNA
caggatagctgacacacaaagtggcaaagaaatgctgctcgatccaggtattccatacttttcaactgcaaactgaagctgaaggtgaaattgttgcttgtacatgtgtatacgtgtcatttcgttccttgttttcatagttttgtgaaaataataaaaaatgttttaaaatgttaaattatagctacacacttgtttaccaattggaaaacccttttgtattttctgcaatcatcattgaatttgagaacctggctagatctcattctcatcagatcactatataccattttggtcttcagagcattgaagtagctaacttacatgtatgttcttacttcataagcatggactatttgtacattttgccacatcatctttatgtcttgatgcagaacaaaaagctgaatactatagaaggatgcgagatgatttgaagaaggctgacaaggaagacaagcttatagagcgtcagcggcttagagaaaaaagaatcaagcagaagttgaaatggaaggctgggaatgcggaagaggacgaccaagatgatatatctgggtcagaagtagatgagactgtagatagacggcataaaaagagtaaaatatactttgatagtgacagtgatgagggtgaaagaaatgaagtttcagggaatgcaggtataactaccggtgcggttacgttggaggagcaagaagctctggctttgaaattgttaaattccatgcactcataggatatgcgtaattcagctggacattttgtgacagcaaaaactggtgaaaagagagcaattcctgaaacattttaggagttctgttaacaaaaaattttgatctcccaattttttctgtcatctctttcaagaaacgtatacatagaatcaatttactgcttttatttattttaactaaaaaaaatgggactaaaggacatccttgtgtgtaccaaacacgatattaaatattattaggagatattcataatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgttttggaattaagacaacaataaaaggatatggacataatttcaattatttatgagaaacaagaactatcaacgggagcttttttggtttgaaaaaaaaaaaacttttggatttctgtttattacaactattataaataagaacttaattattacaactattaaattaaattaagggtttagattaaatacttaattacaaatcttgtcattgataatattttttacaaattttgtgtattttcttttaaatggagatgatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaatttatgaaagagagaaagagaagtagggaggcattggccattaggaggagtcaacgagacaagcaagccaccttctgctacttctcatccatcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctacattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaatgagtgaaagtcggtcaagtatatgctgtggagctgtgtgactaattgattgtaacatcaataactgcccttgaattcagttgtgcctatcaatgttgtttttacatccagttcaattcctaaggtttattgtattccttctaaatcttgttcaattctcgaggtctgttgtgttttttctacatcttgtttcaatgttttgtgttgattagataaaaaaaacaagtggtactattttgttttgtcatattggcattaggtaaggcaccgtgaaacaaaatttaaacacatcgccttctactcacaaaaataaaaacagagtatacaaaatctatggtaaggttaaataagttttcatcattcatctatttcagaattccatttttaattctctcaatcttctttatccaattttagtctctattttttgaagtaagtttagtctcccacgttaaataaaaactatcaaatacttatatgtccaacaaaattacatgtcacaacacacatgatgtaatcatgtgacaccgatgtgggtgatactaacatgtttagtcacatggtatcatgttttctatgtctttagaaatttttgaaatttaaattttagttcctaaaaaattatcatctttcaattttagtcaatataaatttgttttatttaatttaagtcctttaaaaaaaatttagtttttaatccttaattaatttttttaatcttagtataattatgctttagtgatttgttggtcatgagttcgaatatgtaaaaagtttatttacatatgcaagagtaaggttgtgtacaatatctttttccttatttttgtataacaaggagcctatgaacaatgtgatacataatttttttttctagtttaggtatttctagaatatttaatctcttcattattcaccttggtatcacaaatatcatcacaagtatgttcaaaccattatttggttaggtcctacattttgtcccaattttggattggatttttcttcctacaccagctagtgttcccatacacccaacaataaaatgaaaaactgaaaatacccttccgtaaggttcatacatattggccaatccgtatcctttgttgtcgtcaagctcagtgccctcccagccttt

SEQ ID NO: 43 - DNA, 5221 bp, Glycine canescens
  source 1..5221: mol_type = other DNA
cacctctaaccgcatggaagagaaggcccactccctcggcatccccctctctatcctcgaccacaatccccacctcgatctcgccatcgacagcatcgacgaggtcgatgaggcgatctgtaatccataagattttacgaatttgcaatccataagattcttacggatttttcaatttgtatgaaccttacggattgtcaatccgtatgaatcttatggaagaatattttttatttttcgttgtctttggtgggtgtatgaagaaatttgtctagtgcatgaaaaaaaatgcttatggatttctgttattggaggttcattttgagctatgactcttgcactttatttttattttgaatagaatatacaattaataatttctatatgttcaaagtaattggatccacatttagacaattatattttttttctctctatttggcgaaaatattgtgtaagtttgtaattaattagaatttaggttgaaactaattattttatttgatgtggttacttattattatatattttaatgatttcatcgtattattaattgcttgtgatacaaagttaattaatagttgtgatgtaaagttacttatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgatatttttttcttgtgacaattataattagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaaatgatagtgtcagcacatcatataatatcttatgttatctgcttaatagtttcattgcgatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcgaaaaggacctgattggacatgaaaggcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatatatatattttacttttagttttaaaattactttaaatagtactctagatagtaaaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaaaatggcttaaatcgatcaagccctcgctcaagtgtcaactagaaggcagaactgatcaattcaacagattacagatggttctatgagcagtcattagctcacaaattgatagtaatagtcgtaatgcttacaatcagcactgaggaattgatttggaagtctgaattttacttagaacattagcaatttctagaaaggtgaatatatatgtcctgagggccatggccagaggcctgtcatcttgaaattgtgacacacactctcgtttaagtcatatttactttgatttcccacatgtcatgcatatacaaataattgtatgagttagattaaagaagctttagcattaaacagattgaagacaattacccagttaagatgctgaatgatgtttttgttgagaatcaaccgacatagttattcgtgatgtatataatcgaatttcgtaagacacaccaaacatatccttgcatgataacttttgattttggggtgtatcccgccgggaagatattcaagattgtgcagtgtaacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcatagaagaactttaaaataaaagagtattgacgaaaataaagtgcgcaaaaaaaaagatacaaaaaattaaaggagtaaaaagtttggagaagaaattgagattgaaaaaagatgaagatttaaagggaagtataacaaatgtaaaagaaaacgataaaatgaataagataaagtgctgaaattaaaatgtttgagagtattgtaataattcattgatccacaagtgtcacgatgagacaagaggatatgtgagtgtttacaagtttcaactcttattacaaatttttaaatcttcctcatcgataactttaatattcaatgtcaatcattaatgtcctttgtctttggtattcgtttttaatatattcttcaagaggtatttgtagatttttgttcctctaatggttaaactcaaatgctttatgaaattattgcttaaaaacatgttattttcccgtgtcaggtgacctctaaaaaagatcaagctcaatactgggctgattcttcaaagccatataaatttatttcagtgccggagattgcataagcctttaaaaattccagatttggaaagtcggtagaatccatgtgtactgctccctttgataaattaaagagtcatccttcagctttgcccagaactagatttgttgtgccaaaatgggagctctttaaggcgtgtttctcgcgcgagagaccttgatcaacaggcataagtt

SEQ ID NO: 44 - DNA, 4624 bp, Glycine canescens
  source 1..4624: mol_type = other DNA
caggatagctgacacacaaagtggcaaagaaatgctgctcgatccaggtattccatacttttcaactgcaaactgaagctgaaggtgaaattgttgcttgtacatgtgtatacgtgtcatttcgttccttgttttcatagttttgtgaaaataataaaaaatgttttaaaatgttaaattatagctacacacttgtttaccaattagaaaacccttttgtattttctgcaatcatcattgaatttgagaacctggctagatctcattctcatcagatcactatataccattttggtcttcagagcattgaagtagctaacttacatgtatgttcttacttcataagcatggactatttgtacattttgccacatcatctttatgtcttgatgcagaacaaaaagctgaatactatagaaggatgcgagatgatttgaagaaggctgacaaggaagacaagcttatagagcgtcagcggcttagagaaaaaagaatcaagcagaagttgaaatggaaggctgggaatgcggaagaggacgaccaagatgatatatctgggtcagaagtagatgagactgtagatagacggcataaaaagagtaaaatatactttgatagtgacagtgatgagggtgaaagaaatgaagtttcagggaatgcaggtataactaccggtgcggttacgttggaggagcaagaagctctggctttgaaattgttaaattccatgcactcataggatatgcgtaattcagctggacattttgtgacagcaaaaactggtgaaaagagagcaattcctgaaacattttaggagttctgttaacaaaaaattttgatctcccaatttttttctgtcatctctttcaagaaacgtatacatagaatcaatttactgcttttatttattttaactaaaaaaaatgggactaaaggacatccttgtgtgtaccaaacacgatattaaatattattaggagatattcataatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgttttggaattaagacaacaataaaaggatatggacataatttcaattatttatgagaaacaagaactatcaacgggagcttttttggtttgaaaaaaaaaaacttttggatttctgtttattacaactattataaataagaacttaattattacaactattaaattaaattaagggtttagattaaatacttaattacaaatcttgtcattgataatattttttacaaattttgtgtattttcttttaaatggagatgatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaatttatgaaagagagaaagagaagtagggaggcattggccattaggaggagtcaacgagacaagcaagccaccttctgctacttctcatccatcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaatgagtgaaagtcggtcaagtatatgctgtggagctgtgtgactaattgattgtaacatcaataactgcccttgaattcagttgtgcctatcaatgttgtttttacatccagttcaattcctaaggtttattgtattccttctaaatcttgttcaattctcgaggtctgttgtgttttttctacatcttgtttcaatgttttgtgttgattagataaaaaaaacaagtggtactattttgttttgtcatattggcattaggtaaggcaccgtgaaacaaaatttaaacacatcgccttctactcacaaaaataaaaacagagtatacaaaatctatggtaaggttaaataagttttcatcattcatctatttcagaattccatttttaattctctcaatcttctttatccaattttagtctctattttttgaagtaagtttagtctcccacgttaaataaaaactatcaaatacttatatgtccaacaaaattacatgtcacaacacacatgatgtaatcatgtgacaccgatgtgggtgatactaacatgtttagtcacatggtatcatgttttctaagtctttagaaatttttgaaatttaaattttagttcctaaaaaattatcatctttcaattttagtcaatataaatttgttttatttaatttaagtcctttaaaaaaaatttagtttttaatccttaattaatttttttaatcttagtatagttatgctttagtgatttgttggtcatgagttcgaatatgtaaaaagtttatttacatatgcaagagtaaggttgtgtacaatatctttttccttatttttgtataacaaggagcctatgaacaatgtgatacataatttttttttctagtttaggtatttctagaatatttaatctcttcattattcaccttggtatcacaaatatcatcacaagtatgttcaaaccattatttggttaggtcctacattttgtcccaattttggattggatttttcttcctacaccagctagtgttcccatacacccaacaataaaatgaaaaactgaaaatacccttccgtaaggttcatacatattggccaatccgtatcctttgttgtcgtcaagctcagtgccctcccagcctttggccaactcaccgacatcgt

SEQ ID NO: 45 - DNA, 1026 bp, Cajanus cajun
  source 1..1026: mol_type = other DNA
atgaagatgggggatgaaaaagcgaagtggtgctacgatgtttttctggctttcagagggcaagacacgcgctacaacttcaccggtaatctctacgatgctttgcggcgcgctagaatcagtactttcatggatgacagtgccttgaagggtggtgaccaaatctcagcctctcttctcagagcgcttgaagcgtccaggatttcaatcgttgttctctccgaaaactatgcgttttccacgtggtgccttgaagaacttgtgaacatcgtccggtgtagaaggacctatgcccaagtcgttttgccaatcttttacgacgtggatccctccgatgtaaggaagcagacaggtaggtttgatgaagccatggtccgacatatacgtcgcttcggagataaatcggagaagataaaagagtggaggtcagctttgactgaaatcgccaacttgcgtggatgggatttccgatatggggaccgatatgaatataaactcatccaagacattgttagatgggtcaccaacactgtgtcccgctatagcatttttctgagtttttgtggaaaagatacccgctactcttttacaggttttctctacaatgctttgtctcggagtggattccaaaccttcatgaacgatggggaccacatttcacaatctaatattgaagttgaagcgattgaaaattcaaggctttccatcattgtattttctgaaaactatgcacgttcctcctcgtgtcttgatgagcttctgacgatcctcgagtgtatgaagctcaaaaaccaactagtttggcctgtcttttacaaagtggaaccatcggatttaaggcatcaaagaaatagttggggtgaagccatgattgaacatgaaaacatgttgggcaacgactctcagaaggtgcagaaatggaggttagctttacttgaagtcgccaacttgaaagggtggcaccacaaaaccgggtacgaatatgaattaattgaaaaaattgtggaaatggccactaaaatttaa

SEQ ID NO: 46 - protein, 341 aa, Cajanus cajun
MKMGDEKAKWCYDVFLAFRGQDTRYNFTGNLYDALRRARISTFMDDSALKGGDQISASLLRALEASRISIVVLSENYAFSTWCLEELVNIVRCRRTYAQVVLPIFYDVDPSDVRKQTGRFDEAMVRHIRRFGDKSEKIKEWRSALTEIANLRGWDFRYGDRYEYKLIQDIVRWVTNTVSRYSIFLSFCGKDTRYSFTGFLYNALSRSGFQTFMNDGDHISQSNIEVEAIENSRLSIIVFSENYARSSSCLDELLTILECMKLKNQLVWPVFYKVEPSDLRHQRNSWGEAMIEHENMLGNDSQKVQKWRLALLEVANLKGWHHKTGYEYELIEKIVEMATKI

SEQ ID NO: 47 - DNA, 1035 bp, Cajanus cajun
  source 1..1035: mol_type = other DNA
atgttgatggcgaatggtgccaccgaggaacaaaggagcagcaacaagtacgatgtttttctcagttttgcaggtatagacaaccgctacaccttcaccggtaatctctacaacgccttgcgcagcaagagaatcaacacctttttcaatgaaaatggtgataccaaagctcttgaagcaattaaagaatcgaggatttcgatcgttgtgctctccccaaactatgcatcctcctcatggaggcttgatgaacttgtgaccatccttgagtgtatgaaaaccaagggccaactggtgtggcccatctattacgaagtggaaccgtcggaagtaaggcgtcagagcggtatctatggcgaagccatgtctgaatttgaacaaaaatttggacatgagagtgagatggtgtggaaatggaggagtgctttgactgaagtcagcagcttgagtggatgggtttacgaaactgggtacgaatataaattcatccgaaagattgtgcgattagcagtggaatctttgccccgatatgatgttttcctgagttttagcggagaagatactcgctactctttcacaggttttctctataatgcctttaggcgggaggggttcaatatcttcatggatgatgagggattggagggtggcaaccaaatttcagaaactctcatgcgagcaattgaaatgtcaagactttcaattgtcgtcttctctgaaaactatgcatattccacgtggtgtcttgatgaacttgccaagatcatcgagtgcaagaagaccaagaatcagatggtttggccaatatttcactatgtggaaaagtctgatgtgtgcaatcaaacaaaaagttatggtgaagccatggctgcacatgaagaaagatttggaaaggactctgagaaggtgcaaaactggaggtctgctttgtctgaaattgccaacttggatggacaccatttcagagaaaatgagtaccaatatgaatttatcgaaagggttgtggatctggccattgctatcgggaatcagtag

SEQ ID NO: 48 - protein, 344 aa, Cajanus cajun
MLMANGATEEQRSSNKYDVFLSFAGIDNRYTFTGNLYNALRSKRINTFFNENGDTKALEAIKESRISIVVLSPNYASSSWRLDELVTILECMKTKGQLVWPIYYEVEPSEVRRQSGIYGEAMSEFEQKFGHESEMVWKWRSALTEVSSLSGWVYETGYEYKFIRKIVRLAVESLPRYDVFLSFSGEDTRYSFTGFLYNAFRREGFNIFMDDEGLEGGNQISETLMRAIEMSRLSIVVFSENYAYSTWCLDELAKIIECKKTKNQMVWPIFHYVEKSDVCNQTKSYGEAMAAHEERFGKDSEKVQNWRSALSEIANLDGHHFRENEYQYEFIERVVDLAIAIGNQ

SEQ ID NO: 49 - DNA, 495 bp, Glycine clandestina
  source 1..495: mol_type = genomic DNA
gatttggtagtagttcccagcccaagaatccaactctctcgtactgcaaattttgctccacctggatgatatgtgaaagtctgaaaccaaacacaccttctcaagacatttttttccaatgctatcgtggcgttgatggcgaaattcaatatgaaaccgcgttttgctgcaccgaaaattcaaagtcaaaccggcttttctagaatgataatgtattttattacacgtggtactagaaaaatgcatatgcattgctgtcaaccacaccaactttcaactttaaaaggcacttaccgtggaaaacgattctactttctagaactgtgacgtaatgctgattattttaagaattgcgtggaaggtgtgttattgaaacgttgaattcaacgcgtatgataattgtatggtattaggtagaagcaagttatgtgtctcttgagcaaaaaagaaagtgattagtttgtaaacttgctgagactgagaggaacagaagaa

SEQ ID NO: 50 - DNA, 1422 bp, Arabidopsis thaliana
  source 1..1422: mol_type = other DNA
ccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcag

SEQ ID NO: 51 - DNA, 762 bp, Phakopsora pachyrhizi
  source 1..762: mol_type = genomic DNA
atgatgaaatttttgaacattgtactattttcccctttatttttgatgctttcttctaattctagtccattactaagctcccggcaaagtattccgatttataatcaatgtcgagtcgagaatagcttttccttgacaattgatgatggattttcagcacttagcaaaagcttgacacaggtctttgatgataaccaagttcgagctacattctttattaatgggaacaatgctgcttgcatatactacaatgctgatgatttgaaagcaagacatagaagtggtcatcttcttgccaaccacggttggagtcatgcacacatgacacagctaagtcgtgcgaatatggttagagaagtggaaagagtagaacaggcatttattaagatccttgggcttaaacctctttacttccgtccaccttacggagaatacaacgatgttttgctatcggtactatctgaaagaggctacaagggacttataatgtggaatcaagactctggcgatagctctagtccccctactccagcggaagacattttagctgcttatgataagtatcccgcaaaatcaattgttcttaatcatgaaattactcagaaatctgtacaaaccgttttgccaactgttatcccaatattgaagaggaaattttcattgcaaacttcggctgaatgtttagggttaggaagcaaaacaagtgattattatcaatacgtatccgagcctggaactccagatagcacctgggtctgctaa

SEQ ID NO: 52 - DNA, 978 bp, Phakopsora pachyrhizi
  source 1..978: mol_type = genomic DNA
atgtctaaactttttctcattgctgctattgctgcattcgctccctcaaactcttatggaagctttaatatacctgctgcaccacaaggtagtttcggaaacatatctccattcaaggacaacgtcctcagcttgagctgtggactgaatcttttagttggtggaagagttgccccattagcacaatctggctacgcaaatttatgccttcagtgtaaaattgacctctttggtgttcgcgtgagctttgcctacaccaacgctctttcagaatctatcaacactcatggtttatcggttagcgacacctcccgcctccagggcttcgttcagggacggcttttagactctgctgctcaatcttcaaccagcaatgagtgctcgcagagatgctcagacgatcaattctgtcaatccagtattttcagtgatgggcgctgcacttttacagctaaaaacctagccagcaacaacaaaaacggcgccctcctagcccaagcatttacacaagtcttcaaccaaggaggaagtcagtattgcggattttgcagtggcgattcatcatgtgtcaaccctagcgctcgtgcacgaagcttcaagcgccatgcggaaattgcacggagaagtcaattgcaggcaaaatgtcccactggcctcactgcctgtccaattgctcaaatgaatcagacggcagctgaagtctggagtccatcaccagcagtgtcttatgagtgcttgaaggtagacgaggaattaagctcctgcggtggttgcgcaacgctaggcaaagggacagactgttcagctgttgagggtgttttgtctgctggctgctcagccggtaaatgctatactttctcttgtcaagacggatacaaactttcaaagatgacgaacaaatgcgcccttataaattctaaacctaatctgcaccaaaagtaccccgtagttcaatcaggaaaaatatttagtcgttga

SEQ ID NO: 53 - DNA, 660 bp, Phakopsora pachyrhizi
  source 1..660: mol_type = genomic DNA
atgcacatctctgtcttttcagttgttcttttggccttagcacaattcaccgtgggccaccctcttagtgaaagatctgaaaaaaaaatatcacatgaaaattcttctgagaaatgcatcggcaattgtggctctggcggcattggtcctggatttgggggaccaattaatggaggtcttggaaacactctagccggcacgagtgctcctaatcaagtgggaggaaactttgacagctcacttactggcacttctgcccctagtcaagtgggaggaaactttggcagctctatcaatggcgctaatacccctaatcaagtgggaagtaactttggcaactcactattgggtgcaaatggtcttaaccaagtaggaggaaactttggtgatacactcattggcgctaatgcccttaacagagtaggaggaaactttggtaacacgcttattggggctaacgcgttgaatggattaggtggtaatttcggagacacacttattggagttaatgcactaaacggcgcaggtggtaattttggcaattcactcgttggtgtcaatgctctcaatggagcgcctgggaacttcggtaattcgcttgtgggcatcaatgttctgaatggacttggagccttgaagggacgaaaggaggatgcataa

SEQ ID NO: 54 - DNA, 687 bp, Phakopsora pachyrhizi
  source 1..687: mol_type = genomic DNA
atgtggcacttattttttctagcagcttttttgctttcatccatttcgtgccaaactacatccacaagtttttctggtgttcaaggaacttactggggttcctcccagtgggctaacggtaattgcaaatttcaagattggccccagcctaatgggctacccactgccgcagtcgctggaaatctttattctggtgcaaagtactgtggtgcatgcatcgacgtaacttccaaagcgggaatcacaaagcggggaataattagtgataattgtcccggatgcccttcaaacgctttggatctttctcccgatttgtggaactctgtgacaaacaacgaaagtcctggtattgaaactttaaattggaaaatagttgattgtggctatacgagccctataagtttgattaccaaagacggcgtcacggtcagctggtttgcgatgcaagctgcaggccataatgagcctatttcatctttagaggttaagccagccggaggttctacttggctaaaagccagcagagaggagtataactatttcacattaggagatggccagaagagtatgaataatgctaagacggcatctatcagagtaacttgttccaatggtaaaacaatcactactgaatcggtcccgattgaccaaccgcaaaaggttacttcagcatctggaaattgttaa

SEQ ID NO: 55 - DNA, 450 bp, Phakopsora pachyrhizi
  source 1..450: mol_type = genomic DNA
atgcatacagtaatatggagcataaaaataattgactcgttctcaggtatggcttgtctggcgtccacagtcgccagcatcttaccagagcaaagcggccggcaaatcaacaactctcaaccttccagtgtggctcggcctataaagtgggaggcagtcacggcagcgccactcgttgagcgagctgaagttaaagctgcacaaggtgatactttggaaaagcgctttgggtgcattgacccactagttagcgttgatgtactaaaccgtggcaatggagcgagaggaagtcttctagacgtaaacatcttgaacggattcttgaaagatgcccaaaagaagtctcagttaacaccattttcaatcacacgccaagctataccgcaaacttacggtgagcatatccatcgaaccaattgtcatgcagaagcgtcatcagtattcatataa

SEQ ID NO: 56 - DNA, 732 bp, Soybean cyst nematode
  source 1..732: mol_type = genomic DNA
atggataggagattcactgtcttccttgtgatcgcattggttacttctatttatgaagtgcttagtaatggaaatttgaacgatggcgacgactcatttaaacaattcgatgaactcgaagaaaacccagcgcataaatattcaaaagaggcccagaaggggttcgaaatggaggaggaggaagtcacaatccgagaaccttcggggacgaaggaatcgttcaaattgcccatcaatatgccgccagtgaaattttctttttgtgtctcatgtggttatcgccaagcatatgaacagtttgcgcagattttacgcgaaaaatatcccggaattgacatccacggcgaaaactatcctcccggcattcttcgcacggtgggagcgcaggtcatcggaatggtcaaaattgcactgattgtgtgcgttgtgtccggaaggagtcctttccccaccttgggacttgaaaccccaactttcttccaatggatgctcagcaatcgtttgtctgccgcacttatgctttttttattttccaatgccatcgaagggatgctccaatccacaggagccttcgaaatttacattgaaagcgaacgaatttggtccaaattggaaagtggtcgcgttccgtcacctccggaattgttccaggccattgacagtcatttggcaattcgcaggggcggcgccggaaggtttggttcgtcttcctctttcggcatcgatgggtca

SEQ ID NO: 57 - DNA, 360 bp, Soybean cyst nematode
  source 1..360: mol_type = genomic DNA
atgaatcactttgggctgacttttctgttcgtcgccgtttctctgctggtaattgcgcccggctgcgatggcatgcaaattttcgtgaagacattgacgggcaaaacgatcactttggaggtggagagctcggacactgtggacaatgtgaaggagaagatccaagagaaggagggcattccgccggatcagcaacggctgatcttcgccggcaaacagctcgaggacggacgaacgttggccgactacaacatacagaaggagtccacgctccacttggtcctccgtctccggggcggaaatgggaagagaaacacgggtaagaacaagaaaagcaacaaaaagcttgatcagaattga

SEQ ID NO: 58 - DNA, 1614 bp, Soybean cyst nematode
  source 1..1614: mol_type = genomic DNA
atggccctctctgcccttctgcttcttctccccctgcttctcaatgttcaaaatatcccagatgagtcagtccaatcggatatgaaggctgtttattcggctatttcatcgccggaacaatggaagaactcacaaaattcattggcttcactcgaatcacaactgacagagccccaaagagcactggcaaaaatgcattgggaattggagaccatccaaaaggaaaagccggaggcaccgccacaattcgacttgggacttttctcggaagctttggaagtgatggtcgaaatggacgaagaagcgaaagaagtgaagctgaggaaggacaaactgatccgaatgggcaggaggagaggaagcaaaaaaaaattgaaggaaaagatgacggaggaggacacagtgccggaagtgagggtgaacgagaatggtaaggttgaagtgaaggacggcgccggaggggaaggacggttggaagtcagacgaacaaaggacgagaaaggcagagagcaggtggtgatcacccttatgaagaatggcggaacggagggaccagcggaaggaaccgcggagaagccacaagagaaggccaagacggaggaggaggtacagaaaaagaatgatgacaaaagtacacgacaagaacaggaggaagcgaagaagacggagcaggaaaacgccggaggggtgccaaaaactgactcggccaacagccacattccggtaatgccaatgcacaccattttgtcatccccgtctccaccggtggaggagaagggcaaagcgagtgcagaggaggcacaaaaaaaggaggaggacaaaaacacacgacgtggaggggaggaggcaaacaacacggagcagaacaaagtcggaggagtgccaaaaatgtcagttgacagtcctaagtcggtcgtgccaatgccaatgcacaccattttgtcctcaccagccccaccggtcgaggagcaggacaaggcaagggatgcgctcacagaaggagcaaatggaaggaaaaaggcgcaaaacaacgaagaaatgttgctggttgcgacggagaacaacggaagcatgagaaatacgaataatggaggaatatttgattttgtccgaaaatttatctccaatgtgtttggacgtaagaaaagggacacggnaagcggtgcacagagaaaatatgacgggggaacgaaggaaaattcgcagcattcaaaatttgattatgagcactgtagggaagtccgactcagagccgaagagcgaagacaaacagcagcaggaagagaaaaagaaggcggagatcaagcagtacctggagaagggagtggtcaacacgggtggaatcaaaaaggccgagaaactggcatatgcttggtactcggagcttctgtactggacaaccaagtggatcgaggcattagaaaatcgggtggcgggagtcaaacccgaattggcacagcaattcttattctccaaaacgggatcagctgcttaccaggagctgaaggaagaggtggacaaatgcgaggcgaagttggccaaactaaaggaatggatcggcgactctttcaagtgaaaatggaagaaacggctcttagaggtgtatttttctga

SEQ ID NO: 59 - DNA, 1102 bp, Pseudomonas syringae
  source 1..1102: mol_type = genomic DNA
atgatgaaaacagtcagcaatcactcgatacccagtacaaatctcgtcgtggatgcgggaacggaaacttcggcgcagaaatcccagccggtttgcagcgaaatccagcgtaacagcaagatcgaaaaagcagtcatcgaacacattgccgaccacccggcagcgaaaatgacaataagcgcgctggttgacacgttgacagacgtttttgtcagggctcatggggaggttaaggggtgggccgaaatcgtccaggcagtctctcgccctcatgacagtaatcgacacggcagtggagtgctcagcccgcgctttgatgtaatggggagtgttggttggaatgcggcagctatccgggccaccagtcgcgtcgggacgcttcgagagaaaggtacactgttcactaaccttatgctcagtaacaactttaaacatttgcttaaacgagtggttaacgatccagccttgcagcaaaagctcgacggtgggttagacctcaactatctgaaggcttgtgaaggcgatctttatgtcatgtcagggtgggctgcacgggctagcgaaagtcgtgaacaaattggcaaagcccggtatgaaacggcatcaaatcttagccagacgctgatcagtgcacgtgagttggcttttcatcgtcacaatccggttaatcatccgtctgcccaaacgaaagtgggcttcgataagggtttgcctgaggaatctgatctgcaggttctgagaggccatggcagcagtgtatggagtgtaaaaccgggcagcgatttcgcaaagcgtgctgaagtttctggaaagcctattatcgccggcccgtccggtaccgcttcgcgcatggtcgctgttgcgcgttttctggcaccggcttgtttgaaaagcctgggtattgagagtgagcagaacctgaaagagcttgtgcggtatgcctgctatgcctatttcggtcaggacagccaccattcgatgcttgaagtgaatcttggtgtcgcttcccatggaatgccggaacaatgggacgacacgctttataacgagcctttcagtaattcaattaaaggtcgcgggtttggtatagacaatctcgcgc

SEQ ID NO: 60 - DNA, 1170 bp, Pseudomonas syringae
  source 1..1170: mol_type = genomic DNA
atgatcaaaacagtcagcgataactcgatacctggtacgtatggcatcgcgtttacgagggtggatactgcggcgcagatatccaggccggttcccagtgatatccagcgcaacagcagtatcgaaaaggcagtcatcgaacacattgctgaccacccggcggcgaaggtggtaatgagtgcgttggtagaggcattgacaggtgttttcgtaaaggctcaaggtgaaatcaagggctgggcggaaatcgtccaggctgcttcgcgtccccatgacagcaaccgtaagggcactggtgtgctgagtccgcgtttcgatgtaatgggctgtgtgggttggaatgctgcaacgattcgggctacctgccgggttggcacactgcgtgaaaaaggcacgttgttcactaaccttatgttgagtgacaacttcaagcggatactcgagcgttcgatgaccgatcctgccttgcaacgcaagctggaaggcttggtggatctggactatctgaagaccacggatggaaatctgtacgcaatgtccggctgggcagcccgcgccagtgaatctcgtgaacagattggcaaggcggcatttgagtctgcttcaaacctcggctcggcacagatcagcgcacgggagttggcttttcatcgccataatccggttaatcatccgtcgaatcaggcaagagtgggtttcggcatatcgtccccagcgaataatgatctgcaagttctaagaggtcatggcagcagcgtatggaaggtgaagccaggcagcgattttgcccggctggccagtgcatcgggtaagccagtgatcgcaggtccatccggtaccgcgtcgcgcttcatggcggtagcacgctttatttcacccggctgcctgcgtgacttggggcttgatagtgagcaagcattcaaagagctggtccgctatgcgtgctacggttatttcggtcaggacgaccaccactcgatgctggaggtcaatctgggtattgctccccacggtctggatgagcagtgggacgataagctatacaccgagcctttcagtcacgtcatcatggggcgcggcttcagtgttgacaatgcagctcagcagcacatcgtagcgcgtgcgaccgacgagccagtcgagcattcagccgctgatcgggtaggctga

SEQ ID NO: 61 - protein, 379 aa, Pseudomonas syringae
MMKTVSNHSIPSTNLVVDAGTETSAQKSQPVCSEIQRNSKIEKAVIEHIADHPAAKMTISALVDTLTDVFVRAHGEVKGWAEIVQAVSRPHDSNRHGSGVLSPRFDVMGSVGWNAAAIRATSRVGTLREKGTLFTNLMLSNNFKHLLKRVVNDPALQQKLDGGLDLNYLKACEGDLYVMSGWAARASESREQIGKARYETASNLSQTLISARELAFHRHNPVNHPSAQTKVGFDKGLPEESDLQVLRGHGSSVWSVKPGSDFAKRAEVSGKPIIAGPSGTASRMVAVARFLAPACLKSLGIESEQNLKELVRYACYAYFGQDSHHSMLEVNLGVASHGMPEQWDDTLYNEPFSNSIKGRGFGIDNLAHRQVVRQAAQKS

SEQ ID NO: 62 - protein, 389 aa, Pseudomonas syringae
MIKTVSDNSIPGTYGIAFTRVDTAAQISRPVPSDIQRNSSIEKAVIEHIADHPAAKVVMSALVEALTGVFVKAQGEIKGWAEIVQAASRPHDSNRKGTGVLSPRFDVMGCVGWNAATIRATCRVGTLREKGTLFTNLMLSDNFKRILERSMTDPALQRKLEGLVDLDYLKTTDGNLYAMSGWAARASESREQIGKAAFESASNLGSAQISARELAFHRHNPVNHPSNQARVGFGISSPANNDLQVLRGHGSSVWKVKPGSDFARLASASGKPVIAGPSGTASRFMAVARFISPGCLRDLGLDSEQAFKELVRYACYGYFGQDDHHSMLEVNLGIAPHGLDEQWDDKLYTEPFSHVIMGRGFSVDNAAQQHIVARATDEPVEHSAADRVG

SEQ ID NO: 63 - DNA, 333 bp, Erysiphe pisi
  source 1..333: mol_type = genomic DNA
atgtttgctagcaggtcatcttttatgttgttattttacttgacttggagagctagaatgcagttttctattctttttatcacatttatacttcagttccgcagtgcctttgcttctcctactgatccatggcaattagtcggtgaagtaggcgcaacaccttttccggggcaaaacaatgtaaagggctacgaatgtggcaatgatttctttagtgattccaaggtagaagaggccctaactatgtaccttcaacgtataaatcaggctcatcatagccctctggaataccattctcaccaaggatatcctaatttttcagcagaatcataa

SEQ ID NO: 64 - DNA, 387 bp, Erysiphe pisi
  source 1..387: mol_type = genomic DNA
gtggtacacaagaacatatactgtgtttctgctaagtcgccatccaacacaacaagtgacattcagtcctcaaatatctcaataagtgatgttcagccgtcgaatattgattggattccgcacgacaaagccacagagcaagcctgtttgtcttacaagcggcgtaatgaaggcaagaatcattgggatagatgtccagattgtgagactgctatcgtaaatggagtactgcagtgtaaatcacagactgttcgtttaaacgacattctgttccaggactattgcaaaatttttggcgcagcttacaccgagccccgatcaaccgaaaaaaaaagtttatggagctctgtaaaacgacggtttaaaatgcttctatggtggcactaa

SEQ ID NO: 65 - protein, 110 aa, Erysiphe pisi
MFASRSSFMLLFYLTWRARMQFSILFITFILQFRSAFASPTDPWQLVGEVGATPFPGQNNVKGYECGNDFFSDSKVEEALTMYLQRINQAHHSPLEYHSHQGYPNFSAES

SEQ ID NO: 66 - protein, 148 aa, Erysiphe pisi
MYFSRKHILIAIFGYQLVSAVVHKNIYCVSAKSPSNTTSDIQSSNISISDVQPSNIDWIPHDKATEQACLSYKRRNEGKNHWDRCPDCETAIVNGVLQCKSQTVRLNDILFQDYCKIFGAAYTEPRSTEKKSLWSSVKRRFKMLLWWH

SEQ ID NO: 67 - DNA, 510 bp, Plasmopara halstedii
  source 1..510: mol_type = genomic DNA
atgcgtacatttttgatcctggtcttacttgcagctacacttgtagtgaatgatgccgtgttatccacgactgctgacaacgacacgcaagcaacgactttctcgttgcggacaaactctctggaggacgctcaaaaggaagaaagagtcggctcttcctcttcctcttcgtattcgaaactaacagctgtggaacggaagtggaaagctaatttgtcgagtacagctttagaggaacacggagagctgaacaaaaacaaccaagcttacatgtttccctttctctacactttaaaagaaaacggtgtaactcttgatgaagtgttgaaactagtagaccacaatttagacgccgtgaccgtaagcaatttacaaaagtaccaagaagtggttgaggaaacgaatcatggagtagttgtgtcgaccaataggtctcaactggccaaagcattcgcaaaaaccattaaagaagaacagaagcccttgtggaaaaaattatttttcaaataa

SEQ ID NO: 68 - DNA, 879 bp, Plasmopara halstedii
  source 1..879: mol_type = genomic DNA
atgggtcatacctttgtgttcgtgcatgggatagtggtagctctactatctattgtagctgctacaaatggcacaatacccatacagagtgtgccatttactgatgtgggtgtcatcaagaaggaagtgagtgaagcggtgccatttgacgtcagaagcgaagatcgagggcttcaaaaagcatggtggaagttgattcatgcgaatcccgagaaagtttataaaaagctaaagctccacataacagaacaagatattttcaagcaccccgagctggaatggtttgccaactatatgcaaagatattacgtgaatcaggactctgatctcttattggctaagatggtggcaaaatctcataaggatggttcgctgatgagatatatcgaggaagcaaaagtgcgatacaatactcgagacaagaccgatgagccggaggcggcaacttttattcgcaacgtagaaagaatcgaagctggtttgcatagatactgggaactccagcgctacacagtgaaggatttggtggatttttttattcaaagtggtgaattatccatggaaaatgtaaaacgagcatcacattttttatttagaaagaacttgttcacagttctgaaagcttatgcgattaaaacaaacgtggaggatccagaaaaggctgtagctttggctatagcggatcaagttggccttgataatatggctttgatgatccaagaggcagataatcccctcgcaatcaaaactttgacgatcttgtacgaagatttagagtcaaaaaagaatatgctatcagacgttttttatcgacttcaattgggcagctttgagcttgacaagaaacaatttttcaatgcccacaaatttttttcttga

SEQ ID NO: 69 - protein, 169 aa, Plasmopara halstedii
MRTFLILVLLAATLVVNDAVLSTTADNDTQATTFSLRTNSLEDAQKEERVGSSSSSSYSKLTAVERKWKANLSSTALEEHGELNKNNQAYMFPFLYTLKENGVTLDEVLKLVDHNLDAVTVSNLQKYQEVVEETNHGVVVSTNRSQLAKAFAKTIKEEQKPLWKKLFFK

SEQ ID NO: 70 - protein, 292 aa, Plasmopara halstedii
MGHTFVFVHGIVVALLSIVAATNGTIPIQSVPFTDVGVIKKEVSEAVPFDVRSEDRGLQKAWWKLIHANPEKVYKKLKLHITEQDIFKHPELEWFANYMQRYYVNQDSDLLLAKMVAKSHKDGSLMRYIEEAKVRYNTRDKTDEPEAATFIRNVERIEAGLHRYWELQRYTVKDLVDFFIQSGELSMENVKRASHFLFRKNLFTVLKAYAIKTNVEDPEKAVALAIADQVGLDNMALMIQEADNPLAIKTLTILYEDLESKKNMLSDVFYRLQLGSFELDKKQFFNAHKFFS

SEQ ID NO: 71 - DNA, 24566 bp, synthetic construct
  source 1..24566: mol_type = other DNA
aaccctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaaggtaccggcgcgccaccggtgagctcgggacccaatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgttttggaattaagacaacaataaaaggatatggacataatttcaattatttatgagaaacaagaactatcaacgggagcttttttggtttgaaaaaaaaaaacttttggatttctgtttattacaactattataaataagaacttaattattacaactattaaattaaattaagggtttagattaaatacttaattacaaatcttgtcattgataatattttttacaaattttgtgtattttcttttaaatggagatgatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaatttatgaaagagagaaagagaagtagggaggcattggccattaggaggagtcaacgagacaagcaagccaccttctgctacttctcatccatcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaatgagtgaaagtcggtcaagtatatgctgtggagctgtgtgactaattgattgtaacatcaataactgcccttgaattcagttgtgcctatcaatgttgtttttacatccagttcaattcctaaggtttattgtattccttctaaatcttgttcaattctcgaggtctgttgtgttttttctacatcttgtttcaatgttttgtgttgattagataaaaaaaacaagtggtactattttgttttgtcatattggcattaggtaaggcaccgtgaaacaaaatttaaacacatcgccttctactcacaaaaataaaaacagagtatacaaaatctatggtaaggttaaataagttttcatcattcatctatttcagaattccatttttaattctctcaatcttctttatccaattttagtctctattttttgaagtaagtttagtctcccacgttaaataaaaactatcaaatacttatatgtccaacaaaattacatgtcacaacacacatgatgtaatcatgtgacaccgatgtgggtgatactaacatgtttagtcacatggtatcatgttttctatgtctttagaaatttttgaaatttaaattttagttcctaaaaaattatcatctttcaattttagtcaatataaatttgttttatttaatttaagtcctttaaaaaaatttagtttttaatccttaattaatttttttaatcttagtatagttatgctttagtgatttgttggtcatgagttcgaatatgtaaaaagtttatttacatatgcaagagtaaggttgtgtacaatatctttttccttatttttgtataacaaggagcctatgaacaatgtgatacataatttttttttctagtttaggtatttctagaatatttaatctcttcattattcaccttggtatcacaaatatcatcacaagtatgttcaaaccattatttggttaggtcctacattttgtcccaattttggattggatttttcttcctacaccagctagtgttcccatacacccaacaataaaacgaaaaactgaaaatacccttccataaggttcatacatattggccaatccgtatcctttgttgtcgtcaagctcagtgccctcccagcctttggccaactcaccgacatcgtcggggtccccacctctaaccgcatggaagagaaggcccactccctcggcatccccctctctatcctcgaccacaatccccacctcgatctcgccatcgacagcatcgacgaggtcgatgaggcgatctgtaatccataagattttacgaatttgcaatccataagattcttacggatttttcaatttgtatgaaccttacggattgtcaatccgtatgaatcttatggaagaatattttttatttttcgttgtctttggtgggtgtatgaagaaatttgtctagtgcatgaaaaaaaatgcttatggatttctgttattggaggttcattttgagctatgactcttgcactttatttttattttgaatagaatatacaattaataatttctatatgttcaaagtaattggatccacatttagacaattatattttttttctctctatttggcgaaaatattgtgtaagtttgtaattaattagaatttaggttgaaactaattattttatttgatgtggttacttattattatatattttaatgatttcatcgtattattaattgcttgtgatacaaagttaattaatagttgtgatgtaaagttaattatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgatatttttttcttgtgacaattataattagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaaatgatagtgtcagcacatcatataatatcttatgttatctgcttaatagtttcattgcgatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaaaatggcttaaatcgatcaagccctcgctcaagtgtcaactagaaggcagaactgatcaattcaacagattacagatggttctatgagcagtcattagctcacaaattgatagtaatagtcgtaatgcttacaatcagcactgaggaattgatttggaagtctgaattttacttagaacattagcaatttctagaaaggtgaatatatatgtcctgagggccatggccagaggcctgtcatcttgaaattgtgacacacactctcgtttaagtcatatttactttgatttcccacatgtcatgcatatacaaataattgtatgagttagattaaagaagctttagcattaaacagattgaagacaattacccagttaagatgctgaatgatgtttttgttgagaatcaaccgacatagttattcgtgatgtatataatcgaatttcgtaagacacaccaaacatatccttgcatgataacttttgattttggggtgtatcccgccgggaagatattcaagattgtgcagtgtaacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcatagaagaactttaaaataaaagagtattgacgaaaataaagtgcgcaaaaaaaaagatacagaaaattaaaggagtaaaaagtttggagaagaaattgagattgaaaaaagatgaagatttaaagggaagtataacaaatgtaaaagaaaacgataaaatgaataagataaagtgctgaaattaaaatgtttgagagtattgtaataattcattgatccacaagtgtcacgatgagacaagaggatatgtgagtgtttacaagtttcaactcttattacaaatttttaaatcttcctcatcgataactttaatattcaatgtcaatcattaatgtcctttgtctttggtattcgtttttaatatattcttcaagaggtatttgtagatttttgttcctctaatggttaaactcaaatgctttatgaaattattgcttaaaaacatgttattttcccgtgtcaggtgacctctaaaaaagatcaagctcaatactgggctgattcttcaaagccatataaatttatttcagtgccggagattgcataagcctttaaaaattccagatttggaaagtcggtagaatccatgtgtactgctccctttaataaattaaagagtcatccttcagctttgcccagaactagatttgttgtgccaaaatgggagctctttaaggcgtgtttctcgcgcgagagaccttgatcaacaggcataagtttctctatatttttaggacctgccaggtatgtatatatactcaagtaaataaggatgcacttgtttctatatatagttttcaattcttaagtttttttacaagtagaattcatgataagttcggaccgctaattagctaagagacacttgtgtgattgagagaaacactaatcttgtgaggactgaagtttggtgattatttcttgtgatctgtcgacaaaaatatcaaatggggtttcttttacaaattatttacctaaatgaatctgttttgaaaatatttactccattgggtctatttttttattacaaagcgtctccctgaagggcgcgttccccgtgaaagtgacacgtggcaggacttgggacgtgccctgcgtacaggcgcgatagttagtgttgttacagcaggcgcatcgggtcgtgttggggaccaaggtacgacaggtcgcgctgggtgacccagacacgacccaattgggtcgcactttatttaatattttttatattttgtatattgtttttatttaatatatttttatattattttatttaatttttttatattttatataatagtttctatattaaataaattcttagcattatgtatgattttaaagtcataaataattttttatattgtttttatttactatattttttatattttatttaatatttatatattaaataaatccttcatattagaaaaaataaagaaaatattaaataaaatataaaatataaaaaagtaaaaaatattaaataaaataatataaaaaatattataaaaacaatataaaaaatataaaaatatttaataaaataataaaaaaaatattattttaaataaaattatttatgactttaaactctaaagttgaattttaaaaaaatataatttttttacgattttagtaaaaaaaaaatacaagccgcacaatacaagtcgccttctcaaacccttcctcacgacattctcggaccttatgacaccgtcaccaaaacaatgatccacgcgatattaggcgcgtgcaaatcactctaatccgaaactagtagacatgggaagcacgagctatacgcgagcgtttcaattgccgccacgaaagcagagaaggccagaaacggaaccacggtaaaatggtaagggtattttcgtaaacagaagaaaagagttgtagctataaataaaccctctaacccacggcgcactatttctcttcactccttcgttcactcttcttctcttgcggctagggttttagcgcagcttcttctaggttcgttctcttccgccgctctatggattttaaaccttcgaatcatgtttattccattgaattatgttgcttgcagtttatattttctgaatctgtagttgttgtcttcaatttatcctatgctttatagatcaatcttttgtgtgtgtagtacgtaatttttgttctttttgcttttcgttcaagttgttgggaataatcggggtatcatgttttgatattgtttgttttcttttttgactgcttaataatttttaagttggttttggttttggggttttatgtgcttgttatattcaaatctttgtgatccagatcttacaaaagttttgggtttaaggatgtttttggctgatgatgaatagatctataaactgttccttttaatcgattcaagcttaggattttactaggcttttgcgaataaatacgtgacagtaagctaattatgtcctttttttgtctcaatcatatctgtctgggtgtgccataatttgtgatatgtctatctggtagaatcttgtgttttatgctttacgatttggtatacctgtttttgaacttgttgtatgatgggtatttagatcaccctatcttttttatgcttctggaagttttatgtaaatgtcgaatatcttaatgttgttgaacttataatgttgtgttgatgtatgtatgatggttttgacaacttttttcactggttctgaaagttttatgtaaattgcaaatatgttaatgttgttgaacttattttttttccttcgatgttgttttgatgtatgtatgatggttttcaccgtagtttctatggctaatatcttaatgttgttgagcttatttttttccttatatgttgtgttgatgtattgtatgatggttttgacaacttttttagtttctttgcagatttaaggaaggatcaaaaatggctgctgctgctgcagctccatctccatcattctctaagaccttgtcctcttcatcctctaagtcctctactcttttgccaaggtctactttcccattcccacatcatccacataagactactccaccaccacttcatcttaccccaacccatattcactctcagagaagaaggttcaccatctctaacgttatctccaccacccaaaaggtttcagagactcaaaaggctgagactttcgtttctaggttcgctccagatgagccaagaaagggatctgatgttcttgttgaggctcttgaaagggaaggtgttactgatgttttcgcttatccaggtggcgcttctatggaaattcatcaagctcttaccaggtcctccatcattagaaacgttttgccaagacatgagcagggtggtgttttcgctgctgaaggatatgctagagctactggattccctggtgtgtgtattgctacttctggaccaggtgctaccaaccttgtttctggacttgctgatgctctccttgattctgttccaattgtggctattaccggacaagttgctagaaggatgattggaaccgatgctttccaagagactccaattgtggaagtgaccagatctatcaccaagcacaactaccttgtgatggatgttgaggatattccaagggttgtgagagaggcattcttcttggctagatctggtagaccaggaccagttcttattgatgtgccaaaggatattcagcagcagcttgttatcccagattgggatcaacctatgagacttccaggttacatgtctaggcttccaaagcttcctaacgagatgcttcttgagcagattgtgaggcttatttccgagtctaagaagccagttctctacgttggtggtggatgctctcaatcttctgaggaacttagacgtttcgttgagcttaccggaattccagttgcttctactcttatgggacttggagctttcccaactggtgatgaactttctctttccatgcttggaatgcatggaaccgtttatgctaactacgctgtggattcctccgatcttcttcttgctttcggtgttaggttcgatgatagagttaccggaaagcttgaggctttcgcttctagagctaagattgtgcacatcgatatcgattccgctgagattggaaagaacaagcagccacatgtgtccatttgcgctgatattaagcttgcacttcagggactcaactccattcttgaatccaaagagggaaagctcaagcttgatttctctgcttggaggcaagagcttactgttcagaaggttaagtaccccctcaacttcaagactttcggagatgctattccaccacagtacgctattcaagtgcttgatgagcttaccaacggctctgctattatttctactggtgttggacagcatcagatgtgggctgctcaatattacaagtacagaaagccaaggcagtggcttacttctggtggacttggtgctatgggattcggacttccagctgctattggagctgctgttggtagaccagatgaagttgttgtggatatcgatggtgatggctccttcattatgaacgttcaagagcttgccaccatcaaggttgaaaaccttccagtgaagatcatgctccttaacaaccagcatcttggaatggttgtgcaacttgaggacagattctacaaggctaacagggctcatacctatcttggaaacccatctaacgaggctgagattttcccaaacatgcttaagttcgctgaggcttgcggagttcctgctgctagagttactcatagagatgatctcagggctgctattcagaagatgcttgatactccaggaccataccttctcgatgttattgtgccacatcaagagcatgtgctcccaatgattccatctggtggtgctttcaaggatgttattactgagggtgatggaaggtcctcctactaagtgctagcattttgtacattgagtagggaagagagagagatatatataaatactcacaaagcagtgagtatatggcttgcttttgtttctaaacattcctttataaggacttgagataatttgtattgtttaaaagagccattggttctgttgtatcaaagtaatttaattttcagtaccgacttggctctactcatatgtggtggtgtacattgcatctttgcatttgattccatatctattgggatgggaccatacattttttgtttattatttaagtcaaacatatcagataatatgtggtgaccagaaaagtgtctagcaatcaaatgggtctgtgattgcatgtttgagttcaggacatttattaatgagtgacagaattttaaaaagctcgccaataggagttgccacatctgatcgagtcttctgtaaacactaactgtaacttgagtttgataatcagattccccccgttggtaggtaatatgtaaatttcactatagattcttactgaggatgatggtcttgacttcgaagtggtgagtgtaattctctaatctaaactttatatatatataaaaaaagaatgttaaaaacttgagtgacatacagcaaatagcaatgaaagcatgaccattggccttggggggccaaatggccatgtaccacttttccacctacttgagagccattggtcctgaatgaggtccttttgtttcattttttttacaataaagaaagttcatattcttggcaatagcaaggcaaatcatttgtggaagtcacatgaaattcaccatactaattagctaacggacccgatttaaatcggtaccactagtaatattcggaccccctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccaaggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccaaggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtattatttgccgactactttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaacgcttccctcatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgagggg

SEQ ID NO: 72 - DNA, 31358 bp, synthetic construct
  source 1..31358: mol_type = other DNA
vcaaccctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaaggtaccggcgcgccaccggtgagctcgggaccccacaattaatttatagtttaattattaaattaaaatttattattataaaatttattatgtaaaattatatataatattaaaaatttaatgttatatatcaatattaactatttttaatataattagtttattagtttaattggagtgttgtgttcaacctcacaagttctagacatgtatgaaacataaggcgaatcatatccataatcacaatagtatggagggaaaaactttatacacccagaaaaagactaatacaccattccagaacagtgtatttccaaaaataccctcacttttattacacttcatacatacggattcgtgaatctgtaagattcttacggatttgcgaatccataattttttttgagttatacggattggacaatccgtatgggccatacggaatctgcggggtcccaaatgaattggcttattcaaaaaaaaccctgttctttggaagatctatctcgtgtctagtactgcatggttccaccctgcaagaactctgaatcattctcttgaagctcatgctcttcatcataaatgatccgcttgtcccaaaatgacctggcccaatagggaaatcccaattcattgggcctttcaatacaatcaaatagaaagccccaagggcgccatattctaggagcccaaacaatgtgattgaaaaaatcctcctctatctgttgtgggtcgaggactccctccccttcttcaaactccgattcatatttttcatagagaaatctctgatcaacgatagaataagatccattttgaatcatatttaagggattccttggttcggaccgaagaaacaatgtcactcgatccttatcaaactcactgcaatctttttctgtacgtgaggatcccaccagagcgccttctacttctaataggccatgaactagatcagaattattctcaacgagtccataagatgtgatcccatttttttcatcaggtctgggtagagaccaaaggtcttgagcgaccgatctggcagaacaactcaaaaggtaaagaagtatcgttaatttattcatactcgttccaagttcgaagtaccatttgtatccgtatggcccatacggattgtccaatccgtaagtaagtttttttttaagttaaaatatatttttccaaataatttaaaacaatggcgcatgtaggtctcttattaacgtaacgctccaaccaactgagctaataggccaattatgctaaaaaccaattaatgttatacatgtaacactaaattttttaatgtatatttaatacacgtataagtttacataataaattttgtaacaattaattttgatttaataattaaagtattttttacatataaaaatcatagttttaaataaaaatcataattttaataaaaatttgcaggactaataaaaattatatttgtatttacaggttaaattaaaaattatatctgtatttatagtttattaaacttgcaaatttttattaaacttatgatttttatttaaatctatgatttttatatgtaaaaaatactttaattattatatcaaaattaattgttacaaaatttattatgtaaacttatacgtgtattaaatatatattaaaaaatttaatgttatatgtatcaacattaattagcttttagtataattgatctattagctcatttggttgaagcgtcgtgttaataacttgaaggttaaaggttcgaaacttacatgtgtcattatttggaaaaatattttttaacttaaaaaaaaaacttacttacgtaaggttcatacggattcacgaatccataagaatcttatgtattagtcttttgctgggtgtataaagtttttccctagtatggaggaaaataaaaatgtgtacaaaaataatggcaatggattaatcggaccagcccaatcattcatcactcggcccattacgtagctcatcgtcgttcacaaaaatgaagcttaaccgccaacaattcctgcactgcaaactctttctgccgcaacaacacgcaacacacaaaccataaacctagccccaactttccttttcatcttcaccttcaccttcgttttcaagctacagcaatgagaaggccgaaatcgaaggagtttcgcaagcaacagcgagtttcagaggaggaagagataagcctcttgaattcatggatccagtttcagccaccggactccggttcgaacccaatgtcgctgccgcgtctccccaaaaactcccccgttggccgcctcgaagataacacctactcgcgctacgccggcgcgtcacgctttgatcaatttccactctcgaagaaaaccaaggacgccctaagagagtccaagttcgtggccatgaccgacatccagagagcgtctcttccccacgcgctctgtggccgcgacatcctcggcgccgccaaaactggctccggtaaaaccctcgctttcattatccctgtaagtgcttcatttctcagctccattctcttcggattagggtcccctgtatttctttttatttaactaatacaaatattatgataataatataatcacaagattagctcaaagtgagaaaatatatagtaaatagaatatgatatattgaattgtgatgtttatgcttgttattatttgtttatcgtggtttctgaatgcaaatattatgataataatataatcataacattttactaataagtgtggaaaatcaacatcgaaagttcaaagccatgagaagctgatacgtgtttgggcaaggtcattgatttaatatgtagtgggtgaatagatggtatattgaattgtagtatttgtgcttattgttatttgtttatggtgaattttgaatgataggtgttggaaaagttgtatagagagagatggggacctgaagatggtgttggcagcatcatcatatcgcctacaagagaattggctggtcagttatttgatgtgttgaaggttgttggcaaacaccataattttagtgctggccttctcattggtggtcgaaaggacgttgacatggagaaagaacgtgtaaatgagctcaacatattgatttgcacgcccggtagactgctgcaacacatggatgaaactcccaattttgattgttcacagacgcaggttaatataactttttttcttatatgtgatagattaacctcaatctgactctgaaattagagttacaattaagttgtttgttttatatcactatctgtttcctcgtgtctgtttttaattgtacttgggtatgtcaagattttctatccttaacaaccttcagaaaaatttgcttccatgcctaggtgatttagcaacagtggttgaagcagagtataggtgccgtttaatttttatatgctttaggcccttgtaaagcataaattttttattttgtcttccaaagtacgtgtttatttgggtcaaaccagaaaacagatatgtatcattagatttgatcaatatagctgatggttgggtggacttcccggatataggttttggtactagatgaggctgatcggattctagacagtgggttcaagaaggaactaaatgcaataatttcccaactaccaaagcgtagacaaaccttgcttttctctgcaactcaaacaaagtcaattcaagatcttgcaagactaagtttgaaggatccagagtatctgagtgtgcacgaagaatctgtgacttccactcctactctgttgaagcaaattgtgatgattgttcccctggaaaataagttagatatgttgtggagtttcataaagacacatctacagtcgaaaacacttgtctttctttcaagctgtaagcaggtaactccaactccccttttctgtcctgtttcaagaaattctttttcttgttgaagggaattgtatgttataggtttctattacaattttactgtcatgccacttgaaaagttgcaccctttgatgcttagccaatgcatggctattggctagtcatcattttcaagattttttgtgcaattggagggaatgtagtttgattttatgattgaaacttgaaagttgtctcattagtttcatattcaatggccaagtaaataattgttttgttttatttgtagctacatgatgaagttcacatgcgtagaacttctgttatggttttcagttgcgctggttatggttattgtaatgagaaaatttttaaaggatttttaggatgaatttttccataattctttcatgtaaacagggaaaatgtttaagatgtggtttttggtgcaaattccctcttttgagcttgactatcttccataatgttcacatccctgcttaataggtaaaatttgtctttgaagcatttaagaaactgcaccctggcataccattgaagtgccttcacgggaggatgaaacaggaaagaagaatggcaatatattctgagttttgtgagaagcgctctgttctcttctcaactgatgtggccgcaagaggccttgattttaataaggcagttgactgggttgttcaggtgagtctcatcctgcttttggtttcctgtcttggagcaagttttagggctttgtggtggggtgttggaagcaaaaagcttgtttttatgttcagtctctcacttctctcttgcatttgtaggtggattgtcctgaaaatgtagcttcttacatacatagagttggtcgtactgctcgatataaatctgatggaaagtcagttttatttctgttgccttcagaaattcaaatgctggaaaagttaaaagcagcaaaggttccagtgcattttaataaggtacattttccctgcaaattaatagtatgcatgcaattccaattttcatttgaacatcaaggtgtcacttttaaatctgcttcacttcagtcttatgtttgtctgacatttgctttccatgtttttgcaattgttgattattcagccaaggcaagaactactgcaaccagtttctagtttgttggcatctttactggccaaatacccagacatgcagcatcgggctcagagggcattcatcacatatttgagatccatccatatccaaaaggataaagacatatttgatgttatgaaactacctattgacgagtattcagcatcattaggtttaccaatgacccccaaaatccgttttctaaaccagaaaattaaatctaaagatgtgacaactaaatcaactttagttgagccagaggattcagacaaggaaactatctttgaggtttccaggaagaagctagacacagttgctttcaaggatgaggaaaccgaaaatgatattcttcagttagcggatactgcaaatgaaggtgaaattaagtcatctgaaattgaagaaataatgtacgttcccatatacatttttacagtggtacagattaaaaaattaggctgctttctgataatgctgcattagtttttttatatatgattatggcaaggggctacactagtcttctggtttcaaaattgctgagacaaattgatatttgaatataacaccttatgatgtggctaatagaaaaaaaattcaaacttagtgttgggtataaagttggatctttcatatttcttggctggagccattcatctggagctttgaatttcttcctccattattattcttaccaatttagttctttcctatgcagaccagcaactcgtgtattgaagaaaaagaaactcaagattaatgtacacaggccacttgggactagggttgtatttgatgatgacggccacacacttcctccactagccaggatagctgacacacaaagtggcaaagaaatgctgctcgatccaggtattccatacttttcaactgcaaactgaagctgaaggtgaaattgttgcttgtacatgtgtatacgtgtcatttcgttccttgttttcatagttttgtgaaaataataaaaaatgttttaaaatgttaaattatagctacacacttgtttaccaattagaaaacccttttgtattttctgcaatcatcattgaatttgagaacctggctagatctcattctcatcagatcactatataccattttggtcttcagagcattgaagtagctaacttacatgtatgttcttacttcataagcatggactatttgtacattttgccacatcatctttatgtcttgatgcagaacaaaaagctgaatactatagaaggatgcgagatgatttgaagaaggctgacaaggaagacaagcttatagagcgtcagcggcttagagaaaaaagaatcaagcagaagttgaaatggaaggctgggaatgcggaagaggacgaccaagatgatatatctgggtcagaagtagatgagactgtagatagacggcataaaaagagtaaaatatactttgatagtgacagtgatgagggtgaaagaaatgaagtttcagggaatgcaggtataactaccggtgcggttacgttggaggagcaagaagctctggctttgaaattgttaaattccatgcactcataggatatgcgtaattcagctggacattttgtgacagcaaaaactggtgaaaagagagcaattcctgaaacattttaggagttctgttaacaaaaaattttgatctcccaatttttttctgtcatctctttcaagaaacgtatacatagaatcaatttactgcttttatttattttaactaaaaaaaatgggactaaaggacatccttgtgtgtaccaaacacggtattaaatattatttggagatattcataatagttgtacttcaaaatctgaatcgtttacaggaaaaaaactagcaccaaaatggtgtctttgccccctgttagttagagaccccaatgaagtctatagaaatgtcagatcaaatgttttgtggaaatgggccgagtttatggtctcgtacagtcgtacttattccgttgcattgtataggcgtggaagaaacttgagtgttctcccgttgcttagactgcgttttggaattaagacaacaataaaaggatatggacataatttcaattatttatgagaaacaagaactatcaacgggagcttttttggtttgaaaaaaaaaaacttttggatttctgtttattacaactattataaataagaacttaattattacaactattaaattaaattaagggtttagattaaatacttaattacaaatcttgtcattgataatattttttacaaattttgtgtattttcttttaaatggagatgatagtaatgacatgactggtttgttgataagaaaatggagaagaaaacaaaaactccatccatccatctggtccagtcaacccatgttcatgtattttatttcagcttgacattgaagtggggcgcagcaaggaaattaagcaaaaccgcgtagggcatcgtgttaatattttaaatttatgaaagagagaaagagaagtagggaggcattggccattaggaggagtcaacgagacaagcaagccaccttctgctacttctcatccatcatgtcgaatgaactcaaaaactacgatgtttttctcagttttcatggcgaagattcccgttacaccttcactggtactctctataatgctttgcgcagcaagagaatcaagacctttttcacagaacatgaatatgatcgtaaactacacactcatgacagccaaattccacactctactcttgaagcaatcaaggaatcaaggatttcgatggttgttttctcggaaaactatgcatcctcctcaagatgtcttgatgaacttgtggccatccttgagtgtaacaggacgataaaccaactggtgtggcccatcttttacggagtgtatccgtggcaagtaaggcaccagggcggtaactatggagcatctatgtctaaattagaagaaatatttagagattccaatgagagggtgaagcaatggagagcagctttatttgaagtcaccaacttgaatggatggtgttaccaaactgggtatgcttaattttagttgattaagttccattattaatctctacaactgctagcatagcatggcaccacttctttattacttattgtatctgtaatctgctgaacagtatgtctgatcttctaattatcgttttagaaagtcactttgttttgtttaaccgagagaaatacatgtgtgctgtaaattaaaggttctgtttccagtttcagtgtagcagtagcaatctgttctgtgcatacatatagattagtcaagcttaagtcaacttataattctgattaatagaaacaatatggttgtccatgctcgaatacccctggtgaattaaaaagacttgtgcttttataggaggtaccagtatgaatatgaattcatcgaaaggatcgtggaatcagccgtgcaagccttgcccggatatgatgtttttctgagttttaccggagaggatacccgctatactttcacaggttttctctataatgcctttcgccgagagggattcaaaatcttcatggacgatgaggaattggagagtgggaaccaaatatcacagaagcttatgggagcaattgaaagttcaaggatttcaattgttgtgctttctgaaaactatgcatattccacgtggtgtcttgatgaacttgccaagatcattgagtgtatgaagaccaacaatcaaatggtttggccaatattttacaatgtgcaaaagtcggatgtatgcaatcaaacaaaaagttatggtgaagccatgactgaacatgaaaaaagatttggaaaggactctgagaaggtgcagaaatggagttctgctttgtctgaaatcaacaatttggaaggagaccatgtcaaagaaaatgagtacgtaatgcttttaattagttacttaatatatgcttccctcttaagtgatattgaccatctatgaaatgctaaacatggcatagttcttgtccgagacatggggtattgtgtgttgttttgtttggagcagtttttgtattttgtacggcaatctgttagtgagaagattaattagtcgagcttaatattgttgttttattgacataaagtctgattaggagaaataaagtgaacacagcggatgaatgtcttaaaagaccttgcacttttgtaggtaccaacatgaatttatcgaaaggattgtacaaaaggtcattaaacttgaaggtagcaagcatatagcaaatcctttccttttatccaatgacagctacgaggaagaatgagtgaaagtcggtcaagtatatgctgtggagctgtgtgactaattgattgtaacatcaataactgcccttgaattcagttgtgcctatcaatgttgtttttacatccagttcaattcctaaggtttattgtattccttctaaatcttgttcaattctcgaggtctgttgtgttttttctacatcttgtttcaatgttttgtgttgattagataaaaaaaacaagtggtactattttgttttgtcatattggcattaggtaaggcaccgtgaaacaaaatttaaacacatcgccttctactcacaaaaataaaaacagagtatacaaaatctatggtaaggttaaataagttttcatcattcatctatttcagaattccatttttaattctctcaatcttctttatccaattttagtctctattttttgaagtaagtttagtctcccacgttaaataaaaactatcaaatacttatatgtccaacaaaattacatgtcacaacacacatgatgtaatcatgtgacaccgatgtgggtgatactaacatgtttagtcacatggtatcatgttttctatgtctttagaaatttttgaaatttaaattttagttcctaaaaaattatcatctttcaattttagtcaatataaatttgttttatttaatttaagtcctttaaaaaaatttagtttttaatccttaattaatttttttaatcttagtatagttatgctttagtgatttgttggtcatgagttcgaatatgtaaaaagtttatttacatatgcaagagtaaggttgtgtacaatatctttttccttatttttgtataacaaggagcctatgaacaatgtgatacataatttttttttctagtttaggtatttctagaatatttaatctcttcattattcaccttggtatcacaaatatcatcacaagtatgttcaaaccattatttggttaggtcctacattttgtcccaattttggattggatttttcttcctacaccagctagtgttcccatacacccaacaataaaacgaaaaactgaaaatacccttccataaggttcatacatattggccaatccgtatcctttgttgtcgtcaagctcagtgccctcccagcctttggccaactcaccgacatcgtcggggtccccacctctaaccgcatggaagagaaggcccactccctcggcatccccctctctatcctcgaccacaatccccacctcgatctcgccatcgacagcatcgacgaggtcgatgaggcgatctgtaatccataagattttacgaatttgcaatccataagattcttacggatttttcaatttgtatgaaccttacggattgtcaatccgtatgaatcttatggaagaatattttttatttttcgttgtctttggtgggtgtatgaagaaatttgtctagtgcatgaaaaaaaatgcttatggatttctgttattggaggttcattttgagctatgactcttgcactttatttttattttgaatagaatatacaattaataatttctatatgttcaaagtaattggatccacatttagacaattatattttttttctctctatttggcgaaaatattgtgtaagtttgtaattaattagaatttaggttgaaactaattattttatttgatgtggttacttattattatatattttaatgatttcatcgtattattaattgcttgtgatacaaagttaattaatagttgtgatgtaaagttaattatatttttgcatgcaattaactatatctaatagtaaataatttttagtcacagtaaaatcaactatatttatacatacaatataatttctgcattctgttataattgtatttttaggttgttatatataaatagttgaaaaagaatgataattttatatcatcattaatttatttataaattttattgtctattattaatctcataagagatataaataaaaaaagataattaatattaccttaaaataataataaaataattattttgatatttttttcttgtgacaattataattagattaaggaaataatattttttttcaagtggtgatgaaatgcacaaatttgagtgggacaactataattttcgaatagaaaatggagaagaaaacaaggactccatccatgtattttatttccaggaagcagcaaggaaattaagcaaaagcgcgtaaatgatagtgtcagcacatcatataatatcttatgttatctgcttaatagtttcattgcgatggcggatgaagggggaattgcgaagtggcgctacgatgtatttctgtgttttagaggggaagacacgcgccacaccttcacaggtaatctctatgctgctttgcggaaggcgagattgaggacattcttcgatgttgggttgaagggtggtgaccaaatctttgaagctattgttcaagcattggaagcatccaggatttcaatcgttgttctctccaaaagcttcgcgtcttcctcgtggtgccttgaagaacttgtcaagatccttgagttaagggcaacaaagaagcaactagttattccaatcttttaccacgtggatccctcccatgtaaggaaacagactggtagtttcggagagtacctgattagacatgaaagtcgcttgggaaaggactccgagaaaatacacaagtggaagtcagctttgtatgaagttgccaccttggccggatggtgtttcggagacggaagctgcaggtatggcgttttttcttcttcttaatttagctagtaccgcttcttaattttaaaaaaagaaaatgaaaataataattttttgtaggcattcaatactgccttacactttgagagaaaaaagaaacacagaacaacgtgacattaataagagagataatgaaaaatataggtgtaaatatgtttttaatagtttagtgttttttttttaatttttgtaatttttgttatttttagtctttataaattatatatatatatattttacttttagttttaaaattactttaaatagtactctagatagtaaaaaaaaaagtcctataagaaccaaaataaaaataaacattgtttgtaaggattaaaacaaaaaataattttataaaaacaaaaaaataaatattaaattataagaattaaaaagatatttaagttaaaaataaaatgtttcgatccaatgaatttttagcctaggatggtgtttatgtatcattgttgaaattaaaatttcatatactaagttaagtaactcatgccgattattattgctgaagtcatgtaaacttgcattttaatcctcaaagaccaaaaccacttgtgatatttgcacgaagcttttttccttgatttataccttattgaatattctaacttctgagttgtcaaatgactcgttaacatgggcttgagccagtttatgtctagaattgcaaggctttggccaatattcttaaagtgacaatctaacgtaactagttaattctcctttcattcttaaaagaaaaacaaaatagaaaccaacaataattatactctttgataaggaatttgttttatgaattatcacactagaatttattattggtaagaaacaatctaagcttttagctaatttatcaagttaattttactttgttttgacttacgagttttaaggtttgaaatctttggggagattaattattcaaactcaattttattatattaaaaaaaattcttttaagcactctttattaaaattataaaatttatagatctcactttttacttaataattttcattcataatttttaattaattgtaagaaatgtgcaagtgtgtgagagagtttcttgcttgaacccgtcttaaagtaatcaataatagataaagaaaaacgtaaccagtgcatgaatgaattaaaaggtcatgtacttttttgtaggtaccaatatgaatatgaattcatcgaaaacattgtgcgagaagccatcgccattgtaccccgctatagcatttttctgagttttagtggaaacgatacccgctcctttacaggttttctgaacaatgcattgtgccggagtaggtaccaaaccttcatgaatgatggggaccaaatttcacaatctactaatggagttattgaagaatcaaggctttcgatcattgtattttctgaaaactatgcacgttcctcgtcctgtcttgatgtgcttttgaccatccttcagtgtatggagacgaaaaaccaactgatttgtcccatcttttacaaagtgttaccgtcggatttaaggcatcaaagaaatagctatggtgaagccatgactgagcatgaaaatatgttgggtaaggactctgagagggtgaagaaatggaggtcagctttgtttgatgtcgccaacttgaaaggattttacttgaaaaccgggtacaacacatattattcttttactttttgcttattttcatgattcatgtctattatactagtaatagttgatgataataatcacttaatatgtgcatcgttcttgaaattctatgaaccgtttaatttgaaatccgttcatttaaatttgactttgttaacaaggaataacgttgtgagagacattggggtacggagttaaggctttgtttagagtttgcaattctgtttttgttgttgtgtggtaattacttttgtgcggatttactttgataattttagaaagaaaatgaaaattgtgtatgaatgaactaaaaggccttgtatttttgtaggtacgaatatgaatttattgacaaaatcgtggaaatggccagtaaaatttaaaatggcttaaatcgatcaagccctcgctcaagtgtcaactagaaggcagaactgatcaattcaacagattacagatggttctatgagcagtcattagctcacaaattgatagtaatagtcgtaatgcttacaatcagcactgaggaattgatttggaagtctgaattttacttagaacattagcaatttctagaaaggtgaatatatatgtcctgagggccatggccagaggcctgtcatcttgaaattgtgacacacactctcgtttaagtcatatttactttgatttcccacatgtcatgcatatacaaataattgtatgagttagattaaagaagctttagcattaaacagattgaagacaattacccagttaagatgctgaatgatgtttttgttgagaatcaaccgacatagttattcgtgatgtatataatcgaatttcgtaagacacaccaaacatatccttgcatgataacttttgattttggggtgtatcccgccgggaagatattcaagattgtgcagtgtaacaaatacccttaaaaaatgattaacgcattatgattttagttagttaatagtaaaacgattgctgcaagtttaaaaagatttgaaatagtttatagtattttgaccgtgaaattttgtgcttgttttagtcgacacaatcccatagatatttgagaaaaaggataaacattttctctgaaagtcttgtatcttcctcaataggtactgtaccataggttaatgttaaatttttaacatgagaacaaactaaaagtatgtaataaagagtaaagtgcaaattgatcatagaagaactttaaaataaaagagtattgacgaaaataaagtgcgcaaaaaaaaagatacagaaaattaaaggagtaaaaagtttggagaagaaattgagattgaaaaaagatgaagatttaaagggaagtataacaaatgtaaaagaaaacgataaaatgaataagataaagtgctgaaattaaaatgtttgagagtattgtaataattcattgatccacaagtgtcacgatgagacaagaggatatgtgagtgtttacaagtttcaactcttattacaaatttttaaatcttcctcatcgataactttaatattcaatgtcaatcattaatgtcctttgtctttggtattcgtttttaatatattcttcaagaggtatttgtagatttttgttcctctaatggttaaactcaaatgctttatgaaattattgcttaaaaacatgttattttcccgtgtcaggtgacctctaaaaaagatcaagctcaatactgggctgattcttcaaagccatataaatttatttcagtgccggagattgcataagcctttaaaaattccagatttggaaagtcggtagaatccatgtgtactgctccctttaataaattaaagagtcatccttcagctttgcccagaactagatttgttgtgccaaaatgggagctctttaaggcgtgtttctcgcgcgagagaccttgatcaacaggcataagtttctctatatttttaggacctgccaggtatgtatatatactcaagtaaataaggatgcacttgtttctatatatagttttcaattcttaagtttttttacaagtagaattcatgataagttcggaccgctaattagctaagagacacttgtgtgattgagagaaacactaatcttgtgaggactgaagtttggtgattatttcttgtgatctgtcgacaaaaatatcaaatggggtttcttttacaaattatttacctaaatgaatctgttttgaaaatatttactccattgggtctatttttttattacaaagcgtctccctgaagggcgcgttccccgtgaaagtgacacgtggcaggacttgggacgtgccctgcgtacaggcgcgatagttagtgttgttacagcaggcgcatcgggtcgtgttggggaccaaggtacgacaggtcgcgctgggtgacccagacacgacccaattgggtcgcactttatttaatattttttatattttgtatattgtttttatttaatatatttttatattattttatttaatttttttatattttatataatagtttctatattaaataaattcttagcattatgtatgattttaaagtcataaataattttttatattgtttttatttactatattttttatattttatttaatatttatatattaaataaatccttcatattagaaaaaataaagaaaatattaaataaaatataaaatataaaaaagtaaaaaatattaaataaaataatataaaaaatattataaaaacaatataaaaaatataaaaatatttaataaaataataaaaaaaatattattttaaataaaattatttatgactttaaactctaaagttgaattttaaaaaaatataatttttttacgattttagtaaaaaaaaaatacaagccgcacaatacaagtcgccttctcaaacccttcctcacgacattctcggaccttatgacaccgtcaccaaaacaatgatccacgcgatattaggcgcgtgcaaatcactctaatccgaaactagtagacatgggaagcacgagctatacgcgagcgtttcaattgccgccacgaaagcagagaaggccagaaacggaaccacggtaaaatggtaagggtattttcgtaaacagaagaaaagagttgtagctataaataaaccctctaacccacggcgcactatttctcttcactccttcgttcactcttcttctcttgcggctagggttttagcgcagcttcttctaggttcgttctcttccgccgctctatggattttaaaccttcgaatcatgtttattccattgaattatgttgcttgcagtttatattttctgaatctgtagttgttgtcttcaatttatcctatgctttatagatcaatcttttgtgtgtgtagtacgtaatttttgttctttttgcttttcgttcaagttgttgggaataatcggggtatcatgttttgatattgtttgttttcttttttgactgcttaataatttttaagttggttttggttttggggttttatgtgcttgttatattcaaatctttgtgatccagatcttacaaaagttttgggtttaaggatgtttttggctgatgatgaatagatctataaactgttccttttaatcgattcaagcttaggattttactaggcttttgcgaataaatacgtgacagtaagctaattatgtcctttttttgtctcaatcatatctgtctgggtgtgccataatttgtgatatgtctatctggtagaatcttgtgttttatgctttacgatttggtatacctgtttttgaacttgttgtatgatgggtatttagatcaccctatcttttttatgcttctggaagttttatgtaaatgtcgaatatcttaatgttgttgaacttataatgttgtgttgatgtatgtatgatggttttgacaacttttttcactggttctgaaagttttatgtaaattgcaaatatgttaatgttgttgaacttattttttttccttcgatgttgttttgatgtatgtatgatggttttcaccgtagtttctatggctaatatcttaatgttgttgagcttatttttttccttatatgttgtgttgatgtattgtatgatggttttgacaacttttttagtttctttgcagatttaaggaaggatcaaaaatggctgctgctgctgcagctccatctccatcattctctaagaccttgtcctcttcatcctctaagtcctctactcttttgccaaggtctactttcccattcccacatcatccacataagactactccaccaccacttcatcttaccccaacccatattcactctcagagaagaaggttcaccatctctaacgttatctccaccacccaaaaggtttcagagactcaaaaggctgagactttcgtttctaggttcgctccagatgagccaagaaagggatctgatgttcttgttgaggctcttgaaagggaaggtgttactgatgttttcgcttatccaggtggcgcttctatggaaattcatcaagctcttaccaggtcctccatcattagaaacgttttgccaagacatgagcagggtggtgttttcgctgctgaaggatatgctagagctactggattccctggtgtgtgtattgctacttctggaccaggtgctaccaaccttgtttctggacttgctgatgctctccttgattctgttccaattgtggctattaccggacaagttgctagaaggatgattggaaccgatgctttccaagagactccaattgtggaagtgaccagatctatcaccaagcacaactaccttgtgatggatgttgaggatattccaagggttgtgagagaggcattcttcttggctagatctggtagaccaggaccagttcttattgatgtgccaaaggatattcagcagcagcttgttatcccagattgggatcaacctatgagacttccaggttacatgtctaggcttccaaagcttcctaacgagatgcttcttgagcagattgtgaggcttatttccgagtctaagaagccagttctctacgttggtggtggatgctctcaatcttctgaggaacttagacgtttcgttgagcttaccggaattccagttgcttctactcttatgggacttggagctttcccaactggtgatgaactttctctttccatgcttggaatgcatggaaccgtttatgctaactacgctgtggattcctccgatcttcttcttgctttcggtgttaggttcgatgatagagttaccggaaagcttgaggctttcgcttctagagctaagattgtgcacatcgatatcgattccgctgagattggaaagaacaagcagccacatgtgtccatttgcgctgatattaagcttgcacttcagggactcaactccattcttgaatccaaagagggaaagctcaagcttgatttctctgcttggaggcaagagcttactgttcagaaggttaagtaccccctcaacttcaagactttcggagatgctattccaccacagtacgctattcaagtgcttgatgagcttaccaacggctctgctattatttctactggtgttggacagcatcagatgtgggctgctcaatattacaagtacagaaagccaaggcagtggcttacttctggtggacttggtgctatgggattcggacttccagctgctattggagctgctgttggtagaccagatgaagttgttgtggatatcgatggtgatggctccttcattatgaacgttcaagagcttgccaccatcaaggttgaaaaccttccagtgaagatcatgctccttaacaaccagcatcttggaatggttgtgcaacttgaggacagattctacaaggctaacagggctcatacctatcttggaaacccatctaacgaggctgagattttcccaaacatgcttaagttcgctgaggcttgcggagttcctgctgctagagttactcatagagatgatctcagggctgctattcagaagatgcttgatactccaggaccataccttctcgatgttattgtgccacatcaagagcatgtgctcccaatgattccatctggtggtgctttcaaggatgttattactgagggtgatggaaggtcctcctactaagtgctagcattttgtacattgagtagggaagagagagagatatatataaatactcacaaagcagtgagtatatggcttgcttttgtttctaaacattcctttataaggacttgagataatttgtattgtttaaaagagccattggttctgttgtatcaaagtaatttaattttcagtaccgacttggctctactcatatgtggtggtgtacattgcatctttgcatttgattccatatctattgggatgggaccatacattttttgtttattatttaagtcaaacatatcagataatatgtggtgaccagaaaagtgtctagcaatcaaatgggtctgtgattgcatgtttgagttcaggacatttattaatgagtgacagaattttaaaaagctcgccaataggagttgccacatctgatcgagtcttctgtaaacactaactgtaacttgagtttgataatcagattccccccgttggtaggtaatatgtaaatttcactatagattcttactgaggatgatggtcttgacttcgaagtggtgagtgtaattctctaatctaaactttatatatatataaaaaaagaatgttaaaaacttgagtgacatacagcaaatagcaatgaaagcatgaccattggccttggggggccaaatggccatgtaccacttttccacctacttgagagccattggtcctgaatgaggtccttttgtttcattttttttacaataaagaaagttcatattcttggcaatagcaaggcaaatcatttgtggaagtcacatgaaattcaccatactaattagctaacggacccgatttaaatcggtaccactagtaatattcggaccccctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccaaggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccaaggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtattatttgccgactactttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaacgcttccctcatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgagggg